(19) United States
(12) Patent Application Publication
(10) Pub. No.: US 2025/0312474 A1
(43) Pub. Date: Oct. 9, 2025

(54) ANTHRACYCLINS AND CONJUGATES THEREOF

(71) Applicant: SYNAFFIX B.V., Oss (NL)

(72) Inventors: Floris Louis VAN DELFT, Oss (NL); Sander Sebastiaan VAN BERKEL, Oss (NL); Jorin HOOGENBOOM, Oss (NL); Remon VAN GEEL, Oss (NL); Sorraya POPAL, Oss (NL); Mick Petrus Arnoldus VERHAGEN, Oss (NL); Marie NASSIET, Oss (NL); Veronique HENDRIKS, Oss (NL); Çagla KOÇ, Oss (NL)

(21) Appl. No.: 19/052,069

(22) Filed: Feb. 12, 2025

Related U.S. Application Data
(63) Continuation of application No. PCT/EP2023/072484, filed on Aug. 15, 2023.

(30) Foreign Application Priority Data
Aug. 15, 2022 (EP) 22190421.2
Apr. 14, 2023 (EP) 23168104.0

(51) Int. Cl. A61K 47/68; A61P 35/00

(52) U.S. Cl. CPC A61K 47/6809; A61K 47/6855; A61K 47/6889; A61P 35/00

(57) ABSTRACT

The invention concerns analogues of nemorubicin as well as PNU-159,682 with a range of substituents other than 2″-OMe on the morpholino ring that beneficially affected the toxicity of the toxin over the molecules with the 2″-OMe group. In addition, it was found that PNU variants with modified 2″-O-alkyl chain show enhanced tolerability in vivo. Thus, by modification of the 2″-O-alkyl group, ADCs were generated with carefully tailored potency and tolerability to improve the administered dose in patients. The invention thus concerns compounds according to structure (1) and conjugates therewith, as well as pharmaceutical compositions and methods of targeting tumour cells and treating cancer.

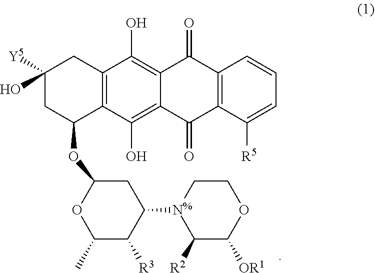

### CROSS-REFERENCE TO RELATED APPLICATIONS

This application is a continuation of International Patent Application No. PCT/EP2023/072484 filed Aug. 15, 2023, which claims priority to European Patent Application No. 22190421.2 filed Aug. 15, 2022, and European Patent Application No. 23168104.0 filed Apr. 14, 2023, the entire disclosures of which are incorporated by reference herein.

### REFERENCE TO SEQUENCE LISTING

The present application contains a Sequence Listing which has been filed electronically in XML format and is hereby incorporated by reference in its entirety. The XML copy created on Feb. 12, 2025, is named 06—Sequence listing.xml and is 10,054 bytes in size.

### FIELD OF THE INVENTION

The present invention is in the field of medicine. More specifically, the present invention relates to anthracyclines and antibody-drug conjugates prepared therewith, in particular to antibody-drug conjugates with analogues of PNU-159,682 as cytotoxic payload, suitable for the treatment of cancer.

### BACKGROUND

Antibody-drug conjugates (ADC), considered as one of the major classes of targeted therapy, are comprised of an antibody to which is attached a pharmaceutical agent. The antibodies (also known as ligands) can be small protein formats (scFv's, Fab fragments, DARPins, Affibodies, etc.) but are generally monoclonal antibodies (mAbs) which have been selected based on their high selectivity and affinity for a given antigen, their long circulating half-lives, and little to no immunogenicity. Thus, mAbs as protein ligands for a carefully selected biological receptor provide an ideal targeting platform for selective delivery of pharmaceutical drugs. For example, a monoclonal antibody known to bind selectively with a specific cancer-associated antigen can be used for delivery of a chemically conjugated cytotoxic agent to the tumour, via binding, internalization, intracellular processing and finally release of active catabolite. The cytotoxic agent may be small molecule toxin, a protein toxin or other formats, like oligonucleotides. As a result, the tumour cells can be selectively eradicated, while sparing normal cells which have not been targeted by the antibody. Similarly, chemical conjugation of an antibacterial drug (antibiotic) to an antibody can be applied for treatment of bacterial infections, while conjugates of anti-inflammatory drugs are under investigation for the treatment of autoimmune diseases and for example attachment of an oligonucleotide to an antibody is a potential promising approach for the treatment of neuromuscular diseases. Hence, the concept of targeted delivery of an active pharmaceutical drug to a specific cellular location of choice is a powerful approach for the treatment of a wide range of diseases, with many beneficial aspects versus systemic delivery of the same drug.

ADCs are prepared by conjugation of a linker-drug to a protein, a process known as bioconjugation. Many technologies are known for bioconjugation, as summarized in G. T. Hermanson, “Bioconjugate Techniques”, Elsevier, 3^{rd} Ed. 2013, incorporated by reference. Conceptually, the method the preparation of an ADC by bioconjugation entails the reaction of x number of reactive moieties F present on the antibody with a complementary reactive moiety Q present on the pharmaceutical drug (the payload), see FIG. 1.

Typically, a chemical linker is present between Q and the payload. This linker needs to possess a number of key attributes, including the requirement to be stable in plasma after drug administration for an extended period of time. A stable linker enables localization of the ADC to the projected site or cells in the body and prevents premature release of the payload in circulation, which would indiscriminately induce undesired biological response of all kinds, thereby lowering the therapeutic index of the ADC. Upon internalization, the ADC should be processed such that the payload is effectively released so it can bind to its target. The linker can also contain a spacer element. There are two families of linkers, non-cleavable and cleavable. Non-cleavable linkers consist of a chain of atoms between the antibody and the payload, which is fully stable under physiological conditions, irrespective of which organ or biological compartment the antibody-drug conjugate resides in. As a consequence, liberation of the payload from an ADC with a non-cleavable linker relies on the complete (lysosomal) degradation of the antibody after internalization of the ADC into a cell. As a result of this degradation, the payload will be released, still carrying the linker, as well as a peptide fragment and/or the amino acid from the antibody the linker was originally attached to. Cleavable linkers utilize an inherent property of a cell or a cellular compartment for selective release of the payload from the ADC, which generally leaves no trace of linker after processing. For cleavable linkers, there are three commonly used mechanisms: (1) susceptibility to specific enzymes, (2) pH-sensitivity, and (3) sensitivity to redox state of a cell (or its microenvironment). The cleavable linker may also contain a self-immolative unit, for example based on a para-aminobenzyl alcohol group and derivatives thereof. A linker may also contain an additional element, often referred to as spacer or stretcher unit, to connect the linker with a reactive group for reaction with the antibody.

The reactive moiety F can be naturally present in the antibody, for example the reactive moiety can be the side chain of lysine or cysteine, which can be employed for acylation (lysine side chain) or alkylation (cysteine side chain).

Acylation of the ε-amino group in a lysine side-chain is typically achieved by subjecting the protein to a reagent based on an activated ester or activated carbonate derivative, for example SMCC is applied for the manufacturing of Kadcyla®.

Various reagents are known for alkylation of the thiol group in cysteine side-chain, see FIG. 2. Amongst the cysteine alkylation strategies, the vast majority is based on the use of maleimide reagents, as is for example applied in the manufacturing of Adcetris®. Besides standard maleimide derivatives, a range of maleimide variants are also applied for more stable cysteine conjugation, as for example demonstrated by James Christie et al., J. Contr. Rel. 2015, 220, 660-670 and Lyon et al., Nat. Biotechnol. 2014, 32, 1059-1062, both incorporated by reference. Other approaches for cysteine alkylation involve for example nucleophilic substitution of haloacetamides (typically bromoacetamide or iodoacetamide), see for example Alley et al., Bioconj. Chem. 2008, 19, 759-765, incorporated by reference, or various approaches based on nucleophilic addition on unsaturated bonds, such as reaction with acrylate reagents, see for example Bernardim et al., Nat. Commun. 2016, 7, 13128 and Ariyasu et al., Bioconj. Chem. 2017, 28, 897-902, both incorporated by reference, reaction with phosphonamidates, see for example Kasper et al., Angew. Chem. Int. Ed. 2019, 58, 11625-11630, incorporated by reference, reaction with allenamides, see for example Abbas et al., Angew. Chem. Int. Ed. 2014, 53, 7491-7494, incorporated by reference, reaction with cyanoethynyl reagents, see for example Kolodych et al., Bioconj. Chem. 2015, 26, 197-200, incorporated by reference, reaction with vinylsulfones, see for example Gil de Montes et al., Chem. Sci. 2019, 10, 4515-4522, incorporated by reference, or reaction with vinylpyridines, see for example Seki et al, Chem. Sci., 2021, 12, 9060-9068 and https://iksuda.com/science/permalink/(accessed Jul. 26, 2020). An alternative approach to antibody conjugation without reengineering of antibody involves the reduction of interchain disulfide bridges, followed addition of a payload attached to a cysteine cross-linking reagent, such as bis-sulfone reagents, see for example Balan et al., Bioconj. Chem. 2007, 18, 61-76 and Bryant et al., Mol. Pharmaceutics 2015, 12, 1872-1879, both incorporated by reference, mono- or bis-bromomaleimides, see for example Smith et al., J. Am. Chem. Soc. 2010, 132, 1960-1965 and Schumacher et al., Org. Biomol. Chem. 2014, 37, 7261-7269, both incorporated by reference, bis-maleimide reagents, see for example WO2014114207, bis(phenylthio) maleimides, see for example Schumacher et al., Org. Biomol. Chem. 2014, 37, 7261-7269 and Aubrey et al., Bioconj. Chem. 2018, 29, 3516-3521, both incorporated by reference, bis-bromopyridazinediones, see for example Robinson et al., RSC Advances 2017, 7, 9073-9077, incorporated by reference, bis(halomethyl)benzenes, see for example Ramos-Tomillero et al., Bioconj. Chem. 2018, 29, 1199-1208, incorporated by reference or other bis(halomethyl)aromatics, see for example WO2013173391. Typically, ADCs prepared by cross-linking of cysteines have a drug-to-antibody loading of ˜4 (DAR4). Another useful technology for conjugation to a cysteine side chain is by means of disulfide bonds, a bioactivatable connection that has been utilized for reversibly connecting protein toxins, chemotherapeutic drugs, and probes to carrier molecules (see for example Pillow et al., Chem. Sci. 2017, 8, 366-370, incorporated by reference).

Besides conjugation to the side chains of the naturally present amino acids lysine or cysteine, a range of other conjugation technologies has been explored based on a two-stage strategy involving (a) introduction on a novel reactive group F, followed by (b) reaction with another complementary reactive group Q. For example, a method can be used to introduce a given number of reactive moieties F onto an antibody, which can be two, four or eight, see FIG. 3.

An example of an unnatural reactive functionality F that can be employed for bioconjugation of linker-drugs is the oxime group, suitable for oxime ligation or the azido group, suitable for click chemistry conjugation. The oxime or the azide can be installed in the antibody by genetic encoding of a non-natural amino acid, e.g. p-acetophenylalanine suitable for oxime ligation, or p-azidomethylphenylalanine or p-azidophenylalanine suitable for click chemistry conjugation, as for example demonstrated by Axup et al. Proc. Nat. Acad. Sci. 2012, 109, 16101-16106, incorporated by reference. Similarly, Zimmerman et al., Bioconj. Chem. 2014, 25, 351-361, incorporated by reference have employed a cell-free protein synthesis method to introduce azidomethylphenylalanine (AzPhe) into monoclonal antibodies for conversion into ADCs by means of metal-free click chemistry. Also, it has also be shown by Nairn et al., Bioconj. Chem. 2012, 23, 2087-2097, incorporated by reference, that a methionine analogue like azidohomoalanine (Aha) can be introduced into protein by means of auxotrophic bacteria and further converted into protein conjugates by means of click chemistry. Finally, genetic encoding of aliphatic azides in recombinant proteins using a pyrrolysyl-tRNA synthetase/tRNA_{CUA} pair was shown by Nguyen et al., J. Am. Chem. Soc. 2009, 131, 8720-8721, incorporated by reference, and labelling was achieved by click chemistry, either by copper-catalyzed alkyne-azide cycloaddition (CuAAC) or strain-promoted alkyne-azide cycloaddition (SPAAC). Besides, CuAAC and SPAAC, bioconjugation of linker-drugs to antibodies (and other biomolecules such as glycans, nucleic acids) can be achieved by a range of other metal-free click chemistries, see e.g. Nguyen and Prescher, Nature Rev. Chem. 2020, 4, 476-489, incorporated by reference. For example, oxidation of a specific tyrosine in a protein can give an ortho-quinone, which readily undergoes cycloaddition with strained alkenes (e.g. TCO) or strained alkynes, see e.g. Bruins et al., Chem. Eur. J. 2017, 24, 4749-4756, incorporated by reference. Besides cyclooctyne, certain cycloheptynes are also suitable for metal-free click chemistry, as reported by Wetering et al. Chem. Sci. 2020, 11, 9011-9016, incorporated by reference. A tetrazine moiety can also be introduced into a protein or a glycan by various means, for example by genetic encoding or chemical acylation, and may also undergo cycloaddition with cyclic alkenes and alkynes. A list of pairs of functional groups F and Q for metal-free click chemistry is provided in FIG. 4.

In a SPAAC bioconjugation, the linker-drug is functionalized with a cyclic alkyne and the cycloaddition with azido-modified antibody is driven by relief of ring-strain. Conversely, the linker-drug can be functionalized with azide and the antibody with cyclic alkyne. Various strained alkynes suitable for metal-free click chemistry are indicated in FIG. 5.

A method of increasing popularity in the field of ADCs is based on enzymatic installation of a non-natural functionality F. For example, Lhospice et al., Mol. Pharmaceut. 2015, 12, 1863-1871, incorporated by reference, employ the bacterial enzyme transglutaminase (BTG or TGase) for installation of an azide moiety onto an antibody. A genetic method based on C-terminal TGase-mediated azide introduction followed by conversion in ADC with metal-free click chemistry was reported by Cheng et al., Mol. Cancer Therap. 2018, 17, 2665-2675, incorporated by reference.

It has been shown in WO2014065661, by van Geel et al., Bioconj. Chem. 2015, 26, 2233-2242, Verkade et al., Antibodies 2018, 7, 12, and Wijdeven at al. MAbs 2022, 14, 2078466, all incorporated by reference, that enzymatic remodelling of the native antibody glycan at N297 enables introduction of an azido-modified sugar, suitable for attachment of cytotoxic payload using metal-free click chemistry, see FIG. 6. Similarly, the enzymatic glycan remodelling protocol can also be employed to install a free thiol group on an antibody (see FIG. 7) for conjugation based on any of the methods described above for cysteine conjugation.

Although ADCs have demonstrated clinical and preclinical activity, it has been unclear what factors determine such potency in addition to antigen expression on targeted tumour cells. For example, drug: antibody ratio (DAR), ADC-binding affinity, potency of the payload, receptor expression level, internalization rate, trafficking, multiple drug resistance (MDR) status, and other factors have all been implicated to influence the outcome of ADC treatment in vitro. In addition to the direct killing of antigen-positive tumour cells, ADCs also have the capacity to kill adjacent antigen-negative tumour cells: the so-called “bystander killing” effect, as originally reported by Sahin et al, Cancer Res. 1990, 50, 6944-6948, incorporated by reference, and for example studied by Li et al, Cancer Res. 2016, 76, 2710-2719, incorporated by reference. Generally spoken, cytotoxic payloads that are neutral will show bystander killing whereas ionic (charged) payloads do not, as a consequence of the fact that ionic species do not readily pass a cellular membrane by passive diffusion. Payloads with established bystander effect are for example MMAE and DXd. Examples of payloads that do not show bystander killing are MMAF or the active catabolite of Kadcyla® (lysine-MCC-DM1).

Currently, cytotoxic payloads include for example microtubule-disrupting agents [e.g. auristatins such as monomethyl auristatin E (MMAE) and monomethyl auristatin F (MMAF), maytansinoids, such as DM1 and DM4, tubulysins], DNA-damaging agents [e.g., calicheamicin, pyrrolobenzodiazepine (PBD) dimers, indolinobenzodiapine dimers, duocarmycins, anthracyclines, topoisomerase inhibitors [e.g. DXd, exatecan, SN-38] or RNA polymerase II inhibitors [e.g. amanitin]. ADCs that have reached market approval include for example payloads MMAE, MMAF, DM1, calicheamicin, SN-38, DXd and PBD dimer, while various pivotal trials are running for ADCs based on duocarmycin or DM4. A larger variety of payloads is still under clinical evaluation or has been in clinical trials in the past, e.g. eribulin, indolinobenzodiazepine dimer, PNU-159,682, amanitin, hemi-asterlin, doxorubicin, vinca alkaloids and others. Finally, various ADCs in late-stage preclinical stage are conjugated to novel payloads for example, KSP inhibitors, MMAD, cryptophycins, and others.

With the exception of sacituzumab govetican (Trodelvy®), all of the clinical and marketed ADCs contain cytotoxic drugs that are not suitable as stand-alone drug. Trodelvy® is the exception because it features SN-38 as cytotoxic payload, which is also the active catabolite of irinotecan (an SN-38 prodrug). Several other payloads now used in clinical ADCs have been initially evaluated for chemotherapy as free drug, for example calicheamicin, PBD dimers and eribulin. but have failed because the extremely high potency of the cytotoxin (picomolar to low nanomolar IC₅₀ values) versus the typically low micromolar potency of standard chemotherapy drugs, such as paclitaxel and doxorubicin.

Another cytotoxin that is receiving increasing interest for application in ADCs is PNU-159,682 (see FIG. 9), an anthracycline derivative >1000× more potent than doxorubicin. PNU-159,682 is one of the oxidative catabolites of nemorubicin (MMDX), which was developed as a synthetic derivative analogue of doxorubicin, however without the cardiotoxicity associated with the latter. PNU-159,682 is a bioactivation product formed from nemorubicin in the human liver under the action of CYP3A, formed after oral administration. Interestingly, two other oxidative catabolites, nemorubicin N-oxide and PNU-159,696 are of similar potency as doxorubicin. Due to its high potency, PNU-159,682 is under active investigation as a payload for ADCs, as for example reported by Dal Corso et al., J. Contr. Rel. 2017, 264, 211-218, incorporated by reference, whom reported that a non-internalizing antibody-drug conjugate, based on an antibody specific for tenascin C, mediates a potent therapeutic activity when equipped with PNU-159,682, connected to the antibody via maleimide-based cysteine alkylation and protease-sensitive cleavable linker based on Val-Cit-PABC and a dimethylethylenediamine (DMEDA) cyclization-cleavage element connected to the PNU-159,682 free 14-hydroxyl via a carbamate group (FIG. 9 top). The ADC was found to be stable in serum but could be efficiently cleaved in the subendothelial extracellular matrix by proteases released by the dying tumour cells, resulting in good tumour regression in various in vivo models. A similar PNU-159,682 ADC based on 14-OH acylation with Val-Cit-PABC-DMEDA was reported by Stefan et al., Mol. Cancer Ther. 2017, 16, 879-892, incorporated by reference, whereby the linker-drug was attached to the C-terminus of various antibodies using sortase-mediated antibody conjugation (SMAC™) to anti-HER2 antibody trastuzumab and the anti-CD30 antibody bretuximab (see FIG. 10). In this study, the DMEDA-conjugated ADC was compared head-to-head with another PNU-159,682 derivative prepared by oxidation of the hydroxy-ketone group to a carboxylic acid, followed by amidation with a diglycyl-ethylenediamine (EDA) linker (FIG. 9 bottom). Characterization of the resulting ADCs showed that they exhibited potencies exceeding those of ADCs based on conventional tubulin-targeting payloads, such as Kadcyla® and Adcetris® based on the same antibodies. However, in the same report it was also shown that the cytotoxic selectivity of PNU-derived ADCs based on the EDA-amide linker were much more selective for target-positive cells than analogous ADCs based on the DMEDA-carbamate linker, likely due to a specific release of PNU-159,682 from the latter ADCs. As a result, the EDA-amide-based technology was selected for further development and has been applied in various clinical programs today, including NBE-002 and SO-N102, for ADCs targeting ROR1 and Claudin18.2, respectively.

A similar method for the generation of ADCs based on oxidation of the hydroxy-ketone of PNU-159,682 followed by coupling of the resulting acid has been disclosed, see WO2016127081. Various derivatives of PNU include amide, hydrazide and acyl hydroxylamine derivatives.

Besides modification and covalent attachment of PNU-159,682 via the hydroxyketone moiety, surprisingly few reports detail the use of the methoxy-morpholino group for attachment to antibody. WO2009099741 shows how PNU-159,682 can be conjugated to an antibody with engineered cysteine via the hydroxy-ketone moiety, and suggestions are made to prepare conjugates by attachment at various positions in the morpholino group, including substitution of the 2″-OMe with a carbamate linker, however none of the morpholine-linked structures were enabled.

Details on the tolerability of NBE-002 in cynomolgus monkeys have been disclosed (AACR2018, abstract #737), indicating that the MTD lies around 3 mg/kg with a dosing schedule of qw3×3, although it was noted that one of the monkeys showed an immune reaction after the 3rd dose. With the phase 1 study currently ongoing (clinical trial NCT04441099), it remains to be seen what the MTD in humans will be. Given the ultra-high potency of PNU in preclinical models (MED as low as 0.033 mg/kg) it is not unlikely that the MTD in human will lie (substantially) below 1 mg/kg. As a consequence, in vivo receptor saturation is likely not to be reached after administration (typically intravenously), leading to suboptimal tumour uptake and enhanced clearance of the ADC.

One approach to raise the administered dose of the ADC in patients is by lowering the drug loading of the antibody for example a DAR1 format with the same payload could be preferable, as the MTD versus the similar DAR2 version will likely be two times higher. Ruddle et al., ChemMedChem 2019, 14, 1185-1195 have recently shown that DAR1 conjugates can be prepared from antibody Fab fragments. The resulting DAR1-type Fab fragments were shown to be highly homogeneous, stable in serum and show excellent cytotoxicity. In a follow-up publication, White et al., MAbs 2019, 11, 500-515, and also in WO2019034764, incorporated by reference, it was shown that DAR1 conjugates can also be prepared from full IgG antibodies using Flexmab technology. It was shown that the Flexmab-derived DAR1 ADCs was highly resistant to payload loss in serum and exhibited potent antitumor activity in a HER2-positive gastric carcinoma xenograft model. Moreover, this ADC was tolerated in rats at twice the dose compared to a site-specific DAR2 ADC prepared using a single maleimide-containing PBD dimer.

While having a DAR1 format of an ADC could be advantageous, no DAR1 technology has so far been reported that improves the therapeutic index versus DAR2 ADCs. Also, no technology has been reported for the generation by DAR1 ADCs from full antibodies without requiring reengineering of the monoclonal antibody, which renders the generation of DAR2 ADCs inherently more facile.

Another approach to raise the level of administered dose of ADCs, and in particular a PNU-based ADC would involve the generation of analogues with reduced potency. For example, Holte et al., Bioorg. Med. Chem. Lett. 2020, 30, 127640, incorporated by reference, have generated a range of PNU analogues with a broad range of cytotoxic activities by oxidation—modification of the hydroxyketone part of the molecule. Structure-activity relationships were explored which led to six linker-drugs being developed for conjugation to antibodies, leading to ADCs showing an increased MED of ˜1 mg/kg in various preclinical models compared to conventional PNU-159,682.

A final approach to modulate PNU-159,682 potency entails modification of the morpholino group, specifically the 2″-OMe group. As a single example, WO2012073217 reports the preparation and in vitro evaluation of a 2″-OEt analogue of PNU-159,682, which showed a 3-8 fold higher in vitro potency compared to the OMe variant in two different cell lines (A2780 and MCF7).

### SUMMARY OF THE INVENTION

The inventors have surprisingly found that analogues of nemorubicin as well as PNU-159,682 with a range of substituents other than 2″-OMe on the morpholino ring show distinctly lower in vitro potency than the molecules with the 2″-OMe group (i.e. where R1=Me). Similar reduction in potency was also observed for various 2″-O-alkyl derivatives of nemorubicin or PNU-159,682, covalently attached to a monoclonal antibody, in the form of an antibody-drug-conjugate (ADC), whereby covalent attachment could be ensured by carbamoylation of the hydroxyketone group or oxidation of the hydroxyketone group, followed by coupling of the resulting carboxylic acid. Moreover, it was found that by installation of a chemoselective handle in the 2″-O-alkyl chain, including but not limited to an amino, thiol or hydroxy group, covalent attachment to the antibody could also be achieved while leaving the hydroxyketone group (as in doxorubicin) or leaving the methylketone group (as in daunorubicin) intact. In addition, it was found that PNU variants with modified 2″-O-alkyl chain show enhanced tolerability in vivo. Thus, by modification of the 2″-O-alkyl group, ADCs were generated with carefully tailored potency and tolerability to improve the administered dose in patients.

The present invention concerns a novel toxin according to structure (1), and conjugates thereof according to structure (2). Related thereto, the invention concerns a process for preparing the conjugate according to the invention. In a further aspect, the invention concerns a method for targeting tumour cells. Related thereto are the first medical use of the conjugate according to the invention, as well as the second medical use for the treatment of cancer.

### DETAILED DESCRIPTION

### Definitions

The verb “to comprise”, and its conjugations, as used in this description and in the claims is used in its non-limiting sense to mean that items following the word are included, but items not specifically mentioned are not excluded.

In addition, reference to an element by the indefinite article “a” or “an” does not exclude the possibility that more than one of the element is present, unless the context clearly requires that there is one and only one of the elements. The indefinite article “a” or “an” thus usually means “at least one”.

A linker is herein defined as a moiety that connects (covalently links) two or more elements of a compound. A linker may comprise one or more spacer moieties. A spacer-moiety is herein defined as a moiety that spaces (i.e. provides distance between) and covalently links together two (or more) parts of a linker. The linker may be part of e.g. a linker-construct, a linker-conjugate, a linker-payload (e.g. linker-drug) or an antibody-conjugate, as defined below.

A “hydrophilic group” or “polar linker” is herein defined as any molecular structure containing one or more polar functional groups that imparts improved polarity, and therefore improved aqueous solubility, to the molecule it is attached to. Preferred hydrophilic groups are selected from a carboxylic acid group, an alcohol group, an ether group, a polyethylene glycol group, an amino group, an ammonium group, a sulfonate group, a phosphate group, an acyl sulfamide group or a carbamoyl sulfamide group. In addition to higher solubility other effects of the hydrophilic group include improved click conjugation efficiency, and, once incorporated into an antibody-drug conjugate: less aggregation, improved pharmacokinetics resulting in higher efficacy and in vivo tolerability.

The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt. The term “pharmaceutically accepted” salt means a salt that is acceptable for administration to a patient, such as a mammal (salts with counterions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “enediyne” or “enediyne antibiotic” or “enediyne-containing cytotoxin” refers to any cytotoxin characterized by the presence of a 3-ene-1,5-diyne structural feature as part of a cyclic molecule as known in the art and include neocarzinostatin (NCS), C-1027, kedarcidin (KED), maduropeptin (MDP), N1999A2, the sporolides (SPO), the cyanosporasides (CYA and CYN), and the fijiolides, calicheamicins (CAL), the esperamicins (ESP), dynemicin (DYN), namenamicin, shishijimicin, and uncialamycin (UCM).

The term “alkylaminosugar” as used herein means a tetrahydropyranyl moiety connected to an alcohol function via its 2-position, thereby forming an acetal function, and further substituted by (at least) one N-alkylamino group in position 3, 4 or 5. “N-alkylamino group” in this context refers to an amino group having one methyl, ethyl or 2-propyl group.

The term “click probe” refers to a functional moiety that is capable of undergoing a click reaction, i.e. two compatible click probes mutually undergo a click reaction such that they are covalently linked in the product. Compatible probes for click reactions are known in the art, and preferably include (cyclic) alkynes and azides. In the context of the present invention, click probe Q in the compound according to the invention is capable of reacting with click probe F on the (modified) protein, such that upon the occurrence of a click reaction, a conjugate is formed wherein the protein is conjugated to the compound according to the invention. Herein, F and Q are compatible click probes.

The term “(hetero)alkyl” refers to alkyl groups and heteroalkyl groups. Heteroalkyl groups are alkyl groups wherein one or more carbon units in the alkyl chain (e.g. CH₂, CH or C) are replaced by heteroatoms, such as O, S, S(O), S(O)₂ or NR⁴. In other words, the alkyl chain is interrupted with one ore more elements selected from O, S, S(O), S(O)₂ and NR⁴. Such interruptions are distinct from substituents, as they occur within the chain of an alkyl group, whereas substituents are pendant groups, monovalently attached to e.g. a carbon atom of an alkyl chain. In a preferred embodiment, the (hetero)alkyl group is an alkyl group, e.g. ethyl (Et), isopropyl (i-Pr), n-propyl (n-Pr), tert-butyl (t-Bu), isobutyl (i-Bu), n-butyl (n-Bu) or n-pentyl. (Hetero) alkyl groups may be linear, branched and cyclic.

Likewise, the term “(hetero)aryl” refers to aryl groups and heteroaryl groups. Heteroaryl groups are aryl groups wherein one or more carbon units in the ring (e.g. CH) are replaced by heteroatoms, such as O, S, N or NR⁴.

An “acylsulfamide moiety” is herein defined as a sulfamide moiety (H₂NSO₂NH₂) that is N-acylated or N-carbamoylated on one end of the molecule and N-alkylated (mono or bis) at the other end of the molecule. In the context of the present invention, especially in the examples, this group is also referred to as “HS”.

A “domain” may be any region of a protein, generally defined on the basis of sequence homologies and often related to a specific structural or functional entity. CEACAM family members are known to be composed of Ig-like domains. The term domain is used in this document to designate either individual Ig-like domains, such as “N-domain” or for groups of consecutive domains, such as “A3-B3 domain”.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene” means a DNA sequence that codes for, or corresponds to, a particular sequence of amino acids which comprises all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

The term “glycoprotein” is herein used in its normal scientific meaning and refers to a protein comprising one or more monosaccharide or oligosaccharide chains (“glycans”) covalently bonded to the protein. A glycan may be attached to a hydroxyl group on the protein (O-linked-glycan), e.g. to the hydroxyl group of serine, threonine, tyrosine, hydroxylysine or hydroxyproline, or to an amide function on the protein (N-glycoprotein), e.g. asparagine or arginine, or to a carbon on the protein (C-glycoprotein), e.g. tryptophan. A glycoprotein may comprise more than one glycan, may comprise a combination of one or more monosaccharide and one or more oligosaccharide glycans, and may comprise a combination of N-linked, O-linked and C-linked glycans. It is estimated that more than 50% of all proteins have some form of glycosylation and therefore qualify as glycoprotein. Examples of glycoproteins include PSMA (prostate-specific membrane antigen), CAL (candida antartica lipase), gp41, gp120, EPO (erythropoietin), antifreeze protein and antibodies.

The term “glycan” is herein used in its normal scientific meaning and refers to a monosaccharide or oligosaccharide chain that is linked to a protein. The term glycan thus refers to the carbohydrate-part of a glycoprotein. The glycan is attached to a protein via the C-1 carbon of one sugar, which may be without further substitution (monosaccharide) or may be further substituted at one or more of its hydroxyl groups (oligosaccharide). A naturally occurring glycan typically comprises 1 to about 10 saccharide moieties. However, when a longer saccharide chain is linked to a protein, said saccharide chain is herein also considered a glycan. A glycan of a glycoprotein may be a monosaccharide. Typically, a monosaccharide glycan of a glycoprotein consists of a single N-acetylglucosamine (GlcNAc), glucose (Glc), mannose (Man) or fucose (Fuc) covalently attached to the protein. A glycan may also be an oligosaccharide. An oligosaccharide chain of a glycoprotein may be linear or branched. In an oligosaccharide, the sugar that is directly attached to the protein is called the core sugar. In an oligosaccharide, a sugar that is not directly attached to the protein and is attached to at least two other sugars is called an internal sugar. In an oligosaccharide, a sugar that is not directly attached to the protein but to a single other sugar, i.e. carrying no further sugar substituents at one or more of its other hydroxyl groups, is called the terminal sugar. For the avoidance of doubt, there may exist multiple terminal sugars in an oligosaccharide of a glycoprotein, but only one core sugar. A glycan may be an O-linked glycan, an N-linked glycan or a C-linked glycan. In an O-linked glycan a monosaccharide or oligosaccharide glycan is bonded to an O-atom in an amino acid of the protein, typically via a hydroxyl group of serine (Ser) or threonine (Thr). In an N-linked glycan a monosaccharide or oligosaccharide glycan is bonded to the protein via an N-atom in an amino acid of the protein, typically via an amide nitrogen in the side chain of asparagine (Asn) or arginine (Arg). In a C-linked glycan a monosaccharide or oligosaccharide glycan is bonded to a C-atom in an amino acid of the protein, typically to a C-atom of tryptophan (Trp).

The term “antibody” (AB) is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multispecific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole antibodies, but also fragments of an antibody, for example an antibody Fab fragment, F(ab′)₂, Fv fragment or Fc fragment from a cleaved antibody, a scFv-Fc fragment, a minibody, a diabody or a scFv. Furthermore, the term includes genetically engineered antibodies and derivatives of an antibody. Antibodies, fragments of antibodies and genetically engineered antibodies may be obtained by methods that are known in the art.

An antibody may be a natural or conventional antibody in which two heavy chains are linked to each other by disulfide bonds and each heavy chain is linked to a light chain by a disulfide bond. There are two types of light chain, lambda (I) and kappa (k). The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties, such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G m3, non-G1m1 [that, is any allotype other than G1m1], G1m17, G2m23, G3m21, G3m28, G3m1.1, G3m5, G3m13, G3m14, G3m10, G3m15, G3m16, G3m6, G3m24, G3m26, G3m27, A2m1, A2m2, Km1, Km2 and Km3) of immunoglobulin molecule. Preferred allotypes for administration include a non-G1 ml allotype (nG1m1), such as G1m17,1, G1m3, G1m3.1, G1m3.2 or G1m3.1.2. More preferably, the allotype is selected from the group consisting of the G1m17.1 or G1m3 allotype. The antibody may be engineered in the Fc-domain to enhance or nihilate binding to Fc-gamma receptors, as summarized by Saunders et al. Front. Immunol. 2019, 10, doi: 10.3389/fimmu.2019.01296 and Ward et al., Mol. Immunol. 2015, 67, 131-141. For example, the combination of Leu234Ala and Leu235Ala (commonly called LALA mutations) eliminate FcγRIIa binding. Elimination of binding to Fc-gamma receptors can also be achieved by mutation of the N297 amino acid to any other amino acid except asparagine, by mutation of the T299 amino acid to any other amino acid except threonine or serine, or by enzymatic Deglycosylation or trimming of the fully glycosylated antibody with for example PNGase F or an endoglycosidase. The immunoglobulins can be derived from any species, including human, murine, or rabbit origin. Each chain contains distinct sequence domains.

A percentage of “sequence identity” may be determined by comparing the two sequences, optimally aligned over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. A sequence “at least 85% identical to a reference sequence” is a sequence having, on its entire length, 85%, or more, for instance 90%, 91%, 92%, 93%, 94%, 95%, 96%, 97%, 98% or 99% sequence identity with the entire length of the reference sequence.

The term “CDR” refers to complementarity-determining region: the specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. Complementarity Determining Regions or CDRs therefore refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region. “CDR”

The term “monoclonal antibody” or “mAb” as used herein refers to an antibody molecule of a single amino acid sequence, which is directed against a specific antigen, and is not to be construed as requiring production of the antibody by any particular method. A monoclonal antibody may be produced by a single clone of B cells or hybridoma, but may also be recombinant, i.e. produced by protein engineering.

The term “chimeric antibody” refers to an engineered antibody which, in its broadest sense, contains one or more regions from one antibody and one or more regions from one or more other antibodies. In an embodiment, a chimeric antibody comprises a VH domain and a VL domain of an antibody derived from a non-human animal, in association with a CH domain and a CL domain of another antibody, in an embodiment, a human antibody. As the non-human animal, any animal such as mouse, rat, hamster, rabbit or the like can be used. A chimeric antibody may also denote a multispecific antibody having specificity for at least two different antigens.

The term “humanised antibody” refers to an antibody which is wholly or partially of non-human origin and which has been modified to replace certain amino acids, for instance in the framework regions of the VH and VL domains, in order to avoid or minimize an immune response in humans. The constant domains of a humanized antibody are most of the time human CH and CL domains. “Fragments” of (conventional) antibodies comprise a portion of an intact antibody, in particular the antigen binding region or variable region of the intact antibody. Examples of antibody fragments include Fv, Fab, F(ab′)2, Fab′, dsFv, (dsFv)2, scFv, sc(Fv)2, diabodies, bispecific and multispecific antibodies formed from antibody fragments. A fragment of a conventional antibody may also be a single domain antibody, such as a heavy chain antibody or VHH.

### The Invention

In a first aspect, the invention concerns conjugates wherein a compound according to structure (1) is conjugated to a cell-binding agent via a linker, wherein structure (1) is as follows:

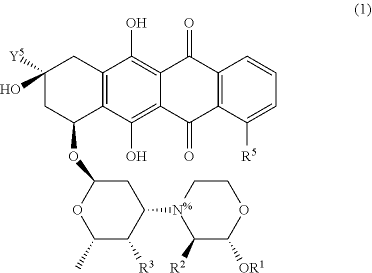

wherein:
R¹ is optionally substituted Et, i-Pr, n-Pr, t-Bu, i-Bu, n-Bu, n-pentyl, cyclopropyl, cyclobutyl, cyclopentyl, C₆₋₁₅ alkyl, C₂₋₁₅ alkenyl, C₂₋₁₅ alkynyl, heterocyclyl, (hetero)aryl, Sp-(hetero)aryl, Sp-heterocyclyl, Sp-X²R⁴, Sp-N₃, Sp-X²-Sp-R¹² or Sp-N(R⁴)₂, wherein the optional substituent is selected from halogen, C₁₋₁₂ (hetero)alkyl, (hetero)aryl, C₂₋₁₅ alkenyl, C₂₋₁₅ alkynyl, X²R⁴, N(R⁴)₂, NO₂, and wherein substituents C₁₋₁₂ (hetero)alkyl and (hetero)aryl may optionally be further substituted with C₁₋₆ (hetero)alkyl, X²R⁴ and N(R⁴)₂; wherein each Sp is individually C₁₋₁₂ (hetero)alkylene, (hetero)arylene, C₁₋₁₂ (hetero)alkylene-(hetero)arylene, or (hetero)arylene-C₁₋₁₂ (hetero)alkylene, wherein the (hetero)alkylene or the (hetero)arylene is optionally substituted with one or more substituents selected from halogen, X²R⁴, N(R⁴)₂, C₁₋₄ alkyl and NO₂, wherein each R⁴ is individually H, C₁₋₄ alkyl or adamantyl and X² is C(O), C(O)O, C(O)NH, O, S, S(O), S(O)₂, S(O)NH or S(O)₂NH, and wherein R¹² is β-glucuronide acid, PO₃⁽²⁻⁾, OPO₃⁽²⁻⁾, CO₂⁽⁻⁾, SO₃⁽⁻⁾ or N(C₁₋₄ alkyl)₃⁽⁺⁾;
R² is H, S(O)₂OH or P(O)₂OH and R³ is OH, or R² and R³ are fused together via an ether moiety to form an oxazolidine ring;
R⁵ is H or OCH₃;
Y⁵ is CH₂—Y, C(O)—Y, C(═N(R²⁰))—Y, C(R⁹)═N—Y, wherein R⁹ is selected from C₁₋₄ alkyl optionally substituted with an OH group or O(CO)C₁₋₆ alkyl, and R²⁰ is NR⁴—C(O)—N(R⁴)₂, NR⁴—C(O)—Sp—N(R⁴)₂, NR⁴—C(O)—R¹², NR⁴—C(O)—Sp—R¹², wherein Sp, R⁴ and R¹² are as defined above;
the compound according to structure (1) is connected to the cell-binding agent through Y. Also envisioned in this aspect are salts of the compound according to structure (1), wherein each ion if present is balanced with one or more pharmaceutically acceptable counter-ions.

In a second aspect, the invention concerns novel toxins according to structure (1):

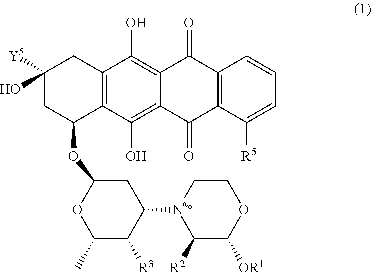

wherein:
   R¹ is optionally substituted Et, i-Pr, n-Pr, t-Bu, i-Bu, n-Bu, n-pentyl, cyclopropyl, cyclobutyl, cyclopentyl, C₆₋₁₅ alkyl, C₂₋₁₅ alkenyl, C₂₋₁₅ alkynyl, heterocyclyl, (hetero)aryl, Sp-(hetero)aryl, Sp-heterocyclyl, Sp—X²R⁴, Sp—N₃, Sp—X²—Sp—R¹² or Sp—N(R⁴)₂, wherein the optional substituent is selected from halogen, C₁₋₁₂ (hetero)alkyl, (hetero)aryl, C₂₋₁₅ alkenyl, C₂₋₁₅ alkynyl, X²R⁴, N(R⁴)₂, NO₂, and wherein substituents C₁₋₁₂ (hetero)alkyl and (hetero)aryl may optionally be further substituted with C₁₋₆ (hetero)alkyl, X²R⁴ and N(R⁴)₂; wherein each Sp is individually C₁₋₁₂ (hetero)alkylene, (hetero)arylene, C₁₋₁₂ (hetero)alkylene-(hetero)arylene, or (hetero)arylene-C₁₋₁₂ (hetero)alkylene, wherein the (hetero)alkylene or the (hetero)arylene is optionally substituted with one or more substituents selected from halogen, X²R⁴, N(R⁴)₂, C₁₋₄ alkyl and NO₂, wherein each R⁴ is individually H, C₁₋₄ alkyl or adamantyl and X² is C(O), C(O)O, C(O)NH, O, S, S(O), S(O)₂, S(O)NH or S(O)₂NH, and wherein R¹² is β-glucuronide acid, PO₃⁽²⁻⁾, OPO₃⁽²⁻⁾, CO₂⁽⁻⁾, SO₃⁽⁻⁾ or N(C₁₋₄ alkyl)₃⁽⁺⁾;
R² is H, S(O)₂OH or P(O)₂OH and R³ is OH, or R² and R³ are fused together via an ether moiety to form an oxazolidine ring;
R⁵ is H or OCH₃;
N % is N or N→O;
Y⁵ is CH₂—Y, C(O)—Y, C(═N(R²⁰))—Y, C(R⁹)═N—Y, C(R⁹)═N(R²⁰), wherein R⁹ is selected from C₁₋₄ alkyl optionally substituted with an OH group or O(CO)C₁₋₆ alkyl, and R²⁰ is NR⁴—C(O)—N(R⁴)₂, NR⁴—C(O)—Sp—N(R⁴)₂, NR⁴—C(O)—R¹², NR⁴—C(O)—Sp—R¹², wherein Sp, R⁴ and R¹² are as defined above;
Y is NR⁴—Sp³—N(R⁴)₂, NR⁴—Sp³—X²(R⁴), N(R⁴)₂, NR⁴—Sp³—X²(R⁴), R¹², Sp³R¹², NR⁴—Sp³—X²—Sp³—R¹², OH, CH₃ or CH₂OH, wherein Sp³ is a spacer;
and wherein R¹ is not unsubstituted ethyl, CH₂CH₂SH or benzyl when Y⁵ is C(O)—CH₂OH;

Also envisioned in this aspect are salts of the compound according to structure (1), wherein each ion if present is balanced with one or more pharmaceutically acceptable counter-ions.

Herein, the compound of structure (1) can be in conjugated form (i.e. conjugated to a cell-binding agent) or in free form (i.e. as small molecule). Unless stated otherwise, everything defined for the conjugate form of structure (1) applies to the free form of structure (1), and vice versa, except for the connection to the cell-binding agent via the linker.

Also contemplated within the present invention are salts, preferably pharmaceutically acceptable salts, of the antibody-conjugate according to structure (1). While the compound according to structure (1) in conjugated form and in free form can be in salt form, the conjugated form of the compound according to structure (1) is typically not in salt form, while the compound according to structure (1) in free form can be in salt form and in neutral form. If the compound of structure (1) is charged, it is typically balanced with one or more pharmaceutically acceptable counter-ions.

Here below, the compound according to structure (1) is first defined. The structural features of the compound according to structure (1) also apply to the conjugate according to structure (2) and the linker-toxin construct according to structure (5). Further, the structural features of the cell-binding agent according to structure (4) also apply to the conjugate according to structure (2). The skilled person understands that any structure feature that is unchanged in the conjugation reaction is defined equally for each of the molecules according to the invention. In the conjugation reaction, only reactive moieties F and Q are transformed into connecting group Z¹ upon reaction of the linker-toxin construct according to structure (5) with an antibody according to structure (3).

In a further aspect, the invention concerns the application of the conjugate according to structure (2), for targeting tumour cells. Related thereto, the invention concerns the first medical use and second medical use of the conjugate according to structure (2).

As will be understood by the skilled person, the definition of the chemical moieties, as well as their preferred embodiments, apply to all aspects of the invention.

### The Compound According to Structure (1)

The invention concerns a compound according to structure (1):

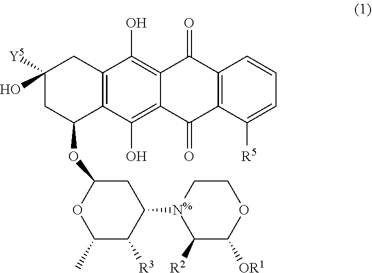

wherein:
R¹ is optionally substituted Et, i-Pr, n-Pr, t-Bu, i-Bu, n-Bu, n-pentyl, cyclopropyl, cyclobutyl, cyclopentyl, C₆₋₁₅ alkyl, C₂₋₁₅ alkenyl, C₂₋₁₅ alkynyl, heterocyclyl, (hetero)aryl, Sp-(hetero)aryl, Sp-heterocyclyl, Sp—X²R⁴, Sp—N₃, Sp—X²—Sp—R¹² or Sp—N(R⁴)₂, wherein the optional substituent is selected from halogen, C₁₋₁₂ (hetero)alkyl, (hetero)aryl, C₂₋₁₅ alkenyl, C₂₋₁₅ alkynyl, X²R⁴, N(R⁴)₂, NO₂, and wherein substituents C₁₋₁₂ (hetero)alkyl and (hetero)aryl may optionally be further substituted with C₁₋₆ (hetero)alkyl, X²R⁴ and N(R⁴)₂; wherein each Sp is individually C₁₋₁₂ (hetero)alkylene, (hetero)arylene, C₁₋₁₂ (hetero)alkylene-(hetero)arylene, or (hetero)arylene-C₁₋₁₂ (hetero)alkylene, wherein the (hetero)alkylene or the (hetero)arylene is optionally substituted with one or more substituents selected from halogen, X²R⁴, N(R⁴)₂, C₁₋₄ alkyl and NO₂, wherein each R⁴ is individually H, C₁₋₄ alkyl or adamantyl and X² is C(O), C(O)O, C(O)NH, O, S, S(O), S(O)₂, S(O)NH or S(O)₂NH; and wherein R¹² is β-glucuronide acid, PO₃⁽²⁻⁾, OPO₃⁽²⁻⁾, CO₂⁽⁻⁾, SO₃⁽⁻⁾ or N(C₁₋₄ alkyl)₃⁽⁺⁾;
R² is H, S(O)₂OH or P(O)₂OH and R³ is OH, or R² and R³ are fused together via an ether moiety to form an oxazolidine ring;
R⁵ is H or OCH₃;
Y⁵ is CH₂—Y, C(O)—Y, C(═N(R²⁰))—Y, C(R⁹)═N—Y, C(R⁹)═N(R²⁰), wherein R⁹ is selected from C₁₋₄ alkyl optionally substituted with an OH group or O(CO)C₁₋₆ alkyl, and R²⁰ is NR⁴—C(O)—N(R⁴)₂, NR⁴—C(O)—Sp—N(R⁴)₂, NR⁴—C(O)—R¹², NR⁴—C(O)—Sp—R¹², wherein Sp, R⁴ and R¹² are as defined above;
Y is NR⁴—Sp³—N(R⁴)₂, NR⁴—Sp³—X²(R⁴), N(R⁴)₂, R¹², Sp³R¹², NR⁴—Sp³—X²—Sp^{3′}—R¹², OH, CH₃ or CH₂OH, wherein Sp³ and Sp^{3′} are spacers;
N^{%} is N or N→O.

The compound according to structure (1) may be connected to a cell-binding agent (i.e. a conjugate), or may comprise a reactive group capable of reacting with an appropriately functionalized cell-binding agent or with a linker that is in a next step to be conjugated to a cell-binding agent (i.e. in free form or a small molecule). For certain preferred embodiments of the compound according to structure (1), the connection to the cell-binding agent or to the reactive moiety may be at any position of the compound. Preferably, this connection is through Y or R¹, most preferably it is through Y. The reactive group that capable of connecting the compound according to structure (1) to a linker or a cell-binding agent may for example be the N(R⁴)₂ or X²(R⁴) group in Y or the X²R⁴ or N₃ group in R¹.

Also contemplated in the present invention are salts, especially pharmaceutically acceptable salts, thereof. Typically, the S(O)₂OH or P(O)₂OH groups as R² or similar groups of R¹² may be present in salt form, containing a pharmaceutically acceptable cation such as Na⁺, K⁺, NH₄⁺ or NEt₄⁺. Salts are particularly contemplated in case the compound according to structure (1) is in free form, not conjugated to a cell-binding agent. Conjugates are less often in salt form.

The nature of R¹ is one the key aspect of the present invention. The inventors found that replacing the methyl group (R¹=Me) in prior art anthracycline-type toxins by larger substituents, the toxicity of the toxin could be beneficially affected. The present inventors are the first to modulate this substituent to improve the toxicity, while conjugating the toxin to a cell-binding agent via Y.

R¹ is selected from optionally substituted Et, i-Pr, n-Pr, t-Bu, i-Bu, n-Bu, n-pentyl, cyclopropyl, cyclobutyl, cyclopentyl, C₆₋₁₅ alkyl, C₂₋₁₅ alkenyl, C₂₋₁₅ alkynyl, heterocyclyl, (hetero)aryl, Sp-(hetero)aryl, Sp-heterocyclyl, Sp—X²R⁴, Sp—N₃, Sp—X²—Sp—R¹² or Sp—N(R⁴)₂. Preferably, R¹ is selected from optionally substituted i-Pr, n-Pr, t-Bu, i-Bu, n-Bu, n-pentyl, cyclopropyl, cyclobutyl, cyclopentyl, n-hexyl, C₇₋₁₂ alkyl, C₃₋₁₂ alkenyl, C₃₋₁₂ alkynyl, Sp-(hetero)aryl, Sp-heterocyclyl, Sp-OR⁴, Sp—N₃, Sp—X²—Sp—R¹² or Sp—N(R⁴)₂.

In an alternative preferred embodiment, R¹ is selected from optionally substituted Et, i-Pr, n-Pr, t-Bu, i-Bu, n-Bu, n-pentyl, C₆₋₁₂ alkyl, (hetero)aryl, Bn, Sp-(hetero)aryl, Sp—X²R⁴, Sp—N₃, Sp—X²—Sp—R¹² and Sp—N(R⁴)₂. In a further preferred embodiment, R¹ is selected from optionally substituted Et, i-Pr, n-Pr, t-Bu, i-Bu, n-Bu, n-pentyl, C₆₋₁₂ alkyl, (hetero)aryl, Bn, Sp-(hetero)aryl, Sp—OR⁴, Sp—N₃, Sp—X²—Sp—R¹² and Sp—N(R⁴)₂. In another preferred embodiment, R¹ is selected from optionally substituted Et, i-Pr, n-Pr, t-Bu, i-Bu, n-Bu, (hetero)aryl, Bn, Sp-(hetero)aryl, Sp—X²R⁴, Sp—N₃, Sp—X²—Sp—R¹² and Sp—N(R⁴)₂. In another preferred embodiment, R¹ is selected from optionally substituted Et, i-Pr, n-Pr, t-Bu, i-Bu, n-Bu, (hetero)aryl, Bn, Sp-(hetero)aryl, Sp—OR⁴, Sp—N₃ and Sp—N(R⁴)₂. In another preferred embodiment, R¹ is selected from optionally substituted i-Pr, n-Pr, t-Bu, i-Bu, n-Bu, (hetero)aryl, Bn, Sp-(hetero)aryl, Sp—OR⁴, Sp—N₃ and Sp—N(R⁴)₂. In one embodiment, R¹ is as defined above but is not Sp—X²—Sp—R¹².

The R¹ group may be optionally substituted with one or more substituent, wherein the optional substituent is selected from halogen, C₁₋₁₂ (hetero)alkyl, (hetero)aryl, C₂₋₁₅ alkenyl, C₂₋₁₅ alkynyl, X²R⁴, N(R⁴)₂, and NO₂, preferably from halogen, X²R⁴ and N(R⁴)₂. Most preferably, the optional substituent(s) is/are selected from OH, SH and NH₂. If present, the substituent may be located at any position of R¹. In a preferred embodiment, the carbon atom that is positioned directly adjacent to the O atom to which R¹ is connected does not bear a substituent, such that it is only connected to carbon and/or hydrogen atoms, which was found to improve the stability of the compound. In an especially preferred embodiment, the R¹ group comprises 0-2 substituents, more preferably 0 or 1 substituent, most preferably the R¹ group is not substituted.

The optional substituents C₁₋₁₂ (hetero)alkyl and (hetero)aryl may themselves also be further substituted with an optional substituent selected from C₁₋₆ (hetero)alkyl, X²R⁴ and N(R⁴)₂. Preferred embodiments for X² and R⁴ equally apply to these optional substituents of the C₁₋₁₂ (hetero)alkyl and (hetero)aryl substituents. In one embodiment, the C₁₋₁₂ (hetero)alkyl and (hetero)aryl substituents do not contain any further substituents.

Herein, X² is C(O), C(O)O, C(O)NH, O, S, S(O), S(O)₂, S(O)NH or S(O)₂NH, preferably X² is O, S, S(O) or S(O)₂. Preferably, X² is not S. Therefore, X² is preferably selected from C(O), C(O)O, C(O)NH, O, S(O), S(O)₂, S(O)NH or S(O)₂NH. In a preferred embodiment, X² is O, S(O) or S(O)₂. Most preferably, X² is O. R⁴ is selected from H, C₁₋₄ alkyl and adamantly. Preferably, R⁴ is H or C₁₋₄ alkyl. Each X² and R⁴, as well as each optional substituent, may be individually selected.

R¹² is β-glucuronide acid, PO₃⁽²⁻⁾, OPO₃⁽²⁻⁾, CO₂⁽⁻⁾, SO₃⁽⁻⁾ or N(C₁₋₄ alkyl)₃⁽⁺⁾. Preferably, R¹² is β-glucuronide acid, PO₃⁽²⁻⁾ or SO₃⁽⁻⁾. Most preferably, R¹² is β-glucuronide acid.

Sp is an alkyl or aryl spacer. More specifically, Sp is selected from C₁₋₁₂ (hetero)alkylene, (hetero)arylene, C₁₋₁₂ (hetero)alkylene-(hetero)arylene or (hetero)arylene-C₁₋₁₂ (hetero)alkylene. The carbon atoms of Sp may be substituted with one or more substituents selected from halogen, X²R⁴, N(R⁴)₂, C₁₋₄ alkyl and NO₂. Preferred embodiments for X² and R⁴ equally apply to these optional substituents of Sp. In a preferred embodiment, the optional substituent is selected from F, CI, Br, OH, OR⁴, SH, NH₂, Et, Me and NO₂. In an especially preferred embodiment, spacer Sp comprises 0-2 substituents, more preferably 0 or 1 substituent, most preferably spacer Sp is not substituted. The (hetero)alkylene and (hetero)arylene groups may be optionally interrupted with one or more elements selected from O, S, S(O), S(O)₂ or NR₄. Each Sp, as well as each optional substituent, may be individually selected.

Preferred options for R¹ are according to structures (D1)-(D67), depicted here below.

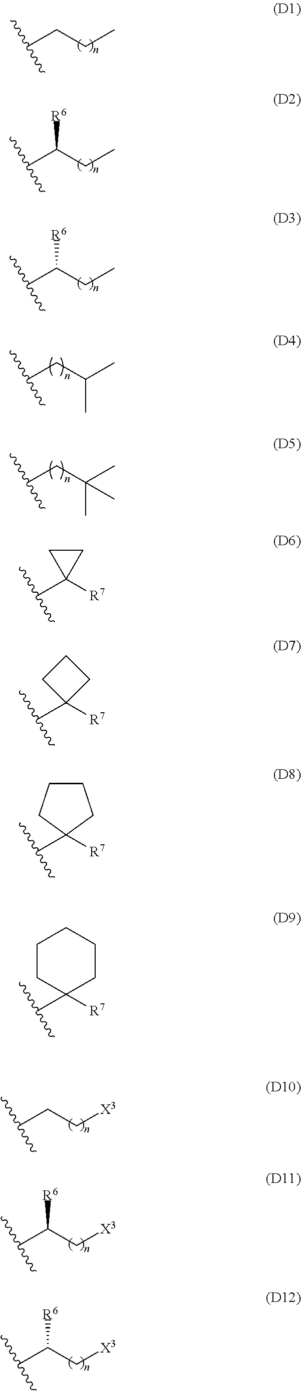

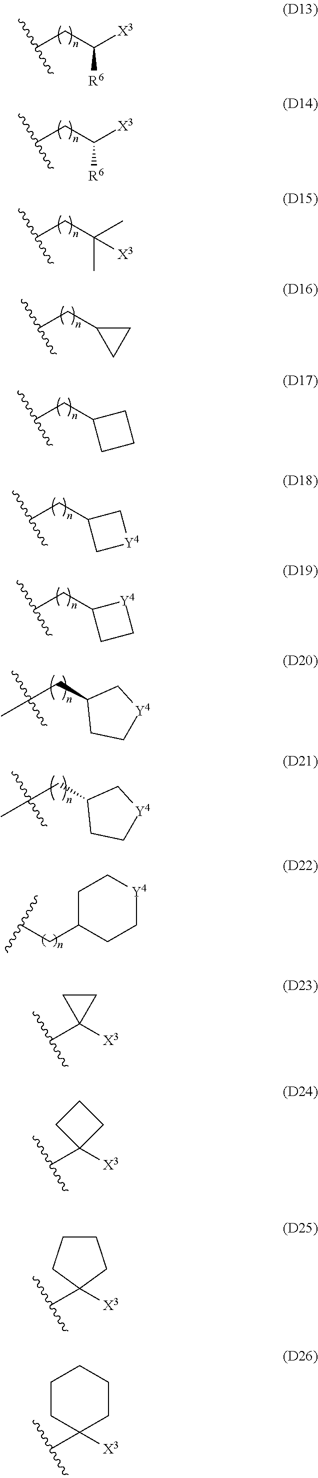

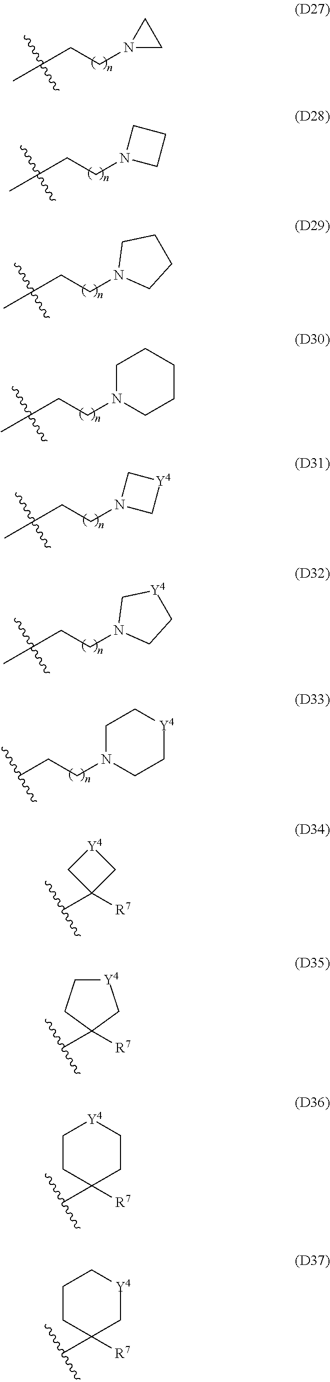

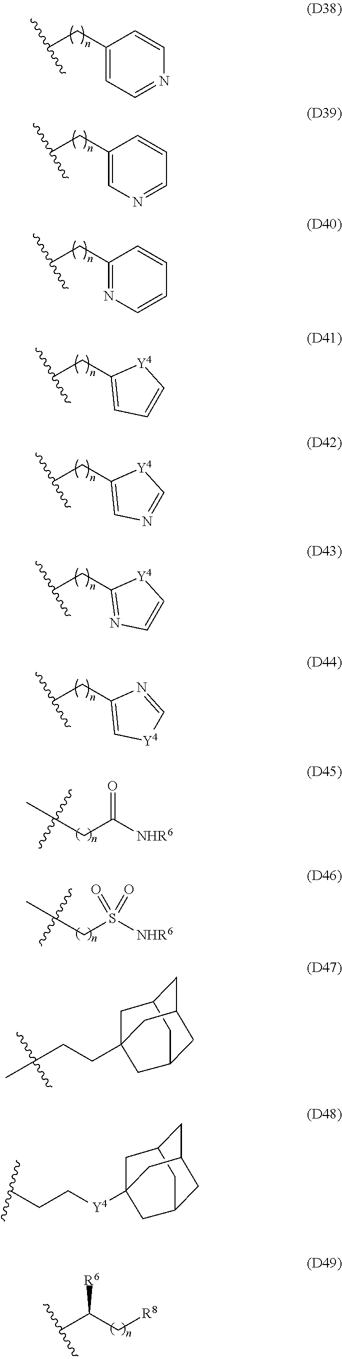

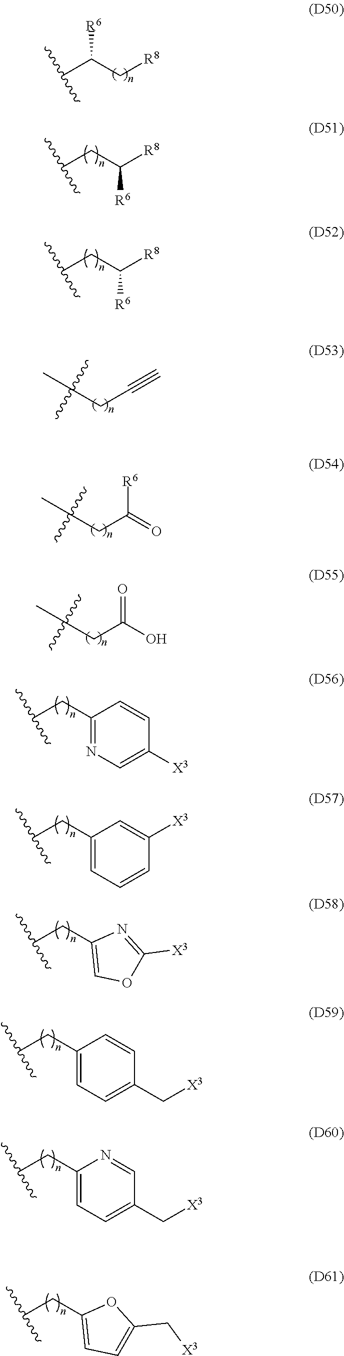

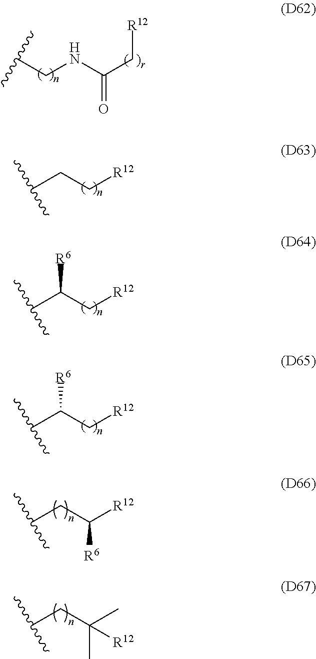

Herein, the following applies:
n and n′ are individually an integer in the range of 0-10, preferably in the range of 1-10, more preferably in the range of 1-5.
X³ is selected from OH, NH₂, OR⁶, N(R⁶)₂, N⁽⁺⁾(R⁶)₃, SR⁶, S(O)R⁶, S(O)₂R⁶, N₃ and SH.
Y⁴ is selected from NH, NR⁶, N⁽⁺⁾(R₆)₂, S(O) and S(O)₂.
Each R⁶ is individually selected from hydrogen, C₁₋₁₂ alkyl, C₂₋₁₂ alkenyl, C₂₋₁₂ alkynyl, C₃-C₁₂ cycloalkane, C₃-C₁₂ cycloalkenyl, C₃-C₁₂ cycloalkynyl, (hetero)aryl and polyethyleneglycol (PEG). Herein, PEG typically has the structure (CH₂CH₂O)ₘR¹⁰, wherein m is 1, 2 or 3 and R¹⁰ is H, CH₃ or CH₂CH₃.
R⁷ is H or (CH₂)ₙCH₃.
R⁸ is a (hetero)aryl group.
R¹² is β-glucorinide acid, PO₃²⁻, OPO₃²⁻, CO₂⁻, SO₃⁻ and N(C₁₋₄alkyl)₃⁺

The preferred options (D1)-(D67) for R¹ also include the halogenated and/or unsaturated versions thereof. Thus, any hydrogen atom directly bound to a carbon atom may be replaced by a halogen, preferably by F or CI, more preferably by F. Most preferably, no hydrogen atom is replaced by a halogen atom. Likewise, any two adjacent saturated carbon atoms may also contain a double or triple bond in between them where possible. In other words, a CH₂—CH₂ fragment may be replaced by a CH═CH fragment or a C≡C fragment, a CH₂—CH fragment may be replaced by a CH═C fragment, and a CH—CH fragment may be replaced by a C═C fragment. Most preferably, no carbon-carbon double or triple bonds are present except for those explicitly indicated in the structures of (D1)-(D67). In a preferred embodiment, R¹ is selected from (D1)-(D61).

Y⁵ typically contains a carbonyl moiety as present in the parent anthracyclines compounds. Alternatively, the carbonyl group may be replaced by a methylene group, an immine group or a hydrazone group. Hydrazones are cleaved under low pH conditions of the endosome and/or lysosome, but are stable in blood circulation. The hydrazone moiety may be introduced by reacting the ketone of the parent anthracycline with Y—C(O)—NH—NH₂. Preferably, Y⁵ contains a carbonyl moiety or a hydrazone moiety, most preferably a carbonyl moiety. Hence, Y⁵ is CH₂—Y, C(O)—Y, C(═N(R²⁰))—Y, C(R⁹)═N—Y, or C(R⁹)═N(R²⁰), preferably Y⁵ is C(O)—Y, C(═N(R²⁰))—Y, C(R⁹)═N—Y, or C(R⁹)═N(R²⁰), most preferably Y⁵ is C(O)—Y.

In case Y⁵ contains an immine or hydrazone moiety, R⁹ is the substituent on carbon and R²⁰ the substituent on nitrogen. R⁹ is selected from C₁₋₄ alkyl optionally substituted with an OH group or an O(CO)C₁₋₆ alkyl group, and R²⁰ is NR⁴—C(O)—N(R⁴)₂, NR⁴—C(O)—Sp—N(R⁴)₂, NR⁴—C(O)—R¹², NR⁴—C(O)—Sp—R¹², wherein Sp, R⁴ and R¹² are as defined above. Herein, Sp, R⁴ and R¹² are as defined above. Preferably, R⁹ is Me, CH₂OH or CH₂OC(O)C₁₋₆alkyl, more preferably R⁹ is Me, CH₂OH or CH₂OC(O)C₄H₉, most preferably R⁹ is Me. R²⁰ is preferably NR⁴—C(O)—R¹² or NR⁴—C(O)—Sp—R¹², most preferably R²⁰ is NR⁴—C(O)—Sp—R¹². Sp, R⁴ and R¹², as well as preferred embodiments thereof, are defined above. In the context of R²⁰, it is preferred that R⁴ is selected from hydrogen and C₁₋₄ alkyl, more preferably from H and Me, most preferably R⁴ is Me. In the context of R²⁰, it is preferred that Sp is C₁₋₄ alkylene, most preferably Sp is CH₂. In the context of R²⁰, it is preferred that R¹² is N(C₁₋₄ alkyl) 3 (+), more preferably N (Me) 3 (+).

In a preferred embodiment, the compound according to structure (1) can be conjugated to a cell-binding agent through Y. In this embodiment, it is preferred that R¹ does not contain a reactive moiety for conjugation to a cell-binding agent. Hence, it is preferred that R¹ is selected from (D1)-(D52), wherein X³ is selected from OR⁶, N(R⁶)₂, N⁽⁺⁾(R⁶)₃, SR⁶, S(O)R⁶, S(O)₂R⁶, and Y⁴ is selected from NR⁶, N⁽⁺⁾(R⁶)₂, S(O) and S(O)₂, wherein each occurrence of R⁶ is individually selected from C₁₋₁₂ alkyl, C₂₋₁₂ alkenyl, C₂₋₁₂ alkynyl, C₃-C₁₂ cycloalkane, C₃-C₁₂ cycloalkenyl, C₃-C₁₂ cycloalkynyl, (hetero)aryl and PEG, i.e. R⁶ is not hydrogen.

In an alternative preferred embodiment, the compound according to structure (1) can be conjugated to a cell-binding agent through R¹. In this embodiment, R¹ comprises a reactive moiety for conjugation to a cell-binding agent. Hence, it is preferred that R¹ is selected from (D10)-(D15), (D18)-(D26), (D31-(D37), (D41)-(D44), (D48) and (D53)-(D61), wherein X³ is selected from OH, NH₂, NHR⁶, N₃, SH and/or Y⁴ is NH.

It is preferred that if the compound is conjugated to a cell binding agent through R¹, Y⁵ is selected from structures (Y¹¹)-(Y¹⁶) depicted here below:

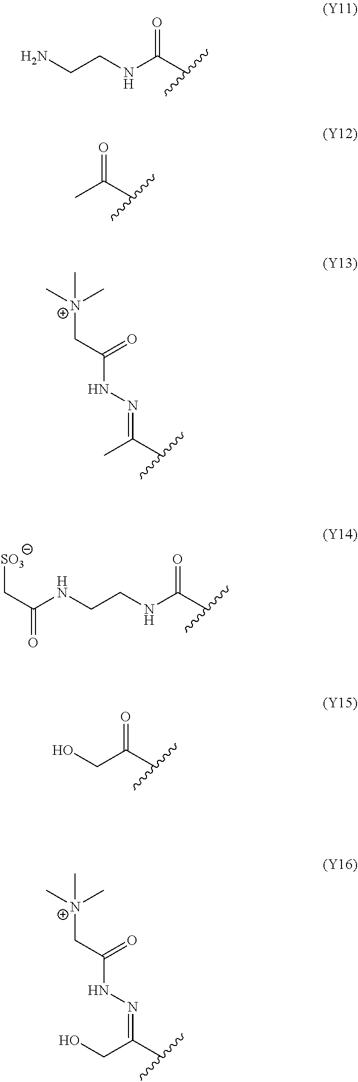

In a preferred embodiment, Y⁵ is C(═N(R²⁰))—Y or C(R⁹)═N(R²⁰), and R²⁰ is NR⁴—C(O)—R¹² or NR⁴—C(O)—Sp—R¹². The combination of the hydrazone and the ionic R¹² group improves the therapeutic window of the conjugate according to the invention, since the ionic cap prevents the payload from entering a cell when the payload is premature released and at the same time the ionic R¹² group reduces aggregation while it is still attached to the antibody.

In one embodiment, R¹ is not CH₂CH₂SH or benzyl in case Y═CH₂OH, more preferably R¹ is not CH₂CH₂SH or benzyl irrespective of Y. In one embodiment, R¹ is not unsubstituted ethyl, CH₂CH₂SH or benzyl in case Y═CH₂OH, more preferably R¹ is not unsubstituted ethyl, CH₂CH₂SH or benzyl irrespective of Y.

The compound according to the invention comprises an oxane ring and a morpholine ring. These may be joined together in a “closed” tricyclic structure comprising an intermediate oxazolidine ring, or the structure may be “open”. R² and R³ are the substituents on the oxane and morpholine rings. In one embodiment, R² and R³ are fused together via an ether moiety, and as such form a five-membered oxazolidine ring. In an alternative embodiment, the cyclic structure is open and R² is H, S(O)₂OH or P(O)₂OH and R³ is OH. Herein, S(O)₂OH and P(O)₂OH may be in salt form. In case of an open form, it is preferred that R² is H and R³ is OH. In a most preferred embodiment, the structure is closed and R² and R³ are fused together via an ether moiety to form an oxazolidine ring.

R⁵ is a substituent on the outer phenyl ring of the tetracyclic moiety. R⁵ is either H or OCH₃. In a preferred embodiment, R⁵ is OCH₃. Preferably R² is H and R³ is OH, or R² and R³ are fused together via an ether moiety to form an oxazolidine ring and R⁵ is OCH₃, more preferably R² and R³ are fused together via an ether moiety to form an oxazolidine ring and R⁵ is OCH₃.

Y is NR⁴—Sp³—N(R⁴)₂, NR⁴—Sp³—X²(R⁴), N(R⁴)₂, R¹², NR⁴—Sp³—X²—Sp³—R¹², OH, CH₃ or CH₂OH. In a preferred embodiment, Y is NR⁴—Sp³—N(R⁴)₂, NR⁴—Sp³—X²(R⁴), N(R⁴)₂, CH₃ or CH₂OH, more preferably Y is NR⁴—Sp³—N(R⁴)₂, N(R⁴)₂, CH₃ or CH₂OH. Herein, X² and R⁴ are as defined above, including preferred embodiments thereof, and Sp³ is a spacer. Spacer Sp³ is preferably selected from C₁₋₁₂ (hetero)alkylene, (hetero)arylene, C₁₋₁₂ (hetero)alkylene-(hetero)arylene, or (hetero)arylene-C₁₋₁₂ alkylene, wherein the alkylene or the (hetero)arylene may be optionally substituted with one or more substituents selected from halogen, X²R⁴, N(R⁴)₂, C₁₋₄ alkyl and NO₂, wherein the C₁₋₄ alkyl substituent may optionally form a cyclic structure by being joined with an NR⁴ moiety, in particular in a pyrrolidine formed with the NR⁴ moiety with the bond labelled with *, and the alkylene may optionally be interrupted with one or more heteroatoms selected from X² and NR⁴ Preferred spacers Sp³ include C₁₋₄ alkylene, which is optionally substituted as defined above and wherein the substituent may be joined together with an R⁴ substituent to form a cyclic structure. A preferred cyclic structure is a pyrrolidine ring, in particular wherein Sp³—N(R⁴)₂ together form a proline amino acids, i.e, wherein Y═NR⁴—CH₂-pyrollidine-N*, wherein N* may comprise an R⁴ substituent are the connection to the cell-binding agent via the linker. Herein, R⁴ is preferably CH₃ or H.

Especially preferred options of Y, when the compound according to general structure (1) is in free form or conjugated to a cell-binding agent not via Y, are selected from NR⁴—(CH₂)—N(R⁴)₂, NR⁴—Sp³—X²(R⁴), N(R⁴)₂, CH₃ or CH₂OH,

The compound according to structure (1), if in free form, may be in amine form (N^{%}═N) or in N-oxide form (N^{%}═N→O). If the compound according to structure (1) is conjugated to a cell-binding agent, it is always in amine form. In a preferred embodiment, the compound in free form is in amine form and N^{%}═N. In an especially preferred embodiment, the compound is in amine form (i.e. N^{%}═N) and the structure is closed (i.e. R² and R³ are fused together via an ether moiety to form an oxazolidine ring).

Especially preferred compounds according to structure (1) contain a moiety R¹ selected from optionally substituted Et, i-Pr, n-Pr, t-Bu, i-Bu, n-Bu, n-pentyl, C₆₋₁₂ alkyl, (hetero)aryl, Bn, Sp-(hetero)aryl, Sp—OR⁴, Sp—N₃ and Sp—N(R⁴)₂. Preferably, R¹ selected from optionally substituted i-Pr, n-Pr, t-Bu, i-Bu, n-Bu, aryl, Bn, Sp—N₃ and Sp—N(R⁴)₂. Herein, the optional substituents, Sp, X² and R⁴ are as defined above, including preferred embodiments thereof. In the context of the present embodiment, it is preferred that R¹ is selected from optionally substituted i-Pr, n-Pr, t-Bu, i-Bu, n-Bu, n-pentyl, C₆₋₁₂ alkyl, (hetero)aryl, Bn, Sp-(hetero)aryl, Sp—OR⁴, Sp—N₃ and Sp—N(R⁴)₂, more preferably from i-Pr, t-Bu, Bn, Sp—N₃ or Sp—NH₂, wherein Sp is C₁₋₄ alkylene or C₁₋₄ alkylene-arylene. More preferably, R¹ is i-Pr, Bn or Sp—N₃, wherein Sp is CH₂CH₂, CH₂CH₂CH₂ or CH₂(Ph). Herein, CH₂(Ph) may be CH₂(2-Ph), CH₂(3-Ph) or CH₂(4-Ph), preferably it is CH₂(4-Ph). In one especially preferred embodiment, R¹ is i-Pr, Bn, CH₂CH₂N₃, CH₂CH₂CH₂N₃ or CH₂ ((4-N₃) Ph).

The compounds according to structure (1) may contain hydrophilic moiety R¹². It is believed that the hydrophilic moiety decreases aggregation of the ADC and also improves efficacy and/or toxicity characteristics. R¹² is selected from β-glucuronide acid, PO₃⁽²⁻⁾, OPO₃⁽²⁻⁾, CO₂⁽⁻⁾, SO₃⁽⁻⁾ and N(C₁₋₄ alkyl)₃⁽⁺⁾, wherein the anions may also be in their protonated form. In a preferred embodiment, the conjugate according to the invention is connected through Y and R¹ is Sp—R¹² or Sp—X²—Sp—R¹². More preferably, each Sp is individually C₁-C₅ alkyl and X² is NHC(O). Most preferably, R¹ is selected from:

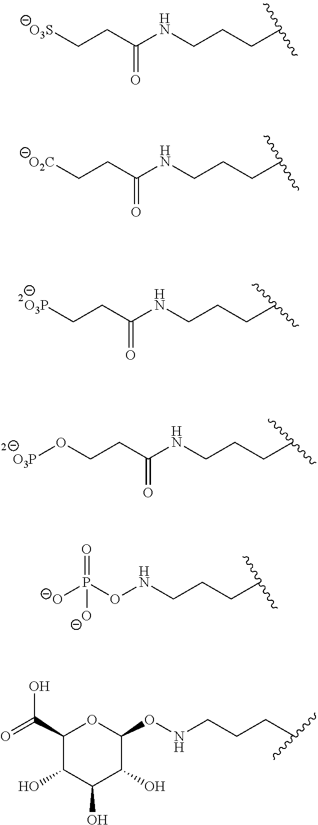

In another embodiment, the compound according to structure (1) comprises hydophilic moiety R¹² and the conjugate is connected through R¹. Herein, Y is preferably R¹², Sp³R¹² or NR⁴—Sp³—X²—Sp^{3′}—R¹². In this embodiment it is preferred that R¹² is SO⁽³⁻⁾ or N(C₁₋₄ alkyl)₃⁽⁺⁾. More preferably, Y is selected from NHCH₂CH₂NHC(O)CH₂SO₃⁽⁻⁾, NHCH₂CH₂NHC(O)CH₂NMe₃⁽⁺⁾, CH₂SO₃⁽⁻⁾ and CH₂NMe₃⁽⁺⁾, even more preferably Y is NHCH₂CH₂NHC(O)CH₂SO₃⁽⁻⁾ or NHCH₂CH—₂NHC(O)CH₂NMe₃⁽⁺⁾ and Y⁵ is C(O)—Y, or Y is CH₂SO₃⁽⁻⁾ or CH₂NMe⁽³⁾ and Y⁵ comprises a hydrazone group.

The inventors have obtained especially beneficial results with compounds according to structure (1) in terms of improved efficacy. Hence, in the context of conjugates according to structure (2), which are further defined below, it is preferred that the payload D is a compound according to this preferred embodiment. In these conjugates, the compounds according to structure (1) may be connected through R¹ or Y. In case R¹ is selected from i-Pr, t-Bu, Bn, Sp—N₃ or Sp—NH₂, wherein Sp is C₁₋₄ alkylene or C₁₋₄ alkylene-arylene. More preferably, R¹ is i-Pr, Bn or Sp—N₃, wherein Sp is CH₂CH₂ or CH₂(4-Ph).

In one embodiment of the compound according to structure (1), R¹ is not unsubstituted ethyl, CH₂CH₂SH or benzyl, when Y is CH₂OH and Y⁵ is C(O)—Y. Preferably, R¹ is not unsubstituted ethyl, CH₂CH₂SH or benzyl, when Y is CH₂OH. More preferably, R¹ is not unsubstituted ethyl, CH₂CH₂SH or benzyl. Preferably, in the context of the present embodiment, R¹ is not unsubstituted or substituted ethyl, CH₂CH₂SH or benzyl.

Preferably, when R² and R³ are fused together via an ether moiety to form an oxazolidine ring R¹ is not an alcohol, thiol or an amine.

### Conjugate of General Structure (2)

In a first aspect, the invention concerns conjugates wherein a compound according to structure (1) is conjugated to a cell-binding agent via a linker. Such a conjugate typically is of general structure (2):

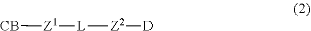

wherein:
CB is the cell-binding agent;
D is the compound according to structure (1);
L is a linker;
Z¹ is a connecting group that connects the cell-binding agent CB to the linker; and
Z² is a connecting group that connects the compound D to the linker.

### Cell-Binding Agent CB

The conjugate according to the invention contains a cell-binding agent, which is capable of targeting cells, for example by interaction with extracellular receptors on the surface of cells. The cell-binding agent is typically a peptide (e.g. an antibody), a small molecule or an aptamer. Preferably, the cell-binding agent is a peptide, like a polypeptide, which is capable of such interaction with a specific receptor and is this able to target specific cells. Advantageously, these specific cells are tumour cells. In a most preferred embodiment, the cell-binding agent (CB) is an antibody (Ab), typically an antibody that is capable of binding to a specific extracellular receptor on the surface of a cell, such that the antibody is able to target that specific cell.

Antibodies are known in the art and include IgA, IgD, IgE, IgG, IgM, Fab, VHH, scFv, diabody, minibody, affibody, affylin, affimers, atrimers, fynomer, Cys-knot, DARPin, adnectin/centryin, knottin, anticalin, FN3, Kunitz domain, Obody, bicyclic peptides and tricyclic peptides. Preferably, the antibody is a monoclonal antibody, more preferably selected from the group consisting of IgA, IgD, IgE, IgG and IgM antibodies. Even more preferably Ab is an IgG antibody. The IgG antibody may be of any IgG isotype. The antibody may be any IgG isotype, e.g. IgG1, IgG2, IgI3 or IgG4. Preferably Ab is a full-length antibody, but Ab may also be a Fc fragment.

The antibody Ab is typically specific for an extracellular receptor on a tumour cell, preferably wherein the extracellular receptor on the tumour cell is selected from the group consisting of 5T4, ADAM-9, AMHRII, ASCT2, ASLG659, ASPHD1, av-integrin, Axl, B7-H3, B7-H4, BAFF-R, BCMA, BMPR1B, Brevican, c-KIT, c-Met, C4.4a, CA-IX, cadherin-6, CanAg, CD123, CD13, CD133, CD138/syndecan-1, CD166, CD19, CD20, CD203c, CD205, CD21, CD22, CD228, CD25, CD30, CD324, CD33, CD37, CD38, CD45, CD46, CD48a, CD56, CD70, CD71, CD72, CD74, CD79a, CD79b, CEACAM5, claudin-18.2, claudin-6, CLEC12A, CLL-1, Cripto, CRIPTO, CS1, CXCR5, DLK-1, DLL3, DPEP3, E16, EGFR, ENPP3, EpCAM, EphA2, EphB2R, ETBR, FAP, FcRH1, FcRH2, FcRH5, FGFR2, fibronectin, FLT3, folate receptor alpha, Gal-3BP, GD3, GDNF-Ra1, GEDA, GFRA1, Globo H, gpNMB, GPR172A, GPR19, GPR54, guanyl cyclase C, HER2, HER3, HLA-DOB, IGF-1R, IL13R, IL20Ra, Lewis Y, LGR5, LIV-1, LRRC15, LY64, Ly6E, Ly6G6D, LY6K, MDP, MFI2, MICA/B, MOSPD2, MPF, MSG783, MUC1, MUC16, NaPi2b, NCA, nectin-4, Notch3, P-cadherin, P2X5, PD-L1, PMEL17, PRLR, PSCA, PSCA hlg, PSMA, PTK7, RET, RNF43, RON, ROR1, ROR2, Sema 5b, SLITRK6, SSTR2, STEAP1, STEAP2, TAG72, TENB2, TF, TIM-1, TM4SF, TMEFF, TMEM118, TMEM46, transferrin, TROP-2, TrpM4, TWEAKR, receptor tyrosine kinases (RTK), tenascin.

The conjugate according to the invention contains a connecting group Z¹, which is formed during a conjugation reaction wherein the cell-binding agent, which may be appropriately modified, is reacted with the linker-toxin construct comprising L-Z²-D. In the bioconjugation reaction reactive group F on the cell-binding agent reacts with reactive group Q on the linker-toxin construct thereby forming a covalent connection between the cell-binding agent and the toxin. Part of the cell-binding agent may be a linker L⁶ that connects the reactive group F or connecting group Z¹ to the peptide part of the cell-binding agent. Preferably, the connecting group Z¹ is connected to the cell-binding agent CB via a lysine residue of CB, a glutamine residue of CB, a cysteine residue of CB, a tyrosine residue of CB, threonine residue of CB, or a glycan of CB.

Thus, the conjugate according to the invention is preferably represented by:

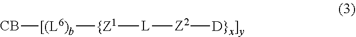

wherein:
b is 0 or 1;
L⁶ is -GlcNAc(Fuc)_{w}-(G)ⱼ-S-(L⁷)_{w}-, wherein G is a monosaccharide, j is an integer in the range of 0-10, S is a sugar or a sugar derivative, GlcNAc is N-acetylglucosamine and Fuc is fucose, w is 0 or 1, w′ is 0, 1 or 2 and L⁷ is —N(H)C(O)CH₂—, —N(H)C(O)CF₂— or —CH₂—;
x is 1 or 2; and
y is 1, 2, 3 or 4.

### Linker L⁶

In case reactive group F is directly connected to CB, linker L⁶ that connects CB to F or Z¹ (for conjugates of structure (1) is absent and b=0. This is for example the case for cysteine conjugation and lysine conjugation. Alternatively, reactive group F may also be introduced onto the antibody using a linker L⁶ that connects CB to F or Z¹, in which case L⁶ is present and b=1. In case L⁶ is present, reactive group F is typically introduced at the glycan of the antibody. This is for example the case for conjugation via an artificially introduced reactive group F, such as for example using transglutaminase, using sortase or by enzymatic glycan modification (e.g. glycosyltransferase or α-1,3-mannosyl-glycoprotein-2-β-N-acetylglucosaminyl-transferase). For example, a modified sugar residue S(F)ₓ may be introduced at the glycan, extending the glycan with one monosaccharide residue S, which introduces x reactive groups F on the glycan of an antibody. In a most preferred embodiment, conjugation occurs via the glycan of the antibody and b=1. The site of conjugation is preferably at the heavy chain of the antibody.

If present, L⁶ is a linker that links CB to F or to Z¹, and is represented by -GlcNAc(Fuc)_{w}-(G)ⱼ-S-(L⁷)_{w}-, wherein G is a monosaccharide, j is an integer in the range of 0-10, S is a sugar or a sugar derivative, GlcNAc is N-acetylglucosamine and Fuc is fucose, w is 0 or 1, w′ is 0, 1 or 2 and L⁷ is —N(H)C(O)CH₂—, —N(H)C(O)CF₂— or —CH₂—. Typically, L⁶ is at least partly formed by the glycan of an antibody. All recombinant antibodies, generated in mammalian host systems, contain the conserved N-glycosylation site at the asparagine residue at or close to position 297 of the heavy chain (Kabat numbering), which is modified by a glycan of the complex type. This naturally occurring glycosylation site of antibodies is preferably used, but other glycosylation sites, including artificially introduced ones, may also be used for the connection of linker L⁶. Thus, in a preferred embodiment, L⁶ is connected to an amino acid of the antibody which is located at a position in the range of 250-350 of the heavy chain, preferably in the range of 280-310 of the heavy chain, more preferably in the range of 295-300 of the heavy chain, most preferably at position 297 of the heavy chain.

The -GlcNAc(Fuc)_{w}-(G)ⱼ- of L⁶ is the glycan, or part thereof. The -GlcNAc(Fuc)_{w}-(G)ⱼ- of the glycan thus typically originates from the original antibody, wherein GlcNAc is an N-acetylglucosamine moiety and Fuc is a fucose moiety. Fuc is typically bound to GlcNAc via an x-1,6-glycosidic bond. Normally, antibodies may (w=1) or may not be fucosylated (w=0). In the context of the present invention, the presence of a fucosyl moiety is irrelevant, and similar effects are obtained with fucosylated (w=1) and non-fucosylated (w=0) antibody conjugates. The GlcNAc residue may also be referred to as the core-GlcNAc residue and is the monosaccharide that is directly attached to the peptide part of the antibody.

S may be directly connected to the core-GlcNAc(Fuc) w moiety, i.e. j=0, meaning that the remainder of the glycan is removed from the core-GlcNAc(Fuc) w moiety before S is attached. Such trimming of glycans is well-known in the art and can be achieved by the action of an endoglycosidase. Alternatively, there are one or more monosaccharide residues present in between the core-GlcNAc(Fuc) w moiety and S, i.e. j is an integer in the range of 1-10, preferably j=2-5.

In a preferred embodiment, (G) j is an oligosaccharide fraction comprising j monosaccharide residues G, wherein j is an integer in the range of 2-5. (G) j is connected to the GlcNAc moiety of GlcNAc(Fuc) w, typically via a β-1,4 bond. In a preferred embodiment, j is 3, 4 or 5. Although any monosaccharide that may be present in a glycan may be employed as G, each G is preferably individually selected from the group consisting of galactose, glucose, N-acetylgalactosamine, N-acetylglucosamine, mannose and N-acetylneuraminic acid. More preferred options for G are galactose, N-acetylglucosamine, mannose. Antibodies and antibody conjugates having j=0 show no or significantly reduced binding to Fc-gamma receptors, while antibodies and antibody conjugates having j in the range of 4-10 do bind to Fc-gamma receptors. Thus, by selecting a certain value for j, the desired extent of binding to Fc-gamma receptors can be obtained. It is thus preferred that j=0, 4, 5, 6, 7, 8, 9 or 10, more preferably j=0, 4 or 5, most preferably the antibody is trimmed and j=0.

S is a sugar or sugar derivative. The term “sugar derivative” is herein used to indicate a derivative of a monosaccharide sugar, i.e. a monosaccharide sugar comprising substituents and/or functional groups. Suitable examples for S include glucose (Glc), galactose (Gal), mannose (Man), fucose (Fuc), amino sugars and sugar acids, e.g. glucosamine (GlcNH₂), galactosamine (GalNH₂) N-acetylglucosamine (GlcNAc), N-acetylgalactosamine (GalNAc), sialic acid (Sia) which is also referred to as N-acetylneuraminic acid (NeuNAc), and N-acetylmuramic acid (MurNAc), glucuronic acid (GlcA) and iduronic acid (IdoA). Preferably, S is selected from Glc, Gal, GlcNAc and GalNAc. In an especially preferred embodiment, S is GalNAc.

x is an integer that denotes the number of connecting groups Z¹ or reactive groups F that are attached to sugar (derivative) S. Thus, the antibody preferably contains a moiety S comprising x reactive moieties F. Each of these reactive moieties F are reacted with a reactive moiety Q of the linker-toxin construct, such that x connecting groups Z are formed and x compounds according to general structure (1) are attached to a single occurrence of S. x is 1 or 2, preferably x=1.

Connecting group Z¹ or reactive group F may be attached directly to S, or there may be a linker L⁷ present in between S and Z¹ or F. Thus, L⁷ may be present (w′=1 or 2) or absent (w′=0). Typically, each moiety Z may be connected to S via a linker L⁷, thus in one embodiment w′=0 of x. Preferably, L⁷ is absent and each connecting moiety Z is directly attached to S. If present, L⁷ may be selected from —N(H)C(O)CH₂—, —N(H)C(O)CF₂— or —CH₂—. In a preferred embodiment, x=1 and w′=0 or 1, most preferably x=1 and w′=0.

y is an integer that denotes the number of sugar(s) (derivative(s)) S, each having x reactive groups F or connected to x connecting groups Z¹, that are connected to CB. y is 1, 2, 3 or 4, preferably y=2 or 4, most preferably y=1. Thus, the antibody contains y moieties S, each of which comprises x reactive moieties F. Each of these reactive moieties F are reacted with reactive moiety Q of the linker-toxin construct, such that x×y connecting groups Z¹ are formed and x×y compounds according to general structure (1) are attached to a single CB. Each linker-toxin construct may contain multiple payloads, e.g. by virtue of a branching nitrogen atom N* in L. It is preferred that each linker-toxin construct contains 1 or 2 occurrences of D, most preferably 1 occurrence of D. In an especially preferred embodiment, linker L¹ contains a branching nitrogen atom N* to which a second occurrence of D is connected.

The amount of toxins D (compounds according to general structure (1)) attached to a single antibody is known in the art as the DAR (drug-to-antibody ratio). In the context of the present invention, it is preferred that DAR is an integer in the range 1-8, more preferably 2 or 4, most preferably DAR=2. Alternatively worded, the DAR is preferably an integer in the range (x×y) to [(x×y)×2], most preferably DAR=[(x×y)×2]. With preferred values for x of 1 and y of 1, the DAR is preferably 2. It will be appreciated that these are theoretical DAR values, and in practice the DAR may slightly deviate from this value, by virtue of incomplete conjugation. Typically, the conjugates are obtained as a stochastic mixture of antibody-drug conjugates, with DAR values varying between individual conjugates, and depending on the conjugation technique used the DAR may have a broad distribution (e.g. DAR=0-10) or a narrow distribution (e.g. DAR=3-4). In case of such mixture, DAR often refers to the average DAR of the mixture. This is well-known in the art of bioconjugation. However, in case the conjugation occurs via the glycan (i.e. b=1 and L⁶ is present), the conjugates according to the invention have a close-to-theoretical DAR. For example, when the theoretical DAR is 4, DAR values above 3.6 or even above 3.8 are readily obtained, indicating that most antibodies in the reaction mixture have reacted completely and have a DAR of 4.

### Connecting Group Z¹

Z¹ is a connecting group, which covalently connects both parts of the conjugate according to the invention. The term “connecting group” herein refers to the structural element, resulting from a reaction, here between Q and F, connecting one part of the conjugate with another part of the same conjugate. As will be understood by the person skilled in the art, the nature of a connecting group depends on the type of reaction with which the connection between the parts of said compound is obtained. As an example, when the carboxyl group of R—C(O)—OH is reacted with the amino group of H₂N—R′ to form R—C(O)—N(H)—R′, R is connected to R′ via connecting group Z, and Z may be represented by the group —C(O)—N(H)—. Since connecting group Z¹ originates from the reaction between Q and F, it can take any form.

Since more than one reactive moiety F can be present or introduced in an antibody, the antibody-conjugate according to the present invention may contain per biomolecule more than one payloads D, such as 1-8 payloads D, preferably 1, 2, 3 or 4 payloads D, more preferably 2 or 4 payloads D. The number of payloads is typically an even integer, in view of the symmetric nature of antibodies. In other words, when one side of the antibody is functionalized with F, the symmetrical counterpart will also be functionalized. Alternatively, in case naturally occurring thiol groups of the cysteine residues of a protein are used as F, the value of m can be anything and may vary between individual conjugates.

In a compound according to structure (1), connecting group Z¹ connects D via linker L to CB, optionally via L⁶. Numerous reactions are known in the art for the attachment of a reactive group Q to a reactive group F. Consequently, a wide variety of connecting groups Z¹ may be present in the conjugate according to the invention. In one embodiment, the reactive group Q is selected from the options described above, preferably as depicted in FIG. 2, 4 or 5, and complementary reactive groups F and the thus obtained connecting groups Z¹ are known to a person skilled in the art. Several examples of suitable combinations of F and Q, and of connecting group Z¹ that will be present in a bioconjugate when a linker-conjugate comprising Q is conjugated to a biomolecule comprising a complementary reactive group F, are shown in FIG. 4.

For example, when F comprises or is a thiol group, complementary groups Q include N-maleimidyl groups, alkenyl groups and allenamide groups. For example, when F comprises or is an amino group, complementary groups Q include ketone groups and activated ester groups. For example, when F comprises or is a ketone group, complementary groups Q include (O-alkyl) hydroxylamino groups and hydrazine groups. For example, when F comprises or is an alkynyl group, complementary groups Q include azido groups. For example, when F comprises or is an azido group, complementary groups Q include alkynyl groups. For example, when F comprises or is a cyclopropenyl group, a trans-cyclooctene group, a cycloheptyne or a cyclooctyne group, complementary groups Q include tetrazinyl groups. In these particular cases, Z is only an intermediate structure and will expel N₂, thereby generating a dihydropyridazine (from the reaction with alkene) or pyridazine (from the reaction with alkyne) as shown in FIG. 4.

Additional suitable combinations of F and Q, and the nature of resulting connecting group Z¹ are known to a person skilled in the art, and are e.g. described in G. T. Hermanson, “Bioconjugate Techniques”, Elsevier, 3^{rd} Ed. 2013 (ISBN: 978-O-12-382239-0), in particular in Chapter 3, pages 229-258, incorporated by reference. A list of complementary reactive groups suitable for bioconjugation processes is disclosed in Table 3.1, pages 230-232 of Chapter 3 of G.. Hermanson, “Bioconjugate Techniques”, Elsevier, 3^{rd} Ed. 2013 (ISBN: 978-O-12-382239-0), and the content of this Table is expressly incorporated by reference herein.

In a preferred embodiment, connecting group Z¹ is obtained by a cycloaddition or a nucleophilic reaction, preferably wherein the cycloaddition is a [4+2] cycloaddition or a 1,3-dipolar cycloaddition or the nucleophilic reaction is a Michael addition or a nucleophilic substitution. Such a cycloaddition or nucleophilic reaction occurs via a reactive group F, connected to S, and reactive group Q, connected to D via L. Conjugation reactions via cycloadditions or nucleophilic reactions are known to the skilled person, and the skilled person is capable of selecting appropriate reaction partners F and Q, and will understand the nature of the resulting connecting group Z¹.

In a first preferred embodiment, Z¹ is formed by a cycloaddition. Preferred cycloadditions are a (4+2)-cycloaddition (e.g. a Diels-Alder reaction) or a (3+2)-cycloaddition (e.g. a 1,3-dipolar cycloaddition). Preferably, the conjugation is the Diels-Alder reaction or the 1,3-dipolar cycloaddition. The preferred Diels-Alder reaction is the inverse electron-demand Diels-Alder cycloaddition. In another preferred embodiment, the 1,3-dipolar cycloaddition is used, more preferably the alkyne-azide cycloaddition, and most preferably wherein Q is or comprises an alkyne group and F is an azido group. Cycloadditions, such as Diels-Alder reactions and 1,3-dipolar cycloadditions are known in the art, and the skilled person knowns how to perform them.

Preferably, Z¹ contains a moiety selected from the group consisting of a triazole, a cyclohexene, a cyclohexadiene, a [2.2.2]-bicyclooctadiene, a [2.2.2]-bicyclooctene, an isoxazoline, an isoxazolidine, a pyrazoline, a piperazine, a thioether, an amide or an imide group. Triazole moieties are especially preferred to be present in Z¹. In one embodiment, Z¹ comprises a (hetero)cycloalkene moiety, i.e. formed from Q comprising a (hetero)cycloalkyne moiety. In an alternative embodiment, Z¹ comprises a (hetero)cycloalkane moiety, i.e. formed from Q comprising a (hetero)cycloalkene moiety. Herein, aromatic rings such as a triazole ring are considered a heterocycloalkane ring, since it is formed by reaction of an alkyne moiety and an azide moiety. In a preferred embodiment, Z¹ has the structure (Z¹):

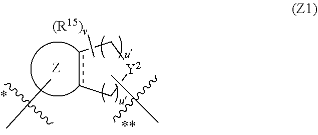

Herein, the bond depicted as [symbol] is a single bond or a double bond. Furthermore:
ring Z is obtained by a cycloaddition, preferably ring Z is selected from (Za)-(Zj) defined below, wherein the carbon atoms labelled with ** correspond to the two carbon atoms of the bond depicted as [symbol] of (Z1) to which ring Z is fused;
R¹⁵ is independently selected from the group consisting of hydrogen, halogen, —OR¹⁶, —NO₂, —CN, —S(O)₂R¹⁶, —S(O)₃ (−), C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero) arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)═NR³¹ or NR³¹, wherein S⁽⁺⁾ is a cationic sulphur atom counterbalanced by B⁽⁻⁾, wherein B⁽⁻⁾ is an anion, and wherein each R³¹ individually is R¹⁵ or a connection with D, connected via L;
u is 0, 1, 2, 3, 4 or 5;
u′ is 0, 1, 2, 3, 4 or 5, wherein u+u′=0, 1, 2, 3, 4, 5, 6, 7 or 8;
v=an integer in the range 8-16;
Ring Z is formed by the cycloaddition, and is preferably selected from (Za)-(Zj).

In a preferred embodiment, u+u′=0, 4, 5, 6, 7 or 8, more preferably 0, 4 or 5. In case the bond depicted as [symbol] is a double bond, it is preferred that u+u′=4, 5, 6, 7 or 8, more preferably u+u′=4 or 5. In case the bond depicted as [symbol] is a single bond, it is preferred that u+u′=0 or 5. Preferably, the wavy bond labelled with * is connected to CB, optionally via L⁶, and the wavy bond labelled with ** is connected to L.

It is especially preferred that Z¹ comprises a (hetero)cycloalkene moiety, i.e. the bond depicted as [symbol] is a double bond. In a preferred embodiment, Z¹ is selected from the structures (Z2)-(Z20), depicted here below:

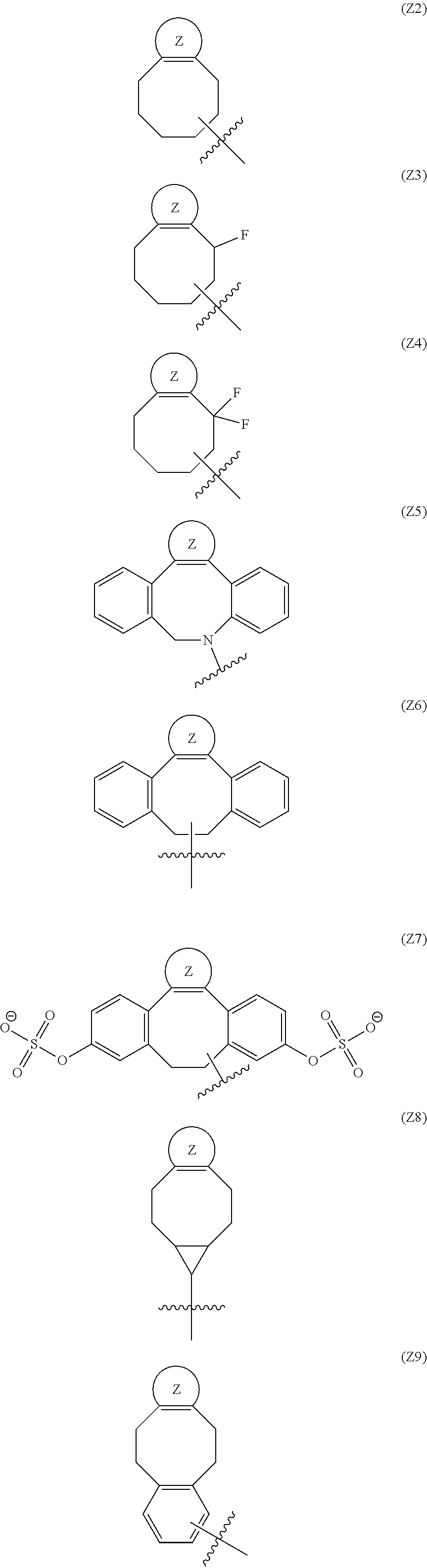

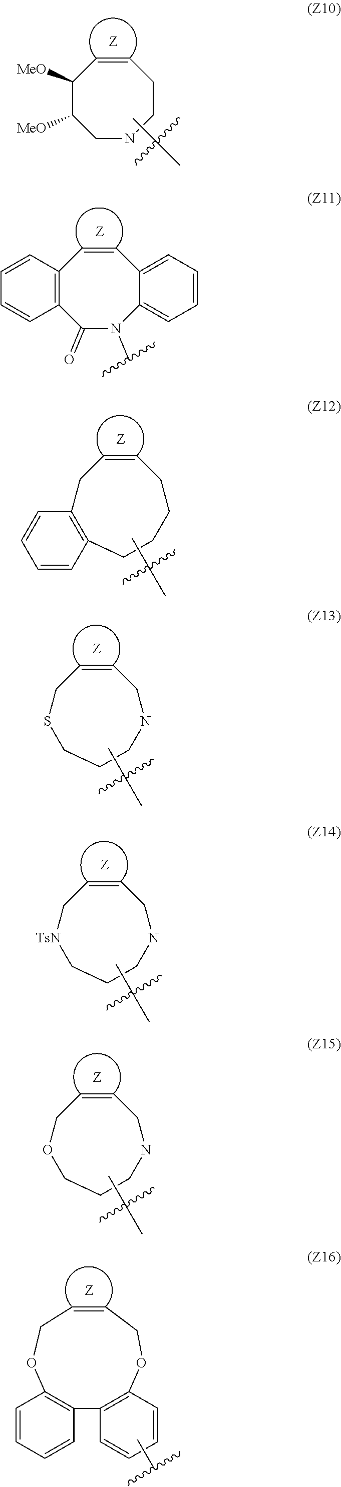

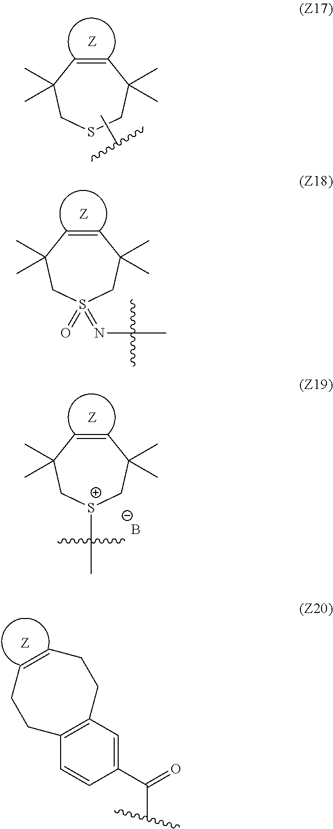

Herein, the connection to L is depicted with the wavy bond. B⁽⁻⁾ is an anion, preferably a pharmaceutically acceptable anion. Ring Z is formed by the cycloaddition reaction, and preferably is a triazole, a cyclohexene, a cyclohexadiene, a [2.2.2]-bicyclooctadiene, a [2.2.2]-bicyclooctene, an isoxazoline, an isoxazolidine, a pyrazoline or a piperazine. Most preferably, ring Z is a triazole ring. Ring Z may have the structure selected from (Za)-(Zj) depicted below, wherein the carbon atoms labelled with ** correspond to the two carbon atoms of the (hetero)cycloalkane ring of (Z2)-(Z20) to which ring Z is fused. Since the connecting group Z is formed by reaction with a (hetero)cycloalkyne in the context of the present embodiment, the bound depicted above as [symbol] is a double bond.

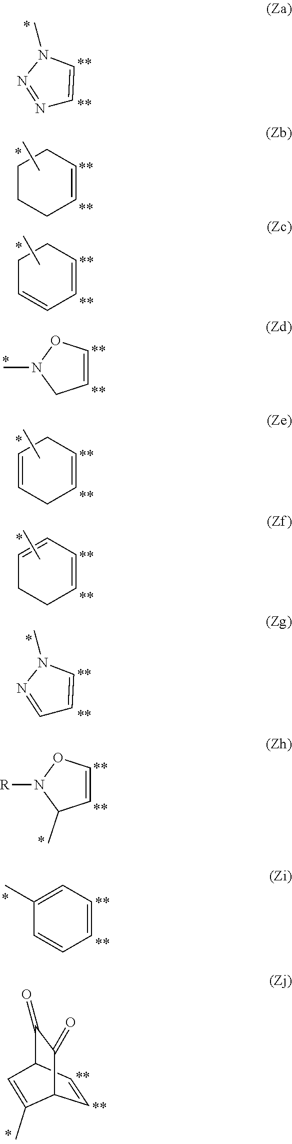

In a further preferred embodiment, Z¹ is selected from the structures (Z21)-(Z38), depicted here below:

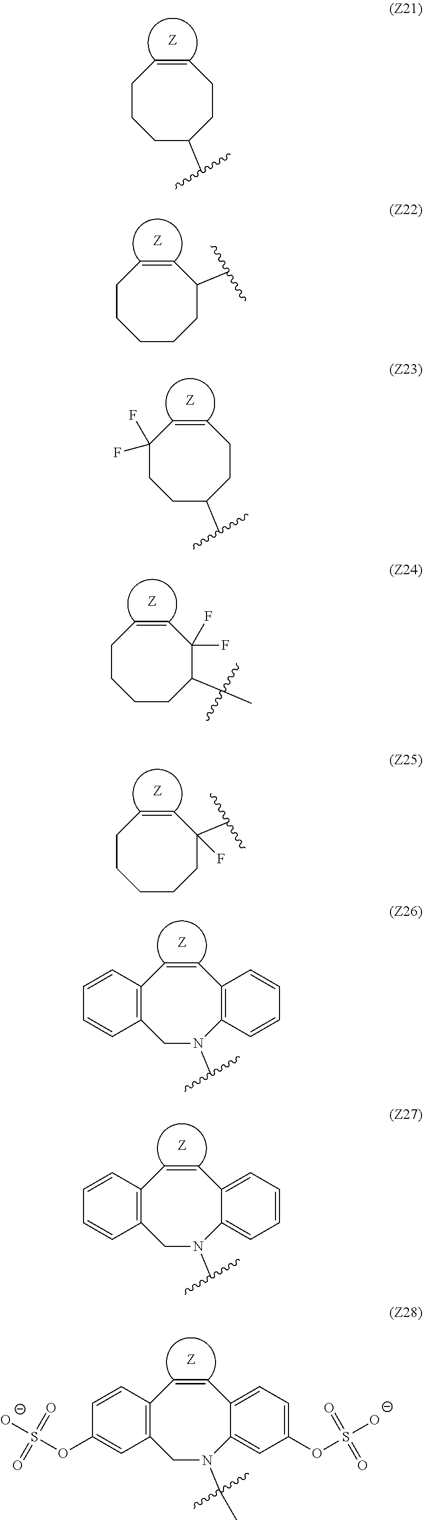

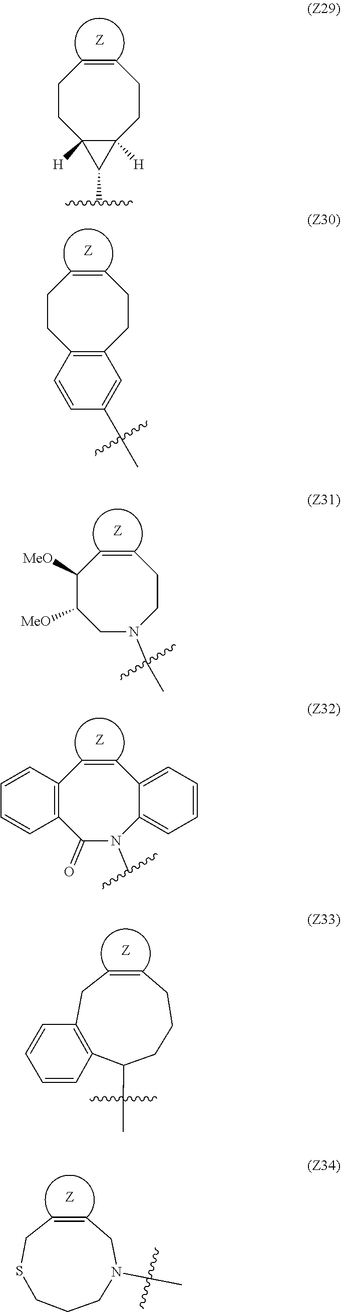

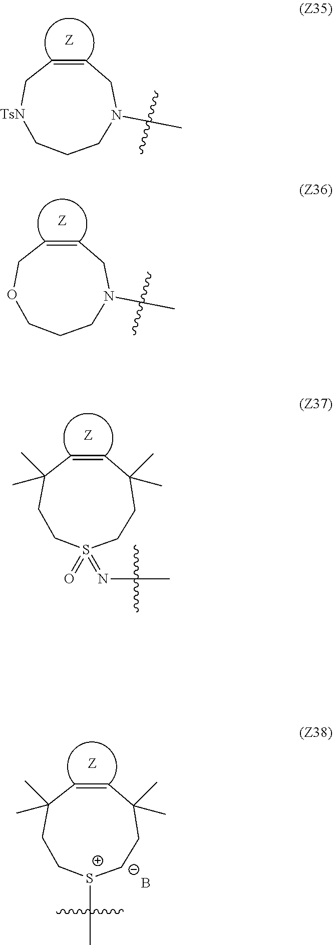

Herein, the connection to L is depicted with the wavy bond. Structure (Z29) can be in endo or exo configuration, preferably it is in endo configuration. In structure (Z38), B⁽⁻⁾ is an anion, preferably a pharmaceutically acceptable anion. Ring Z is selected from structures (Za)-(Zj), as defined above.

In a preferred embodiment, Z¹ comprises a (hetero)cyclooctene moiety or a (hetero)cycloheptene moiety, preferably according to structure (Z8), (Z26), (Z27), (Z28) or (Z37), which are optionally substituted. Each of these preferred options for Z¹ are further defined here below.

Thus, in a preferred embodiment, Z¹ comprises a heterocycloheptene moiety according to structure (Z37), which is optionally substituted. Preferably, the heterocycloheptene moiety according to structure (Z37) is not substituted.

In a preferred embodiment, Z¹ comprises a (hetero)cyclooctene moiety according to structure (Z8), more preferably according to (Z29), which is optionally substituted. Preferably, the cyclooctene moiety according to structure (Z8) or (Z29) is not substituted. In the context of the present embodiment, Z¹ preferably comprises a (hetero)cyclooctene moiety according to structure (Z39) as shown below, wherein V is (CH₂)_{I} and I is an integer in the range of 0 to 10, preferably in the range of 0 to 6. More preferably, I is 0, 1, 2, 3 or 4, more preferably I is 0, 1 or 2 and most preferably I is 0 or 1. In the context of group (Z39), I is most preferably 1. Most preferably, Z¹ is according to structure (Z42), defined further below.

In an alternative preferred embodiment, Z¹ comprises a (hetero)cyclooctene moiety according to structure (Z26), (Z27) or (Z28), which are optionally substituted. In the context of the present embodiment, Z¹ preferably comprises a (hetero)cyclooctene moiety according to structure (Z40) or (Z41) as shown below, wherein Y¹ is O or NR¹¹, wherein R¹¹ is independently selected from the group consisting of hydrogen, a linear or branched C₁-C₁₂ alkyl group or a C₄-C₁₂ (hetero)aryl group. The aromatic rings in (Z40) are optionally O-sulfonylated at one or more positions, whereas the rings of (Z41) may be halogenated at one or more positions. Preferably, the (hetero)cyclooctene moiety according to structure (Z40) or (Z41) is not further substituted. Most preferably, Z is according to structure (Z43), defined further below.

In an alternative preferred embodiment, Z¹ comprises a heterocycloheptenyl group and is according to structure (Z37).

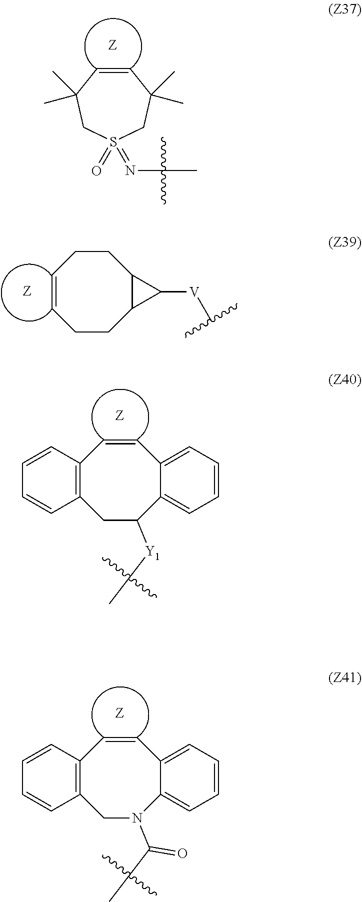

In an especially preferred embodiment, Z¹ comprises a cyclooctenyl group and is according to structure (Z42):

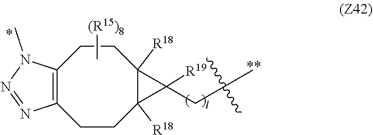

### Herein:

the bond labelled with * is connected to CB and the wavy bond labelled with ** is connected to L;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero) arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of Z (or Q) or D connected via a spacer moiety; and
I is an integer in the range 0 to 10.

In a preferred embodiment of the group according to structure (Z42), R¹⁵ is independently selected from the group consisting of hydrogen, halogen, —OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the group according to structure (Z42), R¹⁸ is independently selected from the group consisting of hydrogen, C₁-C₆ alkyl groups, most preferably both R¹⁸ are H. In a preferred embodiment of the group according to structure (Z42), R¹⁹ is H. In a preferred embodiment of the group according to structure (Z42), I is 0 or 1, more preferably I is 1.

In an especially preferred embodiment, Z¹ comprises a (hetero)cyclooctenyl group and is according to structure (Z⁴³):

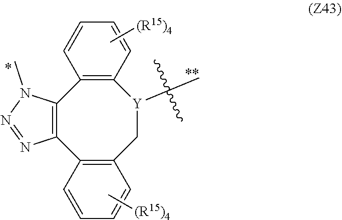

### Herein:

the bond labelled with * is connected to CB and the wavy bond labelled with ** is connected to L;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero) arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR 15.

In a preferred embodiment of the group according to structure (Z⁴³), R¹⁵ is independently selected from the group consisting of hydrogen, halogen, —OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁽⁻⁾. In a preferred embodiment of the group according to structure (Z43), Y is N or CH, more preferably Y=N.

In an especially preferred embodiment, Z¹ comprises a heterocycloheptenyl group and is according to structure (Z³⁷), wherein ring Z is a triazole.

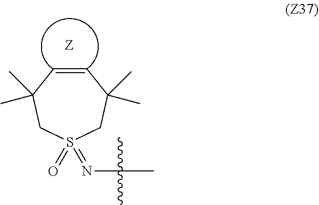

In an alternative preferred embodiment, Z¹ comprises a (hetero)cycloalkane moiety, i.e. the bond depicted as [symbol] is a single bond. The (hetero)cycloalkane group may also be referred to as a heterocycloalkyl group or a cycloalkyl group, preferably a cycloalkyl group, wherein the (hetero)cycloalkyl group is optionally substituted. Preferably, the (hetero)cycloalkyl group is a (hetero)cyclopropyl group, a (hetero)cyclobutyl group, a norbornyl group, a norbornenyl group, a (hetero)cycloheptyl group, a (hetero)cyclooctyl group, which may all optionally be substituted. Especially preferred are (hetero)cyclopropyl groups, (hetero)cycloheptyl group or (hetero)cyclooctyl groups, wherein the (hetero)cyclopropyl group, the (hetero)cycloheptyl group or the (hetero)cyclooctyl group is optionally substituted. Preferably, Z¹ comprises a cyclopropyl moiety according to structure (Z44), a hetereocyclobutane moiety according to structure (Z45), a norbornane or norbornene group according to structure (Z46), a (hetero)cycloheptyl moiety according to structure (Z47) or a (hetero)cyclooctyl moiety according to structure (Z48). Herein, Y³ is selected from C(R²³)₂, NR²³ or O, wherein each R²³ is individually hydrogen, C₁-C₆ alkyl or is connected to L, optionally via a spacer, and the bond labelled [symbol] is a single or double bond. In a further preferred embodiment, the cyclopropyl group is according to structure (Z49). In another preferred embodiment, the (hetero)cycloheptane group is according to structure (Z50) or (Z51). In another preferred embodiment, the (hetero)cyclooctane group is according to structure (Z52), (Z53), (Z54), (Z55) or (Z56).

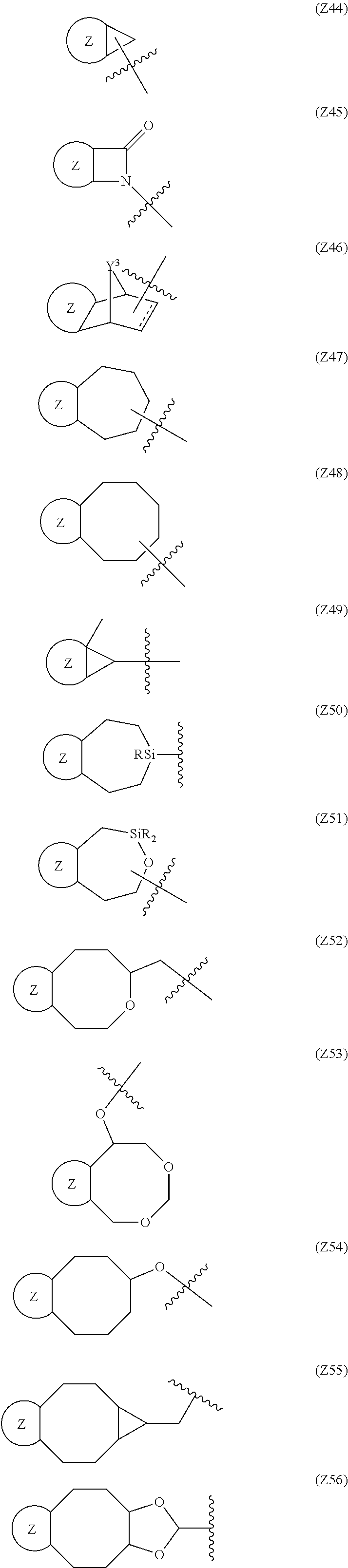

Herein, the R group(s) on Si in (Z50) and (Z51) are typically alkyl or aryl, preferably C₁-C₆ alkyl. Ring Z is selected from structures (Zk)-(Zn), wherein the carbon atoms labelled with ** correspond to the two carbon atoms of the (hetero)cycloalkane ring of (Z44)-(Z56) to which ring Z is fused, and the carbon a carbon labelled with * is connected to CB. Since the connecting group Z¹ is formed by reaction with a (hetero)cycloalkene in the context of the present embodiment, the bound depicted above as [symbol] is a single bond.

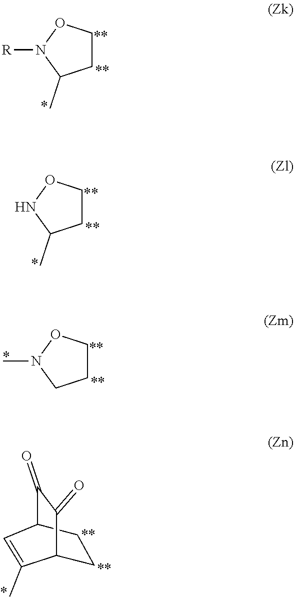

In a second preferred embodiment, Z¹ is formed by a nucleophilic reaction, preferably by a nucleophilic substitution or a Michael addition, preferably by a Michael addition. A preferred Michael reaction is the thiol-maleimide ligation, most preferably wherein Q is maleimide and F is a thiol group, wherein the thiol may be part of a disulphide bridge. Preferably, the thiol is present in the sidechain of a cysteine residue. Such a conjugation reaction with a thiol may also be referred to as thiol alkylation or thiol arylation. In a preferred embodiment, connection group Z¹ comprises a succinimidyl ring or its ring-opened succinic acid amide derivative, which may be formed by hydrolysis of the succinimidyl ring.

Alternatively, Z¹ is formed by nucleophilic reaction at the amino group in the sidechain of a lysine residue (F), which may react with amino reactive groups Q. Such a conjugation reaction with a thiol may also be referred to as amide bond formation or carbamate bond formation. Typical amino reactive groups Q include N-hydroxysuccinimidyl (NHS) esters, p-nitrophenyl carbonates, pentafluorophenyl carbonates, isocyanates, isothiocyanates and benzoyl halides.

Preferred options for connection group Z¹ comprise a moiety selected from (Z57)-(Z71) depicted here below

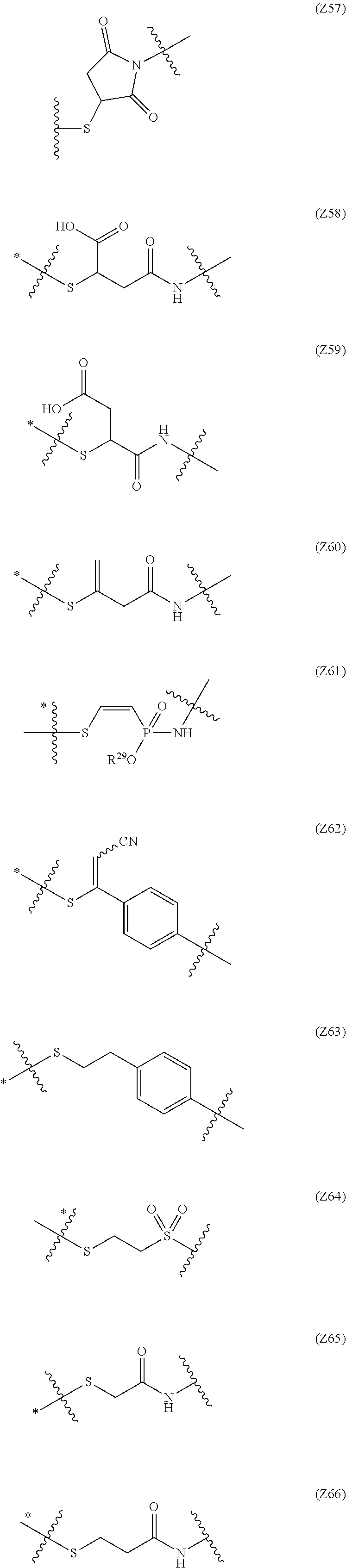

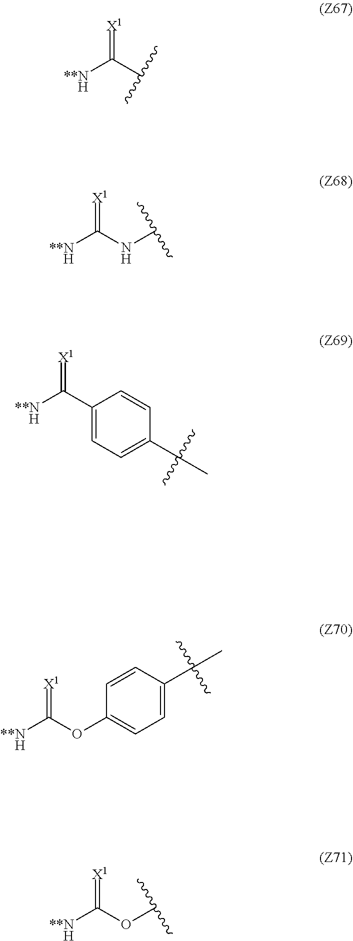

Herein, the wavy bond(s) labelled with an * in (Z57)-(Z66) is connected to CB, and the wavy bond without label to the payload via linker L. In addition, R²⁹ is C₁₋₁₂ alkyl or 1-24 polyethyleneglycol units, preferably C₁₋₄ alkyl or 6-14 polyethyleneglycol units, most preferably ethyl or 12 polyethylene glycol units, and X¹ is O or S, preferably X¹═O. Alternatively, R²⁹ is C₁₋₁₂ alkyl, preferably C₁₋₄ alkyl, most preferably ethyl, and X¹ is O or S, preferably X¹═O. The nitrogen atom labelled with ** in (Z67)-(Z71) corresponds to the nitrogen atom of the side chain of a lysine residue of the antibody, and the wavy bond without label to the payload via linker L. The carbon atoms of the phenyl group of (Z69) and (Z70) are optionally substituted, preferably optionally fluorinated.

In a preferred embodiment, connection group Z¹ comprise a moiety selected from (Z1)-(Z71).

### Linker L

Linker L connects payload D, via connecting group Z², with connecting group Z¹ (in the conjugates according to the invention) or connects payload D with reactive group Q (in the linker-toxin constructs). Linkers are known in the art and may be cleavable or non-cleavable. Linker L preferably contains a self-immolative group or cleavable linker, comprising a peptide spacer and optionally a para-aminobenzyloxycarbonyl (PABC) moiety or derivative thereof.

In a preferred embodiment, linker L as the structure -(L¹)ₙ-(L²)ₒ-(L³)ₚ-, wherein (L³)ₚ is connect to payload D, via connecting group Z², and (L¹) n is connected to Z¹ or Q. Herein L¹, L² and L³ are linkers or linking units and each of n, o and p are individually 0 or 1, wherein n+o+p is at least 1. In a preferred embodiment, at least linkers L¹ and L² are present (i.e. n=1; o=1; p=0 or 1), more preferably linkers L¹, L² and L³ are present (i.e. n=1; o=1; p=1).

Thus, it is preferred that in the conjugate according to the invention L-Z² has the following structure:

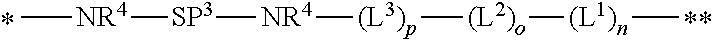

wherein:
the bond labelled with * is connected to the compound according to structure (1);
the bond labelled with ** is connected to connecting group Z¹;
—Sp³ is a is C₁₋₁₂ (hetero)alkylene, (hetero)arylene, C₁₋₁₂ (hetero)alkylene-(hetero)arylene, or (hetero)arylene-C₁₋₁₂ alkylene, wherein the alkylene or the (hetero)arylene may be optionally substituted with one or more substituents selected from halogen, X²R⁴, N(R⁴)₂, C₁₋₄ alkyl and NO₂, wherein the C₁₋₄ alkyl substituent may optionally form a cyclic structure by being joined with an NR⁴ moiety, in particular in a pyrrolidine formed with the NR⁴ moiety with the bond labelled with *, and the alkylene may optionally be interrupted with one or more heteroatoms selected from X² and NR⁴;
R⁴ and X² are as defined in claim 1;
L¹, L² and L³ are each individually linkers that together link Z¹ to D;
n, o and p are each individually 0 or 1, provided that n+o+p=1, 2 or 3.

A linker, especially linker L¹, may contain one or more branch-points for attachment of multiple payloads to a single connecting group. In a preferred embodiment, the linker of the conjugate according to the invention contains a branching moiety. A “branching moiety” in the context of the present invention refers to a moiety that is embedded in a linker connecting three moieties. In other words, the branching moiety comprises at least three bonds to other moieties, typically one bond connecting to Z¹ or Q, one bond to the payload D and one bond to a second payload D. The branching moiety, if present, is preferably embedded in linker L¹, more preferably part of Sp³ or as the nitrogen atom of NR¹³. Any moiety that contains at least three bonds to other moieties is suitable as branching moiety in the context of the present invention. In a preferred embodiment, the branching moiety is selected from a carbon atom, a nitrogen atom, a phosphorus atom, a (hetero) aromatic ring, a (hetero) cycle or a polycyclic moiety. Most preferably, the branching moiety is a nitrogen atom.

### Linker L¹

Linker L¹ is either absent (n=0) or present (n=1). Preferably, linker L¹ is present and n=1. L¹ may for example be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{y′} and NR²¹, wherein y′ is 0, 1 or 2, preferably y′=2, and R²¹ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups.

In a preferred embodiment, linker L¹ contains a polar group. Such a polar group may be selected from (poly)ethylene glycol diamines (e.g. 1,8-diamino-3,6-dioxaoctane or equivalents comprising longer ethylene glycol chains), (poly)ethylene glycol or (poly)ethylene oxide chains, (poly)propylene glycol or (poly)propylene oxide chains and 1,z′-diaminoalkanes (wherein z′ is the number of carbon atoms in the alkane, preferably z′=1-10), —(O)ₐ—C(O)—NH—S(O)₂—NR¹³— (as further defined below, see structure (23)), —C(S(O)₃⁽⁻⁾)—, —C(C(O)₂⁽⁻⁾)—, —S(O)₂—, —P(O)₂⁽⁻⁾—, —O(CH₂CH₂O)ₜ—, —NR³⁰(CH₂CH₂NR³⁰)ₜ—, and the following two structures:

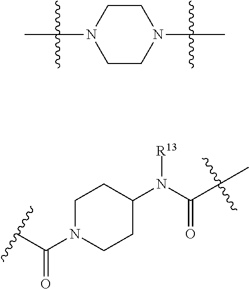

For the polar groups defined here above, it is irrelevant which end is connected to Z¹ and which end to (L²)ₒ.

The polar group may also contain an amino acid, preferably selected from Arg, Glu, Asp, Ser and Thr. Herein, a and R¹³ are further defined below for structure (23). t is an integer in the range of integer in the range of 0-15, preferably 1-10, more preferably 2-5, most preferably t=2 or 4. Each R³⁰ is individually H, C₁₋₁₂ alkyl, C₁₋₁₂ aryl, C₁₋₁₂ alkaryl or C₁₋₁₂ aralkyl. Linker L¹ may contain more than one such polar group, such as at least two polar groups. The polar group may also be present in a branch of linker L¹, which branches off a branching moiety as defined elsewhere. Preferable, a nitrogen or carbon atom is used as branching moiety. It is especially preferred to have a —O(CH₂CH₂O)ₜ— polar group present in a branch.

In a preferred embodiment, Linker L¹ is or comprises a sulfamide group, preferably a sulfamide group according to structure (23):

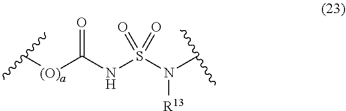

The wavy lines represent the connection to the remainder of the compound, typically to Q and L², L³ or D, preferably to Q and L². Preferably, the (O)ₐC(O) moiety is connected to Q and the NR¹³ moiety to L², L³ or D, preferably to L².

In structure (23), a=0 or 1, preferably a=1, and R₁₃ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups. Alternatively, R¹³ is D connected to N optionally via a spacer moiety, preferably via Sp² as defined below, in one embodiment D is connected to N via-(B)ₑ-(A)_{f}-(B) g-C(O)—. Alternatively, R¹³ is connected to elsewhere in the linker, optionally via a spacer moiety, to form a cyclic structure. For example, R¹³ may be connected to the linker via a CH₂CH₂ spacer moiety to form a piperazinyl ring, where the connection to D is via the second nitrogen of the piperazinyl ring.

In a preferred embodiment, R¹³ is hydrogen, a C₁-C₂₀ alkyl group, preferably a C₁--C₁₆ alkyl group, more preferably a C₁-C₁₀ alkyl group, or connected to a further occurrence of D or to elsewhere in the linker optionally via a spacer moiety. Herein, the alkyl group is optionally substituted and optionally interrupted by one or more heteroatoms selected from O, S and NR¹⁴, preferably O, wherein R¹⁴ is independently selected from the group consisting of hydrogen and C₁-C₄ alkyl groups. In another preferred embodiment, R¹³ is a C₁-C₂₀ alkyl group, more preferably a C₁-C₁₆ alkyl group, even more preferably a C₁-C₁₀ alkyl group, wherein the alkyl group is optionally interrupted by one or more O-atoms, and wherein the alkyl group is optionally substituted with an-OH group, preferably a terminal-OH group. In this embodiment it is further preferred that R¹³ is a (poly)ethylene glycol chain comprising a terminal-OH group. In another preferred embodiment, R¹³ is selected from the group consisting of hydrogen, methyl, ethyl, n-propyl, i-propyl, n-butyl, s-butyl and t-butyl, or connected to a further occurrence of D or to elsewhere in the linker optionally via a spacer moiety, more preferably from the group consisting of hydrogen, methyl, ethyl, n-propyl and i-propyl, or connected to a further occurrence of D or to elsewhere in the linker optionally via a spacer moiety, and even more preferably from the group consisting of hydrogen, methyl and ethyl, or connected to a further occurrence of D or to elsewhere in the linker optionally via a spacer moiety. Yet even more preferably, R¹³ is hydrogen or connected to a further occurrence of D or to elsewhere in the linker optionally via a spacer moiety, and most preferably R¹³ is hydrogen.

In a preferred embodiment, L¹ is according to structure (24):

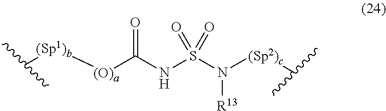

Herein, a and R¹³ are as defined above, Sp¹ and Sp² are independently spacer moieties and b and c are independently 0 or 1. Preferably, b=0 or 1 and c=1, more preferably b=0 and c=1. In one embodiment, spacers Sp¹ and Sp² are independently selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, spacer moieties Sp¹ and Sp², if present, are independently selected from the group consisting of linear or branched C₁-C₁₀₀ alkylene groups, C₂-C₁₀₀ alkenylene groups, C₂-C₁₀₀ alkynylene groups, C₃-C₁₀₀ cycloalkylene groups, C₅-C₁₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Even more preferably, spacer moieties Sp¹ and Sp², if present, are independently selected from the group consisting of linear or branched C₁-C₅₀ alkylene groups, C₂-C₅₀ alkenylene groups, C₂-C₅₀ alkynylene groups, C₃-C₅₀ cycloalkylene groups, C₅-C₅₀ cycloalkenylene groups, C₈-C₅₀ cycloalkynylene groups, C₇-C₅₀ alkylarylene groups, C₇-C₅₀ arylalkylene groups, C₈-C₅₀ arylalkenylene groups and C₉-C₅₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Yet even more preferably, spacer moieties Sp¹ and Sp², if present, are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR¹⁶, preferably O, wherein R¹⁶ is independently selected from the group consisting of hydrogen and C₁-C₄ alkyl groups, preferably hydrogen or methyl.

Most preferably, spacer moieties Sp¹ and Sp², if present, are independently selected from the group consisting of linear or branched C₁-C₂₀ alkylene groups, the alkylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR¹⁶, preferably O and/or S—S, wherein R¹⁶ is independently selected from the group consisting of hydrogen and C₁-C₄ alkyl groups, preferably hydrogen or methyl.

Preferred spacer moieties Sp¹ and Sp² thus include —(CH₂)ᵣ, —(CH₂CH₂)ᵣ—, —(CH₂CH₂O)ᵣ, —(OCH₂CH₂)ᵣ—, —(CH₂CH₂O)ᵣCH₂CH₂—, —CH₂CH₂ (OCH₂CH₂)ᵣ—, —(CH₂CH₂CH₂O)ᵣ—, —(OCH₂CH₂CH₂)ᵣ, —(CH₂CH₂CH₂O)CH₂CH₂CH₂-and-CH₂CH₂CH₂ (OCH₂CH₂CH₂)ᵣ—, wherein r is an integer in the range of 1 to 50, preferably in the range of 1 to 40, more preferably in the range of 1 to 30, even more preferably in the range of 1 to 20 and yet even more preferably in the range of 1 to 15. More preferably n is 1, 2, 3, 4, 5, 6, 7, 8, 9 or 10, more preferably 1, 2, 3, 4, 5, 6, 7 or 8, even more preferably 1, 2, 3, 4, 5 or 6, yet even more preferably 1, 2, 3 or 4.

Alternatively, preferred linkers L¹ may be represented by —(W)ₖ-(A)_{d}-(B)ₑ-(A)ᵣ-(C(O))_{g}—, wherein:
d=0 or 1, preferably d=1;
e=an integer in the range 0-10, preferably e=0, 1, 2, 3, 4, 5 or 6, preferably an integer in the range 1-10, most preferably e=1, 2, 3 or 4;
f=0 or 1, preferably f=0;
wherein d+e+f is at least 1, preferably in the range 1-5; and preferably wherein d+f is at least 1, preferably d+f=1.
g=0 or 1, preferably g=1;
k=0 or 1, preferably k=1;
A is a sulfamide group according to structure (23);
B is a —CH₂—CH₂—O— or a —O—CH₂—CH₂— moiety, or (B) e is a —(CH₂—CH₂—O)ₑ₁—CH₂—CH₂— or a —(CH₂—CH₂—O)ₑ₁—CH₂— moiety, wherein e1 is defined the same way as e;
W is —OC(O)—, —C(O)O—, —C(O)NH—, —NHC(O)—, —OC(O)NH—, —NHC(O)O—, —C(O)(CH₂)ₘC(O)—, —C(O)(CH₂)ₘC(O)NH— or -(4-Ph)CH₂NHC(O)(CH₂)ₘC(O)NH—, preferably wherein W is —OC(O)NH—, —C(O)(CH₂)ₘC(O)NH— or —C(O)NH—, and wherein m is an integer in the range 0-10, preferably m=0, 1, 2, 3, 4, 5 or 6, most preferably m=2 or 3;
preferably wherein L¹ is connected to Q via (W)ₖ and to L², L³ or D, preferably to L², via (C(O))_{g}, preferably via C(O).

In the context of the present embodiment, the wavy lines in structure (23) represent the connection to the adjacent groups such as (W)ₖ, (B)ₑ and (C(O))_{g}. It is preferred that A is according to structure (23), wherein a=1 and R¹³═H or a C₁-C₂₀ alkyl group, more preferably R¹³═H or methyl, most preferably R¹³═H.

Preferred linkers L¹ have structure —(W)ₖ-(A)_{d}-(B)ₑ-(A)_{f}-(C(O))_{g}—, wherein:
(a) k=0; d=1; g=1; f=0; B=—CH₂—CH₂—O—; e=1, 2, 3 or 4, preferably e=2.
(b) k=1; W=—C(O)(CH₂)ₘC(O)NH—; m=2; d=0; (B)ₑ=—(CH₂—CH₂—O)ₑ₁—CH₂—CH₂—; f=0; g=1; e1=1, 2, 3 or 4, preferably e=1.
(c) k=1; W=—OC(O)NH—; d=0; B=—CH₂—CH₂—O—; g=1; f=0; e=1, 2, 3 or 4, preferably e=2.
(d) k=1; W=—C(O)(CH₂)ₘC(O)NH—; m=2; d=0; (B)ₑ=—(CH₂—CH₂—O)ₑ₁—CH₂—CH₂—; f=0; g=1; e1=1, 2, 3 or 4, preferably e1=4.
(e) k=1; W=—OC(O)NH—; d=0; (B)ₑ=—(CH₂—CH₂—O)ₑ₁—CH₂—CH₂—; g=1; f=0; e1=1, 2, 3 or 4, preferably e1=4.
(f) k=1; W=-(4-Ph)CH₂NHC(O)(CH₂)ₘC(O)NH—, m=3; d=0; (B)ₑ=—(CH₂—CH₂—O)ₑ₁—CH₂—CH₂—; g=1; f=0; e1=1, 2, 3 or 4, preferably e1=4.
(g) k=0; d=0; g=1; f=0; B=—CH₂—CH₂—O—; e=1, 2, 3 or 4, preferably e=2.
(h) k=1; W=—C(O)NH—; d=0; g=1; f=0; B=—CH₂—CH₂—O—; e=1, 2, 3 or 4, preferably e=2.

Herein, it is preferred that when d and/or f=1, than a=1 and R¹³═H. Most preferred is the linker is structure (a).

In a preferred embodiment, linker L¹ comprises a branching nitrogen atom, which is located in the backbone between Q or Z and (L²)ₒ and which contains a further moiety D as substituent, which is preferably linked to the branching nitrogen atom via a linker. An example of a branching nitrogen atom is the nitrogen atom NR¹³ in structure (23), wherein R¹³ is connected to a second occurrence of D via a spacer moiety. Alternatively, a branching nitrogen atoms may be located within L¹ according to structure —(W)ₖ-(A)_{d}-(B)ₑ-(A)_{f}-(C(O))_{g}—. In one embodiment, L¹ is represented by —(W)ₖ-(A)_{d}-(B)ₑ-(A)_{f}-(C(O))_{g}—N*[-(A)_{d}-(B)ₑ-(A)_{f}-(C(O))_{g}—]₂, wherein A, B, W, d, e, f, g and k are as defined above and individually selected for each occurrence, and N* is the branching nitrogen atoms, to which two instances of -(A)_{d}-(B)ₑ-(A)_{f}-(C(O))_{g}— are connected. Herein, both (C(O))_{g} moieties are connected to -(L²)ₒ-(L³)ₚ-D, wherein L², L³, o, p and D are as defined above and are each selected individually. In a preferred embodiment, each of L², L³, o, p and D are the same for both moieties connected to (C(O))_{g}.

Preferred linkers L¹ comprising a branching nitrogen atom have structure —(W)ₖ-(A)_{d}-(B)ₑ-(A)_{f}-(C(O))_{g}—N*[-(A′)_{d′}-(B′)_{e′}-(A′)_{f′}-(C(O))_{g′}-]₂ wherein:
(i) k=d=g=e′=1; f=d′=g′=0; W=—C(O)—; B=B′=—CH₂—CH₂—O—; A is according to structure (23) with a=0 and R¹³═H; e=1, 2, 3 or 4, preferably e=2.
(j) k=d=g=e′=g′=1; f=d′=0; W=—C(O)—; B=B′=—CH₂—CH₂—O—; A is according to structure (23) with a=0 and R¹³═H; e=1, 2, 3 or 4, preferably e=2.

### Linker L²

Linker L² is a peptide spacer. Linker L² is either absent (o=0) or present (o=1). Preferably, linker L² is present and o=1. The combination of a peptide spacer L² and a cleavable linker L³ is well-known in the art. However, in the conjugates according to the present invention the presence of L³ is not essential, since the same motive may be present in the connection with payload D, in particular within R¹ or Y. For example, In case the conjugate comprises the motive CH₂-Ph-NH-L², wherein CH₂-Ph-NH is formed by R¹, the presence of an additional para-aminobenzyl moiety of L³ is not needed for the linker to be self-immolative. Thus, in one preferred embodiment, L³ is absent and L² is directly bonded to D, preferably via R¹ or Y.

The peptide spacer may also be defined by (NH—CR¹⁷—CO)ₙ, wherein R¹⁷ represents an amino acid side chain as known in the art. Herein, the amino acid may be a natural or a synthetic amino acid. Examples of preferred synthetic amino acids are citrulline and cysteic acid. Preferably, the amino acid(s) are all in their L-configuration. n is an integer in the range of 1-5, preferably in the range of 2-4. Thus, the peptide spacer contains 1-5 amino acids. Preferably, the peptide is a dipeptide (n=2), tripeptide (n=3) or tetrapeptide (n=4), most preferably the peptide spacer is a dipeptide. Although any peptide spacer may be used, preferably the peptide spacer is selected from Val-Cit, Val-Ala, Val-Lys, Val-Arg, AcLys-Val-Cit, AcLys-Val-Ala, Glu-Val-Ala, Asp-Val-Ala, iGlu-Val-Ala, Glu-Val-Cit, Asp-Val-Cit, iGlu-Val-Cit, Phe-Cit, Phe-Ala, Phe-Lys, Phe-Arg, Ala-Lys, Leu-Cit, Ile-Cit, Trp-Cit, Ala-Ala-Asn, Ala-Asn, Phe-Phe, Gly, Gly-Gly, Gly-Gly-Gly, Gly-Gly-Gly-Gly, Leu-Gly, Tyr-Gly, Ala-Gly, Pro-Gly, Phe-Gly, Phe-Gly, Ser-Gly, Gly-Phe-Gly, Gly-Gly-Phe-Gly, Gly-Phe-Gly-Gly, Phe-Gly-Gly-Gly, Gly-Gly-Gly-Phe, Phe-Phe-Gly-Gly, Gly-Gly-Phe-Phe, Gly-Gly-Gly-Phe-Gly and Lys, more preferably Val-Cit, Val-Ala, Glu-Val-Ala, Val-Lys, Phe-Cit, Phe-Ala, Phe-Lys, Ala-Ala-Asn, more preferably Val-Cit, Val-Ala, Ala-Ala-Asn, most preferably Val-Cit or Val-Ala. Herein, AcLys is ε-N-acetyllysine and iGlu is isoglutamate. In one embodiment, L²=Val-Cit. In one embodiment, L²=Val-Ala.

R¹⁷ represents the amino acid side chain, preferably selected from the side chains of alanine, cysteine, aspartic acid, glutamic acid, phenylalanine, glycine, histidine, isoleucine, lysine, acetyllysine, leucine, methionine, asparagine, pyrrolysine, proline, glutamine, arginine, serine, threonine, selenocysteine, valine, tryptophan, tyrosine and citrulline. Preferred amino acid side chains are those of Val, Cit, Ala, Lys, Arg, AcLys, Phe, Leu, Ile, Trp, Glu, Asp and Asn, more preferably from the side chains of Val, Cit, Ala, Glu and Lys. Alternatively worded, R¹⁷ is preferably selected from CH₃ (Ala), CH₂CH(CH₃)₂ (Leu), CH₂CH₂CH₂NHC(O)NH₂ (Cit), CH₂CH₂CH₂CH₂NH₂ (Lys), CH₂CH₂CH₂NHC(O)CH₃ (AcLys), CH₂CH₂CH₂NHC(═NH)NH₂ (Arg), CH₂Ph (Phe), CH(CH₃)₂ (Val), CH(CH₃)CH₂CH₃ (IIe), CH₂C(O)NH₂ (Asn), CH₂CH₂C(O)OH (Glu), CH₂C(O)OH (Asp) and CH₂(1H-indol-3-yl) (Trp). Especially preferred embodiments of R¹⁷ are CH₃ (Ala), CH₂CH₂CH₂NHC(O)NH₂ (Cit), CH₂CH₂CH₂CH₂NH₂ (Lys), CH₂CH₂C(O)OH (Glu) and CH(CH₃)₂ (Val). Most preferably, R¹⁷ is CH₃ (Ala), CH₂CH₂CH₂NHC(O)NH₂ (Cit), CH₂CH₂CH₂CH₂NH₂ (Lys), or CH(CH₃)₂ (Val).

In an especially preferred embodiment, the peptide spacer may be represented by general structure (25):

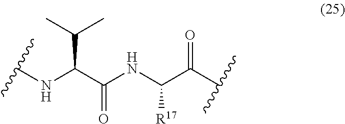

Herein, R¹⁷ is as defined above, preferably R¹⁷ is CH₃ (Ala) or CH₂CH₂CH₂NHC(O)NH₂ (Cit). The wavy lines indicate the connection to (L¹)ₙ and (L³)ₚ, preferably L² according to structure (25) is connected to (L¹)ₙ via NH and to (L³)ₚ via C(O).

### Linker L³

Linker L³ is a self-cleavable spacer, also referred to as self-immolative spacer. Linker L³ is either absent (p=0) or present (p=1). Preferably, linker L³ is present and p=1. However, in case the payload is connected to the linker through Z² being NH, which is connected to the C(O) terminus of the peptide spacer of L² and wherein R¹ or Y (depending on the location of the attachment point to the compound of structure (1)) contain an aromatic ring as defined here below for ring A, than it is preferred that L³ is absent and p=0. The nature of L², Z² and Y or R¹ ensure that the linker L is self-cleavable even without the presence of L³.

Preferably, L³ is para-aminobenzyloxycarbonyl (PABC) derivative, more preferably a PABC derivative according to structure (26):

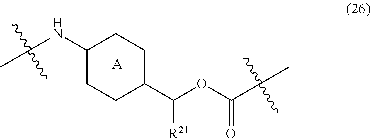

Herein, the wavy lines indicate the connection to Q or Z¹, L¹ or L², and to Z². Typically, the PABC derivative is connected via NH to Q, Z¹, L¹ or L², preferably to L², and via OC(O) to Z².

Ring A is a 5- or 6-membered aromatic or heteroaromatic ring, preferably a 6-membered aromatic or heteroaromatic ring. Suitable 5-membered rings are oxazole, thiazole and furan. Suitable 6-membered rings are phenyl and pyridyl. Ring A may be substituted with a substituent selected from halogen, X²R⁴, N(R⁴)₂, C₁₋₄ alkyl and NO₂. Herein, X² and R⁴ are as defined above, including preferred embodiments thereof. In a preferred embodiment, the optional substituent is selected from F, Cl, Br, OH, OR⁴, SH, NH₂, Et, Me and NO₂. In an especially preferred embodiment, ring A comprises 0-2 substituents, more preferably 0 or 1 substituent, most preferably ring A is not substituted. In a preferred embodiment, ring A is 1,4-phenyl, 1,2-phenyl, 2,5-pyridyl or 3,6-pyridyl. Most preferably, A is 1,4-phenyl.

R²¹ is selected from H, R²⁶, C(O)OH and C(O)R²⁶, wherein R²⁶ is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which are optionally substituted and optionally interrupted by one or more heteroatoms selected from O, S and NR²⁸ wherein R²⁸ is independently selected from the group consisting of hydrogen and C₁-C₄ alkyl groups. Preferably, R²⁶ is C₃-C₁₀ (hetero)cycloalkyl or polyalkylene glycol. The polyalkylene glycol is preferably a polyethylene glycol or a polypropylene glycol, more preferably —(CH₂CH₂O)ₛH or —(CH₂CH₂CH₂O)ₛH. The polyalkylene glycol is most preferably a polyethylene glycol, preferably —(CH₂CH₂O)ₛH, wherein s is an integer in the range 1-10, preferably 1-5, most preferably s=1, 2, 3 or 4. More preferably, R²¹ is H or C(O)R²⁶, wherein R²⁶=4-methyl-piperazine or morpholine. Most preferably, R²¹ is H.

### Connecting Group Z² and Payload D

D, also referred to in the art as the “payload”, represents the compound that is or is to be connected to the CB. D is the compound according to structure (1), as well as preferred embodiments thereof defined above and below.

The conjugates according to the invention may comprise more than one payload D. When more than one payload is present the payloads D may be the same or different, typically they are the same. In the context of the present invention, at least one payload should be the compound according to structure (1). In a preferred embodiment, the conjugate contains 2 or 4 occurrences of D, most preferably 2 occurrence of D. A second occurrence of D may be present within linker L, which may contain a branching moiety, typically a branching nitrogen atom, that is connected to the second occurrence of D. Preferably, both occurrences of D are connected to the branching moiety via the same linker. Likewise, the conjugates according to the invention may contain more than one payload per connecting group Z¹.

The payload is connected to linker L via a connecting group Z², which is formed by reaction of the compound according to structure (1) with a linker unit. Thus, a reactive moiety of the compound according to structure (1) is reacted with a reactive moiety of the linker. The nature of Z² thus depends on the nature of the reactive moieties and the type of reaction that is performed to connect the linker to the payload, and may take any form. Preferably, connecting group Z² is selected from the group consisting of an amide moiety, an ester moiety, a carbamate moiety, a carbonate moiety or a (hetero)aryl moiety, more preferably an amide moiety or a carbamate moiety. In case the payload is connected through Y, connecting group Z² is most preferably an amide moiety. In case the payload is connected through R¹, connecting group Z² is most preferably a carbamate moiety.

Conveniently, the reactive moiety of the compound according to structure (1) is an amine moiety in R¹, in particular when R¹=Sp—N(R⁴)₂, or in Y, in particular when Y=N(R⁴)₂ or NR⁴—Sp³—N(R⁴)₂. Herein, one R⁴ group is replaced by the connection to L. As such the remaining NR⁴ residue is part of Z² that is formed when the amine group is reacted with the linker, typically thus forming an amide moiety or a carbamate moiety according to structure —(O)_{a′}—C(O)—NR⁴—, wherein a′=0 or 1. Alternatively, in case R¹ comprises an azide moiety, a cycloaddition reaction can be performed to create connecting group Z². In this embodiment, Z² comprises a (hetero)aryl moiety, and is as defined for connection group Z¹ above as far as it concerns a cycloaddition reaction with an azide moiety.

In the context of the present invention, the connection between the compound according to structure (1) and linker L is preferably through R¹ or through Y.

Thus, in a preferred embodiment, the compound according to structure (1) is connected to linker L through Y. The inventors found that by conjugating the compound according to structure (1) through Y, substituent R¹ is available for modulating or improving the efficacy of the toxin and as such of the conjugate as a whole. Hence, it is preferred that in the compound according to structure (1) is connected to conjugate according to the invention through Y. The connection through Y is especially preferred in case R¹ is selected from optionally substituted Et, i-Pr, n-Pr, t-Bu, i-Bu, n-Bu, n-pentyl, C₆₋₁₂ alkyl, (hetero)aryl, Bn, Sp-(hetero)aryl and Sp—X²R⁴. In an especially preferred embodiment, the connection to the compound according to structure (1) is through Y and R¹ is selected from (D1)-(D52) as defined above, more preferably from optionally substituted i-Pr, n-Pr, t-Bu, i-Bu, n-Bu, n-pentyl, C₆₋₁₂ alkyl, (hetero)aryl, Bn, Sp-(hetero)aryl, preferably from i-Pr, n-Pr, t-Bu, i-Bu, n-Bu and Bn, more preferably from i-Pr and Bn.

In an alternative embodiment, the compound according to structure (1) is connected to linker L through R¹. Such connection through R¹ is especially preferred in case the compound according to structure (1) is according to one of the preferred embodiments as identified above. The connection through R¹ is especially preferred in case R¹ contains a reactive moiety that is suitable for connection to linker L, such as a N₃, NH₂ or OH moiety. In an especially preferred embodiment, the connection to the compound according to structure (1) is through R¹ and R¹ is selected from (D10)-(D15), (D18)-(D26), (D31-(D37), (D41)-(D44), (D48) and (D53)-(D61), as defined above, more preferably R¹ is Sp—X²R⁴, Sp—N₃ or Sp—N(R⁴)₂, most preferably Sp—N₃ or Sp—N(R⁴)₂.

In an especially preferred embodiment, the conjugate according to the invention comprises a payload D with containing a moiety R¹ selected from optionally substituted Et, i-Pr, n-Pr, t-Bu, i-Bu, n-Bu, n-pentyl, C₆₋₁₂ alkyl, (hetero)aryl, Bn, Sp-(hetero)aryl, Sp—OR⁴, Sp—N₃ and Sp—N(R⁴)₂. Preferably, R¹ selected from optionally substituted i-Pr, n-Pr, t-Bu, i-Bu, n-Bu, aryl, Bn, Sp—N₃ and Sp—N(R⁴)₂. Herein, the optional substituents, Sp, X² and R⁴ are as defined above, including preferred embodiments thereof. In the context of the present embodiment, it is preferred that R¹ is selected from optionally substituted i-Pr, n-Pr, t-Bu, i-Bu, n-Bu, n-pentyl, C₆₋₁₂ alkyl, (hetero)aryl, Bn, Sp-(hetero)aryl, Sp—OR⁴, Sp—N₃ and Sp—N(R⁴)₂, more preferably from i-Pr, t-Bu, Bn, Sp—N₃ or Sp—NH₂, wherein Sp is C₁₋₄ alkylene or C₁₋₄ alkylene-arylene. More preferably, R¹ is i-Pr, Bn or Sp—N₃, wherein Sp is CH₂CH₂, CH₂CH₂CH₂ or CH₂(Ph). Herein, CH₂(Ph) may be CH₂(2-Ph), CH₂(3-Ph) or CH₂(4-Ph), preferably it is CH₂(4-Ph). In one especially preferred embodiment, R¹ is i-Pr, Bn, CH₂CH₂N₃, CH₂CH₂CH₂N₃ or CH₂((4-N₃)Ph).

The inventors have obtained especially beneficial results with these compounds according to structure (1) in terms of improved efficacy. In these conjugates, the compounds according to structure (1) may be connected through R¹ or Y. In case R¹ is selected from i-Pr, t-Bu, Bn, Sp—N₃ or Sp—NH₂, wherein Sp is C₁₋₄ alkylene or C₁₋₄ alkylene-arylene. More preferably, R¹ is i-Pr, Bn or Sp—N₃, wherein Sp is CH₂CH₂ or CH₂(4-Ph).

### Preferred Linkers

According to a preferred embodiment, the linking part of the conjugates according to the invention, as represented by L-Z² has a structure selected from (L¹)-(L³):

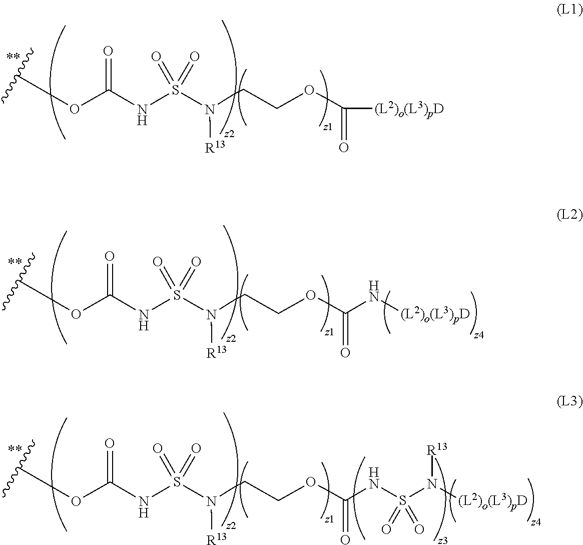

Herein:
the bond labelled with ** is connected to connecting group Z¹;
each R¹³ is individually selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, wherein the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups are optionally substituted and optionally interrupted by one or more heteroatoms selected from O, S and NR¹⁴, wherein R¹⁴ is independently selected from the group consisting of hydrogen and C₁-C₄ alkyl groups, or R¹³ is D connected to N via a spacer moiety, R¹³=hydrogen or D connected to N via a spacer moiety, preferably wherein the spacer moiety is —(B)ₑ-(A)_{f}-(B)_{g}—C(O)— as defined above;
L² is as defined above, preferably L² is a dipeptide, a tripeptide or a tetrapeptide;
o is 0 or 1, preferably o is 1;
L³ is as defined above;
p is 0 or 1;
z1 is an integer in the range of 1-4;
z2 is 0 or 1, preferably z2=1;
z3 is 0 or 1, preferably z3=0;
z4 is 1 or 2, preferably z4=1.

According to an especially preferred embodiment, the linking part of the conjugates according to the invention, as represented by L-Z² has a structure selected from (L4)-(L7):

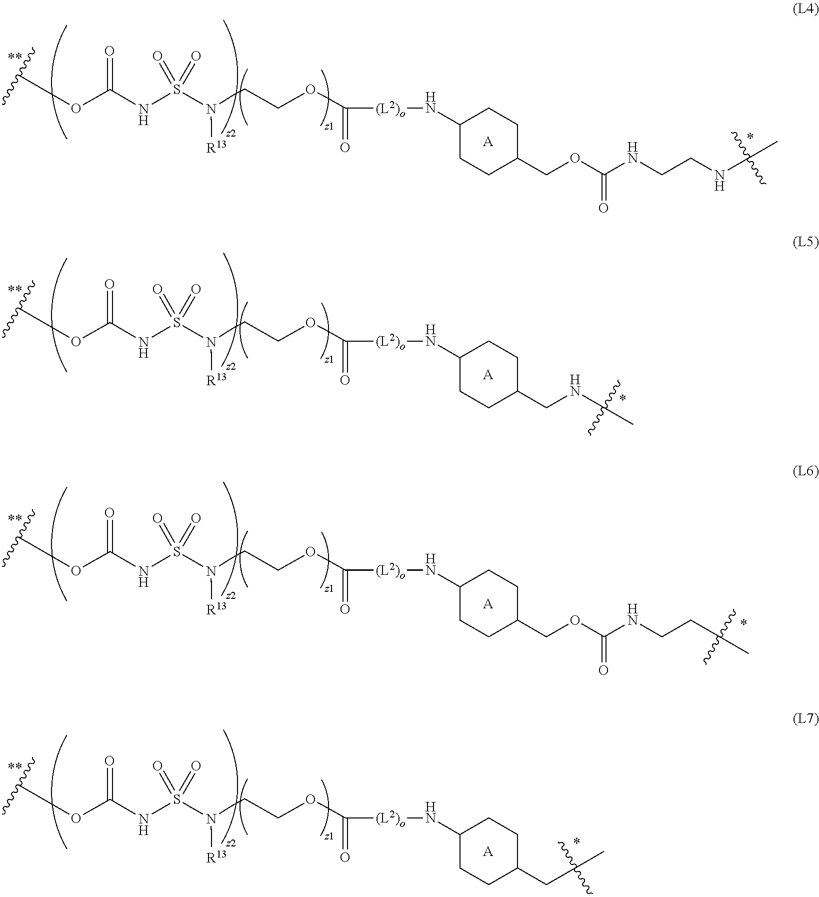

Herein:
the bond labelled with * is connected:
(a) for (L³) and (L⁴) to the C(O) group of moiety Y of the compound according to structure (1), and
(b) for (L⁶) and (L⁷) to the O atom of the OR¹ moiety directly bonded to the morpholine ring of the compound according to structure (1);
the bond labelled with ** is connected to connecting group Z¹;
R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, wherein the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups are optionally substituted and optionally interrupted by one or more heteroatoms selected from O, S and NR¹⁴, wherein R¹⁴ is independently selected from the group consisting of hydrogen and C₁-C₄ alkyl groups, or R¹³ is D connected to N via a spacer moiety, R¹³=hydrogen or D connected to N via a spacer moiety, preferably wherein the spacer moiety is —(B)ₑ-(A)_{f}-(B)_{g}—C(O)— as defined above;
L² is as defined above, preferably L² is a dipeptide, a tripeptide or a tetrapeptide;
o is 0 or 1, preferably o is 1;
ring A is an optionally substituted 5- or 6-membered aromatic or heteroaromatic ring, preferably a 6-membered aromatic or heteroaromatic ring, preferably A is 1,4-phenyl or 1,3-phenyl, most preferably A is 1,4-phenyl;
z1 is an integer in the range of 1-4;
z2 is 0 or 1, preferably z2=1.

An especially preferred combination involves A=1,4-phenyl, R¹³=hydrogen or D connected to N via a spacer moiety, o=1, L² is a dipeptide, z2=1 and z1=2.

According to an especially preferred embodiment, the linking part of the conjugates according to the invention, as represented by L-Z² has a structure selected from (L8)-(L11):

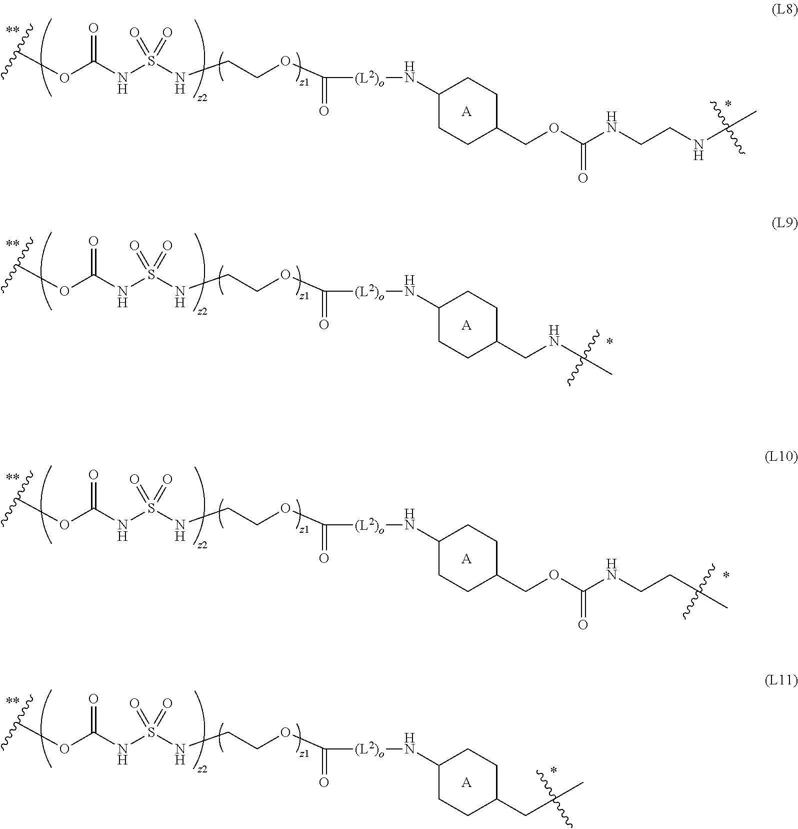

Herein:
the bond labelled with * is connected:
(a) for (L8) and (L9) to the C(O) group of moiety Y of the compound according to structure (1), and
(b) for (L10) and (L11) to the O atom of the OR¹ moiety directly bonded to the morpholine ring of the compound according to structure (1);
the bond labelled with ** is connected to connecting group Z¹;
L² is as defined above, preferably L² is a dipeptide, a tripeptide, or a tetrapeptide;
o is 0 or 1, preferably o is 1;
ring A is an optionally substituted 5- or 6-membered aromatic or heteroaromatic ring, preferably a 6-membered aromatic or heteroaromatic ring, preferably A is 1,4-phenyl or 1,3-phenyl, most preferably A is 1,4-phenyl;
z1 is an integer in the range of 1-4;
z2 is 0 or 1, preferably z2=1.

An especially preferred combination involves A=1,4-phenyl, o=1, L² is a dipeptide, z2=1 and z1=2.

According to an especially preferred embodiment, the linking part of the conjugates according to the invention, as represented by L-Z² has a structure selected from (L¹²)-(L¹⁵):

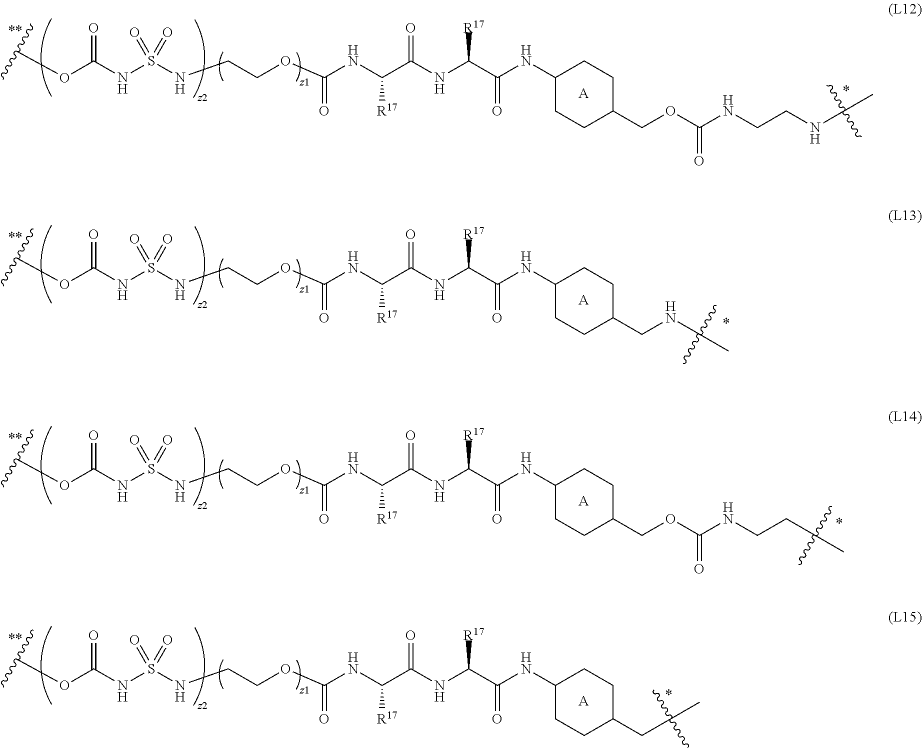

Herein:
the bond labelled with * is connected:
(a) for (L12) and (L13) to the C(O) group of moiety Y of the compound according to structure (1), and
(b) for (L14) and (L15) to the O atom of the OR¹ moiety directly bonded to the morpholine ring of the compound according to structure (1);
the bond labelled with ** is connected to connecting group Z¹;
each R¹⁷ is individually an amino acid side chain, preferably i-Pr, CH₃ or CH₂CH₂CH₂NHC(O)NH₂;
ring A is an optionally substituted 5- or 6-membered aromatic or heteroaromatic ring, preferably a 6-membered aromatic or heteroaromatic ring, preferably A is 1,4-phenyl or 1,3-phenyl, most preferably A is 1,4-phenyl;
z1 is an integer in the range of 1-4;
z2 is 0 or 1, preferably z2=1.

An especially preferred combination involves A=1,4-phenyl, R¹⁷=i-Pr, CH₃ or CH₂CH₂CH₂NHC(O)NH₂, z2=1 and z1=2.

In another preferred embodiment, the linker is a non-cleavable linker according to structures (L16)-(L20):

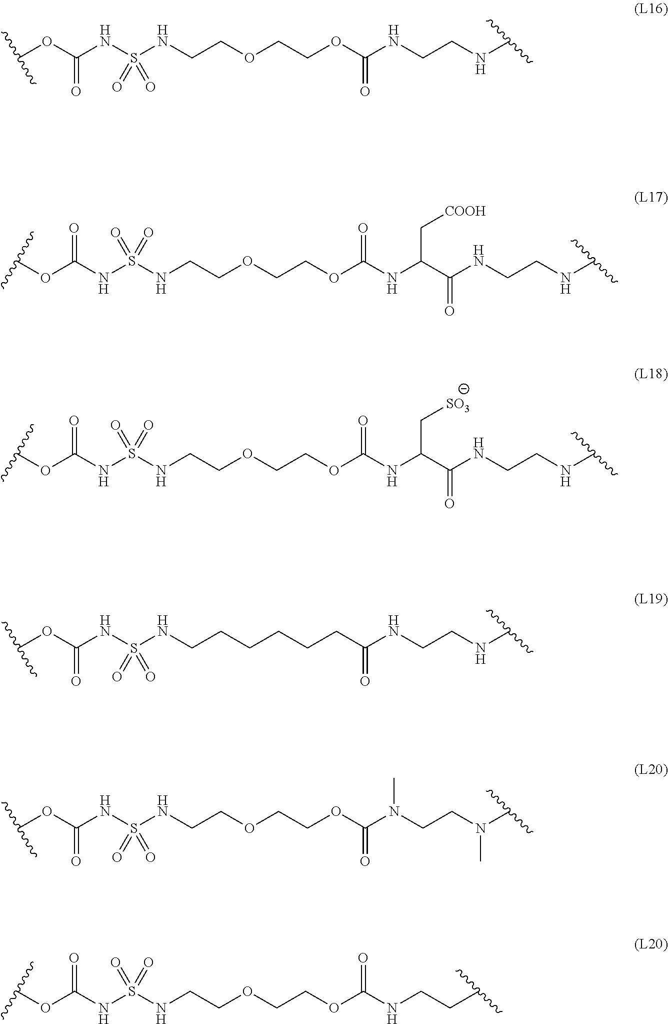

### Preferred Conjugates

Preferred antibody-conjugates according to the first aspect are selected from the group consisting of compounds (I)-(II), more preferably (II). More preferred conjugates are selected from (III)-(V). Even more preferred conjugates are selected from (X)-(XVII). In one especially preferred embodiment, the conjugates is selected from (Xb) and (XIb). The structures of these conjugates are defined here below.

Conjugate (I) has the following structure:

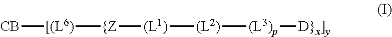

wherein:
CB, L⁶, Z, D, x and y are as defined above;
L¹ is a linker represented by -(A)_{d}-(B)ₑ-(A)_{f}-(C(O))_{g}—, as defined above;
L² is a peptide spacer as defined above, preferably Val-Cit or Val-Ala;
L³ is the PABC derivative according to structure (26);
p=0 or 1.

In the context of antibody-conjugate (I), it is preferred that for L¹, d=1 (A according to structure (23), it is preferred that a=1 and R¹³=H), e=2, f=0 and g=1. In the context of antibody-conjugate (I), it is preferred that L²=Val-Cit or Val-Ala, more preferably Val-Cit. In the context of antibody-conjugate (I), it is preferred that p=1, and then that R²¹═H.

Antibody-conjugate (II) has the following structure:

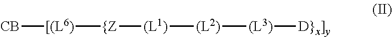

wherein:
CB, L⁶, Z, D, x and y are as defined above;
L¹ is a linker represented by -(A)-(B)ₑ-(C(O))—, as defined above;
L² is a peptide spacer as defined above, preferably Val-Cit or Val-Ala;
L³ is the PABC derivative according to structure (26), wherein R²¹═H.

In the context of antibody-conjugate (II), it is preferred that for L¹, e=2, A according to structure (23), it is preferred that a=1 and R¹³=H. In the context of antibody-conjugate (II), it is preferred that L²=Val-Cit or Val-Ala, more preferably Val-Cit.

According to a preferred embodiment, the conjugate according to the invention has a structure selected from (III)-(V):

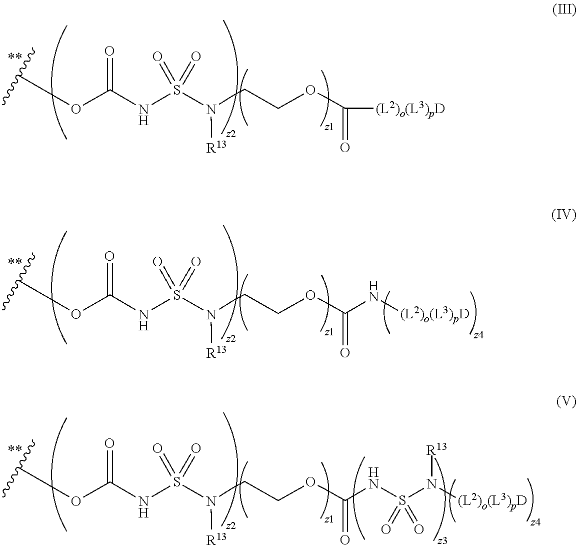

Herein:
the bond labelled with ** is connected to connecting group Z¹;
each R¹³ is individually selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, wherein the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups are optionally substituted and optionally interrupted by one or more heteroatoms selected from O, S and NR¹⁴, wherein R¹⁴ is independently selected from the group consisting of hydrogen and C₁-C₄ alkyl groups, or R¹³ is D connected to N via a spacer moiety, R¹³=hydrogen or D connected to N via a spacer moiety, preferably wherein the spacer moiety is —(B)ₑ-(A)_{f}-(B)_{g}—C(O)— as defined above;
L² is as defined above, preferably L² is a dipeptide, a tripeptide or a tetrapeptide;
o is 0 or 1, preferably o is 1;
L³ is as defined above;
p is 0 or 1;
z1 is an integer in the range of 1-4;
z2 is 0 or 1, preferably z2=1;
z3 is 0 or 1, preferably z3=0;
z4 is 1 or 2, preferably z4=1.

Conjugate (X) has a linker-payload moiety according to the following structure:

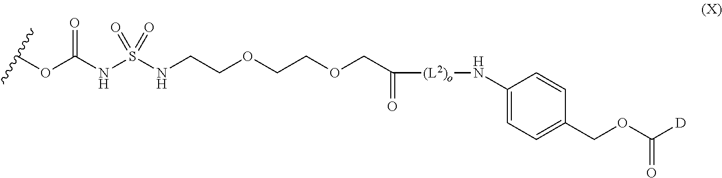

wherein:
the wavy line indicates the connection to Z¹;
L², o and D are as defined above.

L² may be present of absent, preferably L² is present and o=1. For preferred conjugate (Xa), L² is according to structure (25) and R¹⁷ is CH₃. For preferred conjugate (Xb), L² is according to structure (25) and R¹⁷ is CH₂CH₂CH₂NHC(O)NH₂. The conjugate (X) preferably has structure (Xb).

Conjugate (XI) has a linker-payload moiety according to the following structure:

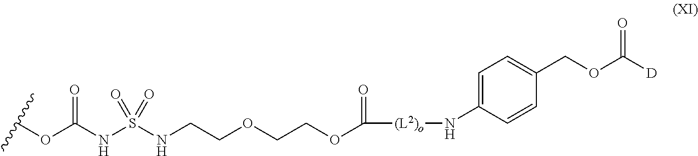

wherein:
the wavy line indicates the connection to Z¹;
L², o and D are as defined above.

L² may be present of absent, preferably L² is present and o=1. For preferred conjugate (XIa), L² is according to structure (25) and R¹⁷ is CH₃. For preferred conjugate (XIb), L² is according to structure (25) and R¹⁷ is CH₂CH₂CH₂NHC(O)NH₂. The conjugate (XI) preferably has structure (XIb).

Conjugate (XII) has a linker-payload moiety according to the following structure:

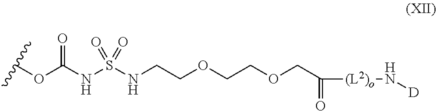

wherein:
the wavy line indicates the connection to Z¹;
L², o and D are as defined above.

L² may be present of absent, preferably L² is present and o=1. For preferred conjugate (XIIa), L² is according to structure (25) and R¹⁷ is CH₃. For preferred conjugate (XIIb), L² is according to structure (25) and R¹⁷ is CH₂CH₂CH₂NHC(O)NH₂. The conjugate (XII) preferably has structure (XIIb).

Conjugate (XIII) has a linker-payload moiety according to the following structure:

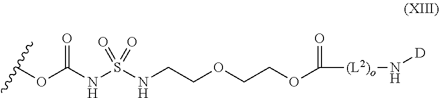

wherein:
the wavy line indicates the connection to Z¹;
L², o and D are as defined above.

L² may be present of absent, preferably L² is present and o=1. For preferred conjugate (XIVa), L² is according to structure (25) and R¹⁷ is CH₃. For preferred conjugate (XIVb), L² is according to structure (25) and R¹⁷ is CH₂CH₂CH₂NHC(O)NH₂. The conjugate (XIII) preferably has structure (XIIIb).

Conjugate (XIV) has a linker-payload moiety according to the following structure:

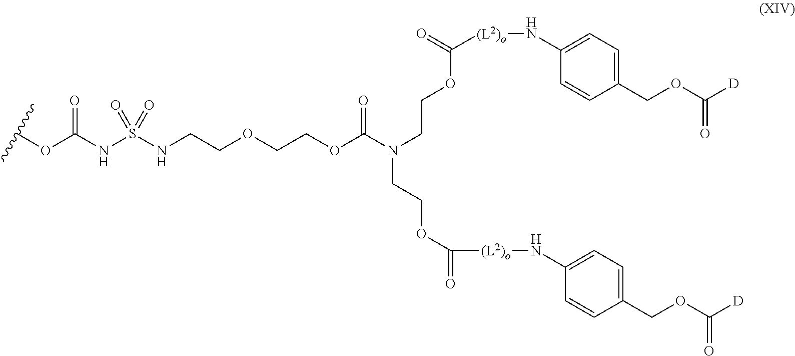

wherein:
the wavy line indicates the connection to Z¹;
L², o and D are as defined above.

L² may be present of absent, preferably L² is present and o=1. For preferred conjugate (XIVa), L² is according to structure (25) and R¹⁷ is CH₃. For preferred conjugate (XIVb), L² is according to structure (25) and R¹⁷ is CH₂CH₂CH₂NHC(O)NH₂. Preferably, R¹⁷=CH₂CH₂CH₂NHC(O)NH₂. In the context of conjugate (XIV), structures (XIVb) is most preferred.

Conjugate (XV) has a linker-payload moiety according to the following structure:

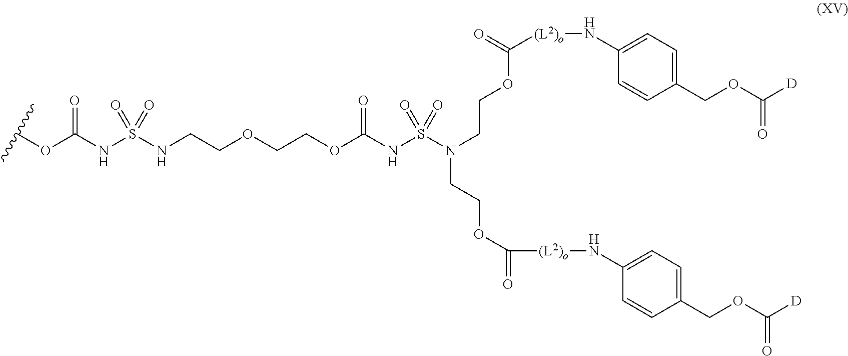

wherein:
the wavy line indicates the connection to Z¹;
L², o and D are as defined above.

L² may be present of absent, preferably L² is present and o=1. For preferred conjugate (XVa), L² is according to structure (25) and R¹⁷ is CH₃. For preferred conjugate (XVb), L² is according to structure (25) and R¹⁷ is CH₂CH₂CH₂NHC(O)NH₂. Preferably, R¹⁷=CH₂CH₂CH₂NHC(O)NH₂. In the context of conjugate (XV), structure (XVb) is most preferred.

Conjugate (XVI) has a linker-payload moiety according to the following structure:

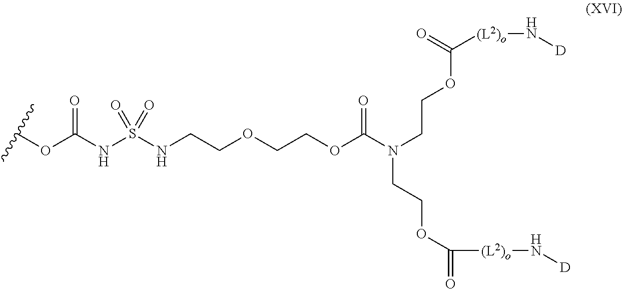

wherein:
the wavy line indicates the connection to Z¹;
L², o and D are as defined above.

L² may be present of absent, preferably L² is present and o=1. For preferred conjugate (XVIa), L² is according to structure (25) and R¹⁷ is CH₃. For preferred conjugate (XVIb), L² is according to structure (25) and R¹⁷ is CH₂CH₂CH₂NHC(O)NH₂. Preferably, R¹⁷=CH₂CH₂CH₂NHC(O)NH₂. In the context of conjugate (XVI), structures (XVIb) is most preferred.

Conjugate (XVII) has a linker-payload moiety according to the following structure:

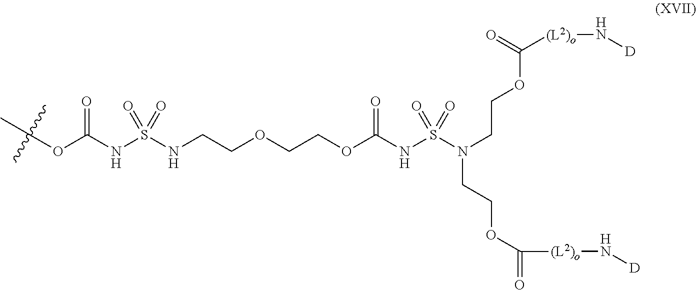

wherein:
the wavy line indicates the connection to Z¹;
L², o and D are as defined above.

L² may be present of absent, preferably L² is present and o=1. For preferred conjugate (XVIIa), L² is according to structure (25) and R¹⁷ is CH₃. For preferred conjugate (XVIIb), L² is according to structure (25) and R¹⁷ is CH₂CH₂CH₂NHC(O)NH₂. Preferably, R¹⁷=CH₂CH₂CH₂NHC(O)NH₂. In the context of conjugate (XVII), structure (XVIIb) is most preferred.

It is further preferred that these preferred conjugates (I)-(V), (X)-(XVII) are conjugated through the glycan, i.e. b=1, more preferably a trimmed glycan, i.e. j=0. Herein, it is further preferred that S=GalNAc and w′=0. Herein, it is further preferred that connecting group Z¹ is formed by an azide-alkyne cycloaddition, preferably connecting group Z¹=(Z39), wherein ring Z¹=(Za) and V═CH₂. Herein, it is further preferred that x=1. Herein, it is further preferred that y=2, more preferably that x=1 and y=2.

In a most preferred embodiment, the conjugate according to the invention is according to structure (Xb) or (XIb) as defined above, wherein b=1, e=0, S=GalNAc, w′=0, connecting group Z¹=(Z39), wherein ring Z═(Za) and V═CH₂, x=1 and y=2.

### Compound According to General Structure (4)

In a further aspect, the invention concerns a linker-toxin construct. The linker-toxin construct comprises the compound according to structure (1) connected to reactive moiety Q via linker L, and can be used in the preparation of the conjugate according to the invention, specifically by reaction with appropriately functionalized cell-binding agent CB-[(L⁶)_{b}-{F}ₓ]_{y} (5), defined further below. In a bioconjugation reaction, reactive moiety Q of the linker-toxin construct reacts with reactive moiety F on the cell-binding agent to create connecting group Z¹.

The linker-toxin construct according to the invention has general structure (4):

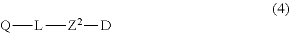

wherein:
Q is a reactive moiety;
L is a linker;
Z² is a connecting group that connects L to D;
D is the compound according to structure (1).

The linker-drug construct contains linker L and payload D of the final conjugate. Compounds according to general formula (4) can be prepared by the skilled person using standard organic synthesis techniques, and as exemplified in the examples. Linker L and payload D are defined above in the context of the conjugate according to structure (2).

Also, the connection between the linker L and the payload D via connecting group Z² is the compound according to structure (4) is the same as defined for the conjugate according to structure (2). Thus, in a preferred embodiment, compound according to structure (1) is conjugated to the cell-binding agent though R¹ or through Y. In case the conjugation occurs through R¹, the connection preferably occurs through the nitrogen atom when R¹=Sp—N(R⁴)₂. In case the conjugation occurs through Y, the connection preferably occurs through the nitrogen atom when Y═N(R⁴)₂ or NR⁴—Sp³—N(R⁴)₂. Herein, one R⁴ group is replaced by the connection to L, such that the remaining NR⁴ residue is part of Z² that is formed when the amine group is connected to the linker. In case the connection is through R¹ it is preferred that Y═CH₃ or CH₂OH.

### Reactive Moiety Q

The compound according to general structure (4) comprises a reactive moiety Q. In the context of the present invention, the term “reactive moiety” may refer to a chemical moiety that comprises a reactive group, but also to a reactive group itself. For example, a cyclooctynyl group is a reactive group comprising a reactive group, namely a C—C triple bond. Similarly, an N-maleimidyl group is a reactive group, comprising a C═C double bond as a reactive group. However, a reactive group, for example an azido reactive group, a thiol reactive group or an alkynyl reactive group, may herein also be referred to as a reactive moiety.

Q serves as chemical handle for the connection to S (F) x. In other words, Q is reactive towards and complementary to F. Herein, a reactive group is denoted as “complementary” to a reactive group when said reactive group reacts with said reactive group selectively, optionally in the presence of other functional groups. Complementary reactive and functional groups are known to a person skilled in the art, and are described in more detail below. As such, the compound according to general structure (4) is conveniently used in a conjugation reaction, wherein a chemical reaction between Q and F takes place, thereby forming an conjugate comprising a covalent connection between payload D and the antibody. This is explained in more detail below in the context of the process for synthesising the conjugate according to the invention.

The exact nature of Q, and F, depends on the type of conjugation reaction that is employed. The skilled person will be able to select the appropriate combination of Q and F. Preferably, Q, and thus also F, is reactive in a cycloaddition or a nucleophilic reaction. Thus, Q preferably comprises a click probe, a thiol, a thiol-reactive moiety, an amine or an amine-reactive moiety, more preferably Q is a click probe, a thiol-reactive moiety or an amine-reactive moiety, most preferably Q is a click probe. The click probe is reactive in a cycloaddition (click reaction) and is preferably selected from an azide, a tetrazine, a triazine, a nitrone, a nitrile oxide, a nitrile imine, a diazo compound, an ortho-quinone, a dioxothiophene, a sydnone, an alkene moiety and an alkyne moiety. Preferably, the click probe comprises or is an alkene moiety or an alkyne moiety, more preferably wherein the alkene is a (hetero)cycloalkene and/or the alkyne is a terminal alkyne or a (hetero)cycloalkyne. Typical thiol-reactive moieties are selected from maleimide moiety, a haloacetamide moiety, an allenamide moiety, a phosphonamidite moiety, a cyanoethynyl moiety, a vinylsulfone, a vinylpyridine moiety or a methylsulfonylphenyloxadiazole moiety. Most preferably, the thiol-reactive moiety comprises or is a maleimide moiety. Typical amine-reactive moieties are selected from N-hydroxysuccinimidyl esters and other activated esters, p-nitrophenyl carbonates and other activated carbonates, isocyanates, isothiocyanates, haloacetamides and benzoyl halides. In a preferred embodiment, Q is selected from an alkene moiety, an alkyne moiety, a thiol-reactive moiety or an amine-reactive moiety, more preferably an alkene moiety or an alkyne moiety, even more preferably an alkyne moiety. Herein, the alkene is preferably a (hetero)cycloalkene and the alkyne is preferably a terminal alkyne or a (hetero)cycloalkyne. Most preferably, Q is a cyclic (hetero)alkyne moiety. Each of these moieties are further defined here below.

Thus, in an especially preferred embodiment, Q comprises a cyclic (hetero)alkyne moiety. The alkynyl group may also be referred to as a (hetero)cycloalkynyl group, i.e. a heterocycloalkynyl group or a cycloalkynyl group, wherein the (hetero)cycloalkynyl group is optionally substituted. Preferably, the (hetero)cycloalkynyl group is a (hetero)cycloheptynyl group, a (hetero)cyclooctynyl group, a (hetero)cyclononynyl group or a (hetero)cyclodecynyl group. Herein, the (hetero)cycloalkynes may optionally be substituted. Preferably, the (hetero)cycloalkynyl group is an optionally substituted (hetero)cycloheptynyl group or an optionally substituted (hetero)cyclooctynyl group. Most preferably, the (hetero)cycloalkynyl group is a (hetero)cyclooctynyl group, wherein the (hetero)cyclooctynyl group is optionally substituted.

In an especially preferred embodiment, Q comprises a (hetero)cycloalkynyl or (hetero)cycloalkenyl group and is according to structure (Q1):

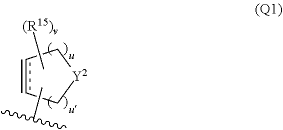

### Herein:

the bond depicted as [symbol] is a double bond or a triple bond;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero) arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)═NR³¹ or NR³¹, wherein S⁽⁺⁾ is a cationic sulphur atom counterbalanced by B⁽⁻⁾ wherein B⁽⁻⁾ is an anion, and wherein each R³¹ individually is R¹⁵ or a connection with D, connected via L;
u is 0, 1, 2, 3, 4 or 5;
u′ is 0, 1, 2, 3, 4 or 5, wherein u+u′=0, 1, 2, 3, 4, 5, 6, 7 or 8;
v=an integer in the range 0-16;
Typically, v=(u+u′)×2 (when the connection to L, depicted by the wavy bond, is via Y²) or [(u+u′)×2]−1 (when the connection to L, depicted by the wavy bond, is via one of the carbon atoms of u and u′).

In a preferred embodiment of structure (Q1), reactive group Q comprises a (hetero)cycloalkynyl group and is according to structure (Q1a):

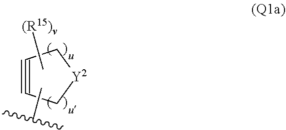

### Herein,

R¹⁵ and Y² are as defined above
u is 0, 1, 2, 3, 4 or 5;
u′ is 0, 1, 2, 3, 4 or 5, wherein u+u′=4, 5, 6, 7 or 8;
v=an integer in the range 8-16.

In a preferred embodiment, u+u′=4, 5 or 6, more preferably u+u′=5.

In a preferred embodiment, v=8, 9 or 10, more preferably v=9 or 10, most preferably v=10.

In a preferred embodiment, Q is a (hetero)cycloalkynyl group selected from the group consisting of (Q2)-(Q20) depicted here below.

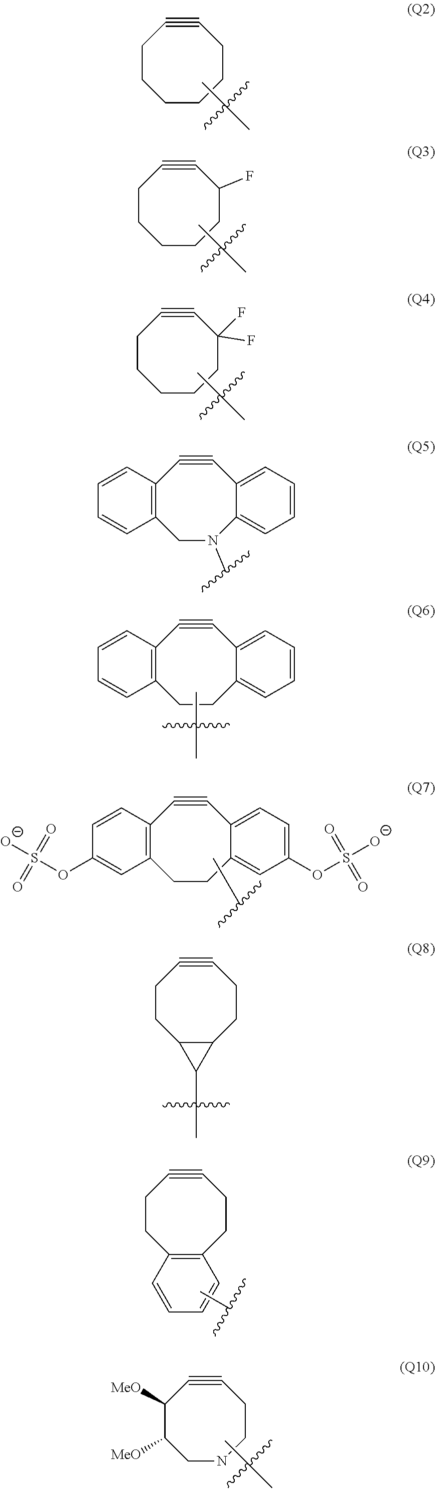

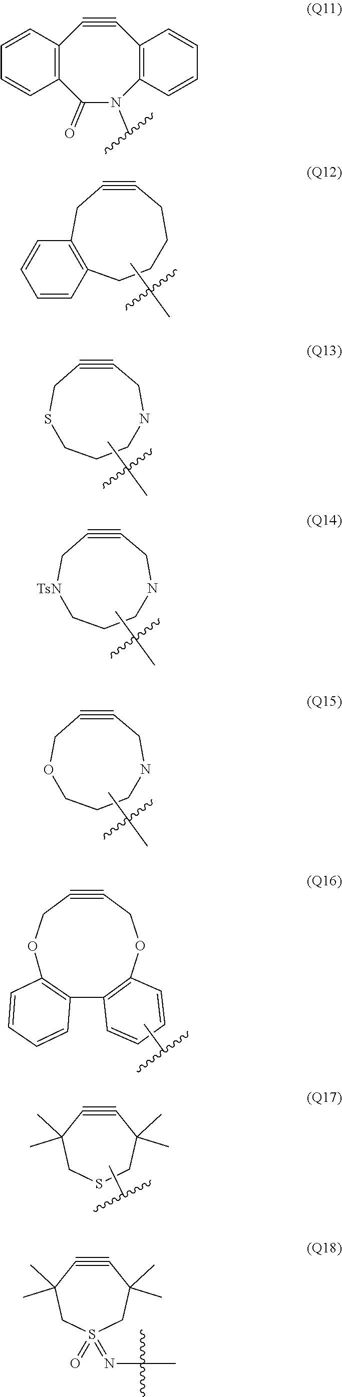

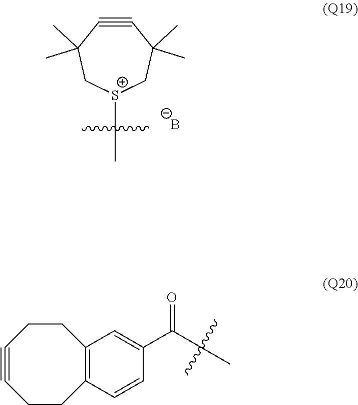

Herein, the connection to L, depicted with the wavy bond, may be to any available carbon or nitrogen atom of Q. The nitrogen atom of (Q10), (Q13), (Q14) and (Q15) may bear the connection to L, or may contain a hydrogen atom or be optionally functionalized. B (−) is an anion, which is preferably selected from ⁽⁻⁾OTf, Cl⁽⁻⁾, Br⁽⁻⁾ or I⁽⁻⁾, most preferably B⁽⁻⁾ is ⁽⁻⁾OTf. In the conjugation reaction, B⁽⁻⁾ does not need to be a pharmaceutically acceptable anion, since B⁽⁻⁾ will exchange with the anions present in the reaction mixture anyway. In case (Q19) is used for Q, the negatively charged counter-ion is preferably pharmaceutically acceptable upon isolation of the conjugate according to the invention, such that the conjugate is readily useable as medicament.

In a further preferred embodiment, Q is a (hetero)cycloalkynyl group selected from the group consisting of (Q21)-(Q38) depicted here below.

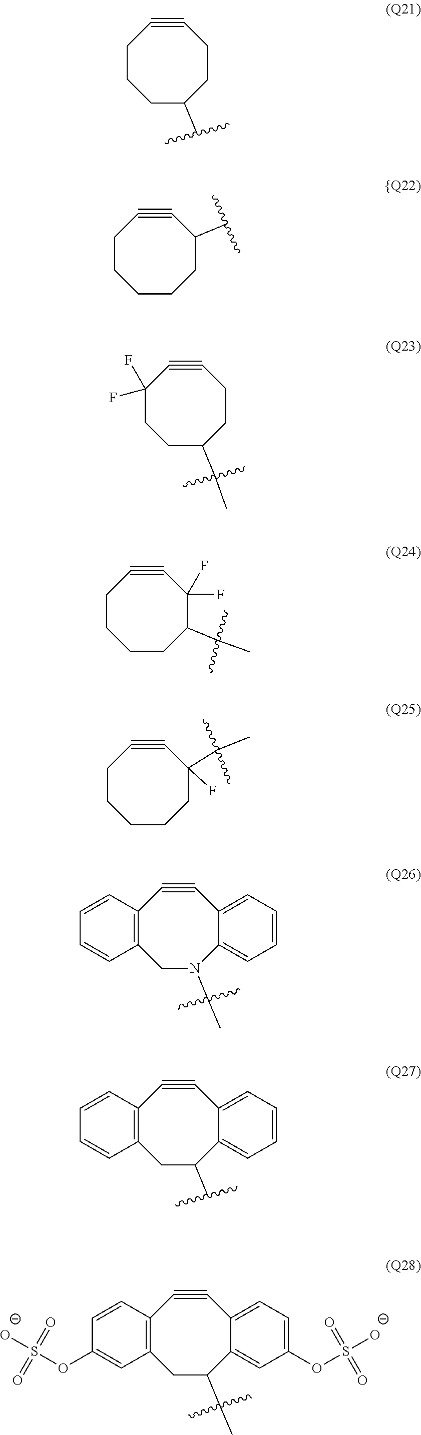

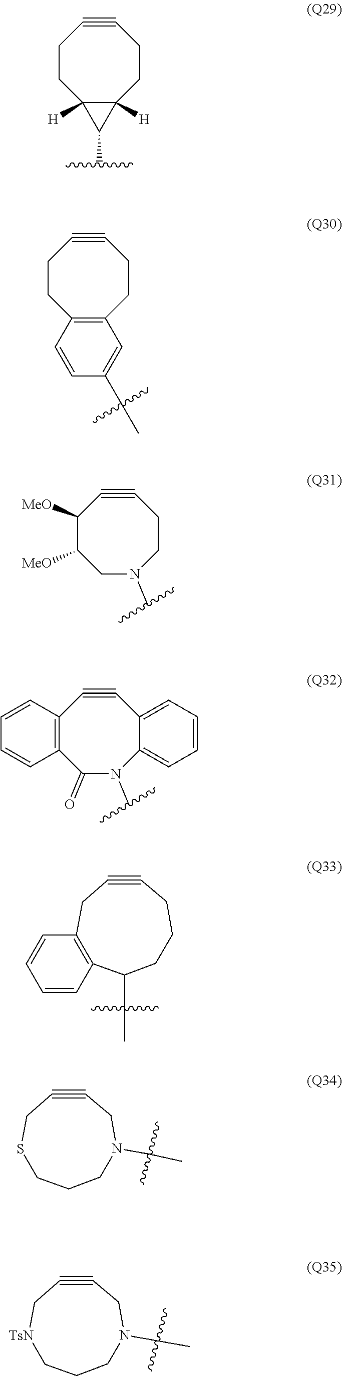

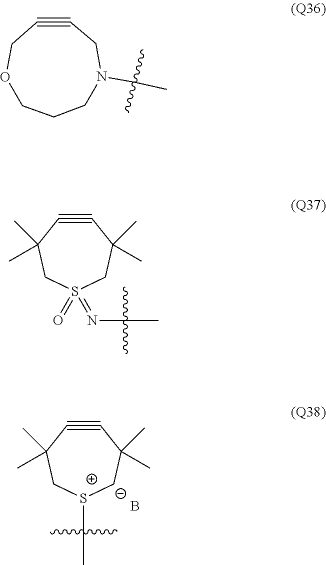

In structure (Q38), B⁽⁻⁾ is an anion, which is preferably selected from ⁽⁻⁾OTf, Cl⁽⁻⁾, Br⁽⁻⁾ or I⁽⁻⁾ most preferably B⁽⁻⁾ is ⁽⁻⁾OTf.

In a preferred embodiment, Q comprises a (hetero)cyclooctyne moiety or a (hetero)cycloheptyne moiety, preferably according to structure (Q8), (Q26), (Q27), (Q28) or (Q37), which are optionally substituted. Each of these preferred options for Q are further defined here below.

Thus, in a preferred embodiment, Q comprises a heterocycloheptyne moiety according to structure (Q37), also referred to as a TMTHSI, which is optionally substituted. Preferably, the heterocycloheptyne moiety according to structure (Q37) is not substituted.

In an alternative preferred embodiment, Q comprises a cyclooctyne moiety according to structure (Q8), more preferably according to (Q29), also referred to as a bicyclo[6.1.0]non-4-yn-9-yl] group (BCN group), which is optionally substituted. Preferably, the cyclooctyne moiety according to structure (Q8) or (Q29) is not substituted. In the context of the present embodiment, Q preferably is a (hetero)cyclooctyne moiety according to structure (Q39) as shown below, wherein V is (CH₂)_{I} and I is an integer in the range of 0 to 10, preferably in the range of 0 to 6. More preferably, I is 0, 1, 2, 3 or 4, more preferably I is 0, 1 or 2 and most preferably I is 0 or 1. In the context of group (Q39), I is most preferably 1. Most preferably, Q is according to structure (Q42), defined further below.

In an alternative preferred embodiment, Q comprises a (hetero)cyclooctyne moiety according to structure (Q26), (Q27) or (Q28), also referred to as a DIBO, DIBAC, DBCO or ADIBO group, which are optionally substituted. In the context of the present embodiment, Q preferably is a (hetero)cyclooctyne moiety according to structure (Q40) or (Q41) as shown below, wherein Y¹ is O or NR¹¹, wherein R¹¹ is independently selected from the group consisting of hydrogen, a linear or branched C₁-C₁₂ alkyl group or a C₄-C₁₂ (hetero)aryl group. The aromatic rings in (Q40) are optionally O-sulfonylated at one or more positions, whereas the rings of (Q41) may be halogenated at one or more positions. Preferably, the (hetero)cyclooctyne moiety according to structure (Q40) or (Q41) is not further substituted. Most preferably, Q is according to structure (Q43), defined further below.

In an alternative preferred embodiment, Q comprises a heterocycloheptynyl group and is according to structure (Q37).

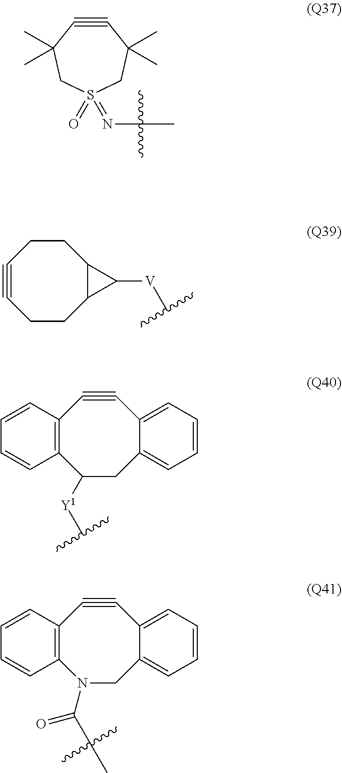

In an especially preferred embodiment, Q comprises a cyclooctynyl group and is according to structure (Q42):

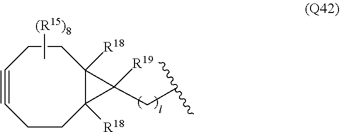

### Herein:

R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero) arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of Q or D connected via a spacer moiety; and
I is an integer in the range 0 to 10.

In a preferred embodiment of the reactive group according to structure (Q42), R¹⁵ is independently selected from the group consisting of hydrogen, halogen, —OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the reactive group according to structure (Q42), R¹⁸ is independently selected from the group consisting of hydrogen, C₁-C₆ alkyl groups, most preferably both R¹⁸ are H. In a preferred embodiment of the reactive group according to structure (Q42), R¹⁹ is H. In a preferred embodiment of the reactive group according to structure (Q42), I is 0 or 1, more preferably I is 1.

In an especially preferred embodiment, Q comprises a (hetero)cyclooctynyl group and is according to structure (Q43):

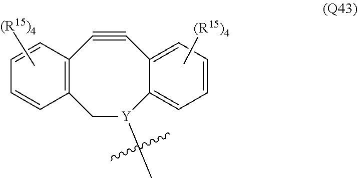

### Herein:

R¹⁵ is independently selected from the group consisting of hydrogen, halogen, —OR¹⁶, —NO₂, —CN, —S(O)₂R¹⁶, —S(O)₃ (−), C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero) arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

In a preferred embodiment of the reactive group according to structure (Q43), R¹⁵ is independently selected from the group consisting of hydrogen, halogen, —OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁽⁻⁾. In a preferred embodiment of the reactive group according to structure (Q43), Y is N or CH, more preferably Y═N.

In an alternative preferred embodiment, Q comprises a cyclic alkene moiety. The alkenyl group Q may also be referred to as a (hetero)cycloalkenyl group, i.e. a heterocycloalkenyl group or a cycloalkenyl group, preferably a cycloalkenyl group, wherein the (hetero)cycloalkenyl group is optionally substituted. Preferably, the (hetero)cycloalkenyl group is a (hetero)cyclopropenyl group, a (hetero)cyclobutenyl group, a norbornene group, a norbornadiene group, a trans-(hetero)cycloheptenyl group, a trans-(hetero)cyclooctenyl group, a trans-(hetero)cyclononenyl group or a trans-(hetero)cyclodecenyl group, which may all optionally be substituted. Especially preferred are (hetero)cyclopropenyl groups, trans-(hetero)cycloheptenyl group or trans-(hetero)cyclooctenyl groups, wherein the (hetero)cyclopropenyl group, the trans-(hetero)cycloheptenyl group or the trans-(hetero)cyclooctenyl group is optionally substituted. Preferably, Q comprises a cyclopropenyl moiety according to structure (Q44), a hetereocyclobutene moiety according to structure (Q45), a norbornene or norbornadiene group according to structure (Q46), a trans-(hetero)cycloheptenyl moiety according to structure (Q47) or a trans-(hetero)cyclooctenyl moiety according to structure (Q48). Herein, Y³ is selected from C(R²³)₂, NR²³ or O, wherein each R²³ is individually hydrogen, C₁-C₆ alkyl or is connected to L, optionally via a spacer, and the bond labelled [symbol] is a single or double bond. In a further preferred embodiment, the cyclopropenyl group is according to structure (Q49). In another preferred embodiment, the trans-(hetero)cycloheptene group is according to structure (Q50) or (Q51). In another preferred embodiment, the trans-(hetero)cyclooctene group is according to structure (Q52), (Q53), (Q54), (Q55) or (Q56).

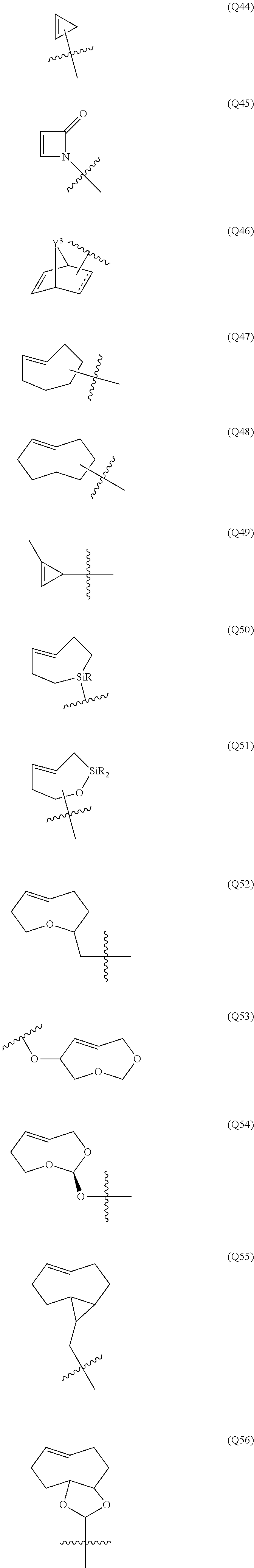

Herein, the R group(s) on Si in (Q50) and (Q51) are typically alkyl or aryl, preferably C₁-C₆ alkyl.

In an alternative preferred embodiment, Q is a thiol-reactive probe. In this embodiment, Q is a reactive group compatible with cysteine conjugation. Such probes are known in the art and may be selected from the group consisting of a maleimide moiety, a haloacetamide moiety, an allenamide moiety, a phosphonamidite moiety, a cyanoethynyl moiety, a vinylsulfone, a vinylpyridine moiety or a methylsulfonylphenyloxadiazole moiety. Most preferably, Q comprises a maleimide moiety. Reagents may be monoalkylation type or may be a cross-linker for reaction with two cysteine side-chains.

In a further preferred embodiment, probe Q is selected from the group consisting of (Q57)-(Q71) depicted here below.

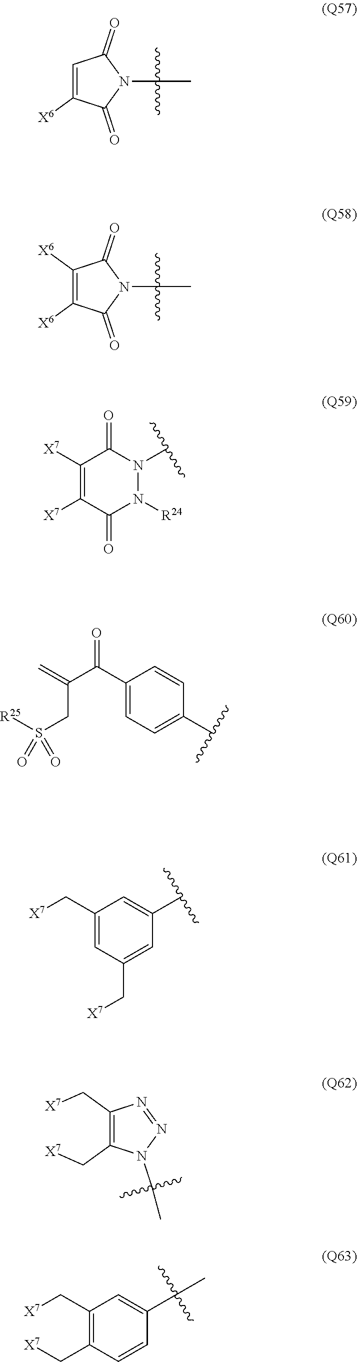

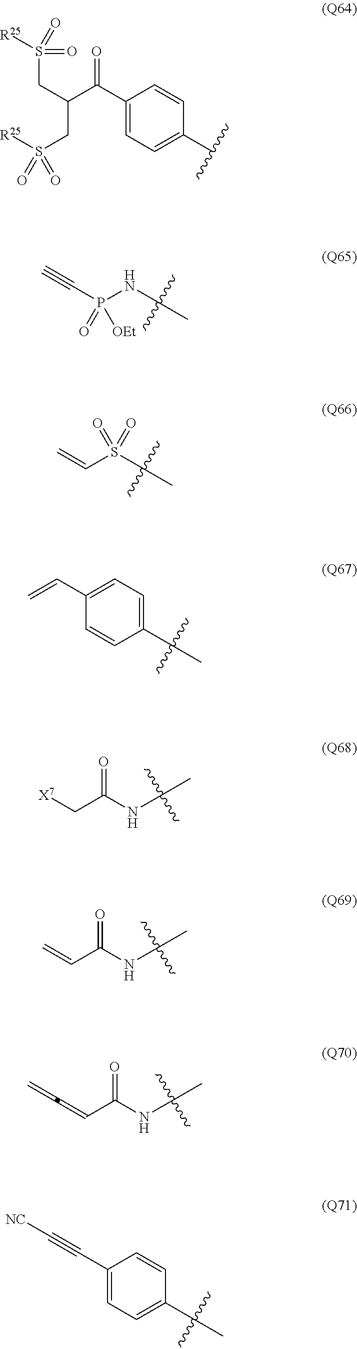

wherein:
X⁶ is H, halogen, PhS, MeS, preferably a halogen, such as Cl, Br, I;
X⁷ is halogen, PhS, MeS, preferably a halogen, such as Cl, Br, I;
R²⁴ is H or C₁₋₁₂ alkyl, preferably H or C₁₋₆ alkyl;
R²⁵ is H, C₁₋₁₂ alkyl, C₁₋₁₂ aryl, C₁₋₁₂ alkaryl or C₁₋₁₂ aralkyl, preferably H or para-methylphenyl;
wherein the aromatic ring of (Q61) and (Q63) may optionally be a heteroaromatic ring, such as a phenyl or pyridine ring.

In a preferred embodiment of thiol-reactive probe (Q57), the probe Q is selected from the group consisting of (Q72)-(Q74) depicted here below.

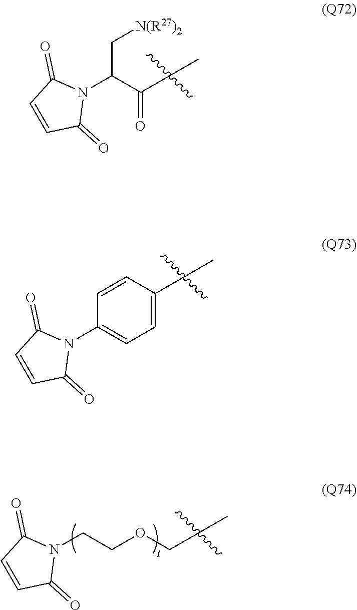

wherein:
R²⁷ is C₁₋₁₂ alkyl, C₁₋₁₂ aryl, C₁₋₁₂ alkaryl or C₁₋₁₂ aralkyl;
t is an integer in the range of 0-15, preferably 1-10.

In an alternative preferred embodiment, Q is an amine-reactive probe. In this embodiment, Q is a reactive group compatible with lysine conjugation. Such probes are known in the art and may be selected from the group consisting of N-hydroxysuccinimidyl groups, p-nitrophenyl carbonates, pentafluorophenyl carbonates, isocyanate groups, isothiocyanate groups and benzoyl halide groups. Most preferably, Q comprises or is an N-hydroxysuccinimidyl ester, a p-nitrophenyl carbonate moiety or a pentafluorophenyl carbonate moiety.

In a further preferred embodiment, probe Q is selected from the group consisting of (Q75)-(Q80) depicted here below.

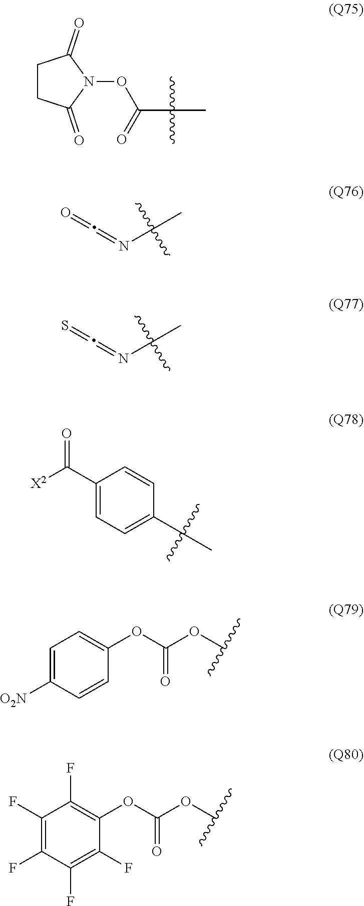

Herein, X² is halogen, preferably F.

In a preferred embodiment, Q is selected from the group consisting of (Q1)-(Q80).

### Cell-Binding Agent According to General Structure (5)

The cell-binding agent that is to be used in the bioconjugation reaction with the linker-toxin construct has general structure (5):

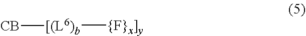

wherein:
CB is a cell-binding agent;
b is 0 or 1;
L⁶ is -GlcNAc(Fuc)_{w}-(G)ⱼ-S-(L⁷)_{w′}-, wherein G is a monosaccharide, j is an integer in the range of 0-10, S is a sugar or a sugar derivative, GlcNAc is N-acetylglucosamine and Fuc is fucose, w is 0 or 1, w′ is 0, 1 or 2 and L⁷ is —N(H)C(O)CH₂—, —N(H)C(O)CF₂— or —CH₂—;
F is a reactive moiety;
x is 1 or 2; and
y is 1, 2, 3 or 4.

The cell-binding agent of general structure (5) may also be referred to as a “(modified) cell-binding”, preferably a “(modified) antibody”, for containing reactive groups F, wherein the reactive groups F are naturally present or the cell-binding agent is modified to incorporate the reactive groups F. The (modified) cell-binding agent or antibody according to general formula (5) can be prepared by the skilled person using standard organic and/or enzymatic synthesis techniques, and as exemplified in the examples. Cell-binding agent CB, linker L⁶, b, x and y are defined above in the context of the conjugate according to structure (2).

### Reactive Moiety F

F is reactive towards Q in the conjugation reaction defined below, preferably wherein the conjugation reaction is a cycloaddition or a nucleophilic reaction. As the skilled person will understand, the options for F are the same as those for Q, provided that F and Q are reactive towards each other. Thus, F preferably comprises a click probe, a thiol, a thiol-reactive moiety, an amine or an amine-reactive moiety, more preferably F is a click probe, a thiol or an amine, most preferably F is a click probe. The click probe is reactive in a cycloaddition (click reaction) and is preferably selected from an azide, a tetrazine, a triazine, a nitrone, a nitrile oxide, a nitrile imine, a diazo compound, an ortho-quinone, a dioxothiophene, a sydnone, an alkene moiety and an alkyne moiety. Preferably, the click probe comprises or is an azide, a tetrazine, a triazine, a nitrone, a nitrile oxide, a nitrile imine, a diazo compound, an ortho-quinone, a dioxothiophene or a sydnone, most preferably an azide. Typical thiol-reactive moieties are selected from maleimide moiety, a haloacetamide moiety, an allenamide moiety, a phosphonamidite moiety, a cyanoethynyl moiety, an ortho-quinone moiety, a vinylsulfone, a vinylpyridine moiety or a methylsulfonylphenyloxadiazole moiety. Most preferably, the thiol-reactive moiety comprises or is a maleimide moiety. Typical amine-reactive moieties are selected from N-hydroxysuccinimidyl esters, p-nitrophenyl carbonates, pentafluorophenyl carbonates, isocyanates, isothiocyanates and benzoyl halides. In a preferred embodiment, F is a click probe or a thiol, more preferably F is an azide or a thiol, most preferably F is an azide.

More than one reactive group F may be present in the antibody. The reactive group F in the antibody may be naturally present or may be placed in the antibody by a specific technique, for example a (bio) chemical or a genetic technique. The reactive group that is placed in the antibody is prepared by chemical synthesis, for example an azide or a terminal alkyne. Methods of preparing modified antibodies are known in the art, e.g. from WO 2014/065661, WO 2016/170186 and WO 2016/053107, which are incorporated herein by reference. From the same documents, the conjugation reaction between the modified antibody and a linker-toxin-construct is known to the skilled person.

Preferably, F is a click probe reactive towards a (hetero)cycloalkene and/or a (hetero)cycloalkyne, and is typically selected from the group consisting of azide, tetrazine, triazine, nitrone, nitrile oxide, nitrile imine, diazo compound, ortho-quinone, dioxothiophene and sydnone. Preferred structures for the reactive group are structures (F1)-(F10) depicted here below.

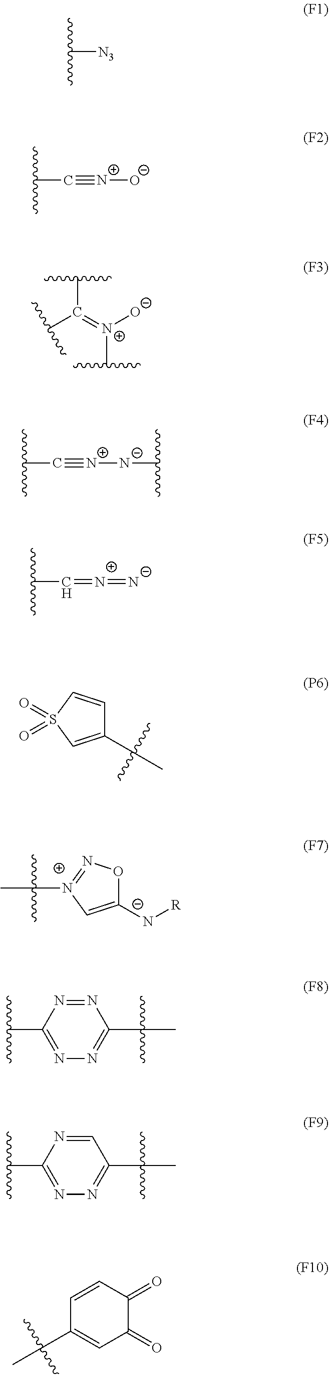

Herein, the wavy bond represents the connection to the payload. For (F3), (F4), (F8) and (F9), the payload can be connected to any one of the wavy bonds. The other wavy bond may then be connected to an R group selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups and C₁-C₂₄ sulfonyl groups, each of which (except hydrogen) may optionally be substituted and optionally interrupted by one or more heteroatoms selected from O, S and NR³² wherein R³² is independently selected from the group consisting of hydrogen and C₁-C₄ alkyl groups. The skilled person understands which R groups may be applied for each of the groups F. For example, the R group connected to the nitrogen atom of (F3) may be selected from alkyl and aryl, and the R group connected to the carbon atom of (F3) may be selected from hydrogen, alkyl, aryl, acyl and sulfonyl. Preferably, the reactive moiety F is selected from azides or tetrazines. Most preferably, the reactive moiety F is an azide.

In a second preferred embodiment, F is a thiol or precursor thereof. Thiol or precursor thereof F is used in the conjugation reaction to connect the linker-toxin-construct to the (modified) cell-binding agent. F is reactive towards thiol-reactive probe Q in a thiol ligation. The thiol preferably the thiol of the side chain of a cysteine amino acid, which are naturally present within the antibody AB, in which case linker L⁶ is not present (b=0), although it may also be synthetically introduced, optionally via a linker L⁶. Thiol precursors in the context of bioconjugation are known in the art, and include disulfides, which may be naturally occurring disulfide bridges present in the antibody or synthetically introduced disulfides, which are reduced as known in the art. Preferably, F is a thiol group of a cysteine side chain.

In a third preferred embodiment, F is an amine or precursor thereof, preferably an amine. Amine or precursor thereof F is used in the conjugation reaction to connect the linker-toxin-construct to the (modified) antibody. F is reactive towards amine-reactive probe Q in nucleophilic substitution. The amine is typically a primary amine, preferably the amine of the side chain of a lysine amino acid, which are naturally present within the antibody AB, in which case linker L⁶ is not present (b=0), although it may also be synthetically introduced, optionally via a linker L⁶. Preferably, F is a primary amine group of a lysine side chain.

### Process for Synthesising the Conjugate According to General Structure (2)

In a further aspect, the present invention relates to a process for the preparation of the conjugate according to the invention, the process comprising the step of reacting Q of the toxin-linker-construct according to the invention with a reactive group F. The linker-toxin-construct according to general structure (4), and preferred embodiments thereof, are described in more detail above. The present process occurs under conditions such that Q is reacted with F to covalently link the cell-binding agent CB (5) to the payload D. In the process according to the invention, Q reacts with F, forming a covalent connection between the cell-binding agent and the compound according to the invention. Complementary reactive groups Q and reactive groups F are known to the skilled person and are described in more detail below.

Any conjugation technique known in the art can be employed to prepare the conjugate according to the invention. Suitable conjugation techniques include thiol ligation, lysine ligation, cycloadditions (e.g. copper-catalysed click reaction, strain-promoted azide-alkyne cycloaddition, strain-promoted quinone-alkyne cycloaddition). Alternatively worded, the conjugation technique is selected form amide bond formation, carbamate bond formation, thiol alkylation, thiol arylation and cycloaddition reaction. Preferred conjugation techniques used in the context of the present invention include nucleophilic reactions and cycloadditions, preferably wherein the cycloaddition is a [4+2] cycloaddition or a [3+2] cycloaddition and the nucleophilic reaction is a Michael addition or a nucleophilic substitution. Suitable conjugation techniques are for example disclosed in G. T. Hermanson, “Bioconjugate Techniques”, Elsevier, 3rd Ed. 2013 (ISBN: 978-0-12-382239-0), WO 2014/065661, van Geel et al., Bioconj. Chem. 2015, 26, 2233-2242, PCT/EP2021/050594, PCT/EP2021/050598 and NL 2026947.

Thus, in a preferred embodiment of the conjugation process according to the invention, conjugation is accomplished via a nucleophilic reaction, such as a nucleophilic substitution or a Michael reaction. A preferred nucleophilic reaction is the acylation of a primary amino group with an activated ester. A preferred Michael reaction is the maleimide-thiol reaction, which is widely employed in bioconjugation.

Thus, in a preferred embodiment of the conjugation process according to the invention, conjugation is accomplished via a cycloaddition. Preferred cycloadditions are a (4+2)-cycloaddition (e.g. a Diels-Alder reaction) or a (3+2)-cycloaddition (e.g. a 1,3-dipolar cycloaddition). Preferably, the conjugation reaction is the Diels-Alder reaction or the 1,3-dipolar cycloaddition. The preferred Diels-Alder reaction is the inverse-electron demand Diels-Alder cycloaddition. In another preferred embodiment, the 1,3-dipolar cycloaddition is used, more preferably the alkyne-azide cycloaddition, and most preferably wherein Q is or comprises an alkyne group and F is an azido group. Cycloadditions, such as Diels-Alder reactions and 1,3-dipolar cycloadditions are known in the art, and the skilled person knowns how to perform them.

The process according to the present aspect preferably concerns a click reaction, more preferably a 1,3-dipolar cycloaddition, most preferably an alkyne/azide cycloaddition. Most preferably, Q is or comprises an alkyne group and F is an azido group. Click reactions, such as 1,3-dipolar cycloadditions, are known in the art, and the skilled person knows how to perform them.

Thus, the process for preparing the conjugate according to the invention according to the invention comprises reacting the modified cell-binding agent of structure (5) with a linker-toxin construct according to structure (4), to obtain the conjugate of structure (2).

In a preferred embodiment, the process for preparing an antibody-conjugate according to the invention comprises:
(i) contacting an antibody comprising y core N-acetylglucosamine (GlcNAc) moieties, wherein y=1, 2, 3 or 4, with a compound of the formula S(F)ₓ—P in the presence of a catalyst, wherein S(F)ₓ is a sugar derivative comprising x reactive groups F capable of reacting with a reactive group Q, x is 1 or 2 and P is a nucleoside mono- or diphosphate, and wherein the catalyst is capable of transferring the S(F)ₓ moiety to the core-GlcNAc moiety, to obtain a modified antibody according to Formula (26):

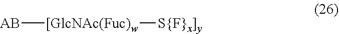

wherein
   AB is an antibody;
   Fuc is fucose;
   w is 0 or 1; and
(ii) reacting the modified antibody with a compound according to structure (4):

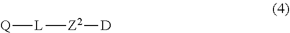

wherein:
   Q is a reactive moiety;
   L is a linker;
   Z² is a connecting group;
   D is a compound according to general structure (1);
to obtain the antibody-conjugate according to structure (2).

### Step (i)

In step (i), an antibody comprising 1, 2, 3 or 4 core N-acetylglucosamine moieties is contacted with a compound of the formula S(F)ₓ—P in the presence of a catalyst, wherein S(F)ₓ is a sugar derivative comprising x reactive groups F capable of reacting with a reactive group Q, x is 1 or 2 and P is a nucleoside mono- or diphosphate, and wherein the catalyst is capable of transferring the S(F)ₓ moiety to the core-GlcNAc moiety. Herein, the antibody is typically an antibody that has been trimmed to a core-GlcNAc residue as described further below. Step (i) affords a modified antibody according to Formula (26).

The starting material, i.e. the antibody comprising a core-GlcNAc substituent, is known in the art and can be prepared by methods known by the skilled person. In one embodiment, the process according to the invention further comprises the deglycosylation of an antibody glycan having a core N-acetylglucosamine, in the presence of an endoglycosidase, in order to obtain an antibody comprising a core N-acetylglucosamine substituent, wherein said core N-acetylglucosamine and said core N-acetylglucosamine substituent are optionally fucosylated. Depending on the nature of the glycan, a suitable endoglycosidase may be selected. The endoglycosidase is preferably selected from the group consisting of EndoS, EndoA, EndoE, EfEndo18A, EndoF, EndoM, EndoD, EndoH, EndoT and EndoSH and/or a combination thereof, the selection of which depends on the nature of the glycan. EndoSH is described in PCT/EP2017/052792, see Examples 1-3, and SEQ. ID No: 1, which is incorporated by reference herein.

Structural features S and x are defined above for the conjugate according to the invention, which equally applies to the present aspect. Compounds of the formula S(F)ₓ—P, wherein a nucleoside monophosphate or a nucleoside diphosphate P is linked to a sugar derivative S(F)ₓ, are known in the art. For example Wang et al., Chem. Eur. J. 2010, 16, 13343-13345, Piller et al., ACS Chem. Biol. 2012, 7, 753, Piller et al., Bioorg. Med. Chem. Lett. 2005, 15, 5459-5462 and WO 2009/102820, all incorporated by reference herein, disclose a number of compounds S(F)ₓ—P and their syntheses. In a preferred embodiment nucleoside mono- or diphosphate P in S(F)ₓ—P is selected from the group consisting of uridine diphosphate (UDP), guanosine diphosphate (GDP), thymidine diphosphate (TDP), cytidine diphosphate (CDP) and cytidine monophosphate (CMP), more preferably P is selected from the group consisting of uridine diphosphate (UDP), guanosine diphosphate (GDP) and cytidine diphosphate (CDP), most preferably P=UDP. Preferably, S(F)ₓ—P is selected from the group consisting of GalNAz-UDP, F2-GalNAz-UDP (N-(azidodifluoro)acetyl-galactosamine), 6-AzGal-UDP, 6-AzGalNAc-UDP (6-azido-6-deoxy-N-acetylgalactosamine-UDP), 4-AzGalNAz-UDP, 6-AzGalNAz-UDP, GlcNAz-UDP, 6-AzGlc-UDP, 6-AzGlcNAz-UDP and 2-(but-3-yonic acid amido)-2-deoxy-galactose-UDP. Most preferably, S(F)ₓ—P is GalNAz-UDP or 6-AzGalNAc-UDP.

Suitable catalyst that are capable of transferring the S(F)ₓ moiety to the core-GlcNAc moiety are known in the art. A suitable catalyst is a catalyst wherefore the specific sugar derivative nucleotide S(F)ₓ—P in that specific process is a substrate. More specifically, the catalyst catalyses the formation of a β(1,4)-glycosidic bond. Preferably, the catalyst is selected from the group of galactosyltransferases and N-acetylgalactosaminyltransferases, more preferably from the group of β(1,4)-N-acetylgalactosaminyltransferases (GalNACT) and β(1,4)-galactosyltransferases (GalT), most preferably from the group of β(1,4)-N-acetylgalactosaminyltransferases having a mutant catalytic domain. Suitable catalysts and mutants thereof are disclosed in WO 2014/065661, WO 2016/022027 and WO 2016/170186, all incorporated herein by reference. In one embodiment, the catalyst is a wild-type galactosyltransferase or N-acetylgalactosaminyltransferase, preferably an N-acetylgalactosaminyltransferase. In an alternative embodiment, the catalyst is a mutant galactosyltransferase or N-acetylgalactosaminyltransferases, preferably a mutant N-acetylgalactosaminyltransferase. Mutant enzymes described in WO 2016/022027 and WO 2016/170186 are especially preferred. These galactosyltransferase (mutant) enzyme catalysts are able to recognize internal sugars and sugar derivatives as an acceptor. Thus, sugar derivative S (F) x is linked to the core-GlcNAc substituent in step (i), irrespective of whether said GlcNAc is fucosylated or not.

Step (i) is preferably performed in a suitable buffer solution, such as for example phosphate, buffered saline (e.g. phosphate-buffered saline, tris-buffered saline), citrate, HEPES, tris and glycine. Suitable buffers are known in the art. Preferably, the buffer solution is phosphate-buffered saline (PBS) or tris buffer. Step (i) is preferably performed at a temperature in the range of about 4 to about 50° C., more preferably in the range of about 10 to about 45° C., even more preferably in the range of about 20 to about 40° C., and most preferably in the range of about 30 to about 37° C. Step (i) is preferably performed a pH in the range of about 5 to about 9, preferably in the range of about 5.5 to about 8.5, more preferably in the range of about 6 to about 8. Most preferably, step (i) is performed at a pH in the range of about 7 to about 8.

### Step (ii)

In step (ii), the modified antibody is reacted with a compound according to general structure (4), comprising a reactive group Q capable of reacting with reactive group F and a payload D, to obtain the conjugate according to the invention, containing connecting group Z¹ resulting from the reaction between Q and F. Such reaction occurs under condition such that reactive group Q is reacted with the reactive group F of the antibody to covalently link the antibody to the compound according to general structure (4). Step (ii) may also be referred to as the conjugation reaction.

In a preferred embodiment, in step (ii) an azide on an azide-modified antibody reacts with an alkynyl group, preferably a terminal alkynyl group, or a (hetero)cycloalkynyl group of the compound according to general structure (4), via a cycloaddition reaction. This cycloaddition reaction of a molecule comprising an azide with a molecule comprising a terminal alkynyl group or a (hetero)cycloalkynyl group is one of the reactions that is known in the art as “click chemistry”. In the case of a linker-toxin construct comprising a terminal alkynyl group, said cycloaddition reaction needs to be performed in the presence of a suitable catalyst, preferably a Cu (I) catalyst. However, in a preferred embodiment, the linker-toxin construct comprises a (hetero)cycloalkynyl group, more preferably a strained (hetero)cycloalkynyl group. When the (hetero)cycloalkynyl is a strained (hetero)cycloalkynyl group, the presence of a catalyst is not required, and said reaction may even occur spontaneously by a reaction called strain-promoted azide-alkyne cycloaddition (SPAAC). This is one of the reactions known in the art as “metal-free click chemistry”.

### Application

The toxins according to the present invention, having structure (1), are especially suitable in the preparation of conjugates, such as the conjugates according to the present invention, which are in turn especially suitable in the treatment of cancer. The compound according to structure (1) are furthermore suitable for the killing of cells. In that light, the invention also concerns the use of the compound according to structure (1) for the killing of cells, as well as a method for killing cells comprising the contacting of the cells with the compound according to structure (1). The present use and method is typically ex vivo or in vitro.

The conjugates of the present invention are especially suitable in the treatment of cancer. In that light, the invention further concerns a method for the treatment of cancer, comprising administering to a subject in need thereof the conjugate according to the invention. The subject in need thereof is typically a cancer patient. The use of conjugates, such as antibody-drug conjugates, is well-known in the field of cancer treatment, and the conjugates according to the invention are especially suited in this respect. The method as described is typically suited for the treatment of cancer. In the method according to this aspect, the antibody-conjugate is typically administered in a therapeutically effective dose. The present aspect of the invention can also be worded as a conjugate according to the invention for use in the treatment of cancer. In other words, this aspect concerns the use of a conjugate according to the invention for the preparation of a medicament or pharmaceutical composition for use in the treatment of cancer. In the present context, treatment of cancer is envisioned to encompass treating, imaging, diagnosing, preventing the proliferation of, containing and reducing tumours.

This aspect of the present invention may also be worded as a method for targeting a tumour cell expressing a specific extracellular receptor, comprising contacting the conjugate according to the invention with cells that may possibly express the extracellular receptor, and wherein the antibody specifically targets the extracellular receptor. The method according to this aspect is thus suitable to determine whether the cells are expressing the desired extracellular receptor. These tumour cells may be present in a subject, in which case the method comprises administering to a subject in need thereof the conjugate according to the invention. Alternatively, the method occurs ex vivo or in vitro. In a preferred embodiment, the cells that may possibly express the extracellular receptor are cells that express the extracellular receptor. The targeting of tumour cells preferably includes one or more of treating, imaging, diagnosing, preventing the proliferation of, containing and reducing the tumour cells.

In the context of diagnosis, it is typically unknown whether the cells that are being contacted in fact express the specific extracellular receptor that is being investigated. For example, in the diagnosis of HER2-positive breast cancer, a conjugate containing an antibody that targets HER2, such as trastuzumab, may be contacted with the cells. In case the tumour cells are in fact HER2-expressing, the conjugate will target the cells, while in case the tumour cells are not HER2-expressing, the conjugate will not target the cells. Likewise, in the treatment of a cancer cells specifically expressing an extracellular receptor, the skilled person will understand that a cell-binding agent, such as an antibody, is to be used that targets that specific extracellular receptor.

In the methods of the present invention, it is preferred that the extracellular receptor is selected from the group consisting of 5T4, ADAM-9, AMHRII, ASCT2, ASLG659, ASPHD1, av-integrin, Axl, B7-H3, B7-H4, BAFF-R, BCMA, BMPR1B, Brevican, c-KIT, c-Met, C4.4a, CA-IX, cadherin-6, CanAg, CD123, CD13, CD133, CD138/syndecan-1, CD166, CD19, CD20, CD203c, CD205, CD21, CD22, CD228, CD25, CD30, CD324, CD33, CD37, CD38, CD45, CD46, CD48a, CD56, CD70, CD71, CD72, CD74, CD79a, CD79b, CEACAM5, claudin-18.2, claudin-6, CLEC12A, CLL-1, Cripto, CRIPTO, CS1, CXCR5, DLK-1, DLL3, DPEP3, E16, EGFR, ENPP3, EpCAM, EphA2, EphB2R, ETBR, FAP, FcRH1, FcRH2, FcRH5, FGFR2, fibronectin, FLT3, folate receptor alpha, Gal-3BP, GD3, GDNF-Ra1, GEDA, GFRA1, Globo H, gpNMB, GPR172A, GPR19, GPR54, guanyl cyclase C, HER2, HER3, HLA-DOB, IGF-1R, IL13R, IL20Ra, Lewis Y, LGR5, LIV-1, LRRC15, LY64, Ly6E, Ly6G6D, LY6K, MDP, MFI2, MICA/B, MOSPD2, MPF, MSG783, MUC1, MUC16, NaPi2b, NCA, nectin-4, Notch3, P-cadherin, P2X5, PD-L1, PMEL17, PRLR, PSCA, PSCA hlg, PSMA, PTK7, RET, RNF43, RON, ROR1, ROR2, Sema 5b, SLITRK6, SSTR2, STEAP1, STEAP2, TAG72, TENB2, TF, TIM-1, TM4SF, TMEFF, TMEM118, TMEM46, transferrin, TROP-2, TrpM4, TWEAKR, receptor tyrosine kinases (RTK), tenascin. Likewise, it is preferred that the tumour cells express an extracellular receptor selected from the same group. The skilled person is capable of matching the desired extracellular receptor with a suitable cell-binding agent capable of targeting that extracellular receptor.

In this light, the invention also concerns a pharmaceutical composition comprising the conjugate according to the invention and a pharmaceutically acceptable carrier. The pharmaceutical composition typically contains the conjugate according to the invention in a pharmaceutically effective dose.

The inventors have surprisingly found that the conjugates according to the invention are superior to conventional conjugates having a toxin derived from anthracycline, in terms of safety and/or efficacy, such that the therapeutic index of the antibody-conjugate according to the invention is increased with respect to conventional anthracycline-containing conjugates. In view of the reduced toxicity of the compounds according to structure (1), especially the safety of the conjugates according to the present invention is improved. As such, higher doses of the conjugate may be administered to the subject in need thereof, which in turn has further benefits in the treatment. Conventional conjugates of anthracycline with cell-binding agents such as antibodies need to be administered in very low doses, such that administering a too high dose is not uncommon. This may lead to a specific cell death and thus unwanted side-effects of cancer treatment. Furthermore, administration of anthracycline-antibody conjugates at their conventional low doses negatively affects the biodistribution, such that the targeting of the tumour is less efficient. Hence, the inventors have found anthracycline-based toxins, the compounds according to structure (1), that have a reduced toxicity, such that the therapeutic index, in particular the safety or tolerability, of the conjugates therewith is improved. Improved therapeutic efficacy of the conjugates according to the invention may take the form of a reduction in tumour size and/or a prolonged period of regression, when compared to conventional conjugates. Increase in tolerability of the conjugates according to the invention may take the form of a reduction in signs of toxicity, compared to administration of a conventional conjugate. The reduction in sings may also be referred to as a reduction in symptoms or side-effects of cancer treatment, and may involve one or more clinical signs such as reduced reduction in body weight, reduced reduction in mobility, reduced reduction in food intake and/or one or more toxicity parameters, such as improved blood chemistry, hematology, and/or histopathology.

In a further aspect, the invention concerns a method for modulating, improving or reducing the toxicity of an anthracycline-based toxin, comprising introducing the substituent R¹ as defined above. This aspect of the invention can also be worded as the use of substituent R¹ for modulating, improving or reducing the toxicity of an anthracycline-based toxin, wherein substituent R¹ as defined above. Herein, the conjugation to the compounds according to structure (1) is typically via Y as defined above.

### DESCRIPTION OF THE FIGURES

FIG. 1 shows the general scheme for preparation of antibody-drug conjugates by reaction of a monoclonal antibody (in most cases a symmetrical dimer) containing an x number of functionalities F. By incubation of antibody-(F) x with excess of a linker-drug construct (Q-spacer-linker-payload) a conjugate is obtained by reaction of F with Q, forming connecting group Z.

FIG. 2 depicts a range of reagents suitable for reaction with cysteine side-chains. Reagents may be monoalkylation type (A) or may be a cross-linker (B) for reaction with two cysteine side-chains.

FIG. 3 shows the general process for non-genetic conversion of a monoclonal antibody (mAb) into an antibody containing probes for click conjugation (F). The click probe may be on various positions in the antibody, depending on the technology employed. For example, the antibody may be converted into an antibody containing two click probes (structure on the left) or four click probes (bottom structure) or eight probes (structure on the right) for click conjugation.

FIG. 4 shows a representative (but not comprehensive) set of functional groups (F) that can be introduced into an antibody by engineering, by chemical modification, or by enzymatic means, which upon metal-free click reaction with a complementary reactive group Q lead to connecting group Z. Functional group F may be artificially introduced (engineered) into an antibody at any position of choice. Some functional groups F (e.g. nitrile oxide, quinone), may besides strained alkynes also react with strained alkenes, which as an example is depicted for triazine or tetrazine (bottom line). The pyridine or pyridazine connecting group is the product of the rearrangement of the tetrazabicyclo[2.2.2]octane connecting group, formed upon reaction of triazine or tetrazine with alkyne (but not alkene), respectively, with loss of N₂. Connecting groups Z depicted in FIG. 4 are preferred connecting groups to be used in the present invention.

FIG. 5 shows cyclic alkynes suitable for metal-free click chemistry, and preferred embodiments for reactive moiety Q. The list is not comprehensive, for example alkynes can be further activated by fluorination, by substitution of the aromatic rings or by introduction of heteroatoms in the aromatic ring.

FIG. 6 depicts a specific example of site-specific conjugation of a payload based on glycan remodeling of a full-length IgG followed by azide-cyclooctyne click chemistry. The IgG is first enzymatically remodeled by endoglycosidase-mediated trimming of all different glycoforms, followed by glycosyltransferase-mediated transfer of azido-sugar onto the core GlcNAc liberated by endoglycosidase. In the next step, the azido-remodeled IgG is subjected to an immune cell-engaging polypeptide, which has been modified with a single cyclooctyne for metal-free click chemistry (SPAAC), leading to a bispecific antibody of 2:2 molecular format. It is also depicted that the cyclooctyne-polypeptide construct will have a specific spacer between cyclooctyne and polypeptide, which enables tailoring of IgG-polypeptide distance or impart other properties onto the resulting bispecific antibody.

FIG. 7 depicts a specific example of site-specific conjugation of a payload based on glycan remodeling of a full-length IgG followed by thiol alkylation chemistry. The IgG is first enzymatically remodeled by endoglycosidase-mediated trimming of all different glycoforms, followed by glycosyltransferase-mediated transfer of a thiol-modified (and disulfide-protected) sugar derivative onto the core GlcNAc liberated by endoglycosidase. In the next step, the remodeled IgG is subjected to reduction (to convert the disulfide into thiol), potentially followed by oxidation, then reaction with a payload modified with a suitable thiol-reactive reagent.

FIG. 8 depicts the structures of daunorubicin, doxorubicin, nemorubicin (MMDX), PNU-159,696 and PNU-159,682.

FIG. 9 depicts two linker-modified PNU-159,682 derivatives, one based on carbamoylation of the hydroxyketone group with a linker containing N,N′-dimethylethylenediamine (DMEDA) and maleimide for antibody conjugation to cysteine, the other based on oxidation-amide coupling of the hydroxyketone with a linker containing ethylenediamine (EDA) and glycine-glycine for antibody conjugation under the action of sortase.

FIG. 10 shows an ADC obtained by sortase-mediated conjugation of glycine-glycine-EDA-modified oxidized PNU-159,682.

FIG. 11 shows the structure of linker-drugs based on PNU-159,682 analogues according to the invention, which can be applied for conjugation to antibodies through reactive moiety Z to generate the corresponding ADCs. Class 1 consists of a PNU-analogue modified at the morpholino ring with a substituent that is different from the methyl group present in PNU-159,682, and has the original hydroxyacetone moiety part oxidized to a carboxylic acid to enable activation/attachment of a linker. Class 2 consists of a PNU-analogue that has the original (hydroxy) acetone moiety of doxorubicin/daunorubicin retained and is modified with a linker at the position of the original methyl group present on the morpholino group of PNU-159,682. In either case, the linker is further modified with the reactive group Z, which can be any functionality that enables attachment to an antibody, e.g. a maleimide, an activated carbonyl, a halogenide, a cycloalkyne, an azide, etc.

FIG. 12 shows the synthetic scheme to generate PNU-159,682 analogues 6b-6f with modification at the morpholino ring based on initial TBS-protection of the hydroxyacetone function of doxorubicin,

FIG. 13 shows how N-alkylation of the aminosugar of doxorubicin can be achieved for various constructs 8b-8f without prior O-silylation of doxorubicin. This route is also applicable to daunorubicin.

FIG. 14A shows the structure of compound 9a based on Val-Cit dipeptide and DMEDA linker.

FIG. 14B shows the final step in the preparation of compounds 9c, 9d, 9f and 9g with Val-Ala dipeptide and EDA linker.

FIG. 15 shows the structures of compounds 36 and 39 with Val-Ala dipeptide and conjugation through the anthracycline morpholino group.

FIG. 16 shows the structures of compounds 47 and 53 based on EDA linkers and Gly-Gly-Phe-Gly or Gly-Gly-Gly peptides, respectively.

FIG. 17 shows the in vitro cytotoxicity of trast-9g, trast-9d, trast-9c and trast-36 on four cell lines with variable HER2 expression levels. The To line indicates the number of viable cells at the start of the assay.

FIG. 18A shows the time-dependent average body weight of CD-1 mice administered a single bolus of vehicle (PBS), ADC trastuzumab-9d (20 mg/kg), ADC trastuzumab-36 (20 mg/kg) or reference ADC trastuzumab-9g (5 mg/kg).

FIG. 18B shows the time-dependent body weight of CD-1 mice administered a single bolus of vehicle (PBS), ADC trastuzumab-47 (15 mg/kg), ADC trastuzumab-9c (40 mg/kg) or reference ADC trastuzumab-9g (5 mg/kg).

FIG. 19A shows the tumor volume over time of NOD/SCID mice grafted with JIMT-1 tumor cell line followed by treatment with reference ADC trastuzumab-9g at low (0.3 mg/kg) or high (1 mg/kg) dose.

FIG. 19B shows the tumor volume over time of NOD/SCID mice grafted with JIMT-1 tumor cell line followed by treatment with ADC trastuzumab-9d at low (3 mg/kg) or high (5 mg/kg) dose or ADC trastuzumab-36 at low (0.6 mg/kg) or high (2 mg/kg) dose.

FIG. 19C shows the tumor volume over time of NOD/SCID mice grafted with JIMT-1 tumor cell line followed by treatment with ADC trastuzumab-47 at low (0.6 mg/kg) or high (2 mg/kg) dose or ADC trastuzumab-9c at high dose (2 mg/kg).

FIG. 20 shows the in vitro cytotoxicity of compounds 6a, 6b, 6c, 6d and 6e on four cell lines with variable HER2 expression levels. The To line indicates the number of viable cells at the start of the assay.

FIG. 21 shows the in vitro cytotoxicity of trast-9g, trast-63a, trast-63b and trast-75 on three cell lines with variable HER2 expression levels. The To line indicates the number of viable cells at the start of the assay.

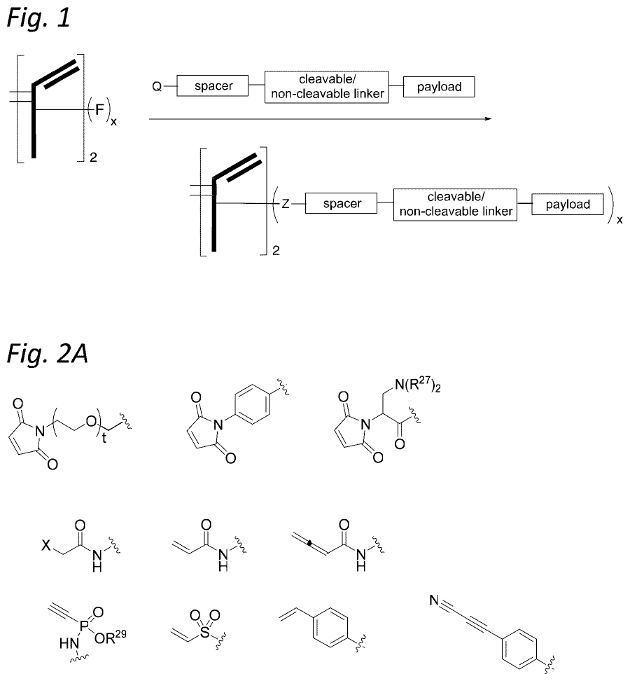

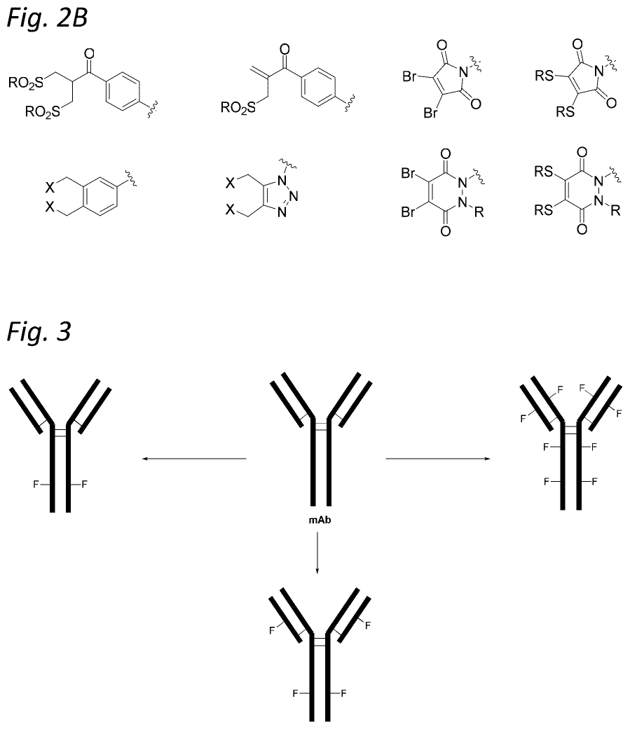

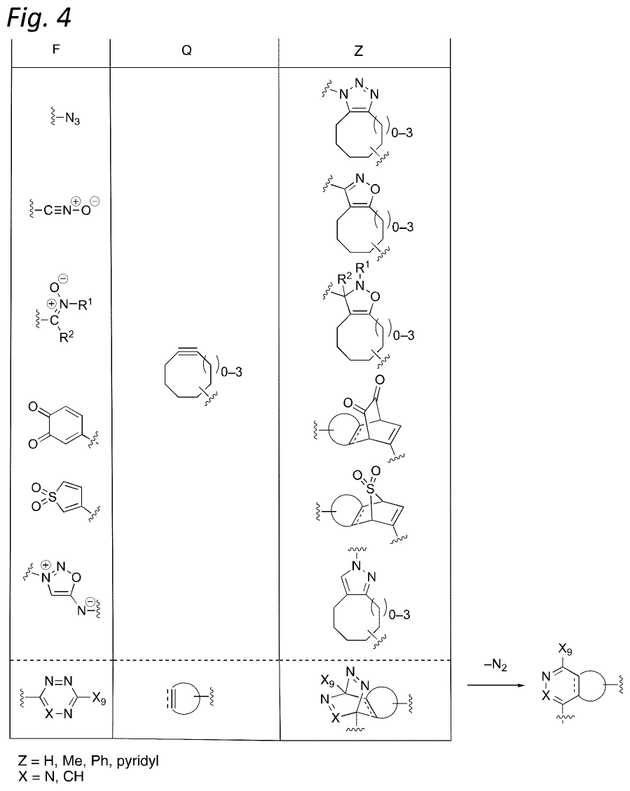

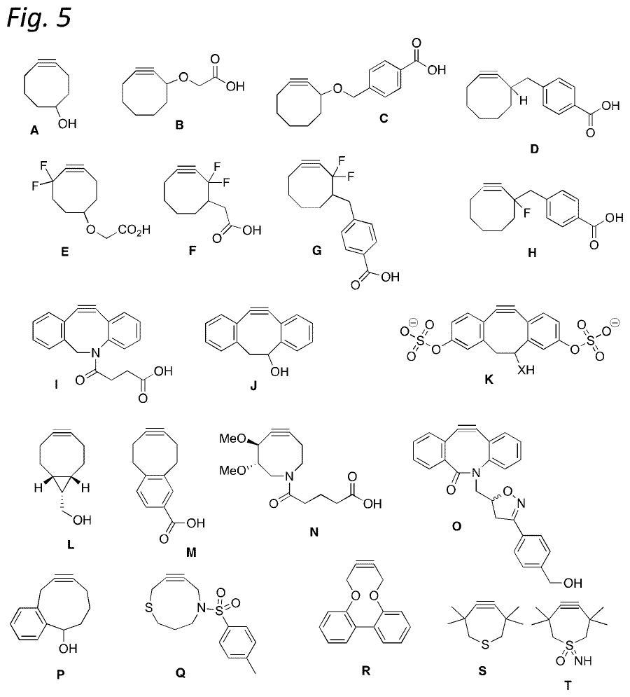

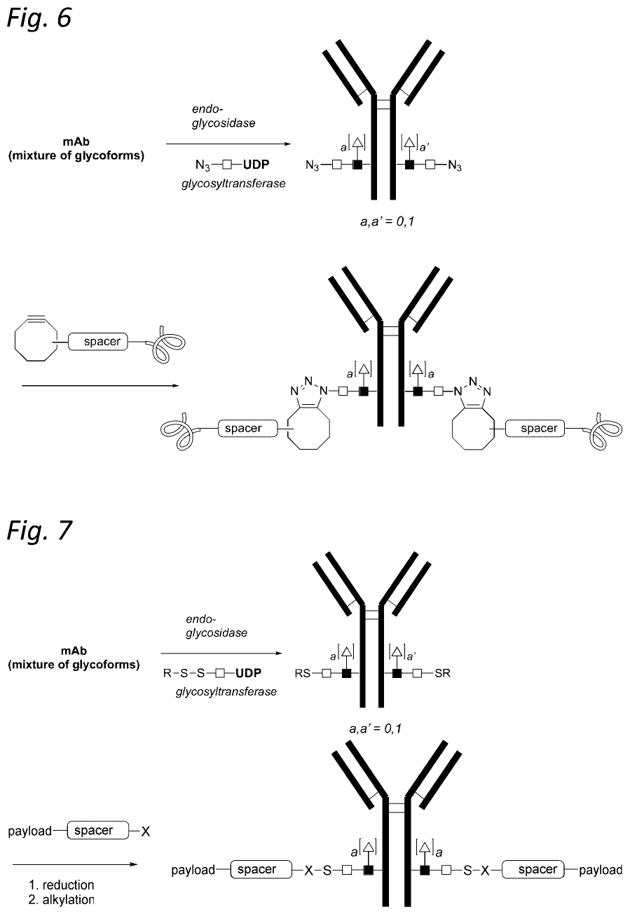

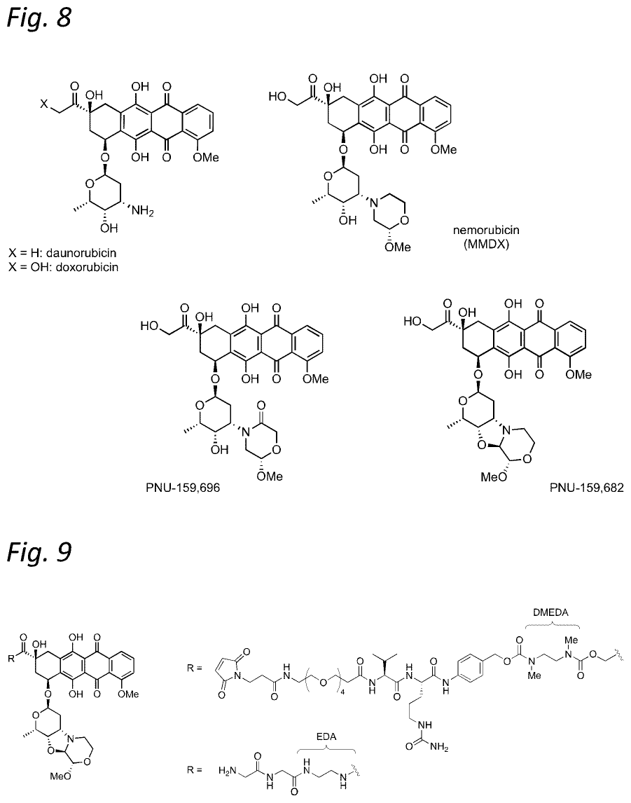

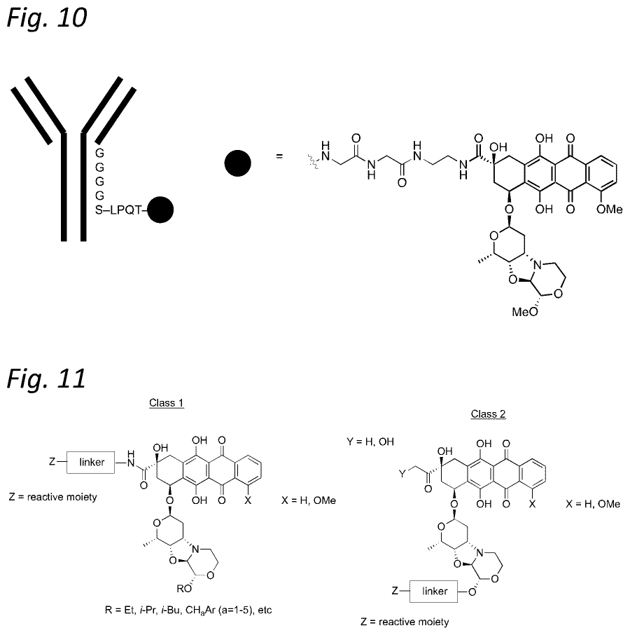

### EXAMPLES

The invention is illustrated by the following examples.

### General Procedure for Analytical RP-UPLC

Prior to RP-UPLC analysis, IgG (10 μL, 1 mg/mL in PBS pH 7.4) was added to 12.5 mM DTT, 100 mM TrisHCl pH 8.0 (40 μL) and incubated for 15 minutes at 37° C. The reaction was quenched by adding 49% acetonitrile, 49% water, 2% formic acid (50 μL). RP-UPLC analysis was performed on an H-class Acquity UPLC system (Waters). The sample (5 μL) was injected with 0.4 mL/min onto a BioResolve™ RP mAb Polyphenyl column (450 Å, 2.7 μm, 2.1×150 mm, Waters) with a column temperature of 70° C. A linear gradient was applied in 9 minutes from 30 to 55% acetonitrile in 0.1% TFA and water.

### General Procedure for Analytical SEC

SE-HPLC analysis was performed on an Agilent 1100 series (Hewlett Packard) using an Xbridge BEH200A column (3.5 M, 7.8×300 mm, PN 186007640, Waters). The sample was diluted to 1 mg/mL in PBS and measured with 0.86 mL/min isocratic method (0.1 M sodium phosphate buffer pH 6.9 (NaHPO₄/Na₂PO₄) containing 10% isopropanol) for 16 minutes.

### General Procedure for Mass Spectral Analysis of Monoclonal Antibodies and ADC

Prior to mass spectral analysis, IgG was treated with IdeS (Fabricator™) for analysis of the Fc/2 fragment. A solution of 20 μg (modified) IgG was incubated for 1 hour at 37° C. with 0.5 μL IdeS (50 U/μL) in phosphate-buffered saline (PBS) pH 6.6 in a total volume of 10 μL. Samples were diluted to 40 μL followed by analysis on a JEOL AccuTOF LC-plus JMS-T100LP system (ESI-TOF) combined with a HPLC system (Agilent 1100 series, Hewlett Packard). On the HPLC system a MassPREP™ On-line Desalting Cartridge (Waters P/N 186002785) is installed.

### General Procedure for LC-MS Analysis of Monoclonal Antibodies and ADC

For analysis of Fc/2 fragments, IgG was treated with IdeS (Fabricator™). A solution of 10 μg (modified) IgG was incubated for 1 hour at 37° C. with 0.5 μL IdeS (50 U/μL) in phosphate-buffered saline (PBS) pH 7.4 in a total volume of 10 μL followed by dilution to 100 μL using MQ. For analysis of reduced samples, IgG was treated with DTT. A solution of 10 μg (modified) IgG was incubated for 15 minutes at 37° C. with DTT (10 mM) in a final volume of 50 μL PBS pH 7.4 followed by addition of 50 μL quench buffer (49% MQ, 49% acetonitrile, 2% FA). Sample were analyzed on a Xevo G2-XS QTof Quadrupole Time-of-Flight Mass Spectrometry system (ESI-QTOF) combined with a UPLC system (Aquity series, Waters). On the UPLC system a bioZen™ 3.6 μm Intact XB-C8, LC column 50×2.1 mm (cat no: 00B-4766-AN) is installed.

### Synthesis of Bis-Iodo Compound 7b

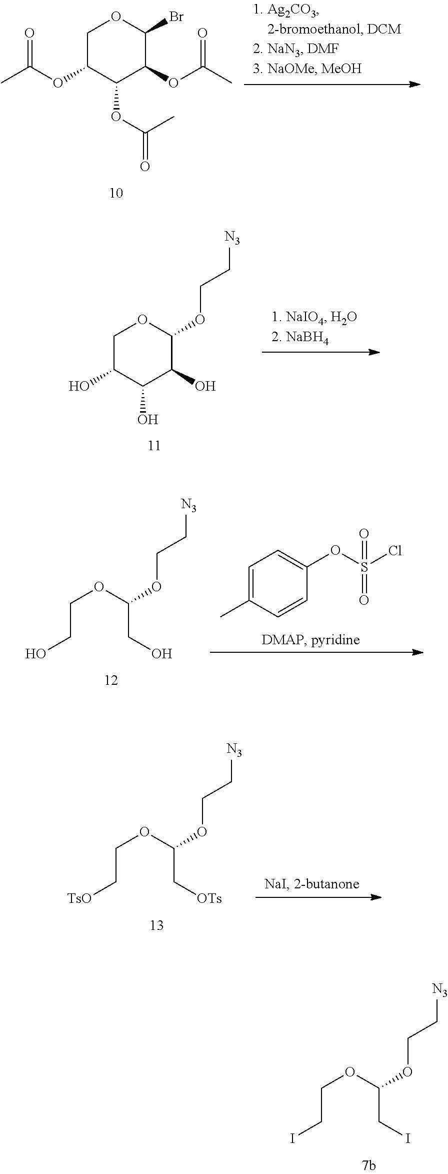 [SMILES: CC(=O)O[C@H]1[C@H](OC(C)=O)[C@H](OC(C)=O)CO[C@H]1Br ; [N-]=[N+]=NCCO[C@H]1OC[C@@H](O)[C@@H](O)[C@@H]1O ; [N-]=[N+]=NCCO[C@H](CO)OCCO ; Cc1ccc(OS(=O)(=O)Cl)cc1 ; Cc1ccc(S(=O)(=O)OCCO[C@@H](COS(=O)(=O)c2ccc(C)cc2)OCCN=[N+]=[N-])cc1 ; [N-]=[N+]=NCCO[C@H](CI)OCCI]

### Example a1. Synthesis of 2,3,4-tri-O-acetyl-β-D-arabinopyranosylbromide (10)

Tetraacetyl arabinose (24.6 g) was dissolved in a solution of HBr in AcOH (33% HBr, 127 mL). Ac₂O (12 mL) was added, and the mixture was stirred at rt overnight. DCM (200 mL) was added, and the mixture was poured onto ice (300 mL). The two phases were separated, and the aqueous layer was extracted with DCM (2×250 mL). The combined organic layers were washed with sat. aq. NaHCO₃ (400 mL) and dried over Na₂SO₄. The mixture was concentrated and recrystallized using Et₂O/heptane to obtain compound 10 (17.66 g, 67.4%). ¹H-NMR data was identical to data reported by Grugel et al. Synthesis, 2010, 19, 3248-3258.

### Example a2. Synthesis of 1-(2-azidoethyl)-α-D-arabinopyranoside (11)

Arabinosyl bromide 10 (2.47 g, 6.45 mmol) was dissolved in dry DCM (0.2 M). Molecular sieves, and 2-bromoethanol (5 eq) were added, and the mixture was cooled to 0° C. After addition of Ag₂CO₃ (1 eq), the reaction mixture was warmed to room temperature and stirred for 3 h. The mixture was filtered through celite, the celite pad was washed with Et₂O and the solvent was evaporated. The crude product was purified using flash column chromatography (0→25% EtOAc in heptane). Fractions containing the product were concentrated, dissolved in DMF (0.2 M), after which NaN₃ (4 eq) was added. The mixture was stirred at 80° C. for 1 h and concentrated, after which it was redissolved in MeOH (0.1 M) and NaOMe (5.4 M in MeOH, 0.1 eq) was added. The reaction was stirred overnight, concentrated, and purified using flash column chromatography (0→10% MeOH in EtOAc) to obtain the product 11 (530 mg, 37.5% over three steps). ¹H-NMR (500 MHz, CDCl₃) δ (ppm) 4.28 (d, J=7.1 Hz, 1H), 4.10-3.99 (m, 2H), 3.97 (s, 1H), 3.95-3.90 (m, 1H), 3.80-3.72 (m, 2H), 3.69 (m, 2H), 3.60-3.53 (m, 2H), 3.45 (ddd, J=13.3, 5.8, 3.6 Hz, 2H). ¹³C-NMR (126 MHZ, CDCl₃) δ (ppm) 103.28, 72.97, 71.42, 68.22, 68.20, 65.91, 50.84.

### Example a3. Synthesis of 1,5-dihydroxy-2 (S)-(2-azidoethoxy)-3-oxa-pentane (12)

Arabinoside 11 (530 mg, 2.42 mmol) was dissolved in H₂O (0.25 M). NaOAc (1.3 eq) was added, followed by NalO₄ (2.5 eq). After stirring for 1 h in the dark, TLC (10% MeOH in EtOAc) showed full consumption of the starting material. The mixture was cooled to 0° C., followed by addition of NaBH₄ in portions. After 1 h, TLC (10% MeOH in EtOAc) showed formation of the diol. EtOAc (10 mL) was added, and the organic layers were separated. The aqueous layer was extracted with EtOAc (10 mL) five times. The combined organic layers were dried over Na₂SO₄ and concentrated, to obtain product 12 (346 mg, 74.8%). ¹H-NMR (400 MHZ, CDCl₃) δ (ppm) 4.72 (t, J=5.3 Hz, 1H), 3.94-3.83 (m, 2H), 3.82-3.76 (m, 2H), 3.77-3.65 (m, 4H), 3.52-3.38 (m, 2H).

¹³C-NMR (101 MHZ, CDCl₃) δ (ppm) 102.88, 68.39, 66.11, 62.34, 61.80, 50.92.

### Example a4. Synthesis of 1,5-di(p-toluenesulphonyl)oxy-2(S)-(2-azidoethoxy)-3-oxa-pentane (13)

Diol 12 (346 mg, 1.81 mmol) was dissolved in dry pyridine (0.1 M), cooled to 0° C. followed by addition of p-TsCl (2.5 eq). The mixture was stirred overnight, concentrated and dissolved in EtOAc (20 mL). The solution was washed with 0.1M HCl (10 mL) and brine (10 mL). The mixture was dried over Na₂SO₄, concentrated, and purified using flash column chromatography (0→50% EtOAc in heptane) to obtain product 13 (368 mg, 40.7%). ¹H-NMR (400 MHZ, CDCl₃) δ (ppm) 7.86-7.77 (m, 4H), 7.47-7.33 (m, 5H), 4.74 (t, J=5.4 Hz, 1H), 4.15 (ddd, J=5.4, 4.0, 1.1 Hz, 2H), 3.97 (dd, J=5.4, 0.8 Hz, 2H), 3.85-3.69 (m, 3H), 3.66-3.58 (m, 1H), 3.36 (dt, J=5.8, 3.9 Hz, 2H), 2.48 (d, J=1.5 Hz, 6H). ¹³C-NMR (101 MHz, CDCl₃) δ (ppm) 145.26, 145.08, 132.82, 132.50, 130.00, 129.94, 128.01, 127.96, 99.40, 68.77, 67.86, 65.71, 64.27, 50.67, 21.69, 21.67.

### Example a5. Synthesis of 1,5-diiodo-2 (S)-(2-azidoethoxy)-3-oxa-pentane (7b)

Bis-tosylate 13 (491 mg, 0.98 mmol) was dissolved in 2-butanone (0.05 M). Nal (7 eq) was added, and the mixture was stirred at 90° C. for 24 h. The mixture was concentrated and dissolved in EtOAc (20 mL). The organic layer was washed with H₂O (20 mL) and brine (20 mL), dried over Na₂SO₄ and concentrated. The crude product was purified using flash column chromatography (0→5% EtOAc in heptane) to obtain compound 7b (260 mg, 64.4%). ¹H-NMR (400 MHZ, CDCl₃) δ (ppm) 4.80 (t, J=5.6 Hz, 1H), 3.98-3.80 (m, 3H), 3.73 (ddd, J=10.4, 5.8, 4.5 Hz, 1H), 3.46 (ddd, J=5.9, 4.2, 1.8 Hz, 2H), 3.37-3.26 (m, 4H). ¹³C-NMR (101 MHZ, CDCl₃) δ (ppm) 101.83, 66.95, 65.05, 50.73, 3.77, 2.33.

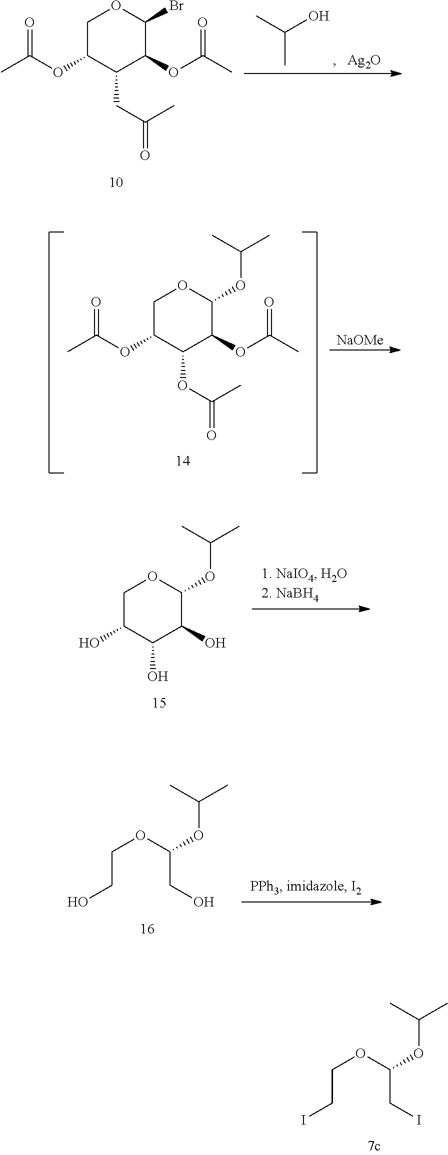

### Example a6. Synthesis of 1-Isopropyl-α-D-arabinopyranoside (15)

Arabinosyl bromide 10 (3.51 g, 10.3 mmol) was dissolved in dry Et₂O (0.25 M). iPrOH (15 eq) was added, followed by Ag₂O (1 eq). The mixture was stirred in the dark for 3 h. The reaction was filtered over celite, the celite pad was washed with Et₂O and the ether was removed by rotary evaporation. The crude mixture was dissolved in MeOH (0.1 M), followed by addition of NaOMe (5.4 M in MeOH, 0.1 eq.) and stirred overnight at rt. The product was purified using flash column chromatography (0→10% MeOH in EtOAc) to obtain product 15 (1.38 g, 69.4% over two steps). ¹H-NMR (400 MHZ, D₂O) δ (ppm) 5.70-5.66 (m, 1H), 5.38 (hept, J=6.2 Hz, 1H), 5.26 (dd, J=12.4, 2.9 Hz, 1H), 5.22 (dt, J=2.9, 1.5 Hz, 1H), 5.00-4.85 (m, 3H), 2.62 (dd, J=13.0, 6.1 Hz, 6H). ¹³C-NMR (101 MHZ, D₂O) δ (ppm) 103.16, 74.35, 72.50, 72.48, 69.72, 66.84, 23.81, 22.07.

### Example a7. Synthesis of 1,5-dihydroxy-2 (S)-isopropyloxy-3-oxa-pentane (16)

Arabinoside 15 (585 mg, 3.04 mmol) was dissolved in H₂O (0.25 M). NaOAc (1.3 eq) was added, followed by NalO₄ (2.5 eq). After stirring for 1 h in the dark, TLC (10% MeOH in EtOAc) showed full consumption of the starting material. The mixture was cooled to 0° C., followed by addition of NaBH₄ in portions. After 1 h, TLC (10% MeOH in EtOAc) showed formation of the diol. EtOAc (10 mL) was added, and the organic layers were separated. The aqueous layer was extracted with EtOAc (10 mL) five times. The combined organic layers were dried over Na₂SO₄ and concentrated, to obtain product 16 (324 mg, 64.8%). ¹H-NMR (500 MHZ, CDCl₃) δ (ppm) 4.72 (dd, J=6.1, 4.7 Hz, 1H), 3.93 (dq, J=12.3, 6.1 Hz, 1H), 3.87-3.75 (m, 3H), 3.71-3.53 (m, 3H), 1.26 (d, J=5.7 Hz, 2H), 1.20 (d, J=6.1 Hz, 3H). ¹³C-NMR (126 MHZ, CDCl₃) δ (ppm) 100.99, 70.29, 67.64, 63.21, 62.02, 23.09, 22.32.

### Example a8. Synthesis of 1,5-diiodo-2 (R)-isopropyloxy-3-oxa-pentane (7c)

Diol 16 (244 mg, 1.49 mmol) was dissolved in dry THF (0.15 M). Imidazole (7 eq), PPh₃ (3 eq) were added, followed by the addition of I₂ (3 eq). The mixture was stirred overnight at rt in the dark. After dilution with EtOAc (20 mL), the organic layer was washed with 10% aq. sodium thiosulfate (20 mL), brine (20 mL) and dried over Na₂SO₄. The mixture was concentrated and purified using column chromatography (0→5% EtOAc in heptane) to obtain the product 7c (220 mg, 38.5%). ¹H-NMR (400 MHZ, CDCl₃) δ (ppm) 4.76 (t, J=5.5 Hz, 1H), 3.94 (hept, J=6.2 Hz, 1H), 3.87-3.71 (m, 0H), 3.33-3.12 (m, 4H), 1.24 (d, J=6.2 Hz, 3H), 1.20 (d, J=6.1 Hz, 3H). ¹³C-NMR (101 MHZ, CDCl₃) δ (ppm) 100.29, 70.13, 65.65, 23.16, 22.06, 5.74, 2.61.

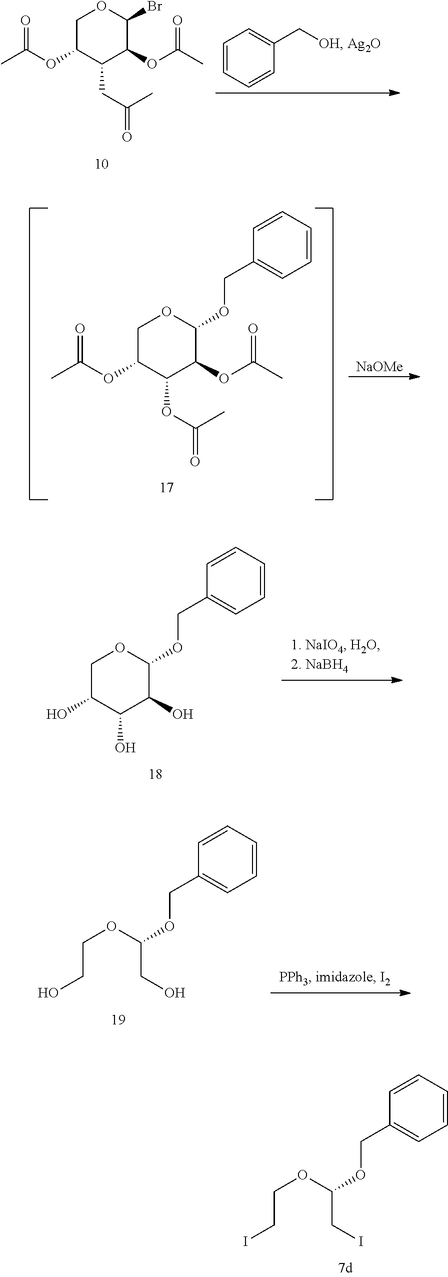

### Example a9. Synthesis of 1-benzyl-α-D-arabinopyranoside (18)

Arabinosyl bromide 10 (2.06 g, 6.09 mmol) was dissolved in dry Et₂O (0.25 M). BnOH (15 eq) was added, followed by Ag₂O (1 eq). The mixture was stirred in the dark for 3 h. The reaction was filtered over celite, the celite pad was washed with Et₂O and the ether was removed by rotary evaporation. The crude mixture was dissolved in MeOH (0.1 M), followed by addition of NaOMe (5.4 M in MeOH, 0.1 eq) and stirred overnight at rt. The product was purified using flash column chromatography (0→10% MeOH in EtOAc) to obtain the product 18 (840 mg, 57.4% over two steps). ¹H-NMR (400 MHZ, D2O) δ (ppm) 7.53-7.37 (m, 5H), 4.92 (dd, J=11.6, 1.4 Hz, 1H), 4.75 (dd, J=11.6, 1.2 Hz, 1H), 4.44 (d, J=7.5 Hz, 1H), 4.02-3.91 (m, 2H), 3.73-3.55 (m, 3H). ¹³C-NMR (101 MHZ, D2O) δ (ppm) 136.63, 128.72, 128.64, 128.45, 102.18, 102.13, 72.35, 71.42, 70.72, 70.69, 68.31, 68.24, 66.28.

### Example a10. Synthesis of 1,5-dihydroxy-2 (S)-benzyloxy-3-oxa-pentane (19)

Arabinoside 18 (259 mg, 1.08 mmol) was dissolved in H₂O (0.25 M). NaOAc (1.3 eq) was added, followed by NalO₄ (2.5 eq). After stirring for 1 h in the dark, TLC (10% MeOH in EtOAc) showed full consumption of the starting material. The mixture was cooled to 0° C., followed by addition of NaBH₄ in portions. After 1 h, TLC (10% MeOH in EtOAc) showed formation of the diol. EtOAc (10 mL) was added, and the organic layers were separated. The aqueous layer was extracted with EtOAc (10 mL) five times. The combined organic layers were dried over Na₂SO₄ and concentrated, to obtain product 19 (198 mg, 86.7%). ¹H-NMR (400 MHZ, CDCl₃) δ (ppm) 7.43-7.17 (m, 5H), 4.75-4.66 (m, 2H), 4.57 (d, J=11.7 Hz, 1H), 3.83 (ddd, J=10.6, 5.3, 3.3 Hz, 1H), 3.77-3.71 (m, 2H), 3.68-3.55 (m, 2H). ¹³C-NMR (101 MHZ, CDCl₃) δ (ppm) 137.49, 128.55, 127.97, 127.85, 102.29, 69.51, 68.31, 62.44, 61.68.

### Example a11. Synthesis of 1,5-diiodo-2 (S)-benzyloxy-3-oxa-pentane (7d)

Diol 19 (579 mg, 2.73 mmol) was dissolved in dry THF (0.15 M). Imidazole (7 eq), PPh3 (3 eq) were added, followed by the addition of 12 (3 eq). The mixture was stirred overnight at rt in the dark. After dilution with EtOAc (20 mL), the organic layer was washed with 10% aq. sodium thiosulfate (20 mL), brine (20 mL) and dried over Na₂SO₄. The mixture was concentrated and purified using column chromatography (0→5% EtOAc in heptane) to obtain the product 7d (723 mg, 61.3%). ¹H-NMR (400 MHZ, CDCl₃) δ (ppm) 7.45-7.31 (m, 5H), 4.83 (t, J=5.6 Hz, 1H), 4.76 (d, J=11.7 Hz, 1H), 4.66 (d, J=11.7 Hz, 1H), 3.94-3.75 (m, 2H), 3.37-3.24 (m, 4H). ¹³C-NMR (101 MHZ, CDCl₃) δ (ppm) 137.16, 128.57, 128.05, 127.99, 101.12, 68.66, 66.72, 4.52, 2.50.

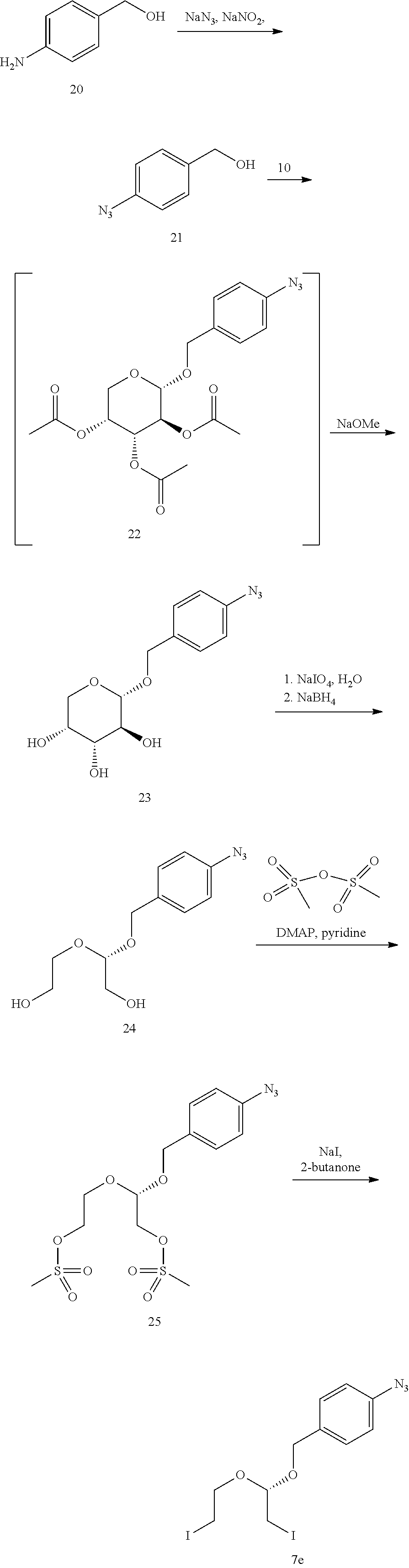

### Example a12. Synthesis of Compound 21

An ice-cold solution of sodium nitrite (842.3 mg, 12.21 mmol) in water (20 mL) was prepared and transferred to a dropping funnel. This mixture was added over 30 min to a cold solution of 4-aminobenzyl alcohol (1 g, 8.12 mmol) in HCl (5 M, 5 mL). The reaction mixture turned from bright yellow to light yellow and finally to off-white. After 30 min, sodium azide (2.1 g, 32 mmol) was added in 5 portions and the mixture was left stirring. After an hour the ice bath was removed, and solids were observed. After 1.5 h, saturated aqueous NaHCO₃ solution (25 mL) was added followed by EtOAc (25 mL). The reaction was transferred to a separation funnel and the organic layer was separated from the water layer. The organic layer was washed with saturated aqueous NaHCO₃ solution (20 mL), washed with brine (25 mL) and dried over Na₂SO₄. The drying agent was filtered off over a glass filter and the yellow filtrate was concentrated. The crude yellow oil was purified by flash column chromatography over silicagel (5%→80% EtOAc in heptane, column pre-conditioned with 5% EtOAc in heptane) to give product 21 in 89% (1.08 g, 7.24 mmol). ¹H-NMR (400 MHZ, CDCl₃) δ (ppm) 7.36 (d, J=8.6 Hz, 2H), 7.08-6.97 (m, 2H), 4.67 (d, J=4.3 Hz, 2H), 1.68 (t, J=5.2 Hz, 1H).

### Example a13. Synthesis of Compound 23

To a solution of arabinosyl bromide 10 (1.34 mg, 3.95 mmol) and compound 21 (872 mg, 5.85 mmol) in diethyl ether (anhydrous, 20 mL) was added silver(I) oxide (916 mg, 3.95 mmol) and the reaction was stirred in the dark at room temperature. After stirring for 10 days, the reaction mixture was filtered over pre-wetted celite and washed through with diethyl ether and concentrated. The crude oil was dissolved in MeOH (15 mL), and sodium methoxide (134.4 mg, 2.48 mmol) was added. After stirring for 3.5 h at room temperature, the reaction mixture was neutralized with a few drops of 1 M aq. HCl solution and concentrated. The excess 21 was removed by precipitating the desired compound in diethyl ether and filtering it over a glass filter covered with filter paper. Compound 23 was obtained in 65% yield (714.4 mg, 2.54 mmol) as an off-white solid. ¹H-NMR (400 MHZ, MeOD) δ (ppm) 7.47 (d, J=8.6 Hz, 2H), 7.10-7.04 (m, 2H), 4.86 (d, J=11.9 Hz, 1H), 4.63 (d, J=11.9 Hz, 1H), 4.31 (d, J=6.9 Hz, 1H), 3.95-3.79 (m, 3H), 3.67-3.49 (m, 4H).

### Example a14. Synthesis of Compound 24

Compound 23 (714.4 mg, 2.54 mmol, 1.0 eq) was dissolved in MeOH (3 mL) and water (5 mL) and cooled down to 0° C. (in the dark). A solution of sodium acetate (270.9 mg, 3.3 mmol, 1.3 eq) in water (3 mL) was added at once followed by sodium periodate (1.35 g, 6.35 mmol, 2.5 eq) in portions. The reaction mixture was stirred on ice for 15 min after which the ice bath was removed and stirring was continued for 4.5 h at room temperature. After stirring for 4.5 h, the reaction mixture was cooled again to −10° C. and sodium borohydride (288.3 mg, 7.62 mmol, 3.0 eq) was added in portions. After stirring for an hour, EtOAc (50 mL) was added, and the reaction mixture was transferred to a separation funnel. The organic layer was separated from the water layer and the water layer was extracted with EtOAc (5×50 mL). The organic layers were combined and dried over Na₂SO₄, filtered over a glass filter with pre-wetted celite and concentrated. Compound 24 was obtained in 87% yield (637.2 mg, 2.2 mmol). ¹H-NMR (400 MHZ, MeOD) δ (ppm) 7.31 (d, J=8.5 Hz, 2H), 6.98-6.91 (m, 2H), 4.63-4.48 (m, 3H), 3.70-3.65 (m, 1H), 3.60-3.56 (m, 2H), 3.53-3.48 (m, 3H).

### Example a15. Synthesis of Compound 25

A solution of 24 (637.2 mg, 2.21 mmol) in dry DCM (8 mL) was cooled to 0° C. (under flow of nitrogen). Pyridine (537 μL, 6.64 mmol, 3.0 eq), methanesulfonic anhydride (964.2 mg, 5.53 mmol, 2.5 eq) and DMAP (27.0 mg, 221.4 mmol, 0.1 eq) were added. After stirring for 3.5 h, the reaction mixture was washed with aqueous saturated NaHCO₃ solution (11 mL). The water layer was extracted twice with DCM (10 mL). The combined organic layers were dried over Na₂SO₄, filtered and concentrated. The crude orange oil was purified by flash column chromatography over silicagel (10%→80% EtOAc in heptane, column pre-conditioned with 10% EtOAc in heptane) to give compound 25 as a clear light-yellow oil in 61% (571.6 mg, 1.4 mmol). ¹H-NMR (400 MHZ, CDCl₃) δ (ppm) 7.34 (d, J=8.5 Hz, 2H), 7.06-7.00 (m, 2H), 4.91 (t, J=5.2 Hz, 1H), 4.72 (d, J=11.7 Hz, 1H), 4.61 (d, J=11.7 Hz, 1H), 4.37 (t, J=4.5 Hz, 2H), 4.24 (dd, J=5.2, 2.1 Hz, 2H), 3.96-3.88 (m, 1H), 3.86-3.80 (m, 1H), 3.06 (s, 3H), 3.05 (s, 3H).

### Example a16. Synthesis of Compound 7e

A solution of compound 25 (571.2 mg, 1.4 mmol, 1.0 eq) and sodium iodide (1.42 g, 9.48 mmol, 7 eq) in 2-butanone (15 mL) was refluxed in the dark. After refluxing for 72 h, the reaction mixture was diluted with EtOAc (30 mL) and washed with water (20 mL). The layers were separated, and the organic layer was washed with brine (20 mL). The combined organic layers were dried over Na₂SO₄, filtered and concentrated. The crude orange oil was purified by flash column chromatography over silicagel (0%→7% EtOAc in heptane) to give compound 7e as an opaque light-yellow oil in 47.5% (313.2 mg, 0.64 mmol). ¹H-NMR (400 MHZ, CDCl₃) δ (ppm) 7.37 (d, J=8.6 Hz, 2H), 7.11-6.93 (m, 2H), 4.79 (t, J=5.6 Hz, 1H), 4.70 (d, J=11.6 Hz, 1H), 4.59 (d, J=11.7 Hz, 1H), 3.90-3.72 (m, 2H), 3.35-3.18 (m, 4H).

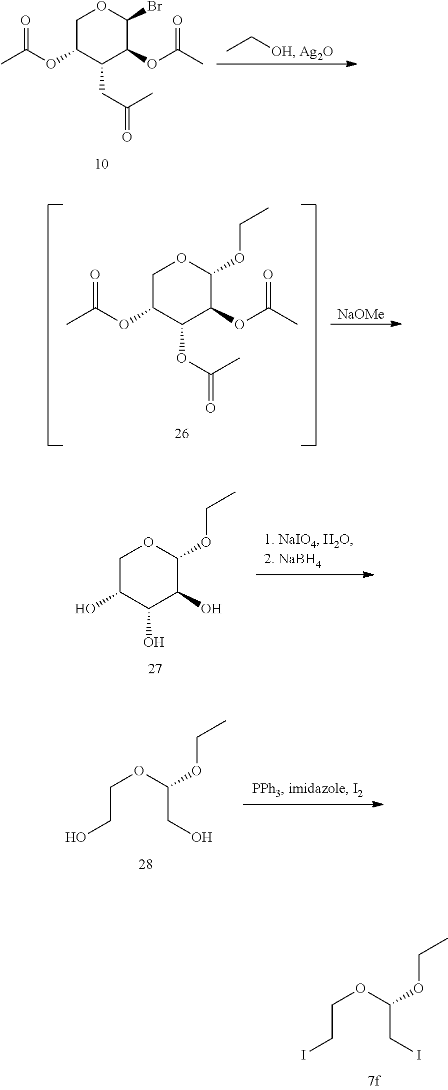

### Example a17. Synthesis of 1-ethyl-α-D-arabinopyranoside (27)

Arabinosyl bromide 10 (5.31 g, 15.66 mmol) was dissolved in dry Et₂O (0.25 M). EtOH (15 eq) was added, followed by Ag₂O (1 eq). The mixture was stirred in the dark for 3 h. The reaction was filtered over celite, the celite pad was washed with Et₂O and the ether was removed by rotary evaporation. The crude mixture was dissolved in MeOH (0.1 M), followed by addition of NaOMe (5.4 M in MeOH, 0.1 eq) and stirred overnight at rt. The product was purified using flash column chromatography (0→10% MeOH in EtOAc) to obtain the product 27 (1.80 g, 64.5% over two steps). ¹H-NMR (400 MHZ, MeOD) δ (ppm) 4.22 (d, J=7.1 Hz, 1H), 3.97-3.79 (m, 3H), 3.67-3.50 (m, 4H), 1.26 (t, J=7.1 Hz, 3H). ¹³C-NMR (101 MHZ, MeOD) δ (ppm) 104.65, 74.33, 72.43, 69.70, 66.91, 65.94, 15.49.

### Example a18. Synthesis of 1,5-dihydroxy-2(S)-ethoxy-3-oxa-pentane (28)

Arabinoside 27 (721 mg, 4.05 mmol) was dissolved in H₂O (0.25 M). NaOAc (1.3 eq) was added, followed by NalO₄ (2.5 eq). After stirring for 1 h in the dark, TLC (10% MeOH in EtOAc) showed full consumption of the starting material. The mixture was cooled to 0° C., followed by addition of NaBH₄ in portions. After 1 h, TLC (10% MeOH in EtOAc) showed formation of the diol. EtOAc (15 mL) was added, and the organic layers were separated. The aqueous layer was extracted with EtOAc (15 mL) five times. The combined organic layers were dried over Na₂SO₄ and concentrated, to obtain product 28 (381 mg, 62.7%). ¹H-NMR (400 MHZ, CDCl₃) δ (ppm) 4.62 (dd, J=6.1, 4.3 Hz, 1H), 3.89-3.81 (m, 1H), 3.79-3.69 (m, 3H), 3.67-3.53 (m, 4H), 1.24-1.19 (m, 3H). ¹³C-NMR (101 MHZ, CDCl₃) δ (ppm) 102.65, 68.44, 63.66, 62.61, 61.83, 15.29.

### Example a19. Synthesis of 1,5-diiodo-2 (S)-ethoxy-3-oxa-pentane (7f)

Diol 28 (381 mg, 2.54 mmol) was dissolved in dry THF (0.15 M). Imidazole (7 eq), PPh3 (3 eq) were added, followed by the addition of 12 (3 eq). The mixture was stirred overnight at rt in the dark. After dilution with EtOAc (20 mL), the organic layer was washed with 10% sodium thiosulfate (20 mL), brine (20 mL) and dried over Na₂SO₄. The mixture was concentrated and purified using column chromatography (0→5% EtOAc in heptane) to obtain the product 7f (170 mg, 18.1%). ¹H-NMR (400 MHZ, CDCl₃) δ (ppm) 4.72 (t, J=5.5 Hz, 1H), 3.93-3.70 (m, 3H), 3.66-3.56 (m, 1H), 3.32-3.23 (m, 4H), 1.26 (t, J=7.1 Hz, 3H). ¹³C-NMR (101 MHZ, CDCl₃) δ (ppm) 101.76, 66.90, 62.52, 15.10, 4.78, 2.48.

General Scheme from Doxorubicin to PNU-159,682 Analogues

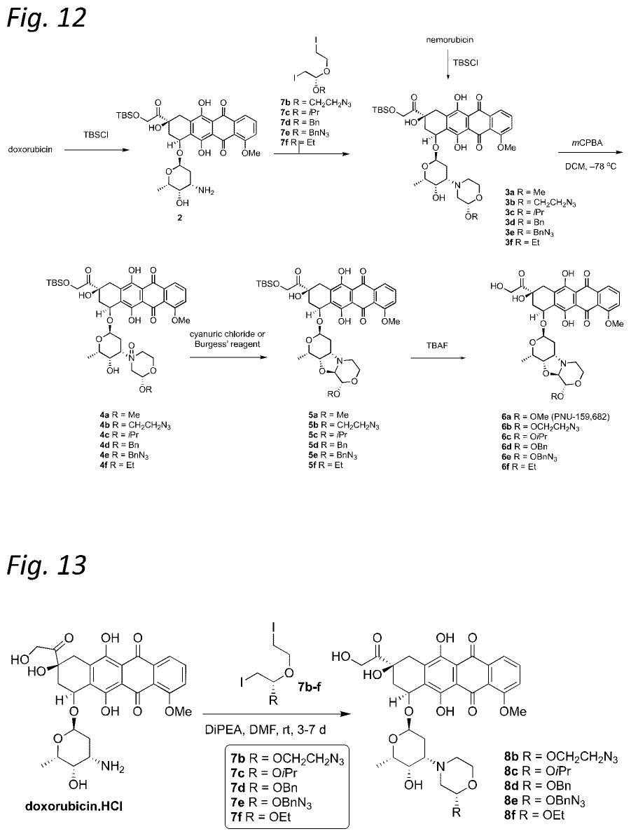

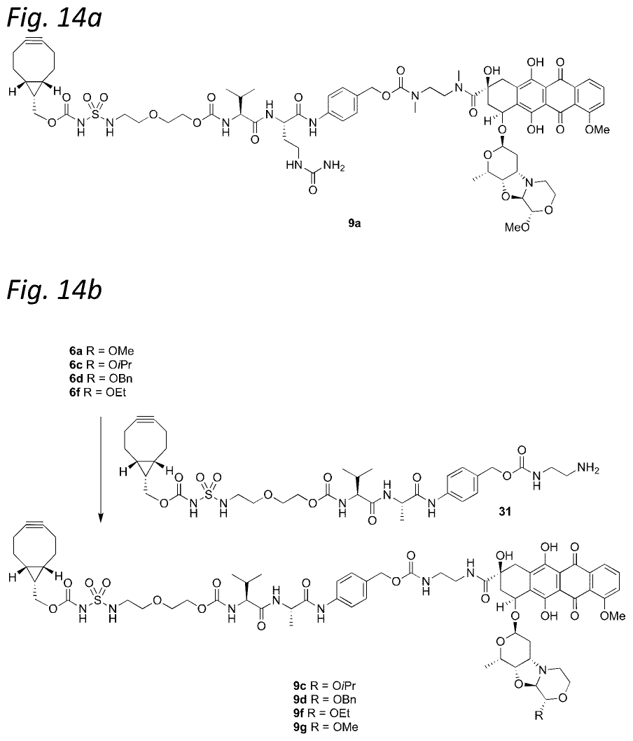

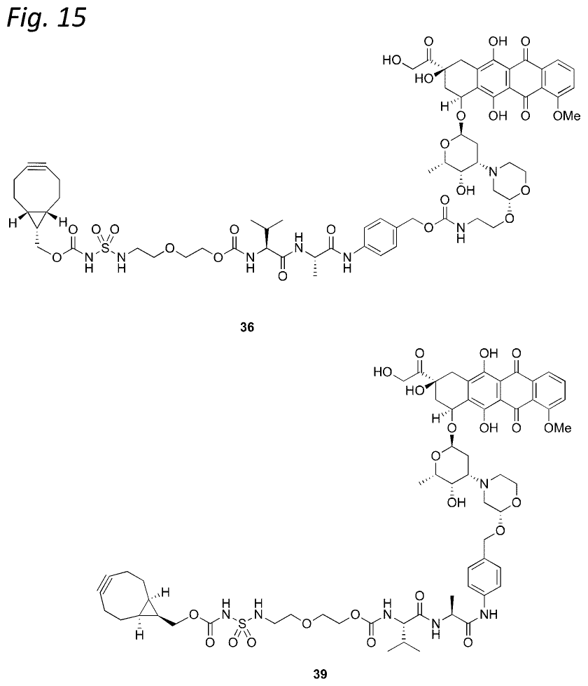

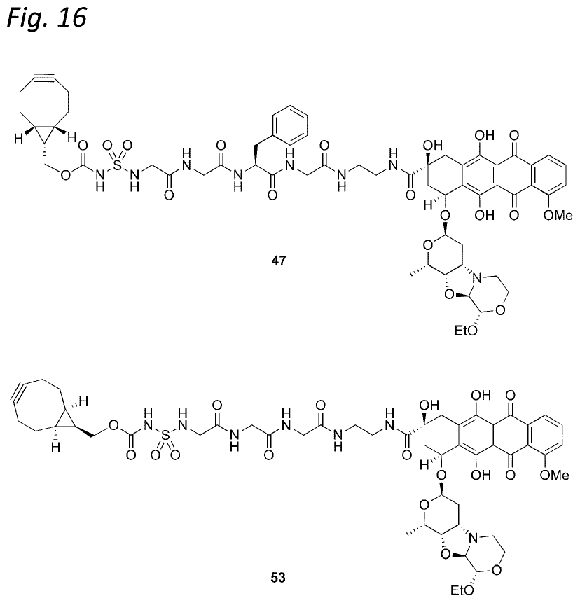

General schemes are depicted in FIGS. 12 and 13. Similar analogues can be prepared from daunorubicin by omitting silylation and desilylation steps.

### Example a20. Synthesis of Compound 2

A solution of doxorubicin HCl (3.09 g, 5.33 mmol) in anhydrous DMF (35 mL) was cooled to 0° C. and imidazole (1.47 g, 21.6 mmol) was added. After stirring for a few minutes, TBDMS-Cl (1.90 g, 12.65 mmol) was added. The reaction mixture was stirred at 0° C. for 5 min after which it was allowed to warm up to room temperature. After stirring for 3.5 h at room temperature, the reaction mixture was purified by flash column chromatography over silicagel (column preconditioned 1% MeOH/DCM, 1%→30% MeOH in DCM) to give compound 2 as a dark red thick oil (3.59 g, 5.4 mmol, 100%). LCMS (ESI+) calculated for C₃₃H₄₄NO₁₁Si⁺ (M+H⁺) 658.27 found 658.44.

### Example a21. Synthesis of Compound 3b

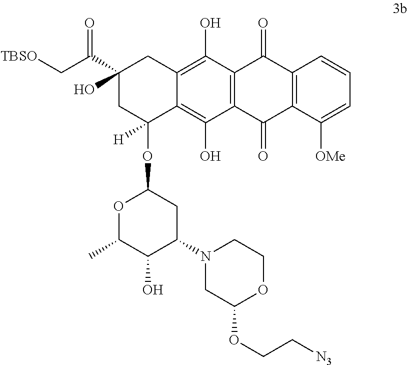 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H](N2CCO[C@H](OCCN=[N+]=[N-])C2)[C@H](O)[C@H](C)O1]

To a stock solution of compound 2 (550 mg, 836 μmol) in anhydrous DMF (1 mL) was added (S)-1-(2-azidoethoxy-2-iodo-1-(2-iodoethoxy)ethane 7b (828.5 mg, 2.06 mmol) and DIPEA (437 μL, 2.51 mmol). The reaction mixture was heated at 42° C. and stirred for 25 minutes after which the heating device was removed, and the reaction mixture was left to react at room temperature. After 72 hours at room temperature, the reaction mixture was diluted with DCM (12 mL) and purified by flash column chromatography over silicagel (0%→3% MeOH in DCM) to give compound 3b as a dark red oil (303 mg, 367 μmol, 43.9%). LCMS (ESI+) calculated for C₃₉H₅₃N₄O₁₃Si⁺ (M+H⁺) 813.34 found 813.51.

### Example a22. Synthesis of Compound 4b

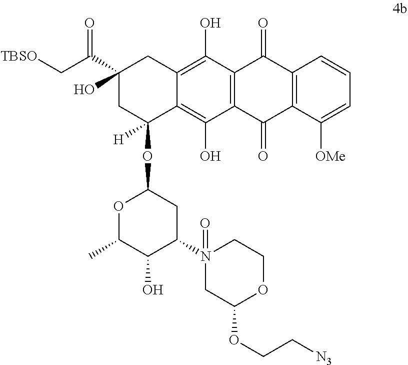 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H]([N+]2([O-])CCO[C@H](OCCN=[N+]=[N-])C2)[C@H](O)[C@H](C)O1]

A solution of compound 3b (303 mg, 253 μmol, 68 wt %) in anhydrous DCM (31 mL) was cooled by dry ice/acetone cooling-bath to a temperature of −78° C. The mixture was vigorously stirred after which a freshly made stock solution of mCPBA in anhydrous DCM (70 mg, 580 mM, 699 μL, 406 μmol) was added dropwise. After stirring for 7 minutes, full conversion was reached. The RM was quenched with an ice-cold solution of acetone (reagent grade, 3.33 mL) and the RM was stirred. After 20 min the cold bath was removed, and the RM was allowed to warm up to room temperature. The RM was transferred to a separation funnel and washed twice with saturated aqueous NaHCO₃ solution (6 mL). The water layers were combined and extracted once with DCM (8 mL). The combined organic layers were dried over Na₂SO₄, filtered through a filter paper, and concentrated until a volume of 30 mL was obtained. No further purification was performed and compound 4b was used as such in the next step. LCMS (ESI+) calculated for C₃₉H₅₃N₄O₁₄Si⁺ (M+H⁺) 829.33 found 829.57.

### Example a23. Synthesis of Compound 5b

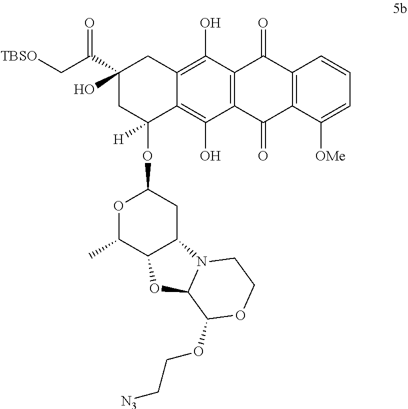 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H]2[C@H](O[C@@H]3[C@@H](OCCN=[N+]=[N-])OCCN32)[C@H](C)O1]

To a solution of compound 4b (188.7 mg, 227.7 μmol) in DCM (27 mL) was added anhydrous acetonitrile (25 mL). The RM was partially concentrated to remove the DCM and to obtain the compound in anhydrous acetonitrile (25 mL). After concentrating most of the solvent, the RM was further diluted with anhydrous acetonitrile (5 mL). Then, potassium carbonate (198 mg, 1.43 mmol) was added, and the RM was cooled to 0° C. after which cyanuric chloride (247.7 mg, 12.1 mL, 111 mM, 1.35 mmol) as a stock solution in anhydrous acetonitrile was added. After stirring for 4 h at 0° C., the RM was quenched with a solution of 3-aminopropane-1,2 diol (489.6 mg, 2.9 mL, 1.85 M, 5.37 mmol) in water. The ice bath was removed after 30 min after which it was allowed to warm up to room temperature. To the RM was added DMF (1.5 mL) and the RM was concentrated until only a solution of DMF/water (8 mL) was left, which was purified by prep-HPLC (40%→100% acetonitrile in 10 mM NH₄HCO₃ in water, column Xbridge prep C₁₈, 5 μM OBD, 30×100 mm). The collected fractions were combined and concentrated until a volume of 6 mL acetonitrile was left. Then it was dried over Na₂SO₄, filtered and the residue was washed through with anhydrous THF (3×750 μL). The combined organic layers were partially concentrated to a volume of 5 mL (acetonitrile/THF) and to give compound 5b as a red solution, which was used without further purification. LCMS (ESI+) calculated for C₃₉H₅₁N₄O₁₃Si⁺ (M+H⁺) 811.32 found 811.51.

### Example a24. Synthesis of Compound 6b

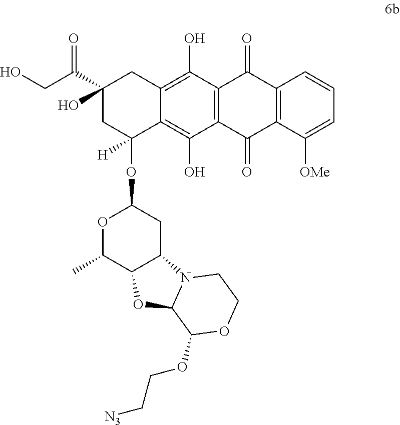 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OCCN=[N+]=[N-])OCCN32)[C@H](C)O1]

To a solution of compound 5b (185 mg, 228 μmol) in a mixture of acetonitrile and THF (5 mL) was added triethylamine acetate (731 μL, 4.56 mmol) and the rm was cooled to −15° C. Then, while the RM was vigorously stirred TBAF (1 M in THF, 1.03 g, 4 mL, 4 mmol) was added in portions (color change from red to green observed and back to dark red). After stirring for 2.5 h, the RM was quenched with water (15 mL), solution changed from dark red to light red. The RM was stirred on ice for 1 min and then left at room temperature for an additional 90 min. The RM was transferred to a separation funnel and extracted with DCM (18 mL). The water layer was extracted with additional DCM (2×6 mL). The combined organic layers were dried over Na₂SO₄, filtered, and further diluted with DCM to 55 mL, and purified by flash column chromatography over silicagel (0%→10% MeOH in DCM). Additional prep-HPLC purification (30%→100% acetonitrile in 10 mM NH₄HCO₃ in water, column Xbridge prep C₁₈, 5 UM OBD, 30×100 mm) was required. The collected fractions were combined and partially concentrated to give compound 6b (0.22 mM based on a doxorubicin-based calibration line for HPLC, 45 mL, 9.82 μmol, 4.3% yield over 3 steps) as a solution in 45 mL acetonitrile/water. LCMS (ESI+) calculated for C₃₃H₃₇N₄O₁₃Si⁺ (M+H⁺) 697.24 found 697.45.

### Example a25. Synthesis of Compound 3c

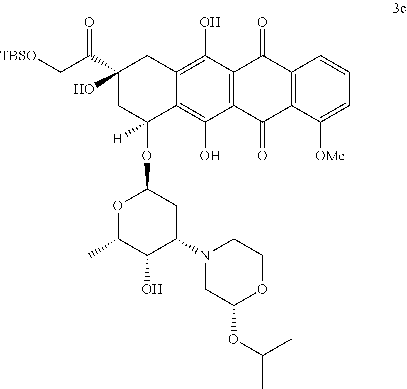 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H](N2CCO[C@H](OC(C)C)C2)[C@H](O)[C@H](C)O1]

To a stock solution of compound 2 (650 mg, 612 μmol) in anhydrous DMF (1.6 mL) was added (R)-2-(2-iodo-1-(2-iodoethoxy)ethoxy) propane 7c (1.13 g, 2.93 mmol) and DIPEA (516 UL, 2.96 mmol). The reaction mixture was heated to 40° C. and stirred for 10 minutes after which the heating device was removed, and the reaction mixture was left to react at room temperature. After 72 at room temperature, the reaction mixture was diluted with DCM (12 mL) and purified by flash column chromatography over silicagel (0%→3% MeOH in DCM) to give compound 3c as a dark red oil (241.8 mg, 307.6 μmol, 31.1%). LCMS (ESI+) calculated for C₄₀H₅₆NO₁₃Si⁺ (M+H⁺) 786.96 found 786.64.

### Example a26. Synthesis of Compound 4c

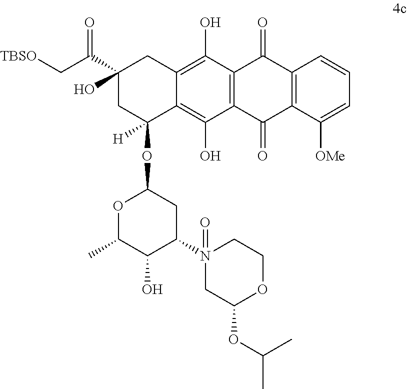 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H]([N+]2([O-])CCO[C@H](OC(C)C)C2)[C@H](O)[C@H](C)O1]

A solution of 3c (125 mg, 159 μmol) in anhydrous DCM (15 mL) was cooled with a dry ice/acetone-cooling bath to a temperature of −78° C. The mixture was vigorously stirred after which a freshly made stock solution of mCPBA in anhydrous DCM (52.1 mg, 580 mM, 521 μL, 302 μmol) was dropwise added. After stirring −78° C. for 7 minutes, full conversion was reached. The RM was quenched with an ice-cold solution of acetone (reagent grade, 1.46 mL) and the RM was stirred. After an hour the cold bath was removed, and the RM was allowed to warm up to room temperature. The RM was transferred to a separation funnel and washed twice with saturated aqueous NaHCO₃ solution (12 mL). The water layers were combined and extracted once with DCM (10 mL). The combined organic layers were dried over Na₂SO₄, filtered through a filter paper, and concentrated until a volume of 12 mL was obtained, providing 4c as a red solution. No further purification was performed and compound 4c was used as such. LCMS (ESI+) calculated for C₄₀H₅₆NO₁₄Si⁺ (M+H⁺) 802.96 found 802.63.

### Example a27. Synthesis of Compound 5c

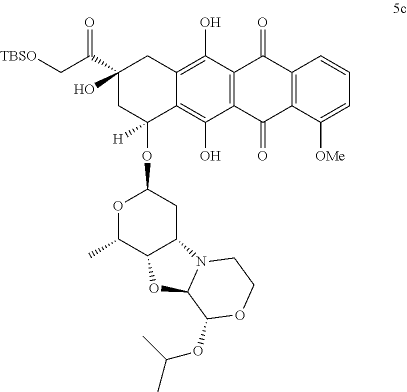 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H]2[C@H](O[C@@H]3[C@@H](OC(C)C)OCCN32)[C@H](C)O1]

To a solution of 4c (128 mg, 159 μmol) in DCM (12 mL) was added anhydrous acetonitrile (6 mL). The RM was partially concentrated to remove the DCM and to obtain the compound in anhydrous acetonitrile (3 mL). After concentrating most of the solvent, the RM was further diluted with anhydrous acetonitrile (20 mL). Then, potassium carbonate (86 mg, 622 μmol) was added, and the RM was cooled to −7° C. after which cyanuric chloride (73.6 mg, 3.59 mL, 111 mM, 399 μmol) as a stock solution in anhydrous acetonitrile was added. After stirring for 2 h at −7° C., the RM was quenched with a solution of 3-aminopropane-1,2 diol (179 mg, 854 μL, 2.3 molar, 1.96 mmol) in water. The ice bath was removed after 15 min after which it was allowed to warm up to room temperature. To the RM was added DMF (1 mL) and the RM was concentrated until only a solution of DMF/water was left, which was purified by prep-HPLC (40%→100% acetonitrile in 10 mM NH₄HCO₃ in water, column Xbridge prep C₁₈, 5 UM OBD, 30×100 mm). The collected fractions were combined and concentrated until a volume of 4 mL was left. The resulting solution consisting of mostly acetonitrile was then dried over Na₂SO₄, filtered and the drying agent was washed through with anhydrous THF (3×750 μL). It was concentrated again to a volume of 3 mL and compound 5c was used as such. LCMS (ESI+) calculated for C₄₀H₅₄NO₁₃Si⁺ (M+H⁺) 784.94 found 786.57.

### Example a28. Synthesis of Compound 6c

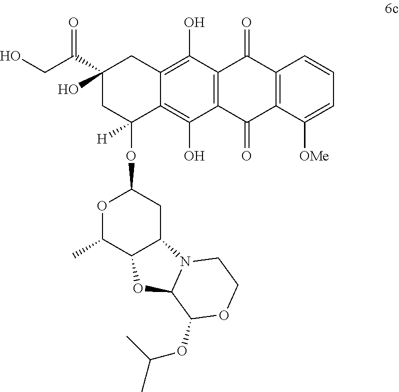 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OC(C)C)OCCN32)[C@H](C)O1]

To compound 5c (121 mg, 154 μmol) in a mixture of acetonitrile and THF (2.88 mL) was added additional dry THF (2.0 mL), and the resulting red solution was cooled to −40° C. Then, while the RM was vigorously stirred TBAF (1 M in THF, 484.5 mg, 1.85 mL, 1.85 mmol) was added (color change from red to green observed). After stirring for 4 h, the RM was quenched with water (3.0 mL), solution changed from green to red. The RM was transferred to a separation funnel and extracted with DCM (3×10 mL). The combined organic layers were dried over Na₂SO₄, filtered, and purified by flash column chromatography over silicagel (0%→5% MeOH in DCM) followed by prep-HPLC (40%→95% acetonitrile in 10 mM NH₄HCO₃ in water, column Xbridge prep C₁₈, 5 μM OBD, 30×100 mm). Compound 6c was obtained as a red solution in DCM (70 mL, 0.086 mM based on a doxorubicin-based calibration line for HPLC, 6,0 μmol, 3,9% yield over 3 steps). LCMS (ESI+) calculated for C₃₄H₄₀NO₁₃⁺ (M+H⁺) 670.25 found 670.51.

### Example a29. Synthesis of Compound 3d

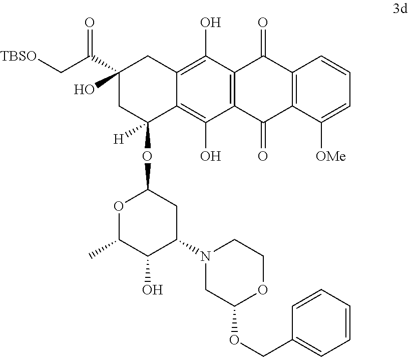 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H](N2CCO[C@H](OCc3ccccc3)C2)[C@H](O)[C@H](C)O1]

To a stock solution of compound 2 (650 mg, 612 μmol) in anhydrous DMF (1.6 mL) was added (S)-((2-iodo-1-(2-iodoethoxy)ethoxy)methyl)benzene 7d (1.27 g, 2.94 mmol) and DIPEA (516 μL, 2.96 mmol). The reaction mixture was heated to 42° C. and stirred for 10 minutes after which the heating device was removed, and the reaction mixture was left to react at room temperature. After 72 at room temperature, the reaction mixture was diluted with DCM (12 mL) and purified by flash column chromatography over silicagel (0%→3% MeOH in DCM) to give compound 3d as a dark red oil (413.5 mg, 479 μmol, 48.5%). LCMS (ESI+) calculated for C₄₄H₅₆NO₁₃Si⁺ (M+H⁺) 834.35 found 834.50.

### Example a30. Synthesis of Compound 4d

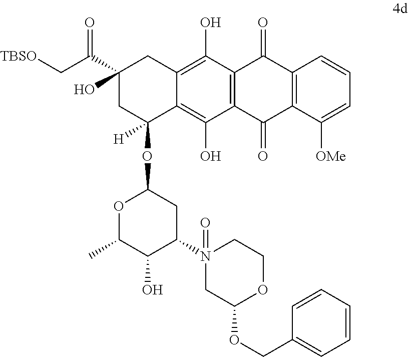 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H]([N+]2([O-])CCO[C@H](OCc3ccccc3)C2)[C@H](O)[C@H](C)O1]

A solution of compound 3d (125 mg, 159 μmol) in anhydrous DCM (15 mL) was cooled by dry ice/acetone cooling-bath to a temperature of −78° C. The mixture was vigorously stirred after which a freshly made stock solution of mCPBA in anhydrous DCM (52.0 mg, 580 mM, 519 μL, 301.5 μmol) was dropwise added. After stirring for 36 minutes, full conversion was reached. The RM was quenched with an ice-cold solution of acetone (reagent grade, 1.38 mL) and the RM was stirred. After 12 min the cooling-bath was removed, and the RM was allowed to warm up to room temperature. The RM was transferred to a separation funnel and washed twice with saturated aqueous NaHCO₃ solution (12 mL). The water layers were combined and extracted once with DCM (10 mL). The combined organic layers were dried over Na₂SO₄, filtered through a filter paper, and concentrated until a volume of 12 mL was obtained. No further purification was performed and compound 4d was used as such. LCMS (ESI+) calculated for C₄₄H₅₆NO₁₄Si⁺ (M+H⁺) 850.35 found 850.60.

### Example a31. Synthesis of Compound 5d

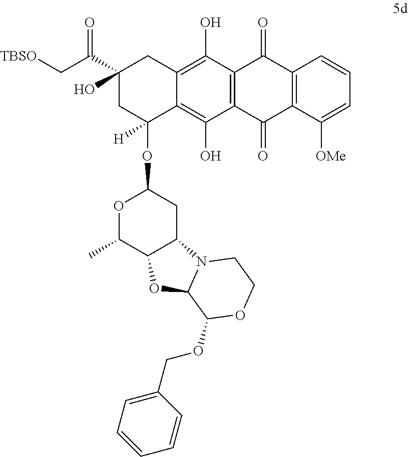 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H]2[C@H](O[C@@H]3[C@@H](OCc4ccccc4)OCCN32)[C@H](C)O1]

To a solution of compound 4d (127 mg, 134 μmol) in DCM (12 mL) was added anhydrous acetonitrile (6 mL). The RM was partially concentrated to remove the DCM and to obtain the compound in anhydrous acetonitrile (3 mL). After concentrating most of the solvent, the RM was further diluted with anhydrous acetonitrile (20 mL). Then, potassium carbonate (73 mg, 530 μmol) was added, and the RM was cooled to −7° C. after which a solution of cyanuric chloride in anhydrous acetonitrile (62.0 mg, 3.01 mL, 111.8 mM, 336 μmol) was added. After stirring for 2 h at −7° C., the RM was quenched with a solution of 3-aminopropane-1,2 diol (151 mg, 1.85 mL, 894 mM, 1.65 mmol) in water. The ice bath was removed after 15 min after which it was allowed to warm up to room temperature. To the RM was added DMF (2 mL) and the RM was concentrated until only a solution of DMF/water was left and purified by prep-HPLC (40%→100% acetonitrile in 10 mM NH₄HCO₃ in water, column Xbridge prep C₁₈, 5 UM OBD, 30×100 mm). The collected fractions were combined and concentrated until a volume of 4 mL was left. The resulting solution consisting of mostly acetonitrile was dried over Na₂SO₄, filtered and the drying agent was washed through with anhydrous THF (3×750 μL). The combined organic layers were again partially concentrated to a volume of 4 mL (acetonitrile/THF) and compound 5d was used as such. LCMS (ESI+) calculated for C₄₄H₅₄NO₁₃Si⁺ (M+H⁺) 832.34 found 832.53.

### Example a32. Synthesis of Compound 6d

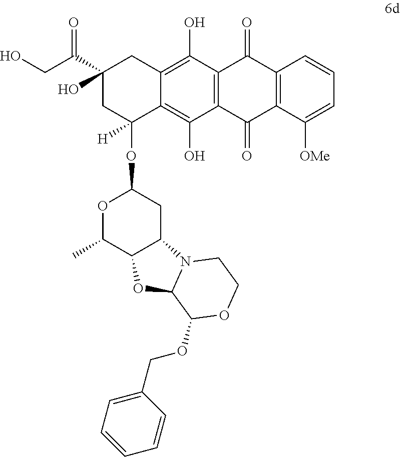 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OCc4ccccc4)OCCN32)[C@H](C)O1]

To compound 5d (25 mg, 30 μmol) in a mixture of acetonitrile and THF (4 mL) was added triethylammonium acetate (97 mg, 96 μL, 600 μmol) and the resulting red solution was cooled to-20° C. Then, while the RM was vigorously stirred TBAF (1 M in THF, 35 mg, 340 μL, 340 mmol) was added (color change from red to green and back to red observed). After stirring for 3 h, the RM was quenched with water (2.4 mL). The RM was transferred to a separation funnel and extracted with DCM (3×5 mL). The combined organic layers were dried over Na₂SO₄, filtered, and concentrated until approximately 10 mL was left (water bath rotary evaporator set 32° C.) and purified by flash column chromatography over silicagel (0%→10% MeOH in DCM) to give compound 6d as a red solution in DCM (4 mL, 0.82 mM based on a doxorubicin-based calibration line for HPLC, 2.36 mg, 3.29 μmol, 11%). LCMS (ESI+) calculated for C₃₈H₄₀NO₁₃⁺ (M+H⁺) 718.25 found 718.50.

### Example a33. Synthesis of Compound 3e

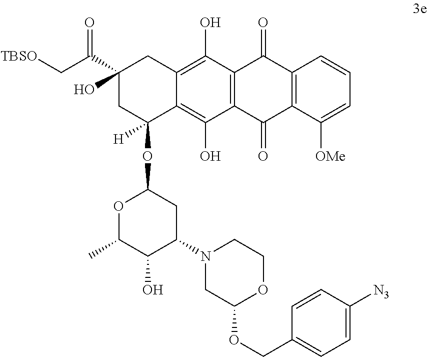 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H](N2CCO[C@H](OCc3ccc(N=[N+]=[N-])cc3)C2)[C@H](O)[C@H](C)O1]

To a stock solution of compound 2 (165 mg, 612 μmol) in anhydrous DMF (410 μL) was added(S)-1-azido-4-((2-iodo-1-(2-iodoethoxy)ethoxy)methyl)benzene 7e (294 mg, 603 μmol) and DIPEA (131 μL, 753 μmol). The reaction mixture was vortexed and was left to react at room temperature. After 120 h at room temperature, the reaction mixture was diluted with DCM (8 mL) and purified by flash column chromatography over silicagel (0%→3% MeOH in DCM) to give compound 3e as a dark red oil (53.9 mg, 61.6 μmol, 24.6%). LCMS (ESI+) calculated for C₄₄H₅₅N₄O₁₃Si⁺ (M+H⁺) 875.35 found 875.61.

### Example a34. Synthesis of Compound 4e

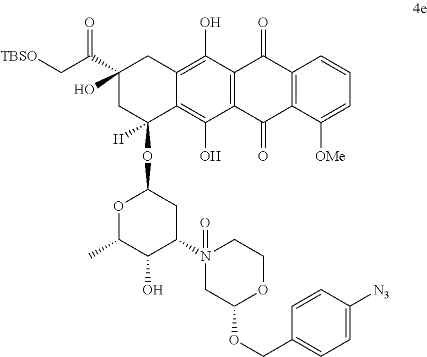 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H]([N+]2([O-])CCO[C@H](OCc3ccc(N=[N+]=[N-])cc3)C2)[C@H](O)[C@H](C)O1]

A solution of compound 3e (53.9 mg, 61.6 μmol) in anhydrous DCM (10 mL) was cooled with a dry ice/acetone cooling-bath to a temperature of −78° C. The mixture was vigorously stirred after which a freshly made stock solution of mCPBA in anhydrous DCM (20.2 mg, 580 mM, 202 μL, 117 μmol) was added dropwise. After stirring for 8 minutes −78° C., full conversion was reached. The RM was quenched with an ice-cold solution of acetone (reagent grade, 565 μL) and the RM was stirred at −78° C. After 45 min the cooling-bath was removed, and the RM was allowed to warm up to room temperature. The RM was transferred to a separation funnel and washed twice with a saturated aqueous NaHCO₃ solution (10 mL). The water layers were combined and extracted once with DCM (10 mL). The combined organic layers were dried over Na₂SO₄, filtered through a filter paper, and concentrated until a volume of 10 mL was obtained. No further purification was performed and compound 4e was used as such. LCMS (ESI+) calculated for C₄₄H₅₅N₄O₁₄Si⁺ (M+H⁺) 891.35 found 891.52.

### Example a35. Synthesis of Compound 5e

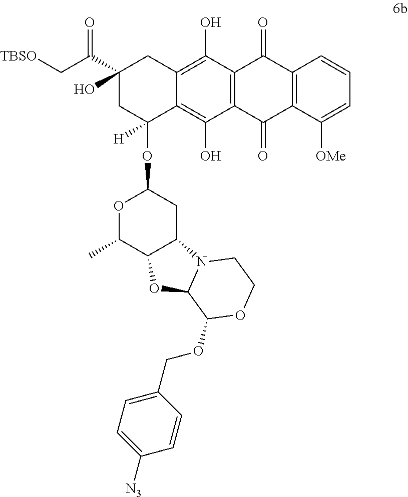 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H]2[C@H](O[C@@H]3[C@@H](OCc4ccc(N=[N+]=[N-])cc4)OCCN32)[C@H](C)O1]

To a solution of compound 4e (54.9 mg, 61.6 μmol) in DCM (10 mL) was added anhydrous acetonitrile (6 mL). The RM was partially concentrated to remove the DCM and to obtain the compound in anhydrous acetonitrile (3 mL). After concentrating most of the solvent, the RM was further diluted with anhydrous acetonitrile (10 mL). Then, potassium carbonate (33.2 mg, 240 μmol) was added, and the RM was cooled to −7° C. after which a solution of cyanuric chloride in anhydrous acetonitrile (28.4 mg, 1.39 mL, 111 mM, 154 μmol) was added. After stirring for 6 h at −7° C., the RM was quenched with a solution of 3-aminopropane-1,2 diol (69 mg, 330 μL, 2.3 M, 758 μmol) in water. The ice bath was removed after 15 min after which it was allowed to warm up to room temperature. To the RM was added DMF (1 mL) and the RM was concentrated until only a solution of DMF/water was left and purified by prep-HPLC (40%→100% acetonitrile in 10 mM NH₄HCO₃ in water, column Xbridge prep C₁₈, 5 UM OBD, 30×100 mm). The collected fractions were combined and concentrated until a volume of 4 mL acetonitrile was left. Then it was dried over Na₂SO₄, filtered and the residue was washed through with anhydrous THF (3×750 μL). The combined organic layers were again partially concentrated to a volume of 4 mL (acetonitrile/THF) and compound 5e was used as such. LCMS (ESI+) calculated for C₄₄H₅₃N₄O₁₃Si⁺ (M+H⁺) 873.34 found 873.46.

### Example a36. Synthesis of Compound 6e

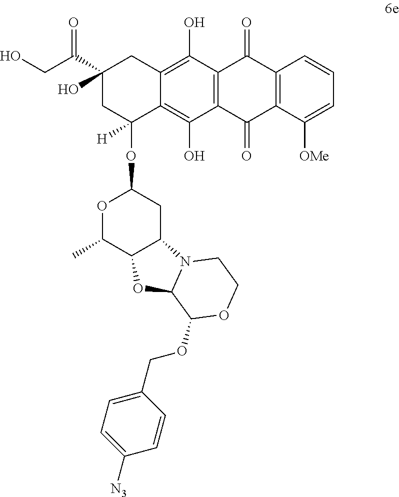 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OCc4ccc(N=[N+]=[N-])cc4)OCCN32)[C@H](C)O1]

Compound 5e (27 mg, 31 μmol) in a mixture of acetonitrile and THF (4 mL) was cooled to −40° C. Then, while the RM was vigorously stirred TBAF (1 M in THF, 80 mg, 300 μL, 300 mmol) was added (color change from red to green observed). After stirring for 2.5 h, the RM was quenched with water (2.5 mL), solution changed from green to red. The RM was transferred to a separation funnel and extracted with DCM (3×7 mL). The combined organic layers were dried over Na₂SO₄, filtered, and purified by flash column chromatography over silicagel (0%→10% MeOH in DCM) to give compound 6e as a red solution in DCM (10 mL, 0.31 mM based on a doxorubicin-based calibration line for HPLC, 2.3 mg, 3.0 μmol, 9.8% over 3 steps). LCMS (ESI+) calculated for C₃₈H₃₉N₄O₁₃⁺ (M+H⁺) 759.25 found 759.40.

### Example a37. Synthesis of Compound 3f

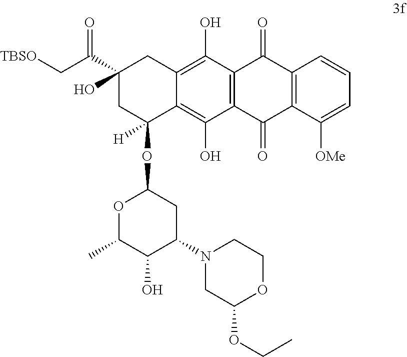 [SMILES: CCO[C@@H]1CN([C@H]2C[C@H](O[C@H]3C[C@](O)(C(=O)CO[Si](C)(C)C(C)(C)C)Cc4c(O)c5c(c(O)c43)C(=O)c3c(OC)cccc3C5=O)O[C@@H](C)[C@H]2O)CCO1]

A stock solution of compound 2 (150 mg, 228 μmol) in anhydrous DMF (373 μL) was added to a vial containing 1,5-diiodo-2 (S)-ethoxy-3-oxa-pentane 7f (1.27 g, 2.94 mmol), followed by DIPEA (119 μL, 684 μmol). The reaction mixture was heated to 42° C. and swirled for 1 minute and then left at room temperature in the dark. After 96 h at room temperature, the reaction mixture was diluted with DCM (4 mL) and purified by flash column chromatography over silicagel (0%→3% MeOH in DCM) to give 3f as a dark red oil (43.3 mg, 56.1 μmol, 24.6%). LCMS (ESI+) calculated for C₃₉H₅₄NO₁₃Si⁺ (M+H⁺) 772.34 found 772.65.

### Example a38. Synthesis of Compound 4f

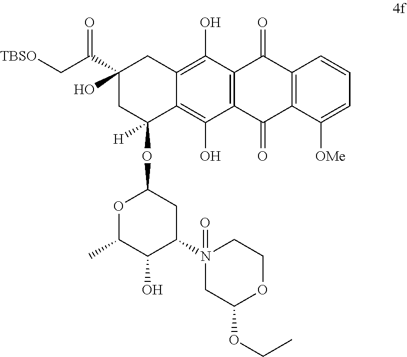 [SMILES: CCO[C@@H]1C[N+]([O-])([C@H]2C[C@H](O[C@H]3C[C@](O)(C(=O)CO[Si](C)(C)C(C)(C)C)Cc4c(O)c5c(c(O)c43)C(=O)c3c(OC)cccc3C5=O)O[C@@H](C)[C@H]2O)CCO1]

A solution of compound 3f (43.3 mg, 56.1 μmol) in anhydrous DCM (700 μL) was cooled with a dry ice/acetone-bath to a temperature of −78° C. The mixture was vigorously stirred after which a freshly made stock solution of mCPBA in anhydrous DCM (6.19 mg, 462 mM, 77.6 μL, 35.9 μmol) was added dropwise. After stirring for 20 minutes, a 2^{nd} solution of mCPBA in anhydrous DCM (26.5 μL, 462 mM, 12.2 μmol) was added dropwise and the resulting red solution was stirred for another 15 minutes. Finally, a 3rd batch of mCPBA in anhydrous DCM (0.24 mg, 462 mM, 3.0 μL, 1.4 μmol) was added. The RM was stirred for another 2 minutes and was then quenched with a pre-cooled (−78° C.) solution of acetone (reagent grade, 300 μL) and the RM was stirred at −78° C. After 12 min the cooling-bath was removed, and the RM was allowed to warm up to room temperature and diluted with additional DCM (3.5 mL). The RM was transferred to a separation funnel and washed twice with saturated aqueous NaHCO₃ solution (2 mL). The water layers were combined and extracted twice with DCM (2 mL). The combined organic layers were dried over Na₂SO₄, filtered through a membrane filter. The filtrate, a red solution containing compound 4f, was used without further purification. LCMS (ESI+) calculated for C₃₉H₅₄NO₁₄Si⁺ (M+H⁺) 788.33 found 788.64.

### Example a39. Synthesis of Compound 5f

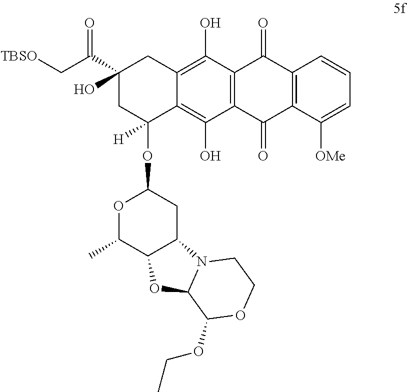 [SMILES: CCO[C@H]1OCCN2[C@@H]1O[C@@H]1[C@H](C)O[C@@H](O[C@H]3C[C@](O)(C(=O)CO[Si](C)(C)C(C)(C)C)Cc4c(O)c5c(c(O)c43)C(=O)c3c(OC)cccc3C5=O)C[C@@H]12]

To a solution of compound 4f (44.3 μmol) in a mixture (circa 10 mL) of mainly DCM and minimal acetone was added anhydrous acetonitrile (2 mL). The RM was partially concentrated—to remove the DCM—to a volume of roughly 6 mL. Next, additional anhydrous acetonitrile (4.0 mL) was added, and the mixture was again partially concentrated to a volume of 4.4 mL. A stirring bar was added, and the RM was analyzed by HPLC-MS to assess the concentration of the starting material 4f (Indicating 44.3 μmol of 4f based on a doxorubicin-based calibration line). Next, potassium carbonate (31.3 mg, 226 μmol) was added, and the RM was cooled to 0° C. after which a solution of cyanuric chloride in anhydrous acetonitrile (20.5 mg, 1.00 mL, 111 mM, 111 μmol) was added. After stirring for 2.5 h at 0° C., the RM was again treated with a solution of cyanuric chloride in anhydrous acetonitrile (80 μL, 111 mmolar, 8.9 μmol). The RM was stirred at 0° C. for another 23 minutes and was then quenched with a solution of 3-aminopropane-1,2 diol (62.9 mg, 345 μL, 2.0 M, 690 μmol) in water. The resulting dark red solution was allowed to slowly warm to room temperature. To the RM was added DMF (3 mL) to give a red solution with a mostly white precipitate. The solution was decanted, and the residue was washed a few times with additional DMF, which was filtered over a membrane-filter before combining with the decanted solution. The solution was partially concentrated in vacuo to mostly remove acetonitrile and was then purified by prep-HPLC (40%→100% acetonitrile in 10 mM NH₄HCO₃ in water, column Xbridge prep C₁₈, 5 UM OBD, 30×100 mm). The collected fractions were combined and concentrated until a volume of 4 mL was left. The resulting solution consisting of mostly acetonitrile was dried over Na₂SO₄, filtered and the drying agent was washed through with anhydrous THF (3×). The combined organic layers were again partially concentrated to a volume of 1.7 mL (acetonitrile/THF) and compound 5f (9.32 mM based on a doxorubicin-based calibration line for HPLC, 15.8 mM, 35.7%) was used as such. LCMS (ESI+) calculated for C₃₉H₅₂NO₁₃Si⁺ (M+H⁺) 770.32 found 770.65.

### Example a40. Synthesis of Compound 6f

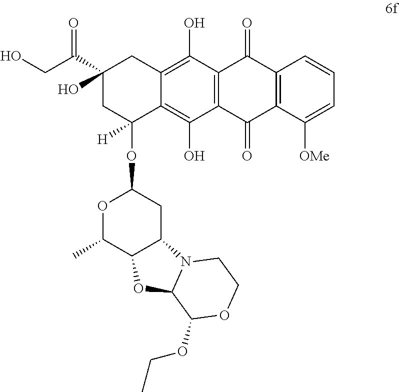 [SMILES: CCO[C@H]1OCCN2[C@@H]1O[C@@H]1[C@H](C)O[C@@H](O[C@H]3C[C@](O)(C(=O)CO)Cc4c(O)c5c(c(O)c43)C(=O)c3c(OC)cccc3C5=O)C[C@@H]12]

To compound 5f (12.9 mg, 15.8 μmol) in a mixture of acetonitrile and THF (1.7 mL) was added triethylammonium acetate (13.8 mg, 13.7 μL, 85.5 μmol) and the resulting red solution was cooled to −15° C. Then, while the RM was stirred TBAF (1 M in THF, 22.4 mg, 85.5 μL, 85.5 μmol) was added (color change from red to green and back to red observed). After stirring for 46 minutes additional triethylammonium acetate (13.8 mg, 13.7 μL, 85.5 μmol) and TBAF (1 M in THF, 22.4 mg, 85.5 μL, 85.5 μmol) were added. The RM was stirred for another 20 minutes before a third batch of TBAF (1 M in THF, 22.4 mg, 85.5 μL, 85.5 μmol) was added, which was stirred for another 30 minutes. Finally, a fourth batch of TBAF (1 M in THF, 10.0 mg, 40.0 μL, 40.0 μmol) was added and the RM was stirred at −10° C. for another 35 minutes before the reaction was quenched with water (1.0 mL). The RM was allowed to warm to rt, diluted with DCM (7.5 mL) and transferred to a separation funnel. The biphasic system was separated, and the water layer was extracted with DCM (2×1 mL). The combined organic layers were dried over Na₂SO₄, filtered then purified by flash column chromatography over silicagel (0%→6% MeOH in DCM). The pure fractions were combined and partially concentrated to a volume of 4.5 mL and then diluted with MeOH (7 mL). Next, the solution was again partially concentrated to a volume of 6 mL and diluted until a volume of 6.7 mL to give compound 6f as a red solution in mostly MeOH (6.7 mL, 1.55 mM based on a doxorubicin-based calibration line for HPLC, 10.36 μmol, 65.6%). LCMS (ESI+) calculated for C₃₃H₃₈NO₁₃⁺ (M+H⁺) 656.23 found 656.59.

### Example a41. Synthesis of Compound 3a

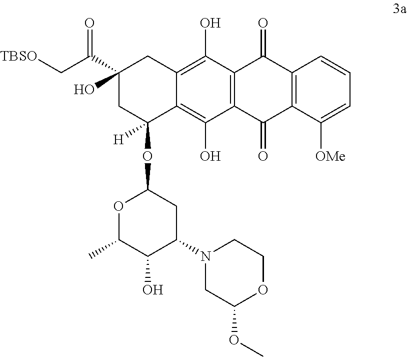 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H](N2CCO[C@H](OC)C2)[C@H](O)[C@H](C)O1]

To a solution of nemorubicin (75 mg, 0.12 mmol) and imidazole (40 mg, 0.58 mmol) in anhydrous DMF (2.0 mL) was added TBDMS-CI (53 mg, 0.35 mmol, 3 equiv.). The resulting red solution was mixed and left at rt for 15 minutes. Next, the mixture was diluted with DCM (22 mL) and purified by flash column chromatography over silicagel (1%→5% MeOH in DCM) to give compound 3a as a dark red oil (134 mg, quant.). LCMS (ESI+) calculated for C₃₈H₅₂NO₁₃Si⁺ (M+H⁺) 758.32 found 758.59.

### Example a42. Synthesis of Compound 4a

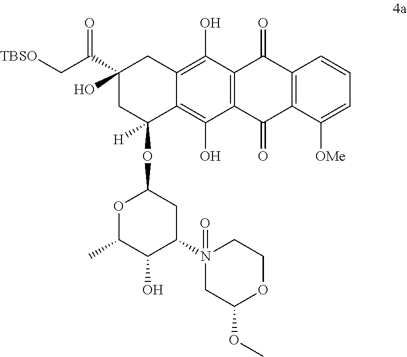 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H]([N+]2([O-])CCO[C@H](OC)C2)[C@H](O)[C@H](C)O1]

A solution of compound 3a (120 μmol) in anhydrous DCM (15.0 mL) was cooled with a dry ice/acetone cooling-bath to a temperature of −78° C. The mixture was vigorously stirred after which a freshly made stock solution of mCPBA in anhydrous DCM (33.1 mg, 580 mM, 331 μL, 192 μmol) was added dropwise, while stirring vigorously. After stirring for 18 minutes the mixture was quenched with a pre-cooled (−78° C.) solution of acetone (reagent grade, 1.1 mL. After 23 min the cooling-bath was removed, and the RM was allowed to warm up to room temperature. The RM was transferred to a separation funnel and washed twice with saturated aqueous NaHCO₃ solution (3 mL). The water layers were combined and extracted once with DCM (4 mL). The combined organic layers were dried over Na₂SO₄ and partially concentrated in vacuo to a volume of 12 mL, affording compound 4a as a red solution in mostly DCM, which was used without further purification. LCMS (ESI+) calculated for C₃₈H₅₂NO₁₄Si⁺ (M+H⁺) 774.32 found 774.50.

### Example a43. Synthesis of Compound 5a

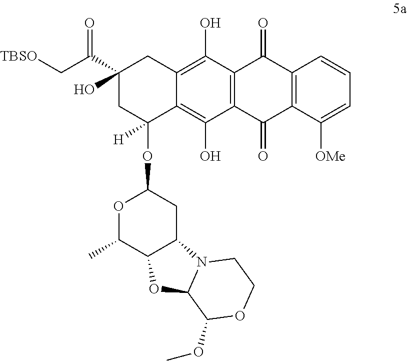 [SMILES: COc1cccc2c1C(=O)c1c(O)c3c(c(O)c1C2=O)C[C@@](O)(C(=O)CO[Si](C)(C)C(C)(C)C)C[C@@H]3O[C@H]1C[C@H]2[C@H](O[C@@H]3[C@@H](OC)OCCN32)[C@H](C)O1]

To a solution of compound 4a (100.6 μmol) in a mixture (10.1 ml) of mainly DCM and minimal acetone was added anhydrous acetonitrile (8 mL). The RM was partially concentrated to remove the DCM to a volume of circa 4 mL. Next, potassium carbonate (54.5 mg, 394 μmol) was added, and the RM was cooled to 0° C. after which a solution of cyanuric chloride in anhydrous acetonitrile (46.4 mg, 2.266 mL, 111 mM, 251.5 μmol) was added. After stirring for 150 minutes at 0° C., the RM was quenched with a solution of 3-aminopropane-1,2 diol (1.33 mL, 929 mM, 1.24 mmol) in water. The resulting dark red solution was allowed to slowly warm to room temperature over 20 minutes. To the RM was added DMF (1.33 mL) and the resulting mixture was partially concentrated and then purified by prep-HPLC (40%→100% acetonitrile in 10 mM NH₄HCO₃ in water, column Xbridge prep C₁₈, 5 UM OBD, 30×100 mm). The collected fractions were combined and concentrated until a volume of 4 mL was left. The resulting solution consisting of mostly acetonitrile was dried over Na₂SO₄, filtered and the drying agent was washed through with anhydrous THF (3×). The combined organic layers were again partially concentrated to a volume of 4.5 mL (acetonitrile/THF) to give compound 5a as a red solution, which was used as such in the next step. LCMS (ESI+) calculated for C₃₈H₄₈NO₁₃Si⁻ (M−H⁺) 754.29 found 754.52.

### Example a44. Synthesis of Compound 6a

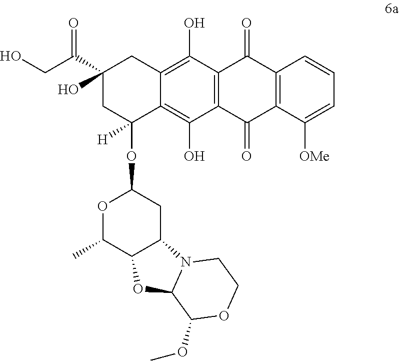 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OC)OCCN32)[C@H](C)O1]

To compound 5a (40 mg, 53 μmol) in a mixture of acetonitrile and THF (4.5 mL) was added triethylammonium acetate (170 μL, 1.06 mmol) and the resulting red solution was cooled to −15° C. Then, while the RM was stirred TBAF (1 M in THF, 65 mg, 0.25 mL, 0.25 mmol) was added (color change from red to green and back to red observed). After stirring for 1 minute additional TBAF (1 M in THF, 63 mg, 0.24 mL, 0.24 mmol) were added. The RM was stirred for another 14 minutes before a third batch of TBAF (1 M in THF, 46 mg, 0.17 mL, 0.17 mmol) was added, which was stirred for another 41 minutes. The reaction was quenched with water (2.4 mL). The RM was allowed to warm to rt over 25 minutes and then combined with a 2^{nd} batch of crude compound 6a, which was obtained in the same manner as described above starting with compound 5a (10 mg, 13.0 μmol). After combining both quenched reaction mixtures DCM (6 mL) was added, and the resulting biphasic system was separated. The water layer was extracted two times (4 mL then 2 mL) and the combined organic layers were dried (Na₂SO₄) and then purified by flash column chromatography over silicagel (0%→6% MeOH in DCM). The pure fractions were combined and partially concentrated to a volume of 8-9 mL and then diluted with additional DCM to a volume of 10.0 mL to give compound 6a as a red solution in mostly DCM (10.0 mL, 1.66 mM based on a doxorubicin-based calibration line for HPLC, 16.6 μmol, 16.5% over 2 steps (assuming quantitative conversion during N-oxide formation)). LCMS (ESI+) calculated for C₃₂H₃₆NO₁₃⁺ (M+H⁺) 642.22 found 642.46.

### Example a45. Synthesis of Compound 8b

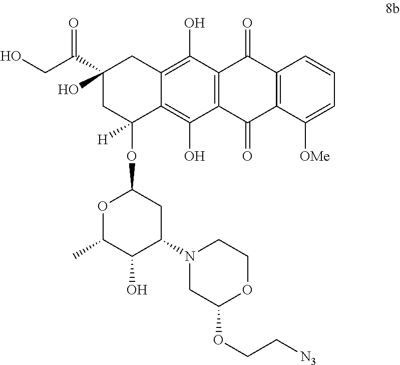 [SMILES: COc1cccc2c1C(=O)c1c(O)c3c(c(O)c1C2=O)C[C@@](O)(C(=O)CO)C[C@@H]3O[C@H]1C[C@H](N2CCO[C@H](OCCN=[N+]=[N-])C2)[C@H](O)[C@H](C)O1]

A mixture of doxorubicin·HCl salt (48.6 mg, 83.8 μmol, 1.00 eq) and(S)-1-(2-azidoethoxy)-2-iodo-1-(2-iodoethyoxy)ethane 7b (101.0 mg, 245.8 μmol, 2.93 eq) were dissolved in anhydrous DMF (175 μL) and DIPEA (58.7 μL, 335 μmol, 4 eq) was added. The red suspension was stirred in the dark at room temperature for 3 days, diluted with DCM (1.8 mL) and purified by flash column chromatography over silicagel (column preconditioned 2% MeOH/DCM, 2%→8% MeOH in DCM) to give compound 8b as a red oil (10.8 mg, 15.5 μmol, 18.4%). LCMS (ESI+) calculated for C₃₃H₃₉N₄O₁₃⁺ (M+H⁺) 669.25 found 699.62.

### Example a46. Synthesis of Compound 9

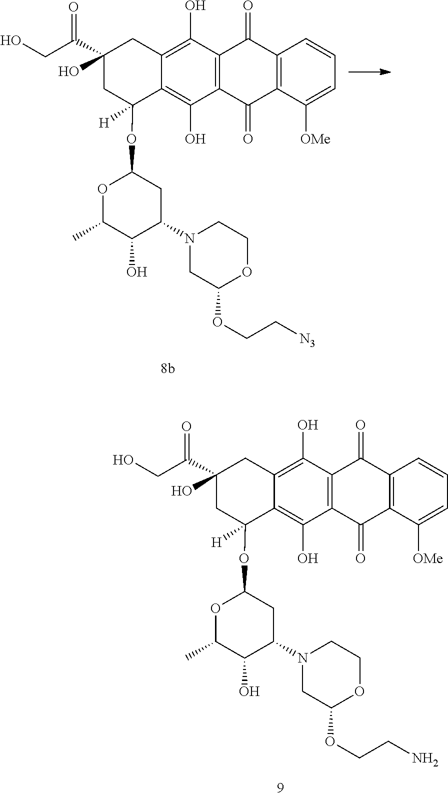 [SMILES: COc1cccc2c1C(=O)c1c(O)c3c(c(O)c1C2=O)C[C@@](O)(C(=O)CO)C[C@@H]3O[C@H]1C[C@H](N2CCO[C@H](OCCN=[N+]=[N-])C2)[C@H](O)[C@H](C)O1 ; COc1cccc2c1C(=O)c1c(O)c3c(c(O)c1C2=O)C[C@@](O)(C(=O)CO)C[C@@H]3O[C@H]1C[C@H](N2CCO[C@H](OCCN)C2)[C@H](O)[C@H](C)O1]

To a solution of 8b (8.1 mg, 12 μmol, 1.0 equiv.) in a 1:1 mixture of MeOH/DCM (150 μL) was added a 200 mmolar solution of PPh3 in DCM (177 μL, 31.9 μmol, 2.7 equiv.) and H₂O (80 μL). The resulting biphasic system was stirred at rt in the dark for 8 hours. Next, the RM was stored in the freezer for 8 days and was then concentrated in vacuo. The residue was taken up in DMF and purified by prep-HPLC purification (5%→90% acetonitrile in water, column Xbridge prep C₁₈, 5 μM OBD, 30×100 mm), affording compound 9 as a red residue (0.5 mg, 0.7 μmol, 6% yield). The impure fractions from the prep-HPLC purification were combined and purified by a 2^{nd} prep-HPLC purification (5%→90% acetonitrile in water, column Xbridge prep C₁₈, 5 UM OBD, 30×100 mm), affording additional compound 9 as a red residue (0.6 mg, 0.9 μmol, 8% yield). LCMS (ESI+) calculated for C₃₃H₄₁N₂O₁₃⁺ (M+H⁺) 673.26 found 673.65.

### Example a47. Synthesis of Compound 8c

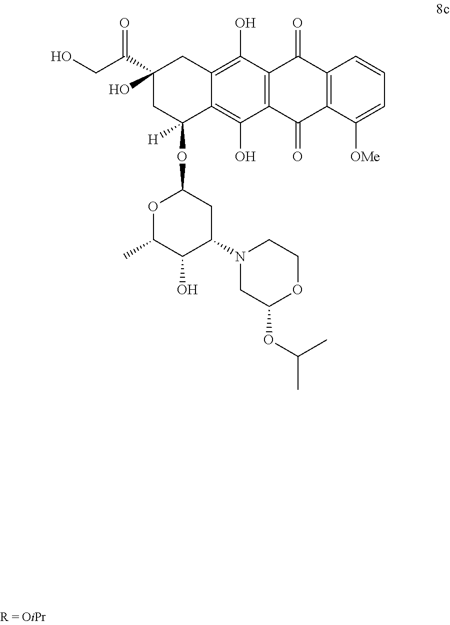 [SMILES: COc1cccc2c1C(=O)c1c(O)c3c(c(O)c1C2=O)C[C@@](O)(C(=O)CO)C[C@@H]3O[C@H]1C[C@H](N2CCO[C@H](OC(C)C)C2)[C@H](O)[C@H](C)O1]

A mixture of doxorubicin·HCl salt (51.3 mg, 88.5 μmol, 1.00 eq) and (R)-2-(2-iodo-1-(2-iodoethoxy)ethoxy) propane 7c (104 mg, 271 μmol, 3.06 eq) were dissolved in anhydrous DMF (175 μL) and DIPEA (61.6 μL, 354 μmol, 4 eq) was added. The red suspension was stirred in the dark at room temperature for 3 days, diluted with DCM (1.8 mL) and purified by flash column chromatography over silicagel (column preconditioned 2% MeOH/DCM, 2%→8% MeOH in DCM) to give compound 8c as a red oil (12.2 mg, 18.2 μmol, 20.5%). LCMS (ESI+) calculated for C₃₄H₄₂NO₁₃⁺ (M+H⁺) 672.27 found 672.60.

### Example a48. Synthesis of Compound 8d

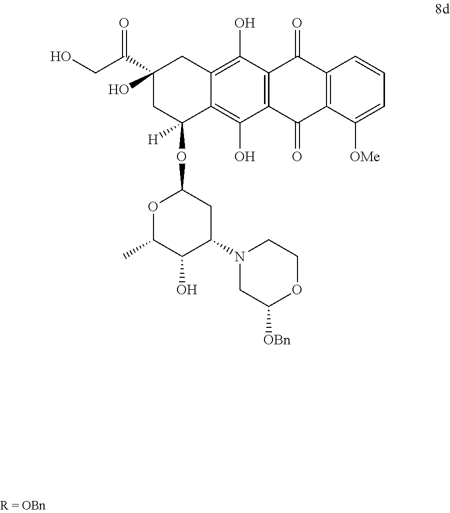 [SMILES: COc1cccc2c1C(=O)c1c(O)c3c(c(O)c1C2=O)C[C@@](O)(C(=O)CO)C[C@@H]3O[C@H]1C[C@H](N2CCO[C@H](OCc3ccccc3)C2)[C@H](O)[C@H](C)O1]

A mixture of doxorubicin·HCl salt (49.7 mg, 85.7 μmol, 1.00 eq) and (S)-((2-iodo-1-(2-iodoethoxy)ethoxy)methyl)benzene 7d (106.5 mg, 246.5 μmol, 2.88 eq) were dissolved in anhydrous DMF (175 μL) and DIPEA (59.7 μL, 343 μmol, 4 eq) was added. The red suspension was stirred in the dark at room temperature for 3 days, diluted with DCM (1.8 mL) and purified twice by flash column chromatography over silicagel (column preconditioned 2% MeOH/DCM, 0%->13% MeOH in DCM) to give compound 8d as a red oil (6.4 mg, 8.9 μmol, 10%). LCMS (ESI+) calculated for C₃₈H₄₂NO₁₃⁺ (M+H⁺) 720.27 found 720.64.

### Example a49. Synthesis of Compound 8f

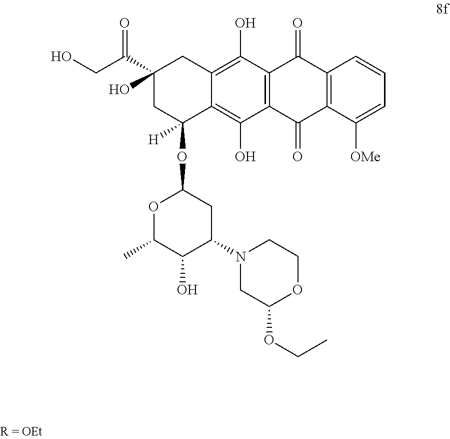 [SMILES: CCO[C@@H]1CN([C@H]2C[C@H](O[C@H]3C[C@](O)(C(=O)CO)Cc4c(O)c5c(c(O)c43)C(=O)c3c(OC)cccc3C5=O)O[C@@H](C)[C@H]2O)CCO1]

A mixture of doxorubicin·HCl salt (52.3 mg, 90.2 μmol, 1.00 eq) and(S)-1-ethoxy-2-iodo-1-(2-iodoethyoxy)ethane 7f (102.1 mg, 276.0 μmol, 3.06 eq) were dissolved in anhydrous DMF (175 μL) and DIPEA (62.8 μL, 361 μmol, 4 eq) was added. The red suspension was stirred in the dark at room temperature for 4 days, diluted with DCM (1.8 mL) and purified by flash column chromatography over silicagel (column preconditioned 2% MeOH/DCM, 2%→8% MeOH in DCM) to give compound 8f as a red oil (11.1 mg, 16.9 μmol, 18.7%). LCMS (ESI+) calculated for C₃H₄₀NO₁₃⁺ (M+H⁺) 658.25 found 658.70.

### Example a50. Synthesis of Compound 31

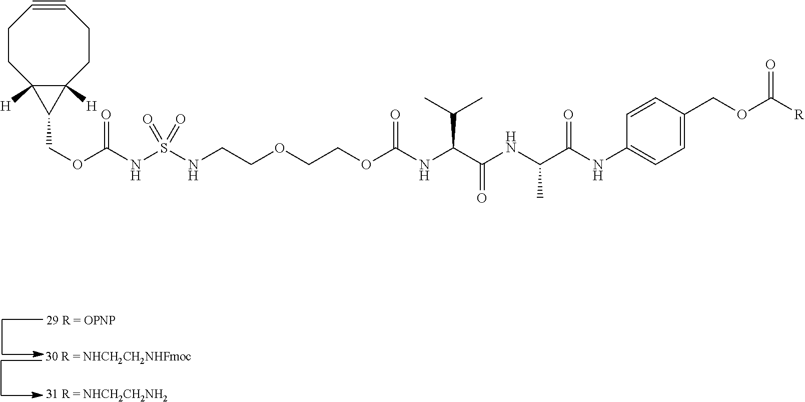

To a solution of Fmoc-N-ethylene-1,2-diamine·HCl (26 mg, 83 μmol) in anhydrous DMF (200 μL) was added a solution of compound 29 (67 mg, 75 μmol) in anhydrous DCM (800 μL) and triethylamine (32 μL, 23 mg, 230 μmol). After stirring for 1 hour at room temperature, the reaction mixture was purified by flash column chromatography over silicagel (0%→30% EtOAc in DCM (to remove p-nitrophenol) followed by 0%→25% MeOH in DCM) to give intermediate 30 as a colorless oil (32.7 mg, 32 μmol, 43%). LCMS (ESI+) calculated for C₄₉H₆₂N₇O₁₃S⁺ (M+H⁺) 988.41 found 988.78.

To a solution of intermediate 30 (16.3 mg, 16.5 μmol) in DMF (150 μL) was added triethylamine (13.8 μL, 10 mg, 99.0 μmol). After stirring for 18 hours at room temperature complete conversion was obtained and the reaction mixture was concentrated to obtain compound 31 as an oil (12.6 mg, 16.5 μmol, 100%). LCMS (ESI+) calculated for C₃₄H₅₂N₇O₁₁S⁺ (M+H⁺) 766.34 found 766.65.

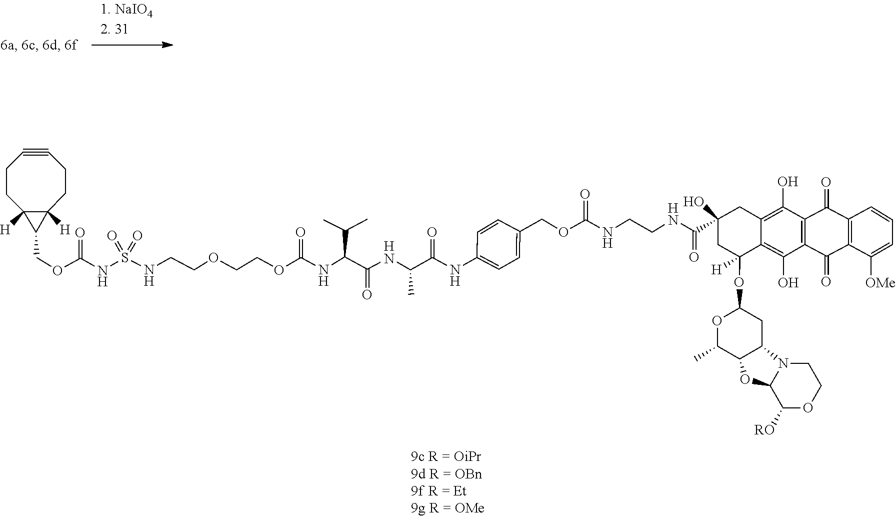

### Example a51. Synthesis of Compound 9c

To a solution of compound 6c (3.26 mg, 4.87 μmol) in DCM was added MeOH (3 mL) and the mixture was concentrated until only MeOH (1.5 mL) was left. Water (200 μL) was added and a solution of sodium periodate in water (60 mM, 282 μL, 16.9 μmol) was added and the reaction mixture was stirred for 41 hours in the dark. Once complete conversion was achieved, DMF (800 μL) was added and the reaction mixture was concentrated till there was 400 μL in DMF was left (2.19 mg, 3.34 μmol, 68.6%). This intermediate was then added to compound 31 (10.2 mg, 13.2 μmol) followed by DiPEA (2 μL, 10 μmol) and HATU (2.1 mg, 28 μL, 200 mM, 5.4 μmol). After stirring for 20.5 hours at room temperature, the reaction mixture was purified by prep-HPLC (40%->95% acetonitrile in 10 mM NH₄HCO₃ in water, column Xbridge prep C₁₈, 5 μM OBD, 30×100 mm). Compound 9c was obtained as a red solution in DMF (250 μL, 3.83 mM based on a doxorubicin-based calibration line for HPLC, 1.34 mg, 0.95 μmol, 28.6%). LCMS (ESI+) calculated for C₆₇H₈₇N₈O₂₃S⁺ (M+H⁺) 1404.51 found 1404.06.

### Example a52. Synthesis of Compound 9d

To a solution of compound 6d (3.4 mg, 4.7 μmol) in DCM was added MeOH (5 mL) and the mixture was concentrated until only MeOH (2.1 mL) was left. Then a solution of sodium periodate in water (60 mM, 288 μL, 17.5 μmol) was added and the reaction mixture was stirred for 68 hours in the dark. Once complete conversion was achieved, DMF (600 μL) was added and the reaction mixture was concentrated till there was 490 μL in DMF was left (1.78 mg, 2.53 μmol, 53%). This intermediate was then added to compound 31 (6.2 mg, 8.1 μmol) followed by DiPEA (1.32 μL, 7.59 μmol) and HATU (1.15 mg, 15.2 μL, 200 mM, 3.04 μmol). After stirring for 21.5 hours at room temperature, the reaction mixture was purified by prep-HPLC (40%→95% acetonitrile in 10 mM NH₄HCO₃ in water, column Xbridge prep C₁₈, 5 μM OBD, 30×100 mm). Compound 9d was obtained as a red solution in DMF (165 μL, 10.14 mM based on a doxorubicin-based calibration line for HPLC, 2.42 mg, 1.67 μmol, 65.9%). LCMS (ESI+) calculated for C₇₁H₈₇N₈O₂₃S⁺ (M+H⁺) 1452.56 found 1452.03.

### Example a52-2. Synthesis of Compound 9f

To a solution of compound 6f (6.8 mg, 10.4 μmol) in a mixture of MeOH (6.7 mL) and water (1.4 mL) was added a solution of sodium periodate in water (62.9 mM, 206 μL, 13.0 μmol) was added and the reaction mixture was stirred at rt for 3 hours in the dark. Additional sodium periodate in water (62.9 mM, 210 μL, 13.2 μmol) was added and the reaction mixture was stirred at rt for another 17 hours. Finally, a 3rd batch of sodium periodate in water (62.9 mM, 50 μL, 3.1 μmol) was added and the mixture was stirred at rt for 80 minutes and was then partially concentrated in vacuo to a volume of 5.4 mL and then left at rt for another 5 hours. DMF (670 μL) was then added, and the resulting red solution was partially concentrated to a volume of circa 350 μL, affording a white residue and a red solution containing crude intermediate. The mixture was diluted with additional DMF to 666 μL and 222 μL (3.45 μmol) of this solution was then treated with a stock solution of compound 31 in DMF (110 mmolar, 62.7 μL, 6.9 μmol) followed by DiPEA (1.79 μL, 10.4 μmol) and a solution of HATU in dry DMF (204 mM, 16.9 μL, 3.45 μmol). The resulting mixture was vortexed and left at room temperature for 31 minutes. Next, additional compound 31 in DMF (110 mmolar, 13.9 μL, 1.53 μmol) and HATU in dry DMF (204 mM, 33.8 μL, 6.90 μmol) were added. The mixture was again vortexed and left at room temperature for 13 minutes and then stored in the freezer for 17 hours. Finally, the mixture was removed from the freezer and purified by prep-HPLC (40%->95% acetonitrile in 10 mM NH₄HCO₃ in water, column Xbridge prep C₁₈, 5 μM OBD, 30×100 mm). Compound 9f was obtained as a red solution in DMF (300 μL, 8.2 mM based on a doxorubicin-based calibration line for HPLC, 3.9 mg, 2.47 μmol, 71.6%). LCMS (ESI+) calculated for C₆₆H₈₅N₈O₂₃S⁺ (M+H⁺) 1389.54 found 1390.07.

### Example a53. Synthesis of Compound 9g (OMe-PNU)

To a solution of compound 6a (2.17 mg, 3.38 μmol) in DCM was added MeOH (3 mL) and the mixture was concentrated until only MeOH (1.35 mL) was left. Water (300 μL) was added and a solution of sodium periodate in water (60 mM, 112.4 μL, 6.8 μmol) was added and the reaction mixture was stirred for 19 hours in the dark. The stirring bar was removed, and the RM was concentrated in vacuo, DMF (200 μL) was added to the residue, affording a white residue and a red solution containing crude intermediate. To this intermediate was then added compound 31 (6 mg, 7.8 μmol) followed by DiPEA (1.8 μL, 10.5 μmol) and HATU (2.05 mg, 5.4 μL, 196 mM, 5.4 μmol). After 25 minutes at room temperature, the reaction mixture was purified by prep-HPLC (40%→95% acetonitrile in 10 mM NH₄HCO₃ in water, column Xbridge prep C₁₈, 5 μM OBD, 30×100 mm). Compound 9g was obtained as a red solution in DMF (122 μL, 14.24 mM based on a doxorubicin-based calibration line for HPLC, 2.4 mg, 1.74 μmol, 80.9%). LCMS (ESI+) calculated for C₆₅H₈₃N₈O₂₃S⁺ (M+H⁺) 1375.53 found 1376.01.

### Example a54. Synthesis of Compound 32

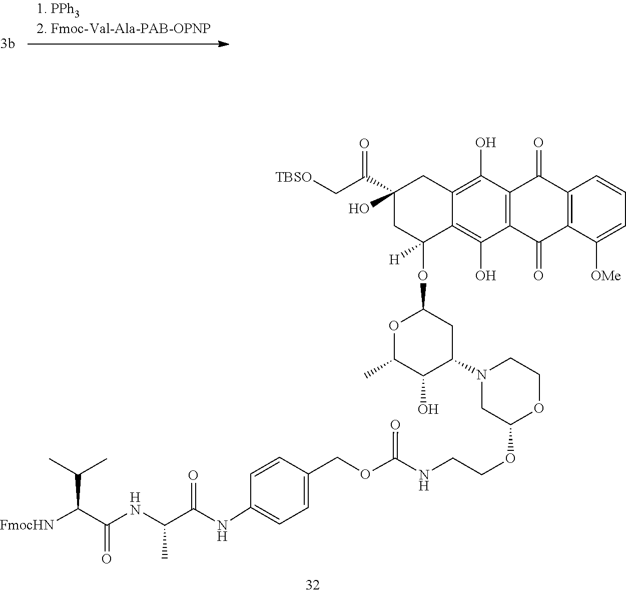

To a solution of compound 3b (80.4 mg, 98.9 μmol) in MeOH (600 μL) was added a solution of triphenylphosphine in DCM (88.2 mg, 967 μL, 348 mM, 336 μmol) and water (450 μL). The biphasic mixture was stirred for 3 hours at room temperature after which Fmoc-Val-Ala-PAB-OPNP (79.4 mg, 117 μmol) was added. After stirring the biphasic mixture for an additional 16 hours at room temperature, an extraction with DCM (2×1 mL) was done to remove the water. The combined organic layers were dried over Na₂SO₄ and immediately purified by flash column chromatography over silicagel (0%→40% EtOAc in DCM (to remove excess Fmoc-Val-Ala-PAB-OPNP) followed by 0%→15% MeOH in DCM) to give compound 32 as a clear red solution in DCM (7.7 mL, 123.6 mg, 93 μmol, 94.1%). LCMS (ESI+) calculated for C₇₀H₈₆N₅O₁₉Si⁺ (M+H⁺) 1329.54 found 1329.00.

### Example a55. Synthesis of Compound 33

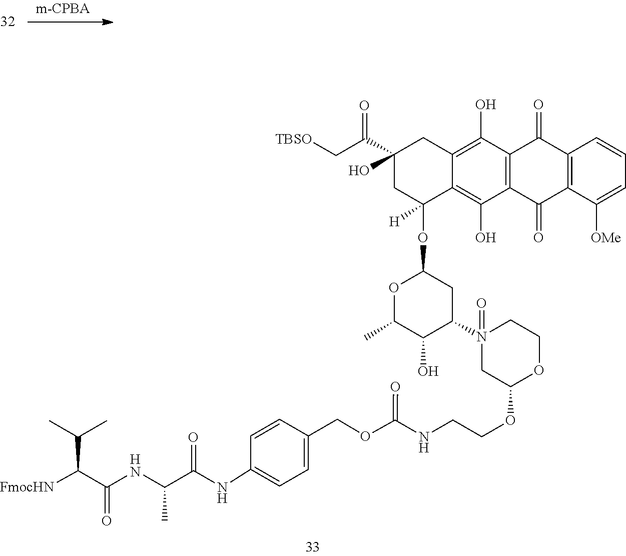

A solution of compound 32 (123.6 mg, 93 μmol) in anhydrous DCM (7.7 mL) was concentrated until a 2 mL solution was left. Next, the reaction mixture was cooled with dry ice/acetone cooling-bath to a temperature of −78° C. The mixture was vigorously stirred after which a freshly made stock solution of m-CPBA in anhydrous DCM (17.66 mg, 580 mM, 176 μL, 102.3 μmol) was dropwise added. After stirring for 17 minutes, full conversion was obtained. The RM was quenched with an ice-cold solution of acetone (reagent grade, 2.5 mL) and the RM was stirred. After an hour the cold bath was removed, and the RM was allowed to warm up to room temperature. The RM was transferred to a separation funnel and washed twice with saturated aqueous NaHCO₃ solution (10 mL). The water layers were combined and extracted twice with DCM (20 mL). The combined organic layers were dried over Na₂SO₄, filtered through a filter paper, and concentrated till a volume of 19 mL was obtained. No further purification was performed and compound 33 (108.3 mg, 80.5 μmol, 86.6%) was used as such. LCMS (ESI+) calculated for C₇₀H₈₆N₅O₂₀Si⁺ (M+H⁺) 1345.54 found 1345.14.

### Example a56. Synthesis of Compound 34 and 35

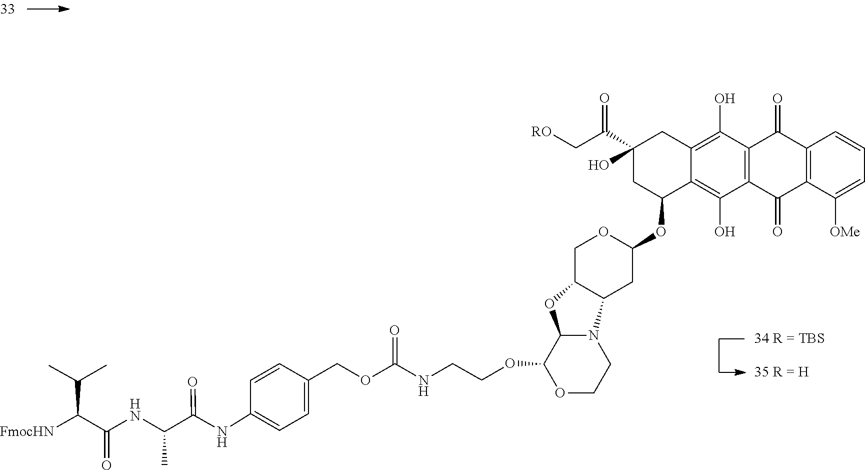

To a solution of compound 33 (108.3 mg, 80.5 μmol) in DCM (19 mL) was added anhydrous acetonitrile (10 mL). The RM was concentrated to remove the DCM and to obtain the compound in anhydrous acetonitrile (5 mL). After concentrating most of the solvent, the RM was further diluted with anhydrous acetonitrile (45 mL). Then, potassium carbonate (65 mg, 463 μmol) was added, and the RM was cooled to −10° C. after which cyanuric chloride (55.7 mg, 2.44 mL, 120 mM, 302.2 μmol) as a stock solution in anhydrous acetonitrile was added. After stirring for 5.5 h at 0° C., the RM was quenched with a solution of 3-aminopropane-1,2 diol (90.3 mg, 1.1 mL, 900 mM, 990 μmol) in water. The ice bath was removed after 15 min, and it was allowed to warm up to room temperature. To the RM was added DMF (3 mL) and the RM was concentrated till only a solution of DMF/water was left and purified by prep-HPLC (40%→100% acetonitrile in 10 mM NH₄HCO₃ in water, column Xbridge prep C₁₈, 5 μM OBD, 30×100 mm). The collected fractions were combined and concentrated till a volume of 11 mL acetonitrile was left. Then it was dried over Na₂SO₄, filtered and the drying agent was washed through with anhydrous THF (3×1 mL). Intermediate 34 was obtained as a solution in acetonitrile/THF (14 mL, 8.7 mg, 6.6 μmol, 8.1%). LCMS (ESI+) calculated for C₇₀H₈₄N₅O₁₉Si⁺ (M+H⁺) 1327.52 found 1327.07.

A solution of intermediate 34 (8.7 mg, 6.6 μmol) in anhydrous acetonitrile/THF (14 mL) was concentrated till 7 mL solution was left and anhydrous THF (1 mL) was added. To this was added triethylammonium acetate (5.3 mg, 5.3 μmol, 33 μmol) and the resulting red solution was cooled to −15° C. Then, while the RM was vigorously stirred TBAF (1 M in THF, 17.2 mg, 66 μL, 66 μmol) was added (color change from red to green observed). After stirring for 3 h, the RM was quenched with water (1.5 mL) and RM turned red again. The RM was transferred to a separation funnel and extracted with DCM (20 mL). The combined organic layers were dried over Na₂SO₄ and directly purified by flash column chromatography over silicagel (0%→10% MeOH in DCM) to give compound 35 as a red solution in DCM (8 mL, 2.3 mg, 1.9 μmol, 29%). LCMS (ESI+) calculated for C₆₄H₇₀N₅O₁₉⁺ (M+H⁺) 1212.47 found 1212.93.

### Example a57. Synthesis of Compound 36

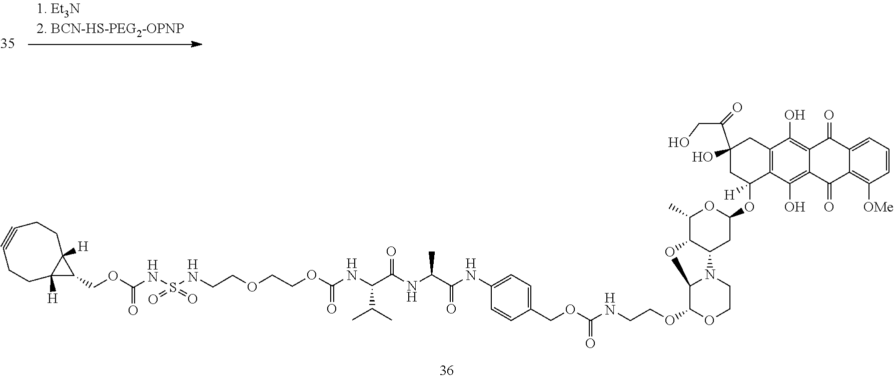 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OCCNC(=O)OCc4ccc(NC(=O)[C@H](C)NC(=O)[C@@H](NC(=O)OCCOCCNS(=O)(=O)NC(=O)OC[C@@H]5[C@@H]6CCC#CCC[C@@H]65)C(C)C)cc4)OCCN32)[C@H](C)O1]

Synthesis of BCN-HS-PEG2-OPNP has been described in WO2021144314A1, which is incorporated herein by reference. To a solution of compound 35 (2.3 mg, 1.9 μmol) in DCM (8 mL) was added DMF (100 μL) and the RM was concentrated to remove the DCM. Then, BCN-HS-PEG2-OPNP (1.2 mg, 25 μl, 93 mM, 2.3 μmol) a solution in DMF was added followed by triethylamine (2.6 μL, 19 μmol). After 30 hours at room temperature, the RM was further diluted with DCM (300 μL) and purified by flash column chromatography over silicagel (0%→15% MeOH in DCM) to give compound 36 as a red solution in DMF (150 μL, 0.64 mM based on a doxorubicin-based calibration line for HPLC, 0.13 mg, 0.09 μmol, 5%). LCMS (ESI+) calculated for C₆₅H₈₂N₇O₂₄S⁺ (M+H⁺) 1377.44 found 1377.04.

### Example a58. Synthesis of Compound 38

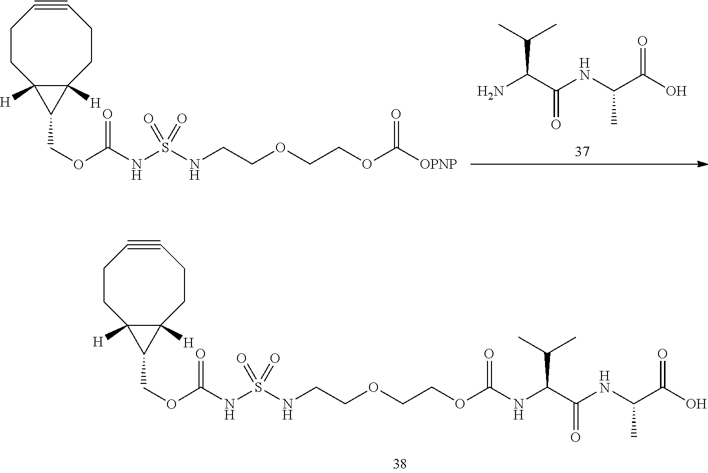

This compound was synthesized according to a literature procedure described in WO2017137457A1.

### Example a59. Synthesis of Compound 39

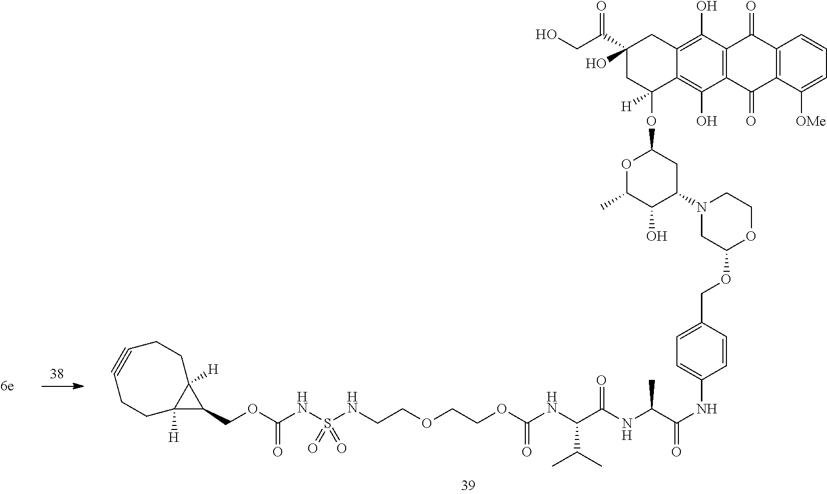 [SMILES: COc1cccc2c1C(=O)c1c(O)c3c(c(O)c1C2=O)C[C@@](O)(C(=O)CO)C[C@@H]3O[C@H]1C[C@H](N2CCO[C@H](OCc3ccc(NC(=O)[C@H](C)NC(=O)[C@@H](NC(=O)OCCOCCNS(=O)(=O)NC(=O)OC[C@@H]4[C@@H]5CCC#CCC[C@@H]54)C(C)C)cc3)C2)[C@H](O)[C@H](C)O1]

To a solution of compound 6e (1.0 mg, 1.3 μmol, 1.00 equiv.) in non-dry THF (90 μL) was added a solution of PPh3 (200 mmolar, 13 μL, 2.6 μmol, 2.0 equiv.) in THF and H₂O (13 μL). The resulting red solution was vortexed and then left at room temperature for 19 hours. The mixture was then transferred to an Eppendorf vial and put in an Eppendorf shaker at 37° C. and 1400 RPM for 6.5 hours and then for another 19 hours at room temperature at 1400 RPM. The reaction mixture was then stored in the freezer for 11 days and then treated with a solution of compound 38 (790 mM, 2.0 μL, 1.6 μmol, 1.2 equiv.) in DMF, followed by the addition of HATU in DMF (517 mM, 1.4 μmol, 1.1 equiv.) and finally a solution of DMAP in DMF (500 mM, 1.1 μL, 0.53 μmol, 0.4 equiv.). The resulting mixture was vortexed and left at room temperature for 3 hours and was then stored in the freezer for 21 hours. The mixture was removed from the freezer and purified by prep-HPLC (40%→100% acetonitrile in 10 mM NH₄HCO₃ in water, column Xbridge prep C₁₈, 5 μM OBD, 30×100 mm). The pure fractions were combined and diluted with DMF and partially concentrated to give compound 39 as a red solution in DMF (85 μL, 1.5 mM based on a doxorubicin-based calibration line for HPLC, 0.17 mg, 0.13 μmol, 10%). LCMS (ESI+) calculated for C₆₂H₇₇N₆O₂₂S⁺ (M+H⁺) 1289.48 found 1289.99.

### Example a60. Synthesis of Compound 42 and 43

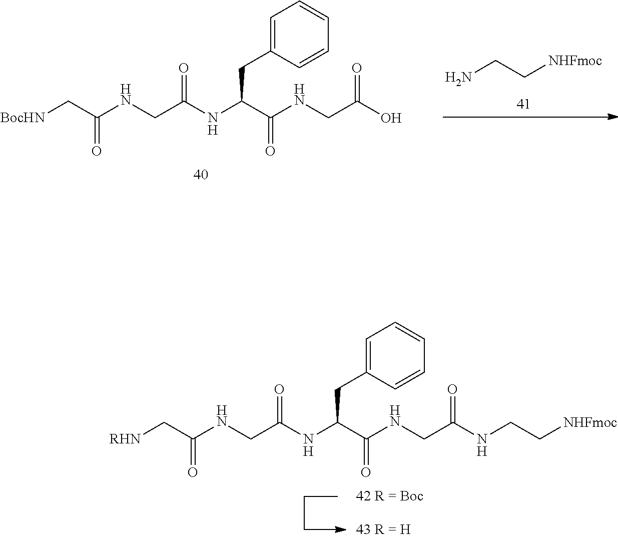

To vial containing Boc-Gly-Gly-Phe-Gly-OH (500 mg, 1.15 mmol, 1.00 equiv.) and Fmoc-EDA-H (476 mg, 1.69 mmol, 1.47 equiv.) was added DMF (1.25 mL), followed by Et₃N (479 UL, 3.44 mmol, 3.00 equiv.) and finally HATU (479 mg, 1.26 mmol, 1.10 equiv.). The resulting mixture was stirred at room temperature for 2 minutes before adding DCM (2 mL), generating a clear yellow solution, which was stirred at room temperature for 110 minutes. The reaction mixture was then stored in the freezer for 16 hours. The mixture was then removed from the freezer and diluted with DCM (15 mL) and purified directly via flash column chromatography (50→100% EtOAc in heptane). Fractions containing the product were concentrated, affording compound 42 as a white solid (357 mg, 509 μmol, 45%). LCMS (ESI+) calculated for C₃₇H₄₅N₆O₈⁺ (M+H⁺) 701.33 found 701.67. Compound 42 (357 mg, 509 μmol, 1.00 equiv.) was suspended in DCM (3.0 mL) and cooled to 0° C. with an ice-bath. Next, TFA (1.37 mL, 17.8 mmol, 35 equiv.) was added dropwise, generating a light-yellow solution that was stirred at 0° C. for 47 minutes. The ice-bath was then removed, and the RM was warmed to room temperature and stirred for another 75 minutes. The mixture was concentrated in vacuo and the residue was taken up in a mixture of DCM (1.5 mL) and toluene (2 mL) and concentrated a 2^{nd} time, affording compound 43 (TFA-salt) as a brittle solid, which was used without further purification in the next step. Alternatively, compound 43 was subjected to a prep-HPLC purification (5%→95% acetonitrile with 1% AcOH in water with 1% AcOH, column Xbridge prep C18, 5 μM OBD, 30×100 mm) to obtain compound 43 as an acetate-salt. LCMS (ESI+) calculated for C₃₂H₃₇N₆O₆⁺ (M+H⁺) 601.28 found 601.60.

### Example a61. Synthesis of Compound 45 and 46

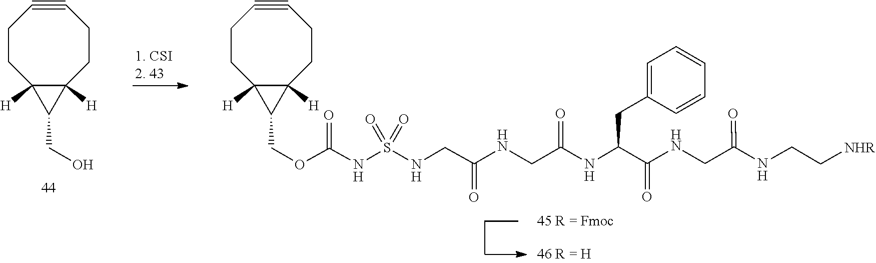

To a solution of BCN-OH 44 (40 mg, 90 wt % according to ¹H-qNMR, 240 μmol, 1.00 equiv.) in dry DCM (2.5 mL) at −40° C. in a dry ice/MeCN bath was added chlorosulfonyl isocyanate (22 μL, 250 μmol, 1.05 equiv.) in one portion, resulting in a pale-yellow solution. The mixture was stirred at −40° C. for 17 minutes, followed by the addition of Et₃N (67 μL, 480 μmol, 2.00 equiv.). The resulting solution was stirred for circa 5 minutes at −40° C. before adding a solution of the acetate-salt of compound 43 (80 mg, 120 μmol, 0.51 equiv.) and DIPEA (23 μL) in DMF (250 μL), followed after 10 minutes by another addition with the TFA-salt of compound 43 (115.4 mg, 161.5 μmol, 0.67 equiv.) as a suspension in a mixture of DIPEA (46 μL) and Et₃N (23 μL) in DMF (5 mL). Finally, dimethylacetamide (1 mL) was added and the reaction mixture was allowed to warm to room temperature over 5 hours. The reaction mixture was then stored in the freezer for 5 days and was then removed from the freezer and concentrated in vacuo. The residue was purified via by prep-HPLC (40%→90% acetonitrile with 1% AcOH in water with 1% AcOH, column Xbridge prep C18, 5 μM OBD, 30×100 mm) to give compound 45 (8.9 mg, 9.5 μmol, 4% yield). LCMS (ESI+) calculated for C₄₃H₅₀N₇O₁₀S⁺ (M+H⁺) 856.33 found 856.66. To a solution of compound 45 (8.9 mg, 9.5 μmol in non-dry DMF (400 μL) was added Et₃N (58 μL, 0.42 mmol, 40 equiv.). The resulting mixture was mixed and left at room temperature for circa 18 hours and was then concentrated in vacuo to a volume of circa 25 μL. This solution was transferred to an Eppendorf vial with additional DMF to give compound 46 as a solution in DMF with final volume of 150 UL, which was used without further purification in the next step. LCMS (ESI+) calculated for C₂₈H₄₀NO₈S⁺ (M+H⁺) 634.27 found 634.54.

### Example a62. Synthesis of Compound 47

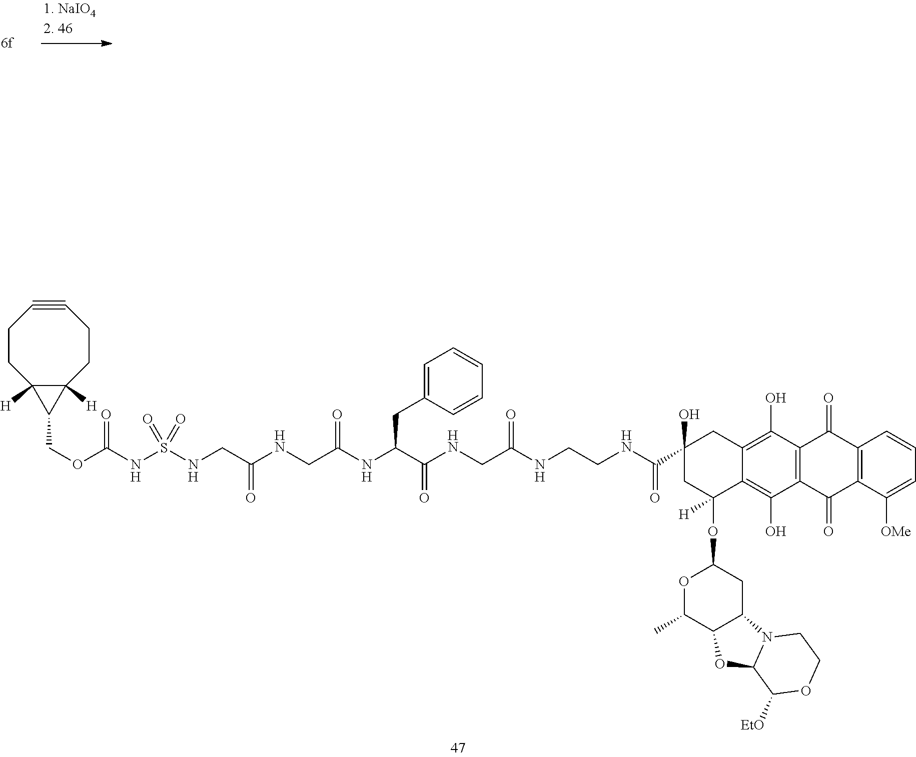 [SMILES: CCO[C@H]1OCCN2[C@@H]1O[C@@H]1[C@H](C)O[C@@H](O[C@H]3C[C@](O)(C(=O)NCCNC(=O)CNC(=O)[C@H](Cc4ccccc4)NC(=O)CNC(=O)CNS(=O)(=O)NC(=O)OC[C@@H]4[C@@H]5CCC#CCC[C@@H]54)Cc4c(O)c5c(c(O)c43)C(=O)c3c(OC)cccc3C5=O)C[C@@H]12]

To a solution of compound 6f (6.8 mg, 10.4 μmol) in a mixture of MeOH (6.7 mL) and water (1.4 mL) was added a solution of sodium periodate in water (62.9 mM, 206 μL, 13.0 μmol) and the reaction mixture was stirred at rt for 3 hours in the dark. Additional sodium periodate in water (62.9 mM, 210 μL, 13.2 μmol) was added and the reaction mixture was stirred at rt for another 17 hours. Finally, a 3rd batch of sodium periodate in water (62.9 mM, 50 μL, 3.1 μmol) was added and the mixture was stirred at rt for 80 minutes and was then partially concentrated in vacuo to a volume of 5.4 mL and then left at rt for another 5 hours. DMF (670 μL) was then added, and the resulting red solution was partially concentrated to a volume of circa 350 μL, affording a white residue and a red solution containing crude intermediate. The mixture was diluted with additional DMF to 666 μL and 222 μL (3.45 μmol) of this solution was then treated with a stock solution of compound 46 in DMF (66.6 mmolar, 150 μL, 9.99 μmol) followed by DiPEA (1.80 μL, 10.4 μmol) and a solution of HATU in dry DMF (490 mM, 7.1 μL, 3.5 μmol). The resulting mixture was vortexed and left at room temperature for 17 minutes. Next, additional HATU in dry DMF (490 mM, 7.1 μL, 3.5 μmol) was added. The mixture was again vortexed and left at room temperature for 23 minutes. A 3rd batch of HATU in dry DMF (490 mM, 7.1 μL, 3.5 μmol) was added. The resulting mixture was vortexed and left at room temperature for 14 minutes. Next, additional compound 46 (2.4 mg, 3.8 μmol) was added to the RM, followed after 14 minutes by additional compound 46 (2.4 mg, 3.8 μmol) and a 4th batch of HATU in dry DMF (490 mM, 7.1 μL, 3.5 μmol). The mixture was stirred at rt for another 29 minutes before adding additional DiPEA (1.80 μL, 10.4 μmol). The mixture was then pushed over a membrane-filter and then treated with a 5th and final batch of HATU in dry DMF (490 mM, 7.1 μL, 3.5 μmol). The mixture was vortexed and left at room temperature for 80 minutes and was then purified by prep-HPLC (40%→95% acetonitrile in 10 mM NH₄HCO₃ in water, column Xbridge prep C18, 5 μM OBD, 30×100 mm). Compound 47 was obtained as a red solution in DMF (196 μL, 3.5 mM based on a doxorubicin-based calibration line for HPLC, 1.0 mg, 0.684 μmol, 20%). LCMS (ESI+) calculated for C₆₀H₇₃N₈O₂₀S⁺ (M+H⁺) 1257.47 found 1357.86.

### Example a63. Synthesis of Compound 49 and 50

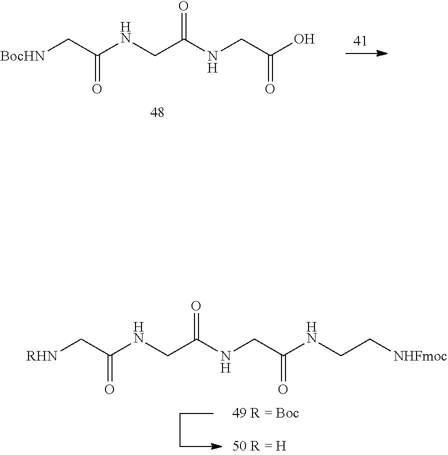

To a suspension of (tert-butoxycarbonyl) glycylglycylglycine (337.4 mg, 1.17 mmol, 1.00 equiv.) and Fmoc-EDA-H (368.8 mg, 1.306 mmol, 1.12 equiv.) in DMF (1 mL) was added triethylamine (488 μL, 3.50 mmol, 3.00 equiv.). To the resulting suspension was added HATU (494.8 mg, 1.301 mmol, 1.12 equiv.). The resulting yellow mixture was stirred at room temperature for 2 minutes, followed by the addition of DCM (2 mL), generating a yellow solution which was stirred at room temperature for another 90 minutes. The mixture was then stored in the freezer for 1 day. The mixture was removed from the freezer and then purified via flash column chromatography (0→10% MeOH in DCM), affording the product 50 (443.5 mg, 801.1 μmol, 68.7%) as a white solid. LCMS (ESI+) calculated for C₂₈H₃₆N₅O₇⁺ (M+H⁺) 554.26 found 554.60. Compound 50 was then dissolved in DCM (2.00 mL) and the resulting mixture was cooled in an ice-bath to 0° C. Next, TFA (400 μL, 5.19 mmol, 7.05 equiv.) was added dropwise to the reaction mixture while stirring. Upon complete addition the ice-bath was removed, and the resulting solution was stirred at room temperature for 85 minutes. Additional TFA (1.6 mL, 21 mmol, 28 equiv.) was added portion-wise and the resulting mixture was stirred at room temperature for another 200 minutes. The mixture was then concentrated in vacuo, and the residue was then purified by prep-HPLC (5%→90% acetonitrile in water, column Xbridge prep C₁₈, 5 μM OBD, 30×100 mm). Compound 50 was obtained as white powder (84.9 mg, 186 μmol, 25%). LCMS (ESI+) calculated for C₂₃H₂₈N₅O₅⁺ (M+H⁺) 454.21 found 454.54.

### Example a64. Synthesis of Compound 51 and 52

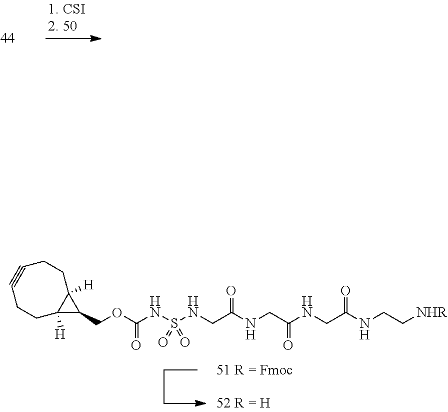

To a solution BCN-OH 44 (30.5 mg, 90 wt % according to ¹H-qNMR, 183 μmol, 1.00 equiv.) in dry DCM (2.1 mL) at −40° C. in a dry ice/MeCN bath was added chlorosulfonyl isocyanate (16.7 μL, 192 μmol, 1.05 equiv.) in one portion resulting in a pale-yellow solution. The mixture was stirred at −40° C. for 5 minutes, followed by the addition of Et₃N (76.4 μL, 548 μmol, 3.00 equiv.). The resulting solution was stirred for circa 14 minutes at −40° C. before adding a solution of compound 50 (84.9 mg, 187 μmol, 1.02 equiv.) in DMF (450 μL). This reaction mixture was stirred at −40° C. for 10 minutes and was then allowed to warm to room temperature over 2 hours. The reaction mixture was then stored in the freezer for 1 day and then removed from the freezer and diluted with DCM and purified via flash column chromatography (0→13% MeOH in DCM), affording impure intermediate 51 (containing Et₃N) as two batches (in total 45 μmol, 25% yield). The two batches were combined and co-evaporated with DMF. The resulting oil was diluted with DCM (10 mL) and washed twice with sat. aq. NH₄Cl (2 mL, 2×). During the extraction a gummy oil was formed which was combined with the organic layer and concentrated in vacuo, affording intermediate 51 as a brown gummy solid. The residue was taken up in 600 μL DMF, followed by the addition of Et₃N (240 μL, 1.70 mmol, 40 equiv.). The resulting mixture was stirred at room temperature for 1 day and was then stored in the freezer for 3 days. The reaction mixture was removed from the freezer and filtered over a membrane-filter, which was washed with additional DMF (3×). The resulting yellow solution was partially concentrated to a volume of circa 240 μL to give compound 52 as a yellow solution in DMF, which was used without further purification in the next step. LCMS (ESI+) calculated for C₁₉H₃₁N₆O₇S⁺ (M+H⁺) 487.20 found 487.55.

### Example a65. Synthesis of Compound 53

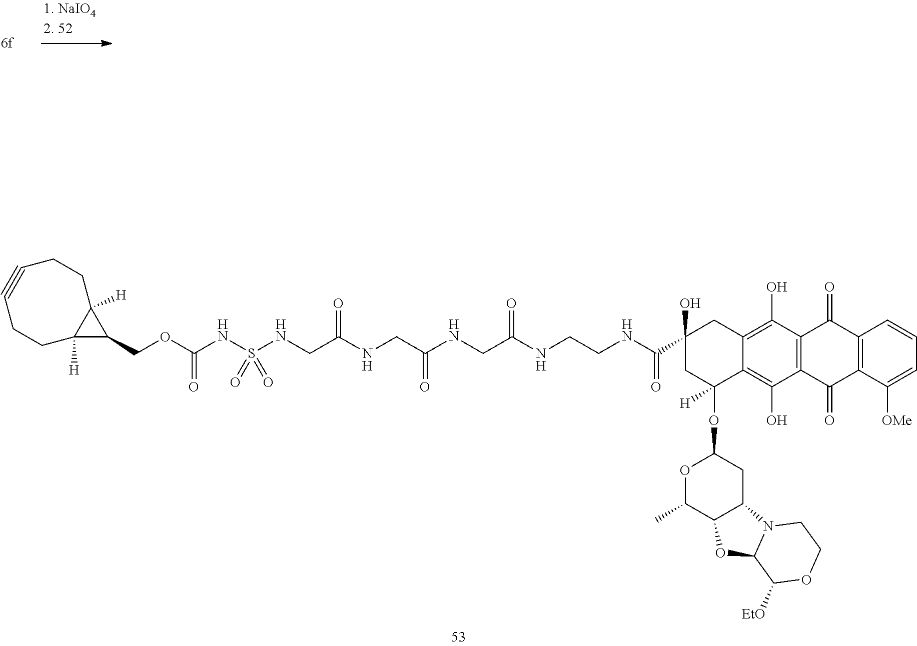 [SMILES: CCO[C@H]1OCCN2[C@@H]1O[C@@H]1[C@H](C)O[C@@H](O[C@H]3C[C@](O)(C(=O)NCCNC(=O)CNC(=O)CNC(=O)CNS(=O)(=O)NC(=O)OC[C@@H]4[C@@H]5CCC#CCC[C@@H]54)Cc4c(O)c5c(c(O)c43)C(=O)c3c(OC)cccc3C5=O)C[C@@H]12]

To a solution of compound 6f (6.8 mg, 10.4 μmol) in a mixture of MeOH (6.7 mL) and water (1.4 mL) was added a solution of sodium periodate in water (62.9 mM, 206 μL, 13.0 μmol) and the reaction mixture was stirred at rt for 3 hours in the dark. Additional sodium periodate in water (62.9 mM, 210 μL, 13.2 μmol) was added and the reaction mixture was stirred at rt for another 17 hours. Finally, a 3rd batch of sodium periodate in water (62.9 mM, 50 μL, 3.1 μmol) was added and the mixture was stirred at rt for 80 minutes and was then partially concentrated in vacuo to a volume of 5.4 mL and then left at rt for another 5 hours. DMF (670 μL) was then added, and the resulting red solution was partially concentrated to a volume of circa 350 μL, affording a white residue and a red solution containing crude intermediate. The mixture was diluted with additional DMF to 666 μL and 222 μL (3.45 μmol) of this solution was then treated with a stock solution of 52 in DMF (175 mmolar, 39.4 μL, 6.9 μmol) followed by DiPEA (1.79 μL, 10.4 μmol) and a solution of HATU in dry DMF (500 mM, 6.90 μL, 3.45 μmol). The resulting mixture was vortexed and left at room temperature for 17 minutes. Next, additional 52 in DMF (175 mmolar, 157.6 μL, 27.6 μmol) was added. The mixture was again vortexed and left at room temperature for 14 minutes, followed by the addition of additional HATU in dry DMF (500 mM, 13.80 μL, 6.90 μmol). The mixture was again vortexed and left at room temperature for 13 minutes and then stored in the freezer for 2 days. Finally, the mixture was removed from the freezer and purified by prep-HPLC (40%→95% acetonitrile in 10 mM NH₄HCO₃ in water, column Xbridge prep C₁₈, 5 μM OBD, 30×100 mm). Compound 53 was obtained as a red solution in DMF (165 μL, 2.5 mM based on a doxorubicin-based calibration line for HPLC, 0.5 mg, 0.41 μmol, 12%). LCMS (ESI+) calculated for C₅₁H₆₄N₇O₁₉S⁺ (M+H⁺) 1110.40 found 1110.85.

### Synthesis of Compound 57

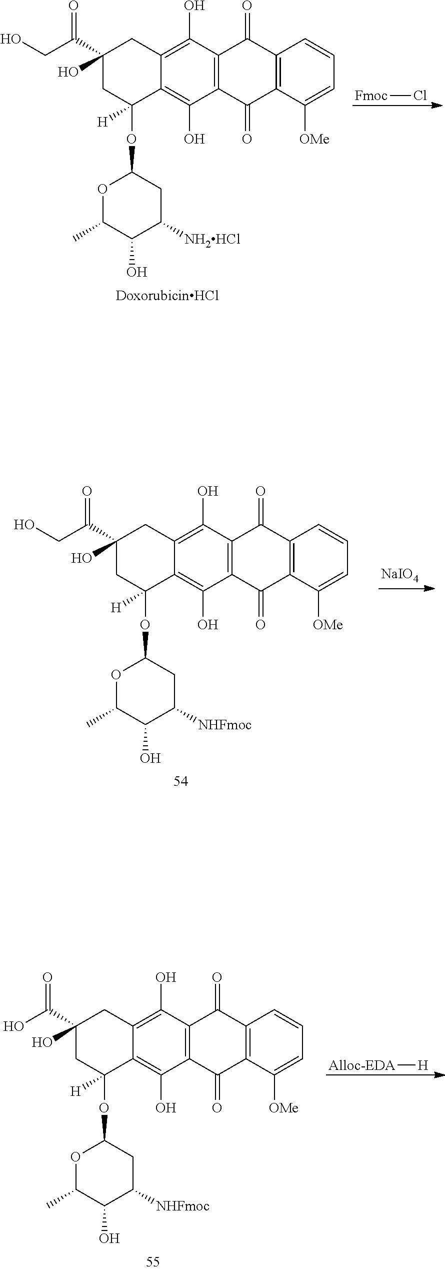

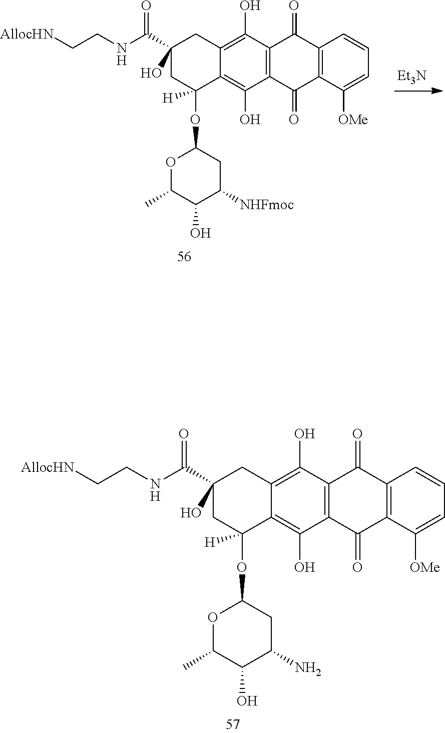

### Example a66. Synthesis of Compound 54

This compound was synthesized according to a literature procedure described by Pawar et al. in the International Journal of Pharmaceutics, Volume 436, Issues 1-2, Pages 183-193.

### Example a67. Synthesis of Compound 55

To a round-bottom flask containing compound 54 (5.241 g, 1 Eq, 6.844 mmol) was added a solution of THF (160 mL) and water (160 mL). The resulting red suspension was cooled to 0° C. and then a cold solution of sodium periodate in H2O (1.464 g, 34.22 mL, 200 mmolar, 1.00 Eq, 6.844 mmol) was added dropwise over 15 minutes. The resulting red solution with some solids at the bottom was stirred on ice for a total of 10 minutes. The ice-bath was then removed, and the RM was allowed to warm to room temperature and stirred for 23 hours. The RM was partially concentrated in vacuo (removing all THF and circa 50% of the water, until 80 mbar), affording a red suspension in mostly water. The mixture was treated with DCM (200 mL) and the resulting suspension was rotated at 43° C. degrees for a few minutes. The bi-phasic system was transferred to a separation funnel. To the remaining residue in the round bottom flask was added additional 300 ml DCM and the resulting mixture was rotated at 43° C. again until the remaining solids had dissolved. The solution was also added to the separation funnel and the resulting bi-phasic system was shaken and separated. The aqueous layer was extracted twice with additional DCM (2×200 ml). The separatory funnel-containing small amounts of dark red residuals—was washed with 10% MeOH in DCM (150 mL), which completely solubilized the residue. This organic layer was washed with the water layer once. The resulting organic layer was then combined with the other organic layers. To the combined organic layers was added MeOH (40 mL) generating a clear solution. The combined organic layers were dried (Na₂SO₄) and then filtered over a glass-filter. The solution was treated with DMF (18 mL) and partially concentrated until mainly DMF was left as a solvent (until 20 mbar), affording intermediate 55 as a solution in DMF (5.30 g, 6.87 mmol) as a dark red solution (18 mL), which was used in the next step without further purification. Quantitative yield was assumed. UPLC-MS (ESI+) calculated for C₄₁H₃₇NO₁₃⁺ [M+H⁺] 752.23 found 752.52.

### Example a68. Synthesis of Compound 56

To a round-bottom flask containing compound 55 (4.10 g, 5.46 mmol, 1.00 equiv.) in DMF (15 ml) was added additional DMF until a total of circa 94 mL was reached. Next, allyl (2-aminoethyl) carbamate (2.14 g, 2.72 Eq, 14.9 mmol) was added in dry DMF (9.0 ml) and the RM was placed in a water-bath. Next, HATU (2.18 g, 1.05 Eq, 5.73 mmol) was added, followed within one minute by the addition of DIPEA (2.12 g, 2.85 mL, 3.00 Eq, 16.4 mmol) and the resulting dark red solution was stirred at rt for circa 30 min. Next, additional HATU (455 mg, 1.20 mmol, 0.22 equiv.) in DMF (1.0 mL) was added, followed after another 55 minutes by a third batch of HATU (509 mg, 1.34 mmol, 0.25 equiv.). The RM was stirred at rt for another 5 minutes and was then partially concentrated in vacuo until a volume of 15 ml. The residue was then diluted with DCM (135 ml) and loaded onto a pre-wetted column. The residue was then purified by flash column chromatography over silicagel (0→20% MeOH/DCM). Fractions containing product were combined and concentrated, affording intermediate 56 (3.61 g, 4.07 mmol, 74.5%, 99% purity) as a dark red thick oil. UPLC-MS (ESI+) calculated for C₄₇H₄₈N₃O₁₄⁺ [M+H⁺] 878.31 found 878.71.

### Example a69. Synthesis of Compound 57

To a dark red solution of compound 56 (3.61 g, 99% Wt, 1.00 Eq, 4.07 mmol) in a total volume of DMF (17.0 mL) was added triethylamine (2.06 g, 2.84 mL, 5.0 Eq, 20.3 mmol). The RM turned very dark red and was left to stir at rt for 18 hours. Next, Et₂O (78 mL) was added (slowly) in one portion, while rapidly stirring. The stirring was stopped and the ether-layer was then decanted and the remaining dark solid was washed a few more times with Et₂O (3×100 ml). The solid was concentrated in vacuo, affording intermediate 57 (4.8 g, 81.9% purity) as a dark red solid that was used without further purification. UPLC-MS (ESI+) calculated for C₃₂H₃₈N₃O₁₂⁺ [M+H⁺] 656.25 found 656.60.

### Example a70. Synthesis of Compound 58

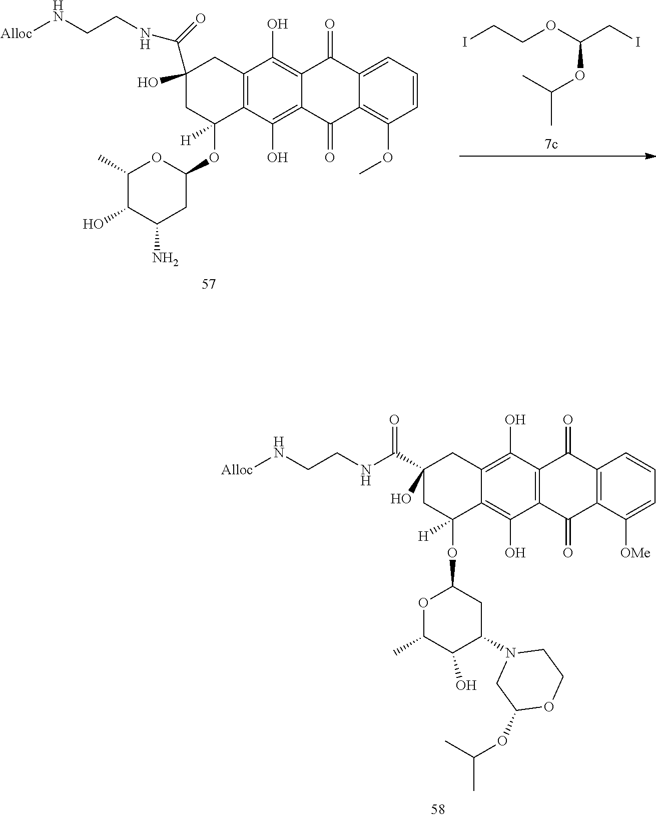

To intermediate 57 (4.0 g, 90 wt %, 5.5 mmol), was suspended in dry DMF (6.0 mL), followed by the addition of bis-iodo-sugar 7c (6.3 g, 16 mmol, 3 equiv.) and DiPEA (2.9 mL, 16 mmol, 3 equiv.). The resulting mixture was rotated at 45° C. for 45 minutes. Next, the remaining lumps were mostly broken up with a spatula and the suspension was stirred at 40° C. for 2 days. The RM was diluted with DCM (100 mL) and the resulting red solution was purified by flash column chromatography over silicagel (0% MeOH/DCM then, 2%→10% MeOH in DCM) to give compound 58 as a red residue (4.03 μmol, 72%). LCMS (ESI+) calculated for C₃₉H₅₀N₃O₁₄⁺ (M+H⁺) 784.33 found 784.78.

### Example a71. Synthesis of Compound 59

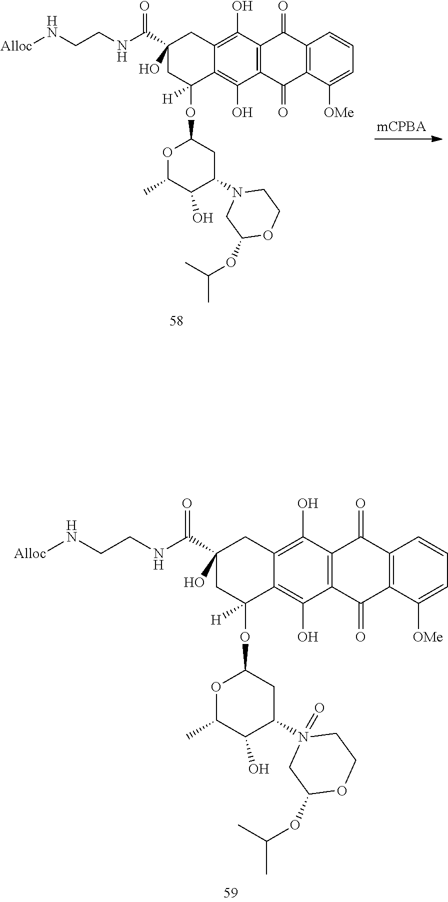

A solution of compound 58 (4.21 g, 4.78 mmol, 89 wt %) in a mixture of DCM (130 mL) and MeOH (10 mL) was cooled by dry ice/acetone-bath to a temperature of −78° C. The mixture was vigorously stirred after which a freshly made stock solution of mCPBA in DCM (580 mM, 9.0 mL, 5.22 mmol) was added dropwise over 5-10 minutes. After stirring for 17 minutes, a 2^{nd} batch of mCPBA in DCM (580 mM, 10.0 mL, 5.80 mmol) was added over 5 minutes and the RM was stirred for another 9 minutes, before finally adding a 3rd batch of mCPBA in DCM (580 mM, 2.5 mL, 1.50 mmol). The reaction mixture was stirred for another 13 minutes at −78° C. and was then quenched with cold (−78° C.) acetone (reagent grade, 43.9 mL) and the RM was stirred. After 90 min the cold bath was removed, and the RM was allowed to warm up to 0° C. over 40 minutes. The RM was diluted with DCM (450 mL) and saturated aqueous NaHCO₃ solution (250 mL) and transferred to a separation funnel. The resulting biphasic system was separated, and the water-layer was extracted twice with DCM (150, 100 mL). The combined organic layers were then washed again with sat. aq. NaHCO₃ solution (100 mL). The new water-layer was extracted with DCM (40 mL) and the combined organic layers were dried over Na₂SO₄ and filtered and then concentrated to give compound 59 (3.30 g, 93% purity, 80% yield, 3.84 mmol), which was used as such in the next step. LCMS (ESI+) calculated for C₃₉H₅₀N₃O₁₅⁺ (M+H⁺) 800.32 found 800.70.

### Example a72. Synthesis of Compound 60

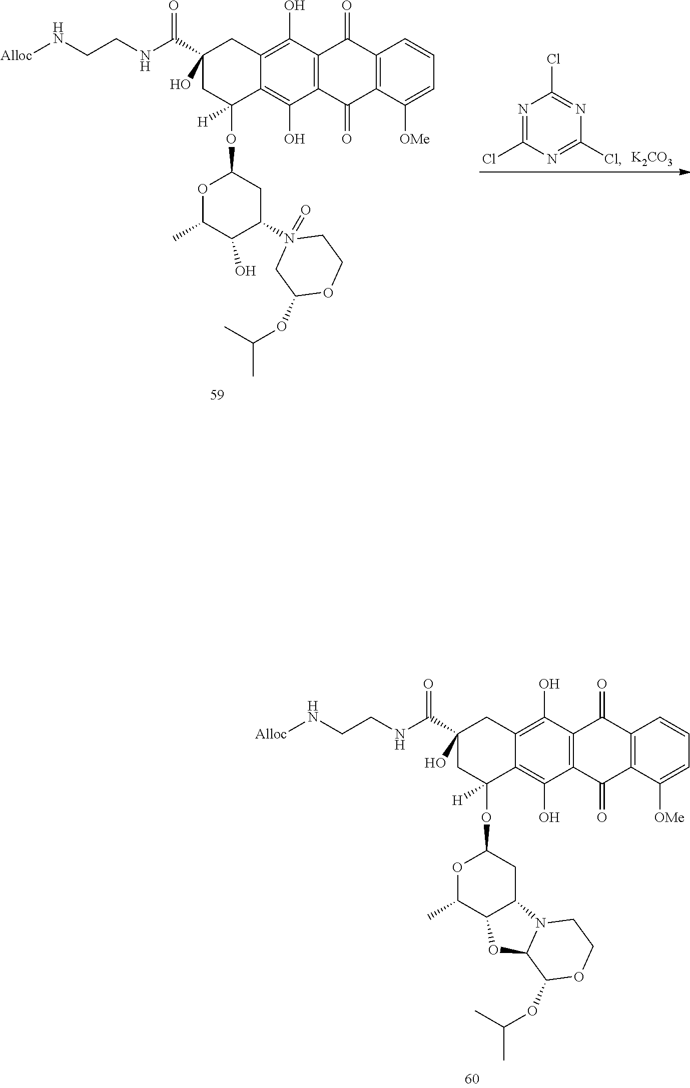

To a solution of 59 (181 mg, 60 wt %, 136 μmol, 1.00 equiv.) in a mixture of dry DCM (1.0 mL) and anhydrous acetonitrile (4.0 mL) was added potassium carbonate (167.8 mg, 1.21 mmol, 8.94 equiv.). The RM was then cooled to 0° C. with an ice-bath after which a solution of cyanuric chloride (62.6 mg, 2.88 mL, 118 mM, 339 μmol, 2.5 equiv.) in anhydrous acetonitrile was added. After stirring for 70 minutes at 0° C. additional cyanuric chloride (863 μL, 118 mM, 102 μmol, 0.75 equiv.) in anhydrous acetonitrile was added. The RM was stirred at 0° C. for another 55 minutes and was then quenched with a solution of 3-aminopropane-1,2 diol (186 mg, 1.02 mL, 2 Molar, 2.04 mmol, 15 equiv.) in water. The ice bath was removed after 30 min and was then diluted with DCM (30 mL) and H₂O (10 mL). The resulting bi-phasic system was separated. The water-layer was extracted twice more with DCM (10 mL, 2×). The combined organic layers were dried (Na₂SO₄), filtered over a phase-separator and concentrated in vacuo. The residue was taken up in DMF and purified by prep-HPLC (50%→70% acetonitrile in 10 mM NH₄HCO₃ in water, column Xbridge prep C₁₈, 5 μM OBD, 30×100 mm). The fractions containing pure product were combined and concentrated to give pure compound 60 (13.5 mg, 17.3 μmol, 12.7% yield) as a red solid. In addition, fractions containing impure product were combined, affording impure 60 (11.4 mg, 63% pure, 9.20 μmol, 6.7% yield). LCMS (ESI+) calculated for C₃₉H₄₈N₃O₁₄⁺ (M+H⁺) 782.82 found 782.64.

### Example a73. Synthesis of Compound 61

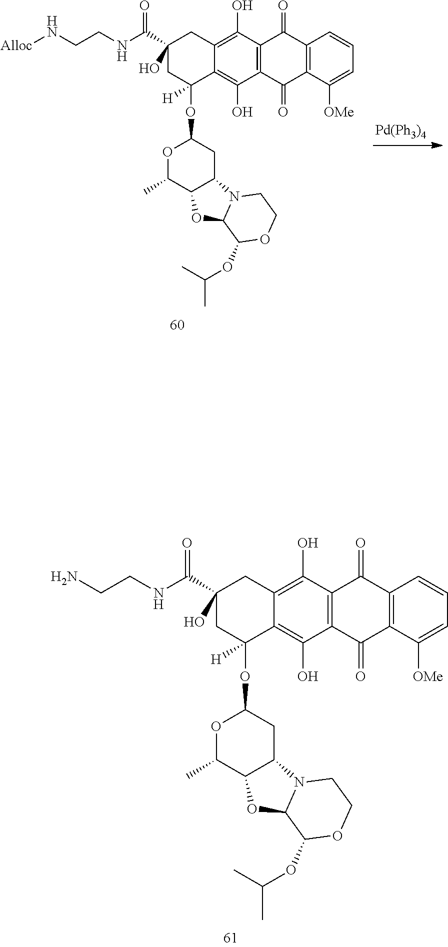

To a vial containing compound 60 (6.75 mg, 1 Eq, 8.63 μmol) was added anhydrous DCM (300 μL) and to the resulting red solution was added pyrrolidine (1.84 mg, 2.13 μL, 3 Eq, 25.9 μmol), generating a very dark red (nearly black) solution instantly. The mixture was mixed, followed by the addition of Pd(PPh₃)₄ in dry DCM (1.50 mg, 64.8 μL, 20 mmolar, 0.15 Eq, 1.30 μmol). The dark red solution was again mixed and left at rt for 11 minutes. Additional 20 mmolar Pd(PPh₃)₄ in dry DCM (64.8 μL, 0.15 Eq, 1.30 μmol) was added, followed after 13 and 12 minutes by a third (100 μL, 0.23 Eq, 2.00 μmol)) and fourth batch (64.8 μL, 0.15 Eq, 1.30 μmol) respectively. Finally, following the fourth addition the RM was stirred at rt for 47 minutes and was then diluted with DMF (300 μL) and was then partially concentrated in vacuo until 30 mbar to give a dark red solution that was purified by prep-HPLC (20%→50% acetonitrile in 10 mM NH₄HCO₃ in water, column Xbridge prep C₁₈, 5 UM OBD, 30×100 mm). The fractions containing pure product were combined and concentrated to give pure compound 61 (2.4 mg, 3.4 μmol, 40% yield) as a red residue. In addition, fractions containing impure product were combined, affording impure 61 (3.3 mg, 53% pure, 2.5 μmol, 29% yield). LCMS (ESI+) calculated for C₃₅H₄₄N₃O₁₂⁺ (M+H⁺) 698.29 found 698.56.

### Example a74. Synthesis of Compound BCN-HS-GGFG-OH (62)

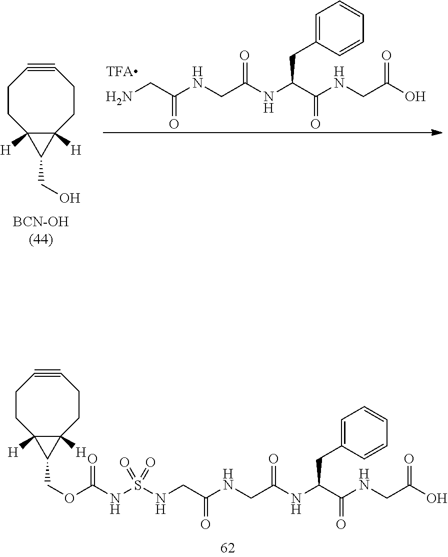 [SMILES: OC[C@H]1[C@H]2CCC#CCC[C@@H]12 ; NCC(=O)NCC(=O)N[C@@H](Cc1ccccc1)C(=O)NCC(=O)O ; O=C(O)CNC(=O)[C@H](Cc1ccccc1)NC(=O)CNC(=O)CNS(=O)(=O)NC(=O)OC[C@@H]1[C@@H]2CCC#CCC[C@@H]21]

A round-bottom flask containing a solution of BCN-OH 44 (150 mg, 92% Wt, 1 Eq, 919 μmol) in Acetonitrile (10 mL) was cooled to 0° C. by ice. Next, Chlorosulfonyl isocyanate (137 mg, 83.9 μL, 1.05 Eq, 965 μmol) was added. The RM was stirred for 20 minutes and was then treated with triethylamine (279 mg, 384 μL, 3 Eq, 2.76 mmol) followed by the addition of H-GlyGlyPheGly-OH·TFA (496 mg, 1.2 Eq, 1.10 mmol) as a solid. Next, Water (1 mL) was added, and the RM was stirred vigorously, followed by the addition of additional triethylamine (186 mg, 256 μL, 2 Eq, 1.84 mmol) was added to aid in solubility. The RM was stirred for circa 3 hours, generating a solution, which was diluted with DCM (20 mL) and water (20 mL) and brine (500 μL). The resulting bi-phasic system was shaken and then separated. The water-layer was extracted with additional DCM (10 mL). The combined water layers were combined and EtOAc (20 mL) was added. Then 1 M aq. HCl was added till the water layer reached a pH of circa 4. The two layers were separated, and the water layer was extracted twice more with EtOAc (2×20 mL). All the EtOAc-based organic layers were combined and dried over Na₂SO₄, filtered and concentrated, affording compound 62 (419 mg, 708 μmol, 77% yield) as a yellow oil. LCMS (ESI+) calculated for C₂₆H₃₄N₅O₉S⁺ (M+H⁺) 592.21 found 592.44.

### Example a75. Synthesis of Compound 63a

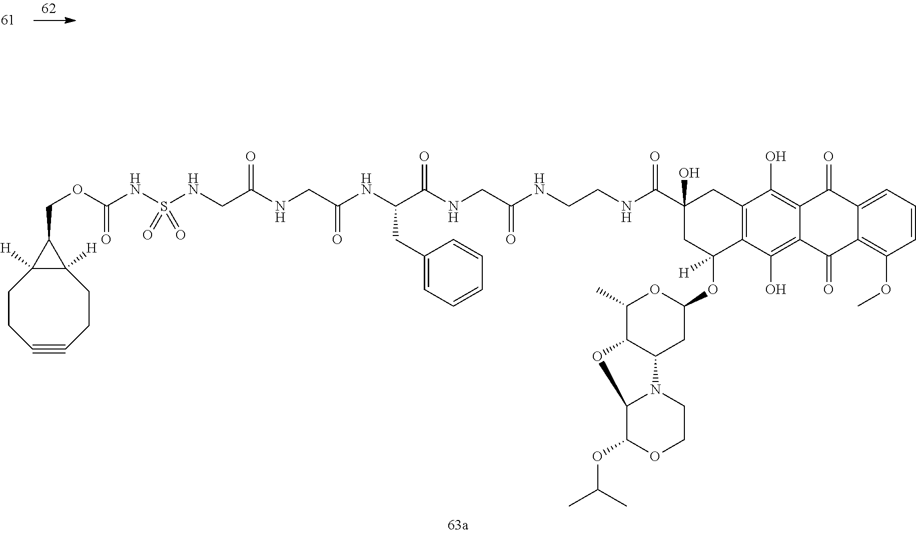 [SMILES: COc1cccc2c1C(=O)c1c(O)c3c(c(O)c1C2=O)C[C@@](O)(C(=O)NCCNC(=O)CNC(=O)[C@H](Cc1ccccc1)NC(=O)CNC(=O)CNS(=O)(=O)NC(=O)OC[C@@H]1[C@@H]2CCC#CCC[C@@H]21)C[C@@H]3O[C@H]1C[C@H]2[C@H](O[C@@H]3[C@@H](OC(C)C)OCCN32)[C@H](C)O1]

To a vial containing a dark red solution of compound 61 (2.4 mg, 1 Eq, 3.4 μmol) in DMF (180 μL) was added a solution of compound 62 (4.1 mg, 8.0 μL, 866 mmolar, 2.0 Eq, 6.9 μmol) in DMF, affording a clear red solution. Next, DIPEA (1.3 mg, 1.8 μL, 3 Eq, 10 μmol) was added, followed by a solution of HATU in DMF (1.4 mg, 7.3 μL, 492 mmolar, 1.05 Eq, 3.6 μmol) and the resulting mixture was mixed and left at rt for circa 75 minutes. The mixture was then purified by prep-HPLC (30%→70% acetonitrile in 10 mM NH₄HCO₃ in water, column Xbridge prep C₁₈, 5 μM OBD, 10×150 mm). The fractions containing pure product were combined and concentrated to give compound 63a (1.2 mg) as a red residue. LCMS (ESI+) calculated for C₆₁H₇₅N₈O₂₀S⁺ (M+H⁺) 1271.48 found 1271.95.

### Example a76. Synthesis of Compound 63b

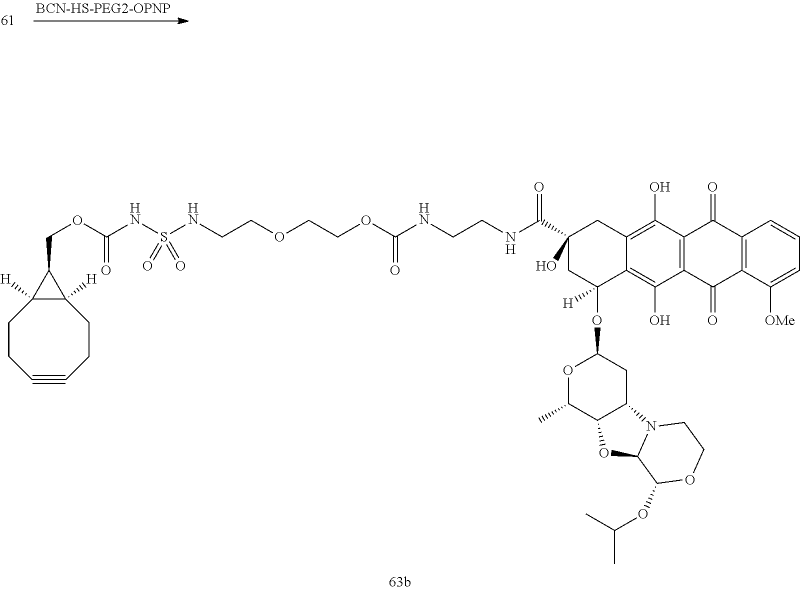 [SMILES: COc1cccc2c1C(=O)c1c(O)c3c(c(O)c1C2=O)C[C@@](O)(C(=O)NCCNC(=O)OCCOCCNS(=O)(=O)NC(=O)OC[C@@H]1[C@@H]2CCC#CCC[C@@H]21)C[C@@H]3O[C@H]1C[C@H]2[C@H](O[C@@H]3[C@@H](OC(C)C)OCCN32)[C@H](C)O1]

Synthesis of BCN-HS-PEG2-OPNP has been described in WO2021144314A1, which is incorporated herein by reference. To a vial containing a dark red solution of compound 61 (4.85 mg, 1 Eq, 6.95 μmol) in dry DCM (300 μL) was added BCN-HS-PEG₂-OPNP (4.37 mg, 92% Wt, 1.1 Eq, 7.65 μmol) followed by triethylamine (2.11 mg, 2.91 μL, 3 Eq, 20.9 μmol). The resulting solution was mixed and left at rt for 4.5 hours and was then stored in the freezer for 1 day. The RM was then removed from the freezer and left at rt for another 4 hours before storing the mixture in the freezer for another 2 days. The material was removed from the freezer a final time and then purified by prep-HPLC (20%→50% acetonitrile in 10 mM NH₄HCO₃ in water, column Xbridge prep C₁₈, 5 μM OBD, 10×150 mm). The fractions containing product were combined and concentrated to give compound 63b (5.1 mg) as a bright red solid. LCMS (ESI+) calculated for C₅₁H₆₆N₅O₁₉S⁺ (M+H⁺) 1084.41 found 1084.91.

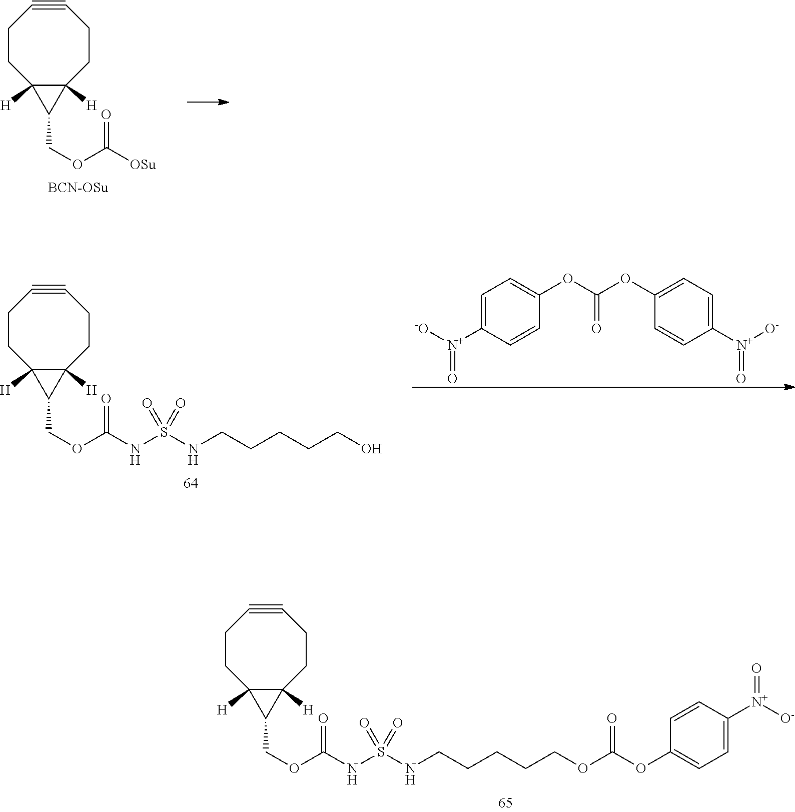

### Example a77. Synthesis of Compound 64

This compound was synthesized according to a literature procedure described in US20210030886A1.

### Example a78. Synthesis of Compound 65

BCN-HS-C5-OH (64) (400 mg, 1 equiv. 1.12 mmol) was dissolved in dry DCM (4 mL) followed by addition of bis(4-nitrophenyl) carbonate (373 mg, 1.1 Eq, 1.23 mmol) and triethylamine (226 mg, 311 μL, 2 Eq, 2.23 mmol) the yellow solution was stirred at rt for 220 minutes. The reaction mixture was concentrated in vacuo and then purified by flash column chromatography over silicagel (0%→10% EtOAc in DCM) to give product BCN-HS-C5-OPNP (65) (1.08 g, 7.24 mmol, 89% yield) as a pale-yellow oil. LCMS (ESI+) calculated for C₂₃H₃₃N₄O₉S⁺ (M+NH₄⁺) 541.20 found 541.50.

### Example a79. Synthesis of Compound 63c

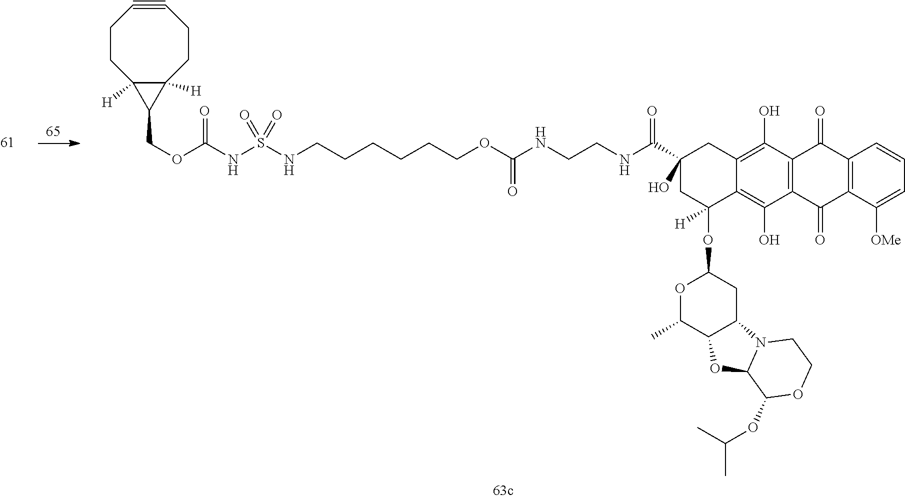 [SMILES: COc1cccc2c1C(=O)c1c(O)c3c(c(O)c1C2=O)C[C@@](O)(C(=O)NCCNC(=O)OCCCCCCNS(=O)(=O)NC(=O)OC[C@@H]1[C@@H]2CCC#CCC[C@@H]21)C[C@@H]3O[C@H]1C[C@H]2[C@H](O[C@@H]3[C@@H](OC(C)C)OCCN32)[C@H](C)O1]

To a vial containing a dark red solution of compound 61 (4.85 mg, 1 Eq, 6.95 μmol) in dry DCM (300 μL) was added a solution of BCN-HS-C5-OPNP (65) (4.82 mg, 83% Wt, 1.1 Eq, 7.65 μmol) in dry DCM (50 μL) followed by triethylamine (2.11 mg, 2.91 μL, 3 Eq, 20.9 μmol). The resulting solution was mixed and left at rt for 3 days. The mixture was then diluted with DMF (250 μL) and partially concentrated in vacuo to remove DCM. The resulting solution was then purified by prep-HPLC (20%→50% acetonitrile in 10 mM NH₄HCO₃ in water, column Xbridge prep C₁₈, 5 μM OBD, 30×150 mm). The fractions containing product were combined and concentrated to give compound 63c (4.6 mg) as a bright red solid. LCMS (ESI+) calculated for C₅₂H₆₈N₅₀₁₈S⁺ (M+H⁺) 1082.43 found 1082.91.

### Example a80. Synthesis of Compound 66

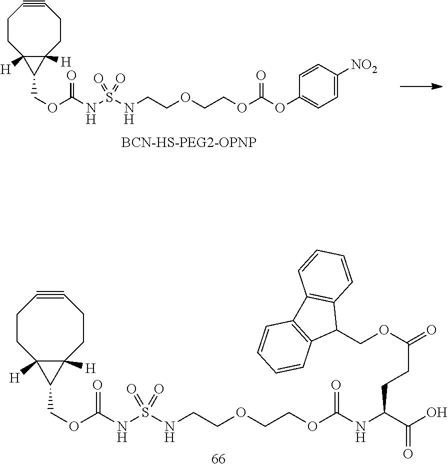 [SMILES: O=C(NS(=O)(=O)NCCOCCOC(=O)Oc1ccc([N+](=O)[O-])cc1)OC[C@@H]1[C@@H]2CCC#CCC[C@@H]21 ; O=C(CC[C@H](NC(=O)OCCOCCNS(=O)(=O)NC(=O)OC[C@@H]1[C@@H]2CCC#CCC[C@@H]21)C(=O)O)OCC1c2ccccc2-c2ccccc21]

Synthesis of BCN-HS-PEG2-OPNP has been described in WO2021144314A1, which is incorporated herein by reference. BCN-HS-PEG2-OPNP (10.2 mg, 1 Eq. 19.4 μmol) was dissolved in dry DCM (400 μL) and dry DMF (100 μL) followed by addition of H-Glu(Fm)-OH (11.2 mg, 1.36 Eq. 26.5 μmol) and DIPEA (15.1 mg, 20.3 μL, 6 Eq, 116 μmol). The resulting solution was mixed and left at rt for 4.5 hours and was then stored in the freezer for 3 days. The RM was then removed from the freezer and left at rt for another 3.5 hours. The RM was then directly loaded onto a pre-wetted column and was then purified using flash column chromatography over silicagel (0→20% MeOH in DCM). Fractions containing product were combined and concentrated in vacuo, affording compound 66 (11.2 mg, 97% purity, 15 μmol, 79%) as a clear oil. ¹H NMR (400 MHZ, DMSO-d6) δ 7.92 (d, J=7.5 Hz, 2H), 7.67 (d, J=7.4 Hz, 2H), 7.44 (t, J=7.5 Hz, 2H), 7.36 (t, J=7.5 Hz, 1H), 4.45-4.30 (m, 2H), 4.27 (t, J=6.8 Hz, 1H), 4.13 (m, 1H), 3.96 (m, 2H), 3.88 (d, J=7.9 Hz, 2H), 3.85-3.77 (m, 1H), 3.53 (t, J=4.5 Hz, 2H), 3.50-3.44 (t, J=5.6 2H), 2.87 (m, 2H), 2.49-2.35 (m, 1H), 2.18 (m 1H), 2.00 (m, 1H), 1.50 (m, 3H), 1.34-1.19 (m, 4H), 0.83 (m, 3H). UPLC-MS (ESI+) calculated for C₃₅H₄₅N₄O₁₁S⁺ [M+NH₄⁺] 729.28 found 729.64.

### Example a81. Synthesis of Compound 63d

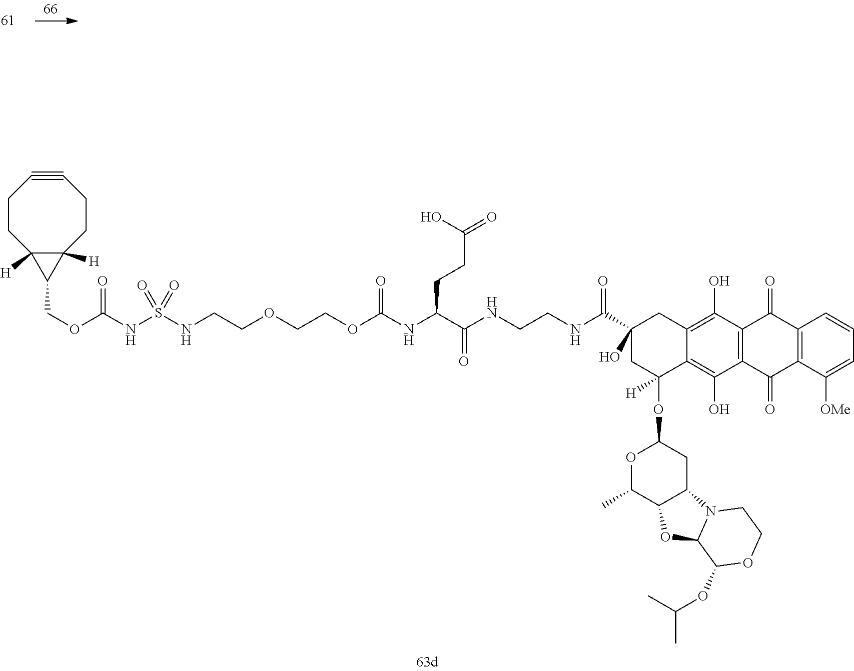 [SMILES: COc1cccc2c1C(=O)c1c(O)c3c(c(O)c1C2=O)C[C@@](O)(C(=O)NCCNC(=O)[C@H](CCC(=O)O)NC(=O)OCCOCCNS(=O)(=O)NC(=O)OC[C@@H]1[C@@H]2CCC#CCC[C@@H]21)C[C@@H]3O[C@H]1C[C@H]2[C@H](O[C@@H]3[C@@H](OC(C)C)OCCN32)[C@H](C)O1]

To a vial containing BCN-HS-PEG2-Glu(Fm)-OH (66) (5.9 mg, 97 wt %, 1.1 Eq, 8.0 μmol) in DCM (200 μL) was added first DIPEA (2.9 mg, 4.0 μL, 3 Eq, 23 μmol) and then HATU (3.2 mg, 1.1 Eq, 8.4 μmol) in 17 μL of DMF. The RM was stirred for 5 minutes. This solution was added to a vial containing a dark red solution of compound 61 (5.3 mg, 1 Eq, 7.6 μmol) in dry DCM (200 μL). The resulting RM was mixed and left at rt for 50 minutes. Next, DMF (350 μL) was added to the RM and the mixture was partially concentrated in vacuo to remove the DCM. The RM was stored in the freezer overnight. The RM was then removed from the freezer and triethylamine (7.7 mg, 11 μL, 10 Eq, 76 μmol) was added. The RM was stirred for 3 hours, followed by the addition of additional triethylamine (7.7 mg, 11 μL, 10 Eq, 76 μmol). The RM was stirred for another 4 hours. Next a third addition of triethylamine (7.7 mg, 11 μL, 10 Eq, 76 μmol) was added and the RM was left stirring for 4 hours. Then the RM was stored in the freezer overnight. The RM was then removed from the freezer and this solution was diluted to 700 μL with additional DMF and purified by prep-HPLC (Column Xbridge prep C₁₈, 5 μm OBD, 30×100 mm, 30%→95% MeCN in 10 mM aq. NH₄HCO₃). Fractions containing the product were combined and concentrated in vacuo. After the fractions were combined and completely concentrated, the red solid was redissolved twice in MeCN and then concentrated and once in DCM and concentrated to give compound 63d (3.9 mg, 3.2 μmol, 42%) as a red solid. UPLC-MS (ESI+) calculated for C₅₆H₇₃N₆O₂₂S⁺ [M+H⁺] 1213.45 found 1213.99.

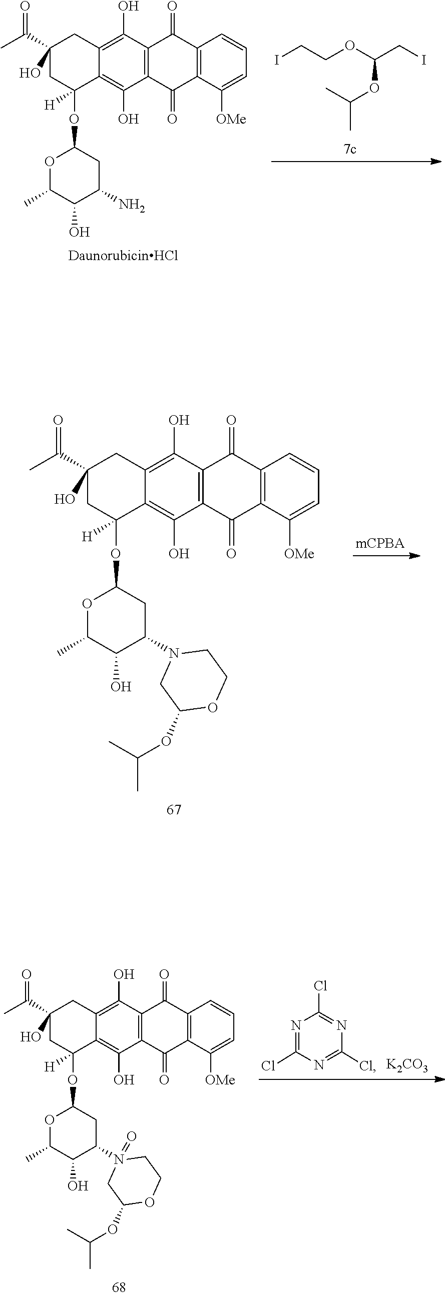

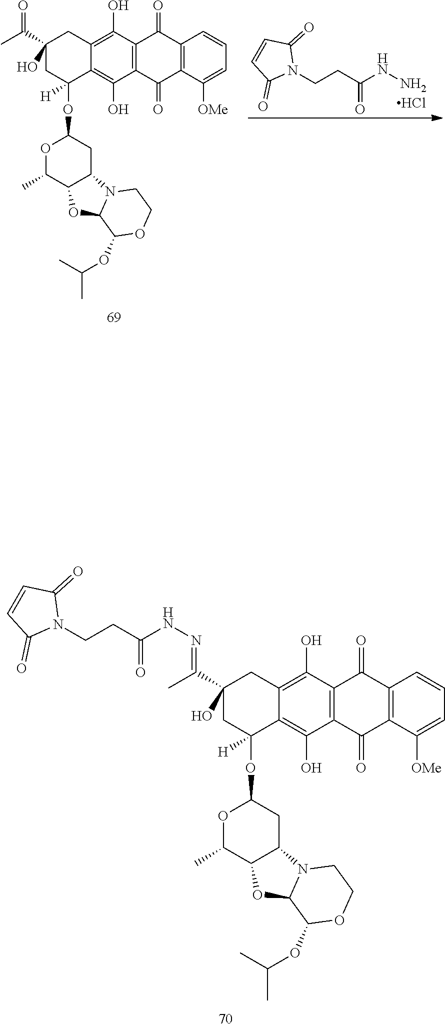

### Example a82. Synthesis of Compound 67

To daunorubicin·HCl (765.2 mg, 1 Eq, 1.357 mmol) was suspended in dry DMF (2.0 mL), followed by the addition of bis-iodo-sugar 7c (1.57 g, 807 μL, 3.01 Eq, 4.09 mmol) and DiPEA (526 mg, 709 μL, 3 Eq, 4.07 mmol). The flask was covered in aluminium foil and the suspension was stirred in the dark for 24 hours. Next, the RM was heated to 37° C. for another 20 hours and was then diluted with DCM (16 mL) and the resulting red solution was purified by flash column chromatography over silicagel (0%→7.5% MeOH in DCM) to give compound 67 as a red oil (662 mg, 1.01 mmol, 74.4%). LCMS (ESI+) calculated for C₃₄H₄₂NO₁₂⁺ (M+H⁺) 656.27 found 656.45.

### Example a83. Synthesis of Compound 68

A solution of compound 67 (662 mg, 1 Eq, 1.01 mmol) in DCM (25 mL) was cooled by dry ice/acetone-bath to a temperature of −78° C. The mixture was vigorously stirred after which a freshly made stock solution of mCPBA in DCM (261 mg, 2.61 mL, 580 mmolar, 1.5 Eq, 1.51 mmol) was added dropwise over 5 minutes. After stirring for 21 minutes at −78° C. the reaction quenched with cold (−78° C.) acetone (reagent grade, 8.00 mL) and the RM was stirred for another 30 minutes at-78° C. Next, the cold bath was removed, and the RM was allowed to warm up to rt. After the RM was allowed to warm to rt the mixture was diluted with DCM (55 mL). The resulting mixture was transferred to a separatory funnel and washed twice with saturated aqueous NaHCO₃ solution (15 mL, 2×). The combined water-layers were extracted with DCM (20 mL, 4×). Next, the combined organic layers were dried (Na₂SO₄) and filtered over a glass-filter and then concentrated in vacuo. The residue was taken up in DCM (100 mL) and H₂O (30 mL). The bi-phasic system was shaken and then separated using a phase-separator. The resulting organic layer was concentrated in vacuo, affording to give compound 68 (450 mg, 0.61 mmol, 60%, 91% Purity), which was used as such in the next step. LCMS (ESI+) calculated for C₃₄H₄₂NO₁₃⁺ (M+H⁺) 672.27.32 found 672.59.

### Example a84. Synthesis of Compound 69

To a solution of 68 (450 mg, 91% Wt, 1 Eq, 610 μmol) in a mixture of dry DCM (2.0 mL) anhydrous acetonitrile (3.0 mL) was added potassium carbonate (758 mg, 9 Eq, 5.49 mmol) was added. The RM was then cooled to 0° C. with an ice-bath after which a solution of Cyanuric chloride in dry MeCN (281 mg, 12.9 mL, 118 mmolar, 2.5 Eq, 1.52 mmol) was added slowly to the stirred solution. After complete addition additional dry DCM (3.0 mL) was added and the RM was stirred at 0° C. for 30 minutes, followed by the addition of another amount of DCM (6 mL). The resulting mixture was stirred for another 25 minutes and was then quenched with a solution of 3-amino-1,2-propanediol in H₂O (870 mg, 15.7 Eq, 9.55 mmol) in H₂O (circa 2.2 mL). The resulting mixture was stirred, and the ice bath was removed after 6 min, followed by the addition of DCM (75 mL) and H₂O (10 mL). The resulting bi-phasic system was separated. The water-layer was extracted with DCM (20 mL). The combined organic layers were concentrated in vacuo and the residue was purified using flash column chromatography (0→10% MeOH in DCM). Fractions containing the product were concentrated to give compound 69 (246 mg, 0.32 mmol, 52%, 85% Purity) as a red residue. LCMS (ESI+) calculated for C₃₄H₄₀NO₁₂⁺ (M+H⁺) 654.25 found 654.60.

### Example a85. Synthesis of Compound 70

To a maleimidopropionic acid hydrazide·HCl (10.9 mg, 1.9 Eq, 49.4 μmol) was dissolved in Anhydrous MeOH (1.63 mL) and added to 69 (20.0 mg, 85 wt %, 1 Eq, 26.0 μmol). Some solids remained therefore RM was swirled in the water-bath 40C till all solids dissolved. The RM was stirred in the dark at RT overnight. RM had 87% conversion to the desired product. RM was heated and stirred at 40° C. for 2 h. UPLC-MS analysis showed an increase in side-product formation. RM was stirred at RT for 4.5 h. As no further conversion was observed, Maleimidopropionic acid hydrazide·HCl (1.14 mg, 0.2 Eq, 5.20 μmol) was dissolved in 100 μl dry MeOH and added to the RM. RM was stirred overnight. 89% conversion to the product was achieved and an increase in side-product formation. The RM was halted, and purification was started. RM was concentrated in vacuo. The crude was re-dissolved in 2 ml 2% MeOH in DCM and loaded on the column and purified using silica (2→40% MeOH in DCM). Fractions containing the product were combined, concentrated, and stored in the freezer. The product still had some impurities; therefore a second purification was performed. The product was re-dissolved in 1 ml in DCM to which 100 μl DMF was added and loaded on the column. The vial it originated from was washed with 2×1 ml DCM and loaded onto the column and purified using silica purification (0→20% MeOH/DCM). Fractions containing the product were combined and concentrated, obtaining compound 66 (9.0 mg, 9.9 μmol, 38%, 90% Purity) as a red residue. UPLC-MS (ESI+) calculated for C₄₁H₄₇N₄O₁₄⁺ [M+H⁺] 819.31 found 819.72.

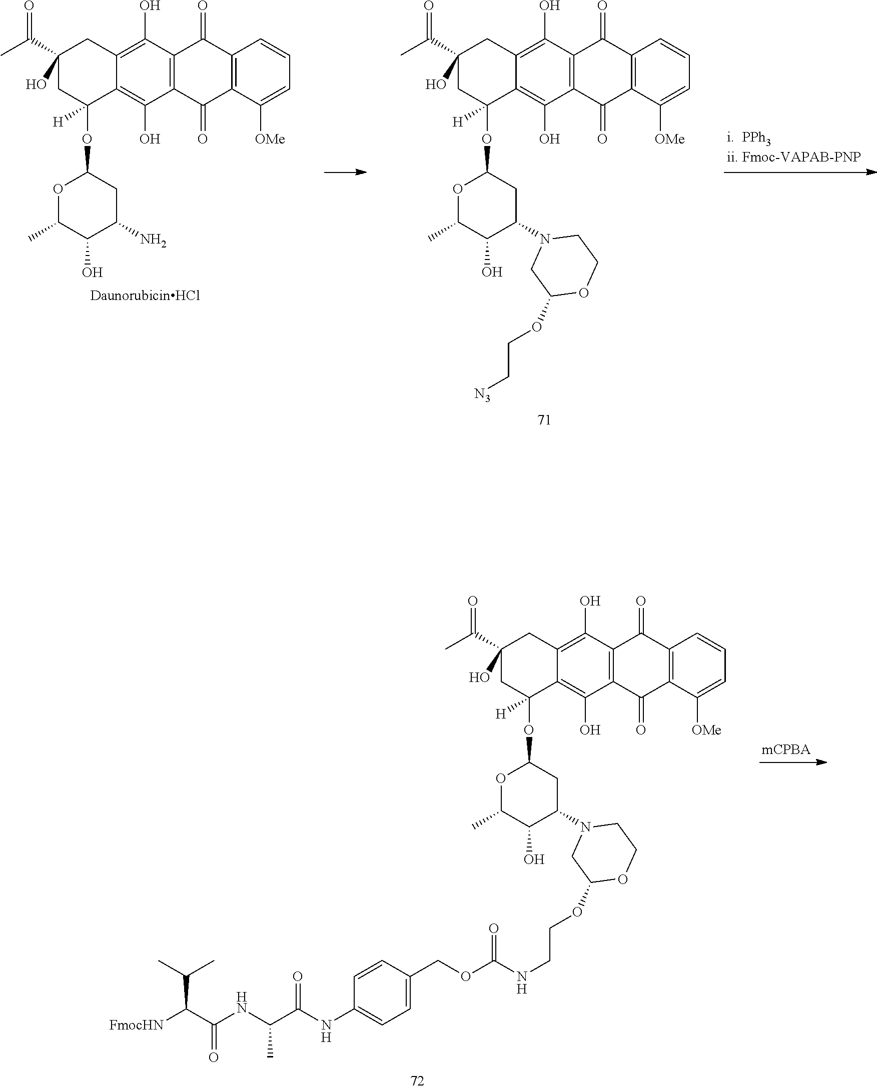

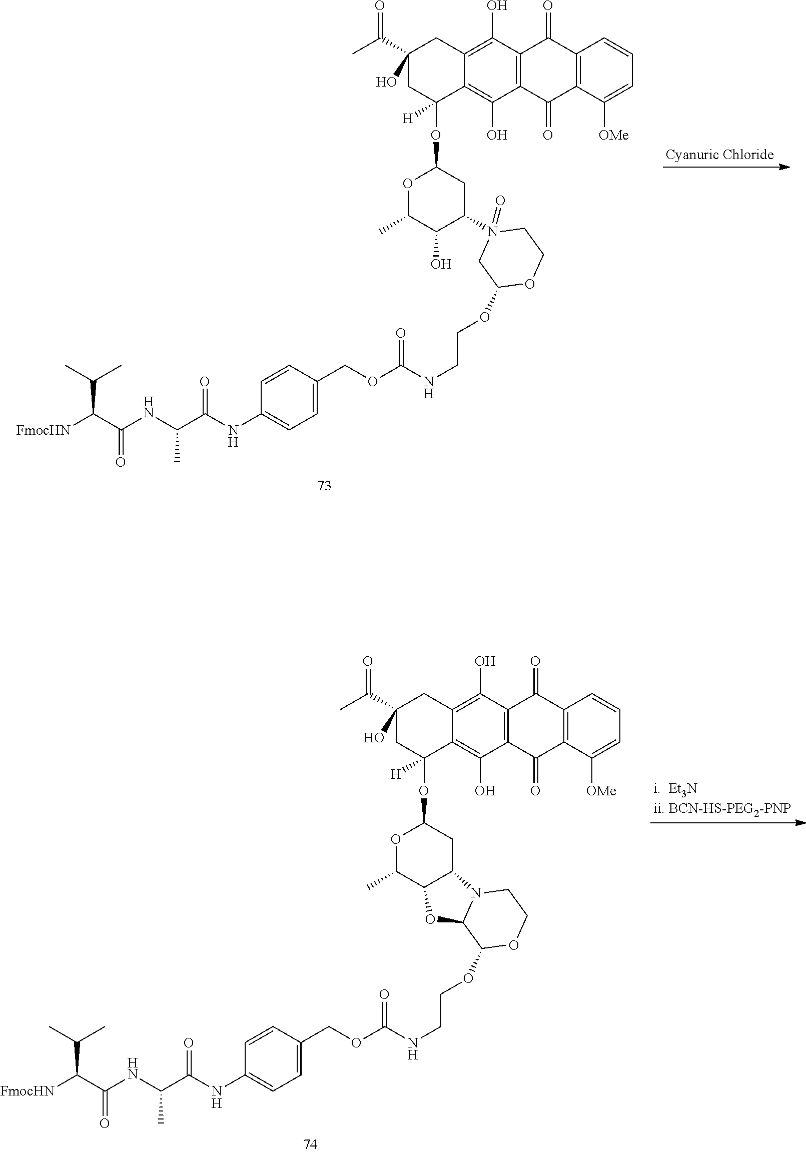

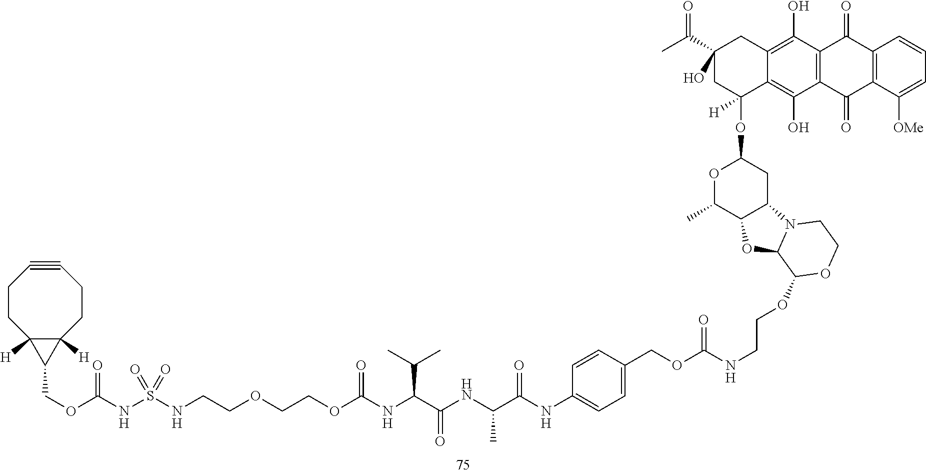

### Example a86. Synthesis of Compound 71

To daunorubicin·HCl (202.6 mg, 1 equiv., 359.2 μmol) was suspended in dry DMF (1.2 mL), followed by the addition of bis-iodo-sugar 7b (442.9 mg, 3 Eq, 1.078 mmol) and DIPEA (139.3 mg, 188 μL, 3 Eq, 1.078 mmol). The flask was covered in aluminium foil and the suspension was stirred in the dark at rt for 4 days, generating a dark red solution. Next, the RM was diluted with DCM (10 mL) and the resulting red solution was purified by flash column chromatography over silicagel (0%->15% MeOH in DCM) to give compound 71 as a red waxy oil (125.1 mg, 95% purity, 170 μmol, 48% yield) and a 2^{nd} batch with slightly less purity (48.2 mg, 88% purity, 62 μmol, 17% yield) also as a red waxy oil. LCMS (ESI+) calculated for C₃₃H₃₉N₄₀₁₂⁺ (M+H⁺) 683.26 found 683.47.

### Example a87. Synthesis of Compound 72

To a suspension of compound 71 (125.1 mg, 95% Wt, 1 Eq, 174.1 μmol) in MeOH (800 μL) was added a solution of triphenylphosphine in DCM (123.3 mg, 1.343 mL, 350 mmolar, 2.7 Eq, 470.0 μmol) and water (400 μL). The biphasic mixture was stirred 85 minutes at room temperature after which Fmoc-Val-Ala-PAB-OPNP (142.2 mg, 1.2 Eq, 208.9 μmol) was added. After stirring the biphasic mixture for an additional 18 hours at room temperature, an extraction with DCM (2×2 mL) was carried out. The combined organic layers were dried over Na₂SO₄ and immediately purified by flash column chromatography over silicagel (0%→40% EtOAc in DCM (to remove excess Fmoc-Val-Ala-PAB-OPNP) followed by 0%→15% MeOH in DCM) to give compound 72 as a red solid (71.3 mg, 59.5 μmol, 34.2%). LCMS (ESI+) calculated for C₆₄H₇₂N₅O₁₈⁺ (M+H⁺) 1198.49 found 1198.75.

### Example a88. Synthesis of Compound 73

A solution of compound 72 (87.3 mg, 72.9 μmol, 1.00 equiv.) in a mixture of anhydrous DCM (1.5 mL) and MeOH (200 μL) was cooled with dry ice/acetone cooling-bath to a temperature of −78° C. The mixture was vigorously stirred after which a freshly made stock solution of mCPBA in anhydrous DCM (13.8 mg, 138 μL, 580 mmolar, 1.1 Eq, 80.1 μmol) was added dropwise via the side of the flask. After stirring for 30 minutes additional mCPBA in anhydrous DCM (3.77 mg, 37.7 μL, 580 mmolar, 0.3 Eq, 21.9 μmol) was added dropwise via the side of the flask. The RM was stirred at −78° C. for another hour and was then quenched with an ice-cold solution of acetone (reagent grade, 2.5 mL) and the RM was stirred. After an hour the cold bath was removed, and the RM was allowed to warm up to room temperature over 105 minutes. Next, the RM was diluted with DCM (15 mL) and transferred to a separation funnel and washed twice with saturated aqueous NaHCO₃ solution (10 mL, 2×). The water layers were combined and extracted twice with DCM (20 mL). The combined organic layers were dried over Na₂SO₄, filtered through a membrane-filter, and concentrated in vacuo to give compound 73 (75.9 mg, 62.5 μmol, 85.8%) as a red solid, which was used as such in the next step. LCMS (ESI+) calculated for C₆₄H₇₂N₅O₁₉⁺ (M+H⁺) 1214.48 found 1214.73.

### Example a89. Synthesis of Compound 74

To a red solution of compound 73 (75.9 mg, 1 Eq, 62.5 μmol) in a mixture of dry DCM (1.0 mL) anhydrous acetonitrile (9.0 mL) was added potassium carbonate (33.7 mg, 3.9 Eq, 244 μmol). The RM was then cooled to 0° C. with an ice-bath after which a solution of cyanuric chloride (28.8 mg, 1.30 mL, 120 mmolar, 2.5 Eq, 156 μmol) in anhydrous acetonitrile was added. After stirring for 155 minutes at 0° C. additional potassium carbonate (17.3 mg, 2 Eq, 125 μmol) and cyanuric chloride (14.4 mg, 651 μL, 120 mmolar, 1.25 Eq, 78.1 μmol) in anhydrous acetonitrile were added. The RM was stirred at 0° C. for another 80 minutes and was then stored in the freezer for circa 18 hours. The RM was then removed from the freezer and placed in an ice-bath at 0° C. again, followed by a final addition of additional potassium carbonate (11.3 mg, 1.31 Eq, 81.8 μmol) and freshly prepared cyanuric chloride (14.4 mg, 651 μL, 120 mmolar, 1.25 Eq, 78.1 μmol) in anhydrous acetonitrile. The RM was stirred at 0° C. for another 280 minutes and was then quenched with a solution of 3-aminopropane-1,2 diol (192.3 mg) in 4.58 mL water. The ice bath was removed after 30 min and the RM was then diluted with DCM (10 mL). The resulting bi-phasic system was separated. The water-layer was extracted with more DCM (15 mL, 3×). The combined organic layers were dried (Na₂SO₄), filtered and then partially concentrated in vacuo to a volume of circa 15 mL. The mixture was then diluted with a little bit DMF (300 μL), and the resulting solution was purified by flash column chromatography over silicagel (0%→20% MeOH in DCM). The fractions containing product were combined and concentrated to give compound 74 (12.7 mg, 10.6 μmol, 17.0%) as a red residue. LCMS (ESI+) calculated for C₆₄H₇₀N₅O₁₈⁺ (M+H⁺) 1196.47 found 1196.82.

### Example a90. Synthesis of Compound 75

Synthesis of BCN-HS-PEG2-OPNP has been described in WO2021144314A1, which is incorporated herein by reference. To a solution of compound 74 (12.7 mg, 1 Eq, 10.6 μmol) in DCM (3 mL) was added DMF (150 μL) and the RM was concentrated to remove the DCM. Then, triethylamine (16.1 mg, 22.2 μL, 15 Eq, 159 μmol) was added followed by a solution of BCN-HS-PEG₂-OPNP (6.70 mg, 143 μL, 89 mmolar, 1.2 Eq, 12.7 μmol) in DMF. The solution was mixed and then left at rt for 21 hours. The RM was then further diluted with DCM (1 mL) and purified by flash column chromatography over silicagel (0%→15% MeOH in DCM) to give impure compound 75. The material was then subjected to a purification by prep-HPLC (30%→95% acetonitrile in 10 mM NH₄HCO₃ in water, column Xbridge prep C₁₈, 5 μM OBD, 30×100 mm). The fractions containing product were combined and concentrated to give compound 75 (2.3 mg). LCMS (ESI+) calculated for C₆₅H₈₂N₇O₂₃S⁺ (M+H⁺) 1360.52 found 1361.07.

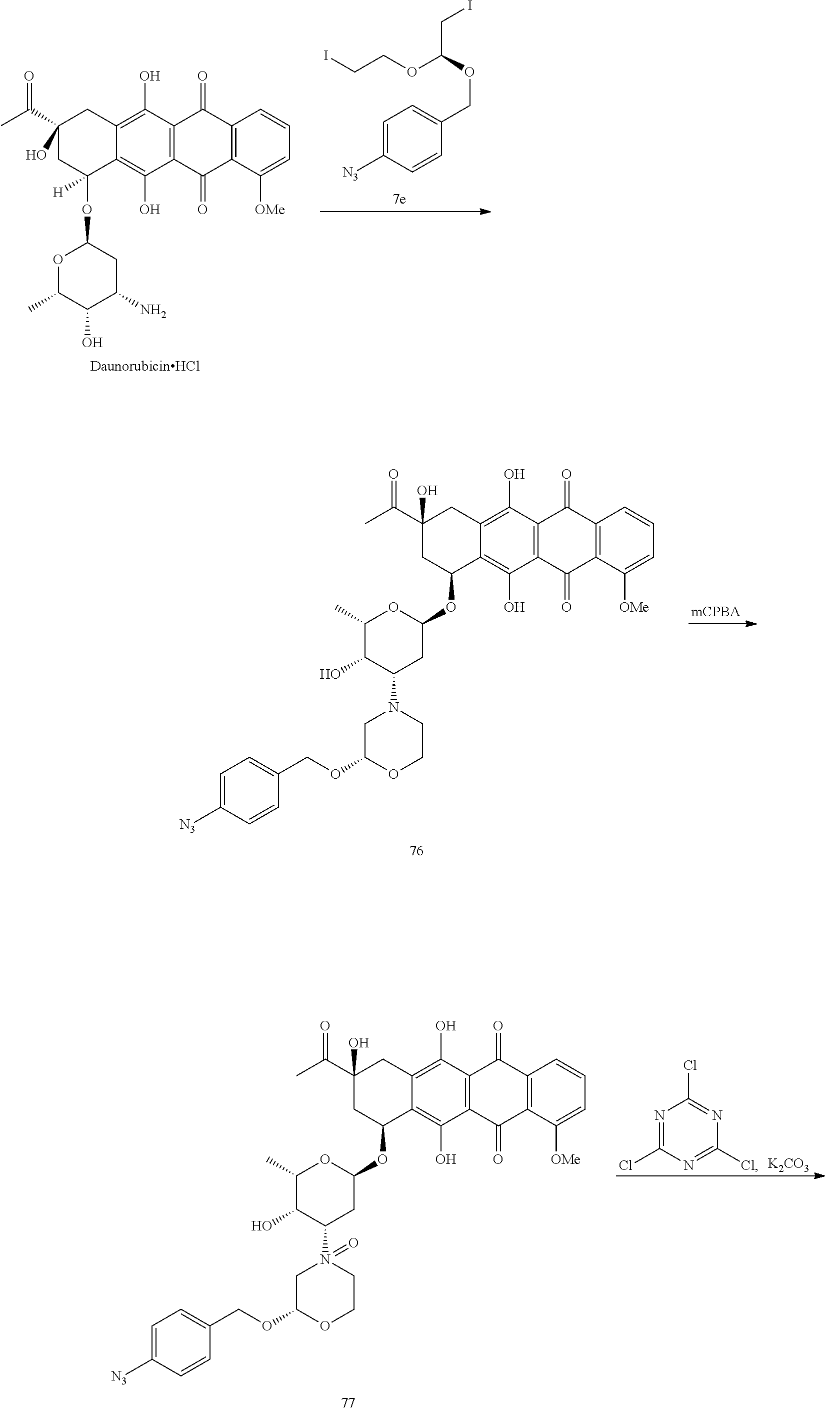

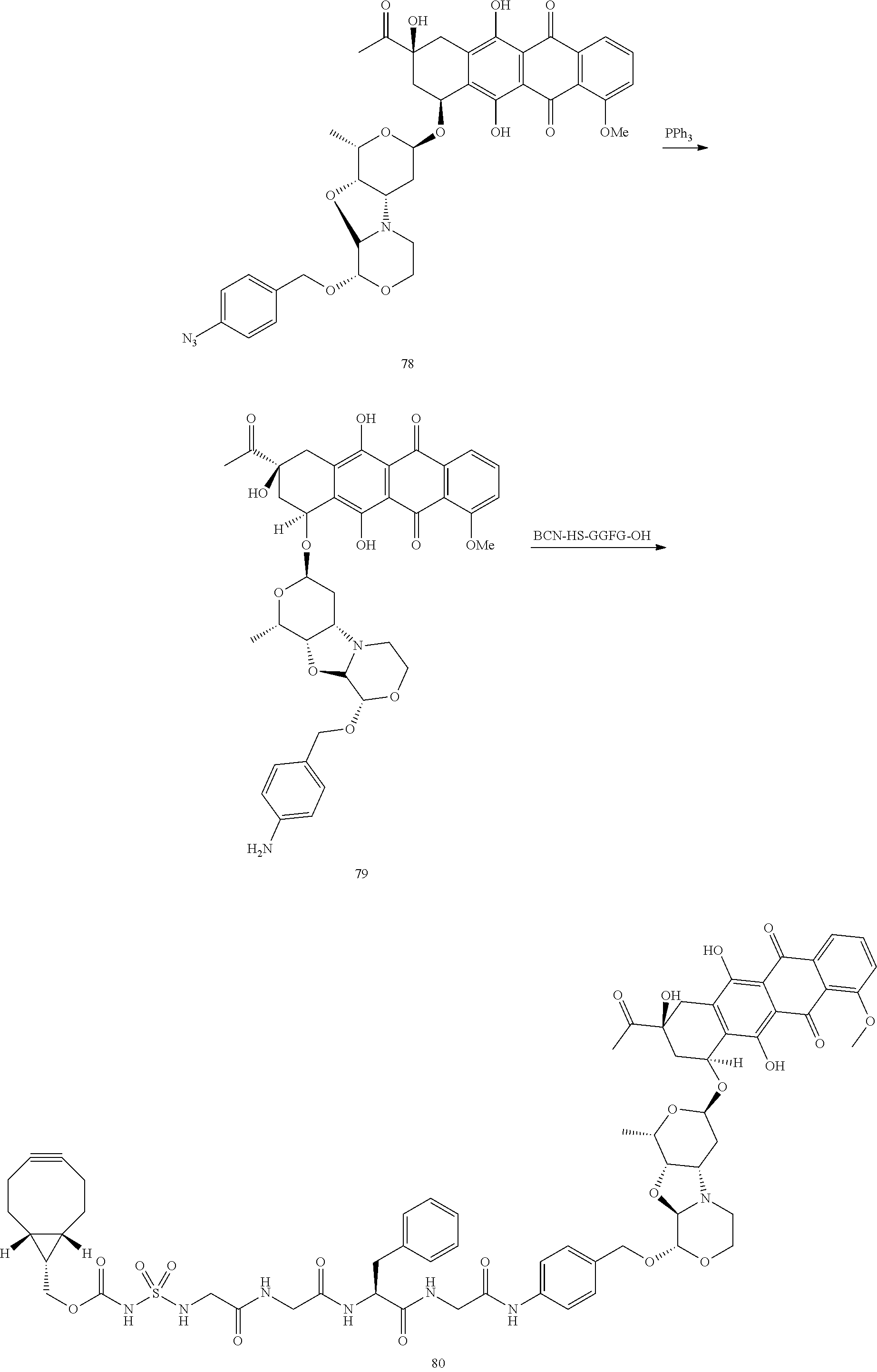

### Example a91. Synthesis of Compound 76

Daunorubicin, HCl (2.0 g, 1 Eq, 3.5 mmol) was dissolved in dry DMF (7.70 mL) after which (S)-1-azido-4-((2-iodo-1-(2-iodoethoxy)ethoxy)methyl)benzene 7e (5.1 g, 3.018 Eq, 11 mmol) and DIPEA (1.4 g, 1.9 mL, 3 Eq, 11 mmol) were added. The flask was covered in aluminium foil and the suspension was stirred in the dark at 40° C., generating a red solution over time. The RM was stirred at 40° C. for roughly 19 hours and analyzed. The RM was then heated at 40° C. while stirring for another 57 hours before it was stored in the freezer. The RM was diluted with DCM (100 mL) to a volume of circa 118 mL. This solution was then transferred onto the column. The residue was then flash column chromatography over silicagel (0-10% MeOH in DCM). Fractions containing product were combined and concentrated in vacuo to yield compound 76 (2.61 g, 3.406 mmol, 96%, 97.2% Purity) as dark red residue. UPLC-MS (ESI+) calculated for C₃₈H₄₁N₄O₁₂⁺ [M+H⁺] 745.27 found 745.63.

### Example a92. Synthesis of Compound 77

Compound 76 (2.609 g, 97.22 wt %, 1 Eq, 3.406 mmol) was dissolved in DCM (80 mL). The mixture was cooled down by a dry-ice/acetone cooling-bath (−78° C.). Then 3-chlorobenzoperoxoic acid (646.5 mg, 6.46 mL, 580 mmolar, 1.1 Eq, 3.75 mmol) was added dropwise added by using a microman pipette and was added via the side of the flask to prevent temperature spike. Meanwhile reagent-grade acetone was cooled in the dry ice/acetone cooling-bath. After 3 min a second addition of mCPBA in DCM (235.09 mg, 2.3489 mL, 580 mmolar, 0.4 Eq, 1.3624 mmol) was added. After 3 min a third addition of mCPBA in DCM (235.09 mg, 2.3489 mL, 580 mmolar, 0.4 Eq, 1.3624 mmol) was added. After another 3 min. the RM was quenched with 68.8 mL cold reagent grade acetone and left stirring in the cooling-bath. After 1 h the cold bath was removed, and the RM was allowed to warm up to −5° C. To the cold RM 160 mL sat. aq. NaHCO₃ aqueous solution was added while stirring, followed by the addition of 200 mL DCM. The RM was then transferred to a separation funnel. The DCM layer was separated and washed twice with 160 mL sat. aq. NaHCO₃ aqueous solution. The organic layer was dried over Na₂SO₄ and filtered through a membrane filter. The solution was concentrated under vacuo to give compound 77 (1.955 g, 2.229 mmol, 65.45%, 86.74% purity) was obtained. UPLC-MS (ESI+) calculated for C₃₈H₄₁N₄O₁₃⁺ [M+H⁺] 761.27 found 761.62.

### Example a93. Synthesis of Compound 78

To compound 77 (0.978 g, 86.74 wt %, 1 Eq, 1.115 mmol) as a solution in mixture of dry acetonitrile (6 mL) and dry DCM (4 mL) was added potassium carbonate (616.2 mg, 4 Eq, 4.459 mmol) and the RM was cooled down with an ice bath (ice-bath 0° C.). Then cyanuric chloride (513.9 mg, 13.93 mL, 200 mmolar, 2.5 Eq, 2.787 mmol) was added at once and the mixture was stirred vigorously. After 2.5 h full the RM was quenched with 3-aminopropane-1,2 diol in H₂O (1.249 g, 6.855 mL, 2 molar, 12.3 Eq, 13.71 mmol). After stirring for 10 min on ice the RM was stored in the freezer for 1 day. The RM was taken out of the freezer and then transferred to a separation funnel and 50 mL DCM was added. The resulting bi-phasic system was separated. The water layer was extracted four more times with 50 mL DCM (50 mL, 4×). The combined organic layers were transferred to a round-bottom flask and concentrated. After concentrating it, a dark red residue was obtained. This was re-dissolved in 15 mL DCM and then loaded onto the column and purified by flash column chromatography over silicagel (0→10% MeOH in DCM). Fractions containing the product were combined and concentrated to give impure compound 78, which was subjected to a second purification by flash column chromatography over silicagel (0→10% MeOH in DCM). Fractions containing the product were combined and concentrated to give compound 78 (163.3 mg, 219.9 μmol, 19.72%) as dark red residue. UPLC-MS (ESI+) calculated for C₃₈H₃₉N₄O₁₂⁺ [M+H⁺] 743.26 found 743.63.

### Example a94. Synthesis of Compound 79

Compound 78 (50.0 mg, 1 Eq, 67.3 μmol) was suspended in MeOH (290 μL), followed by the addition of PPh3 in DCM (47.7 mg, 519 μL, 350 mmolar, 2.7 Eq, 182 μmol), generating a clear red solution. Next, H₂O (145 μL) was added. The resulting biphasic mixture was stirred at rt for 1 hour, followed by the addition of additional H₂O (145 μL). The RM was left to stir at rt for 1 day and was then heated to 40° C. and stirred for a few hours. Some of the DCM seemed to evaporate and 500 μl DCM was added accordingly. The RM was then stirred at 35° C. for 1 day, followed by the addition of DMF (50 μL). The resulting mixture was stirred at 35° C. for another 3 days. The reaction was then further diluted through the addition of DMF (50 μL), and H2O (100 μL) and the RM was stirred at 40° C. for another 2 days. The RM was then partially concentrated in vacuo (until 40 mbar), affording crude compound 79 as a solution in DMF that was used without further purification in the next step. UPLC-MS (ESI+) calculated for C₃₈H₄₁N₂₀₁₂+ [M+H⁺] 717.27 found 717.73.

### Example a95. Synthesis of Compound 80

To BCN-HS-GGFG-OH (62) in DMF (5.4 mg, 91 μL, 100 mmolar, 1.3 Eq, 9.1 μmol) was added first DIPEA (2.7 mg, 3.6 μL, 3 Eq, 21 μmol) and then HATU (3.4 mg, 1.3 Eq, 9.1 μmol). The RM was stirred for 5 minutes. Then, compound 79 in DMF (5.0 mg, 0.10 mL, 67. mmolar, 1.0 Eq, 7.0 μmol) was added. The RM was stirred for 150 minutes. Because of partial conversion to the product, additional BCN-HS-GGFG-OH (62) in DMF (42 μL, 100 mmolar, 0.6 Eq, 4.2 μmol), DIPEA (0.90 mg, 1.2 μL, 1 Eq, 7.0 μmol) and HATU (1.6 mg, 0.6 Eq, 4.2 μmol) and additional DMF (100 μL), were pre-mixed for 5 min and added to the main RM, which was stirred for another 5 h. The RM was then diluted to 500 μL of DMF and purified by Reverse-Phase Prep-HPLC (Column Xbridge prep C₁₈, 5 μm OBD, 30×100 mm, 5%→95% MeCN in 10 mM aq. NH₄HCO₃). Fractions containing the product were combined and concentrated in vacuo to yield 80 (0.6 mg, 0.5 μmol, 7%) as a red solid. UPLC-MS (ESI+) calculated for C₆₄H₇₂N₇O₂₀S⁺ [M+H⁺] 1290.45 found 1291.12.

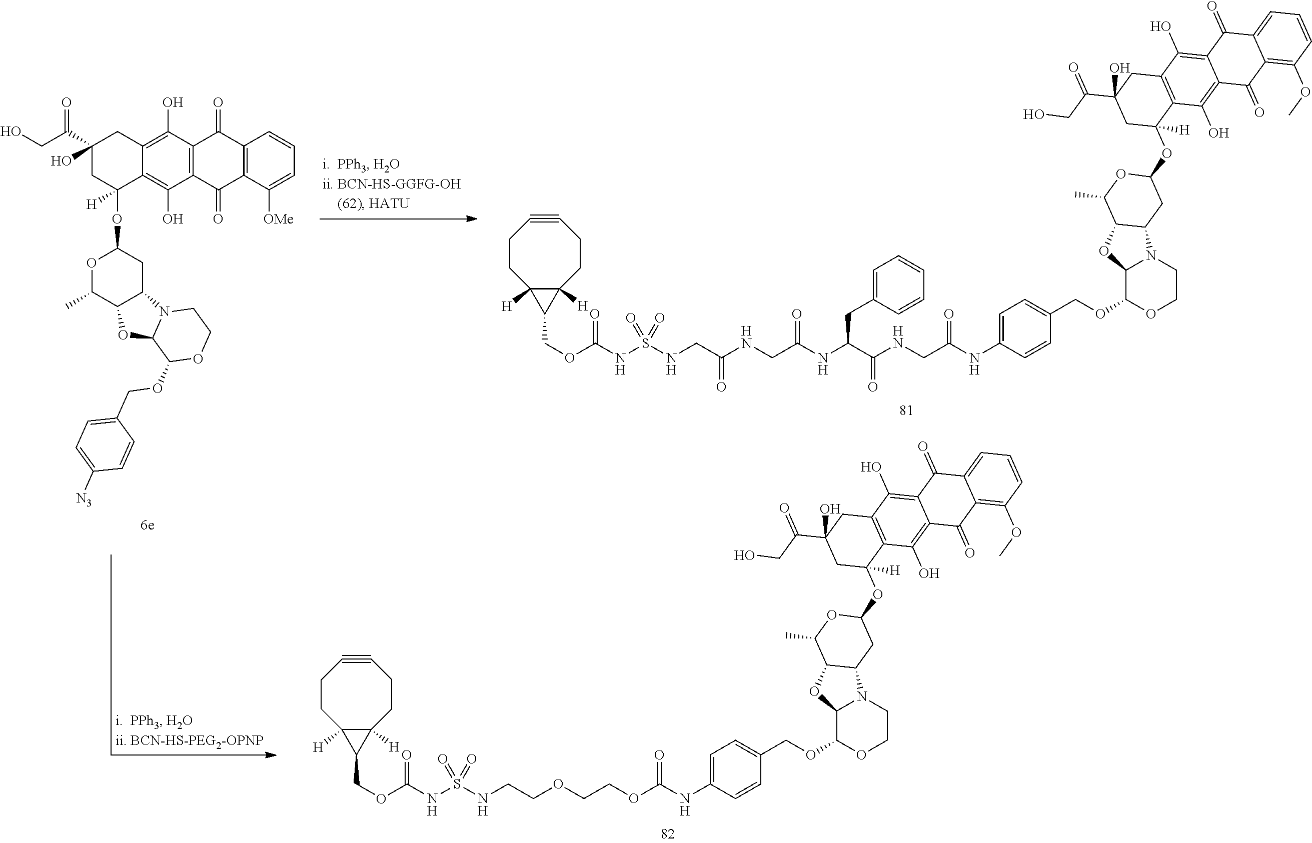

### Example a96. Synthesis of Compound 81

To a round-bottom flask containing a solution of compound 6e (45.6 mg, 80 wt %, 48.1 μmol, 1.00 equiv.) in DCM (1.5 mL) was added a solution of PPh3 in DCM (188 μL, 690 mmolar, 130 μmol, 2.7 equiv.) and H₂O (800 μL). The resulting bi-phasic mixture was stirred vigorously at rt for 3 days, followed by the addition of DMF (500 μL) and additional DCM (500 μL). The resulting mixture was stirred vigorously at rt for another 3 days and was then stored in the freezer. The mixture was removed from the freezer after 3 days and was then diluted with DCM (10 mL). The resulting bi-phasic system was separated, and the water-layer was extracted twice with DCM (10 mL, 2×). The combined organic layers were dried (Na₂SO₄), filtered and then concentrated in vacuo, affording crude amine intermediate (91.5 mg) and part of this material (11.7 mg) was used directly without further purification for the synthesis of compound 81; Compound 62 (11.0 mg, 66 wt %, 1.1 Eq, 12.3 μmol) in DMF (200 μL) was treated with DIPEA (4.33 mg, 5.84 μL, 3 Eq, 33.5 μmol) and HATU (4.46 mg, 1.05 Eq, 11.7 μmol) and the resulting mixture was mixed and then added to a vial containing a solution of solution of crude amine intermediate (11.7 mg) in DMF (200 μL). The resulting mixture was mixed and left at rt for circa 150 minutes. Next, additional compound 62 (11.0 mg, 66% Wt, 1.1 Eq, 12.3 μmol) in DMF (100 μL), DIPEA (4.33 mg, 5.84 μL, 3 Eq, 33.5 μmol) and HATU (4.46 mg, 1.05 Eq, 11.7 μmol) were added to the RM. The resulting mixture was mixed and left at rt for another 110 minutes, before adding a final addition of additional compound 62 (11.0 mg, 66% Wt, 0.939 Eq, 10.5 μmol) was dissolved in 100 μL and DIPEA (4.33 mg, 5.84 μL, 3 Eq, 33.5 μmol) and HATU (4.46 mg, 1.05 Eq, 11.7 μmol) to the RM. The RM was mixed and left at rt for another 40 minutes at rt and was then stored in the freezer for circa 16 hours. Next, the mixture was purified by prep-HPLC (30%→95% acetonitrile in 10 mM aq. NH₄HCO₃ in water, column Xbridge prep C₁₈, 5 μM OBD, 10×150 mm). The fractions containing pure product were combined and concentrated to give compound 81 (4 mg) as a red solid. LCMS (ESI+) calculated for C₆₄H₇₂N₇O₂₁S⁺ (M+H⁺) 1306.45 found 1307.03.

### Example a97. Synthesis of Compound 82

Synthesis of BCN-HS-PEG2-OPNP has been described in WO2021144314A1, which is incorporated herein by reference. To a round-bottom flask containing a solution of compound 6e (45.6 mg, 80 wt %, 48.1 μmol, 1.00 equiv.) in DCM (1.5 mL) was added a solution of PPh3 in DCM (188 μL, 690 mmolar, 130 μmol, 2.7 equiv.) and H₂O (800 μL). The resulting bi-phasic mixture was stirred vigorously at rt for 3 days, followed by the addition of DMF (500 μL) and additional DCM (500 μL). The resulting mixture was stirred vigorously at rt for another 3 days and was then stored in the freezer. The mixture was removed from the freezer after 3 days and was then diluted with DCM (10 mL). The resulting bi-phasic system was separated, and the water-layer was extracted twice with DCM (10 mL, 2×). The combined organic layers were dried (Na₂SO₄), filtered and then concentrated in vacuo, affording crude amine intermediate (91.5 mg) and part of this material (11.7 mg) was used directly without further purification for the synthesis of compound 82. Crude amine intermediate (11.7 mg) was dissolved in dry DCM (400 μL), followed by the addition of BCN-HS-PEG₂-OPNP (7.75 mg, 91 wt %, 1.2 Eq, 13.4 μmol) and triethylamine (3.39 mg, 4.67 μL, 3 Eq, 33.5 μmol). The resulting RM was mixed and left at rt for 23.5 hours, followed by the addition of HOBt (1.51 mg, 25.6 μL, 436 mmolar, 1 Eq, 11.2 μmol). The RM was left at rt for another 200 minutes and then DMAP (1.37 mg, 15.0 μL, 744 mmolar, 1 Eq, 11.2 μmol) was added. The RM was again mixed and left at rt for 5 hours. Finally, additional BCN-HS-PEG₂-OPNP (11.7 mg, 2 Eq, 22.4 μmol), triethylamine (3.39 mg, 4.67 μL, 3 Eq, 33.5 μmol) and DMAP (2.73 mg, 30.0 μL, 744 mmolar, 2 Eq, 22.4 μmol) were added and the resulting mixture was left at rt for 3 days. The RM was then diluted with DMF (200 μL) and partially concentrated in vacuo to remove DCM. The resulting solution was purified by prep-HPLC (30%→95% acetonitrile in 10 mM aq. NH₄HCO₃ in water, column Xbridge prep C₁₈, 5 μM OBD, 30×150 mm). The fractions containing product were combined and concentrated to give compound 82 (2.1 mg) as a red solid. LCMS (ESI+) calculated for C₅₄H₆₃N₄O₂₀S⁺ (M+H⁺) 1119.38 found 1120.14.

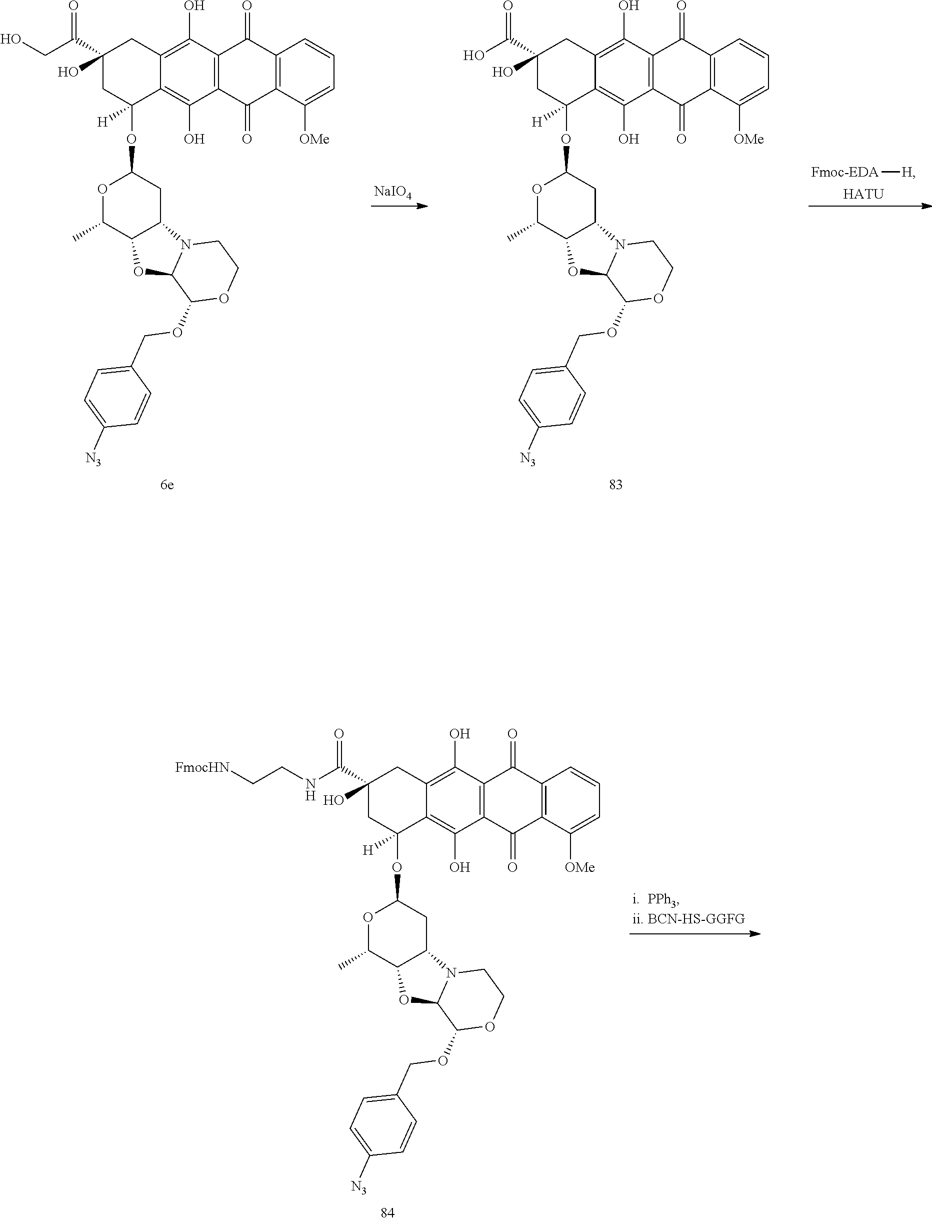

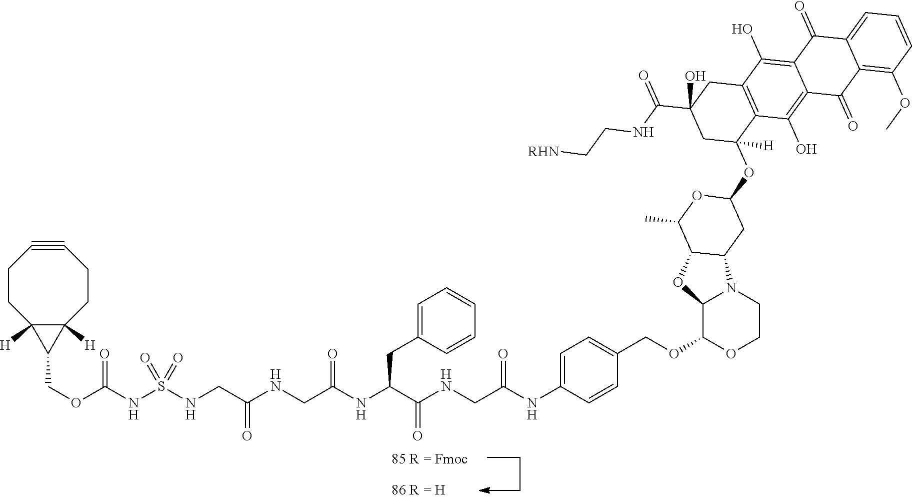

### Example a98. Synthesis of Compound 83

To a suspension of 6e (158 mg, 80% Wt, 1 Eq, 167 μmol) in MeOH (2.0 mL) was added THF (2.0 mL), generating a clear solution. Next, water (500 μL) was added, followed by a drop-wise addition of a solution of sodium periodate in water (200 mM, 1.67 mL, 333 μmol) and the reaction mixture was stirred at rt for 24.5 hours in the dark and was then stored in the freezer for 3h 35 minutes. The mixture was removed from the freezer and DCM (10 mL) was added. The mixture was transferred to a separation funnel together with additional H₂O (1 mL). The resulting bi-phasic system was mixed and separated, and the water-layer was extracted with DCM (10 mL, 2×). The combined organic layers were filtered over a membrane-filter. The resulting solution was concentrated in vacuo, affording compound 83 (132 mg, 70% purity, 120 μmol, 74%) as a red solid. LCMS (ESI+) calculated for C₃₇H37N₄O₁₃⁺ (M+H⁺) 745.24 found 745.63.

### Example a99. Synthesis of Compound 84

(9H-fluoren-9-yl) methyl (2-aminoethyl) carbamate, HCl (51.4 mg, 1.3 Eq, 161 μmol) was dissolved in DMF (500 μL) and then diluted with DCM (700 L), followed by the addition of DIPEA (48.1 mg, 64.8 μL, 3 Eq, 372 μmol). The resulting mixture was added to a vial containing compound 83 (132 mg, 70% Wt, 1 Eq, 124 μmol). Finally, HATU (47.2 mg, 1 Eq, 124 μmol) was added. The resulting mixture was mixed and left at room temperature for 40 minutes. Next, additional (9H-fluoren-9-yl) methyl (2-aminoethyl) carbamate, HCl (25.7 mg, 0.65 Eq, 80.6 μmol) in a mixture of DMF (200 μL) and DCM (200 μL) together with DIPEA (24.1 mg, 32.4 μL, 1.5 Eq, 186 μmol) was added to the main RM, followed by additional HATU (23.6 mg, 0.5 Eq, 62.0 μmol). The RM was mixed again and left for another 65 minutes before adding a final sequence of additional (9H-fluoren-9-yl) methyl (2-aminoethyl) carbamate, HCl (23.7 mg, 0.6 Eq, 74.4 μmol) in a mixture of DMF (200 μL) and DCM (200 μL) together with DIPEA (16.0 mg, 21.6 μL, 1 Eq, 124 μmol) to the main RM, followed by additional HATU (18.9 mg, 0.4 Eq, 49.6 μmol). The resulting mixture was then mixed and left at rt for another 20 minutes and then purified using flash column chromatography (0→30% MeOH in DCM). Fractions containing the product were concentrated to give compound 84 (123.1 mg, 80% purity, 98 μmol, 79%) as a red waxy oil. LCMS (ESI+) calculated for C₅₄H₅₃N₆O₁₄⁺ (M+H⁺) 1009.36 found 1010.01.

### Example a100. Synthesis of Compound 85

To a solution of compound 84 (123.1 mg, 80% Wt, 1 Eq, 97.60 μmol) in DCM (1 mL) was added a solution of PPh3 in DCM (529 mM, 553.5 μL, 292.8 μmol, 3.00 equiv.) and H₂O (600 μL). The resulting bi-phasic mixture was stirred vigorously at rt for 3 days. Next, DMF (600 μL) and additional H₂O (300 μL) was added, and the RM was stirred vigorously for another 3 days. Finally, the RM was diluted with DCM (10 mL) and the resulting bi-phasic system was separated. The water-layer was extracted with DCM (10 mL, 2×). The combined organic layers were dried (Na₂SO₄), filtered and concentrated to give crude amine (205.8 mg) that was directly used without further purification in the next step. LCMS (ESI+) calculated for C₅₄H₅₅N₄O₁₄⁺ (M+H⁺) 983.37 found 983.73. To a solution of compound 62 (18 mg, 66 wt %, 1.3 Eq, 20 μmol) in DMF (100 μL) and DIPEA (5.9 mg, 8.0 μL, 3 Eq, 46 μmol) was added HATU (6.1 mg, 1.05 Eq, 16 μmol). The mixture was mixed and after 3 minutes a solution of crude amine (15 mg, 1 Eq, 15 μmol) in DCM (200 μL) was added. The RM was mixed and left at rt for 1 hour. Next, additional compound 62 (9.2 mg, 66 wt %, 0.67 Eq, 10 μmol) in DMF (100 μL) and DIPEA (3.0 mg, 4.0 μL, 1.5 Eq, 23 μmol) were added, followed by additional HATU (2.9 mg, 0.5 Eq, 7.6 μmol). The RM was mixed and left at rt for circa 30 minutes and was then diluted with DMF (200 μL) and the mixture was then partially concentrated in vacuo to remove most of the DCM. The resulting solution was purified by prep-HPLC (30%→95% acetonitrile in 10 mM NH₄HCO₃ in water, column Xbridge prep C₁₈, 5 μM OBD, 30×100 mm). The collected fractions were combined to give compound 85 (3.9 mg, 2.5 μmol, 16% yield over 2 steps) as a red solid. LCMS (ESI+) calculated for C₈₀H₈₆N₉O₂₂S⁺ (M+H⁺) 1556.56 found 1557.07.

### Example a101. Synthesis of Compound 86

To a solution of compound 85 (3.9 mg, 1 Eq, 2.5 μmol) in DMF (200 μL) was added triethylamine (2.5 mg, 3.5 μL, 10 Eq, 25 μmol). The resulting solution was mixed and left at rt for 16 hours, followed by the addition of additional triethylamine (2.5 mg, 3.5 μL, 10 Eq, 25 μmol). The RM was mixed and left at rt for another 3 hours, followed by the addition of a 3^{rd} batch of triethylamine (2.5 mg, 3.5 μL, 10 Eq, 25 μmol)—and after another 105 minutes—a 4^{th} batch of triethylamine (2.5 mg, 3.5 μL, 10 Eq, 25 μmol). Finally, the resulting RM was left at rt for 140 minutes and was then purified by prep-HPLC (30%→95% acetonitrile in 10 mM NH₄HCO₃ in water, column Xbridge prep C₁₈, 5 μM OBD, 30×100 mm). The collected fractions were combined to give compound 86 (0.7 mg, 0.5 μmol, 20% yield) as a red solid. LCMS (ESI+) calculated for C₆₅H₇₆N₉O₂₀S⁺ (M+H⁺) 1334.49 found 1335.09.

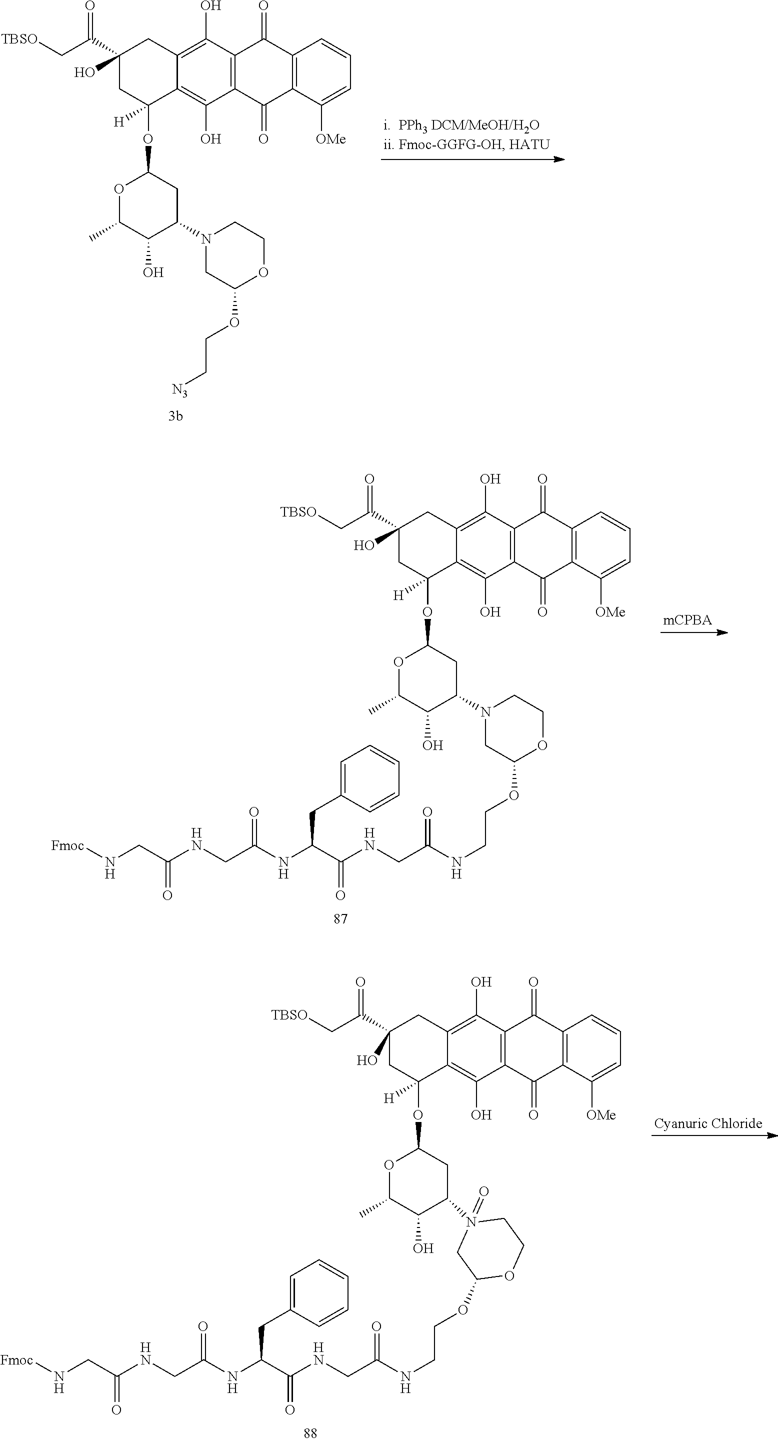

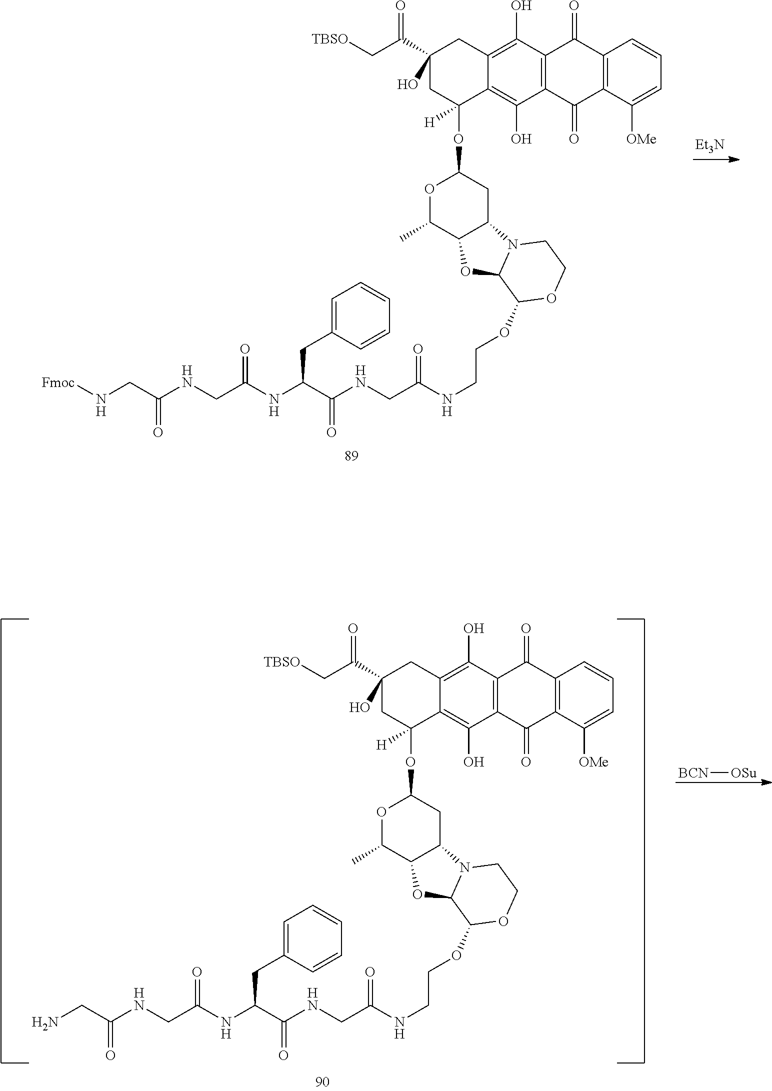

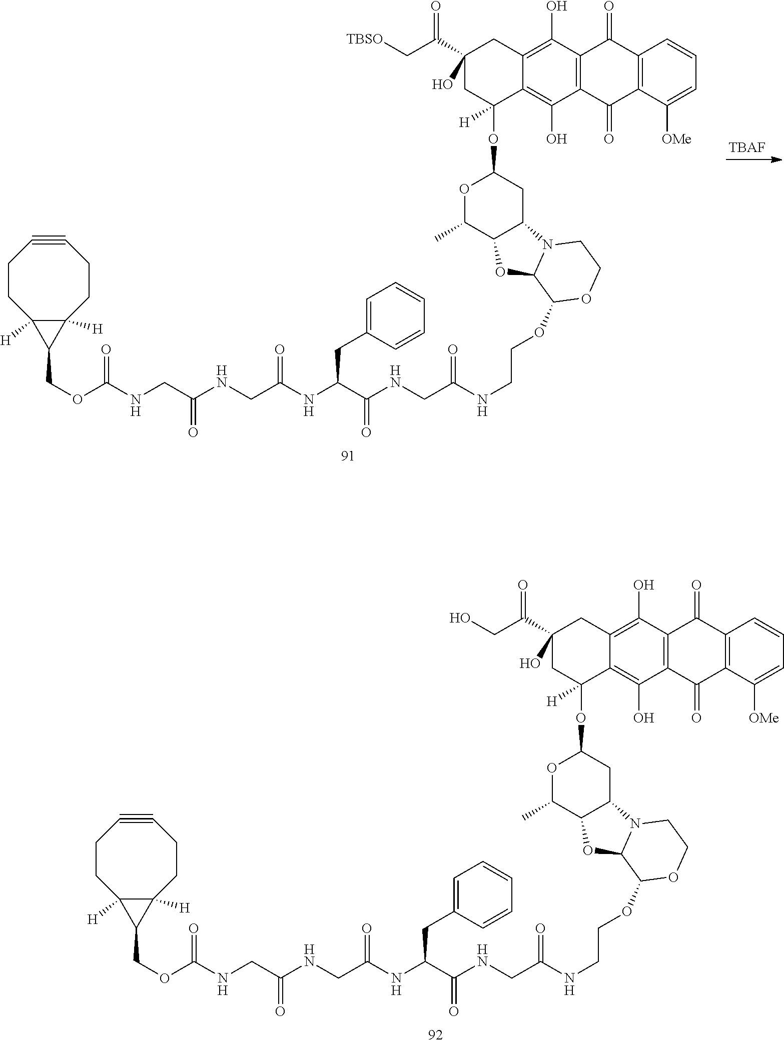

### Example a102. Synthesis of Compound 87

To a solution of compound 3b (224 mg, 0.20 mmol) and triphenylphosphine (180 mg, 0.69 mmol) in DCM (2.86 mL) and MeOH (1.74 mL) was added water (1.29 mL) and the reaction mixture was stirred vigorously at room temperature for 18 h. The mixture was then concentrated to a dark brown/red solid and re-dissolved in DMF (2 mL). Fmoc-Gly-Gly-Phe-Gly-OH (136 mg, 0.24 mmol), DIPEA (65 mg, 88 μL, 0.51 mmol,) and HATU (96 mg, 0.25 mmol) were added, and the reaction mixture stirred at room temperature for 30 min and then concentrated to a red/brown gum. The mixture was extracted into DCM/MeOH (9:1, 40 mL), washed with sat. aqueous brine solution (40 mL), dried through a phase separator and concentrated in vacuo. The residue was taken up in DCM (4 mL) and purified by flash column chromatography (1→15% MeOH/DCM) to afford compound 87 (273 mg, 94%) at 92% purity by LCMS as a dark red/brown solid that was used without further purification. UPLC-MS (ESI+) calculated for C₆₉H₈₃N₆O₁₉Si⁺ [M+H⁺] 1327.5 found 1327.9.

### Example a103. Synthesis of Compound 88

To a cooled solution of compound 87 (147 mg, 0.11 mmol) in DCM (2.2 mL) and MeOH (0.43 mL) at −78° C. was added dropwise a solution of mCPBA (41 mg, 0.17 mmol, 70% purity) in DCM (1 mL) over 5 min and the reaction mixture was stirred at −78° C. for 5 min. The reaction mixture was then quenched with ice-cold acetone (3 mL) and the mixture was stirred at −78° C. for 20 min and then warmed to room temperature. The reaction mixture was diluted with DCM (20 mL) and washed with sat. aq. NaHCO₃ solution (20 mL). The aqueous layer was re-extracted with DCM (25 mL) and the combined organic layers were dried through a phase separator and concentrated to give compound 88 (164 mg, 0.11 mmol, 96%) as a red solid at 87% purity by LCMS that was immediately redissolved in DCM (2 mL) and used in the next step without further purification. UPLC-MS (ESI+) calculated for C₆₉H₈₃N₆O₂₀Si⁺ [M+H⁺] 1343.5 found 1343.8.

### Example a104. Synthesis of Compound 89

To a solution of compound 88 (164 mg, 0.12 mmol) in dry DCM (20 mL) and dry acetonitrile (45 mL) was added cyanuric chloride (32 mg, 0.17 mmol) and the reaction mixture was stirred for 1 h at room temperature. Cyanuric chloride (11 mg, 58 μmol) in MeCN (0.5 mL) was added and stirred continued for 1 hour at room temperature. Next, additional cyanuric chloride (11 mg, 58 μmol) in MeCN (0.5 mL) and DCM (4 mL) were added and stirring continued at room temperature for 3.5 h. The reaction was quenched by addition of a mixture of 3-aminopropane-1,2-diol (106 mg, 1.16 mmol, 90 μL) in DCM (3 mL) and stirred at room temperature for 10 minutes before addition of MeOH (20 mL). The mixture was concentrated to a dark red gum that was extracted into DCM/MeOH (9:1, 25 mL) and insoluble residues were removed by filtration. The filtrate was concentrated to a red solid that was re-dissolved in DCM/MeOH (9:1, 4 mL) and purified by flash column chromatography (1→15% MeOH/DCM) to give compound 89 (24 mg, 89% purity) as a red solid. The solid was re-dissolved in DMF (0.5 mL) and MeCN (1 mL) and purified by flash reversed-phase chromatography (C₁₈, 40→95% MeCN in aq. 10 mM NH₄HCO₃ solution). Fractions containing product were combined, concentrated to ˜10 ml water and extracted with DCM (2×10 mL). The combined organic layers were dried through a phase separator and concentrated to give compound 89 (11 mg, 7%) as a red solid. The aqueous layer was concentrated to dryness to afford additional compound 89 (5 mg, 3%) as a red solid. UPLC-MS (ESI+) calculated for C₆₉H₈₁N₆O₁₉Si⁺ [M+H⁺] 1325.5 found 1325.8.

### Example a105. Synthesis of Compound 91

To a solution of compound 89 (15 mg, 11 μmol) in DMF (1 mL) was added triethylamine (23 mg, 0.23 mmol, 32 μL) and the reaction mixture was stirred at room temperature for 2 h. BCN-OSu (5 mg, 17 μmol) was added, and the reaction mixture stirred at room temperature for 18 h. The reaction mixture was concentrated to yield a red residue that was triturated with diethyl ether (3×4 mL, decanted off) and dried to give compound 91 (39 mg, quant.) as a red gum/solid that was used without purification in the next step. UPLC-MS (ESI+) calculated for C₆₅H₈₃N₆O₁₉Si⁺ [M+H⁺] 1279.5 found 1279.8.

### Example a106. Synthesis of Compound 92

To a cooled solution of compound 91 (39 mg, 11.3 μmol) in dry DMF (0.2 mL) and dry THF (0.6 mL) at −10° C. was added triethylammonium acetate (9 mg, 56 μmol) and stirring was continued at −10° C. for 5 min. Tetrabutylammonium fluoride solution in THF (1 M, 56 μL, 56 μmol) was added and the resulting solution was stirred at −10° C. for 20 min. Additional tetrabutylammonium fluoride solution in THF (1 M, 56 μL, 56 μmol) was added, resulting in a dark green solution that turned to red after two minutes, and stirring continued at −10° C. for 90 minutes. The reaction mixture was quenched by addition of ice-cold water (5 mL) and diluted with DCM (25 mL). Sat. aqueous brine (30 mL) was added, and layers were separated. The water-layer was extracted with DCM (10 mL) and the combined organic layers were dried through a phase separator and concentrated to ˜0.3 mL. The mixture was diluted with DCM (1 mL) and purified by flash column chromatography (0.5→15% MeOH/DCM) to give impure compound 92, which was subjected to a 2^{nd} purification by flash column chromatography (0→15% MeOH/DCM) to give compound 92 (2.0 mg) as a red solid. UPLC-MS (ESI+) calculated for C₅₉H₆₉N₆O₁₉⁺ [M+H⁺] 1165.5 found 1165.4.

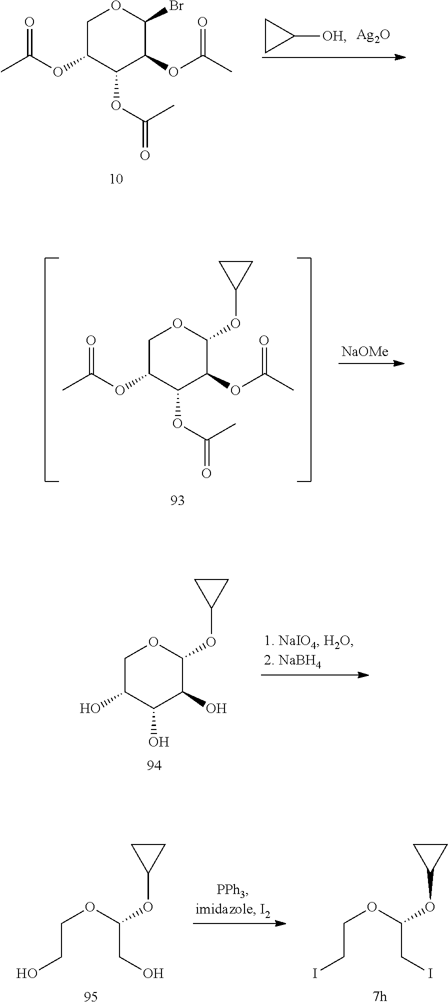

### Example a107. Synthesis of (1-O-(cyclopropyl)-α-D-arabinopyranose (94)

Arabinosyl bromide 10 (3000 mg, 8.85 mmol, 1.0 equiv.) was dissolved in dry Et₂O (36 mL, 0.25 M), this gave a suspension. Cyclopropanol (800 μL, 12.40 mmol, 1.4 equiv.) was added, followed by Ag₂O (2050 mg, 8.85 mmol, 1.0 equiv.). The mixture was stirred for 24 h in the dark. TLC analysis (1:9 v/v EtOAc-DCM) and 1H-NMR showed not full conversion (˜50% conversion) and the reaction was additionally stirred at rt for 24 h. TLC analysis (1:9 v/v EtOAc-DCM) and ¹H-NMR showed not full conversion (˜60% conversion). Cyclopropanol (400 μL, 6.20 mmol, 0.7 equiv.) and Ag₂O (1025 mg, 4.43 mmol, 0.5 equiv.) were added and the reaction was additionally stirred at rt for 24 h. TLC analysis (1:9 v/v EtOAc-DCM) and 1H-NMR showed full conversion and the reaction was filtered over Celite®, the Celite® pad was washed with Et₂O and the ether was removed by rotary evaporation. No further work-up was done and the obtained product 93 was used as such in the next step. The crude mixture of compound 93 (2800 mg, 8.85 mmol, 1.0 equiv.) was dissolved in MeOH (20 mL, 0.45 M), followed by addition of NaOMe (5.4 M in MeOH, 660 μL, 3.56 mmol, 0.4 equiv.). After stirring for 2 h at room temperature, TLC analysis (1:9 v/v MeOH-EtOAc) showed full conversion. The reaction mixture was neutralized with a few drops of HCl solution (1 M, 2.5 mL, 2.5 mmol) whereby it changed from a turbid reaction mixture into a clear solution. The mixture was concentrated. The crude product (2.1 g) was dissolved in MeOH (10 mL), applied onto a silica, and purified by flash column chromatography (0→10% MeOH in EtOAc, column pre-conditioned with PE) to obtain the pure compound 94 (520 mg, 2.73 mmol, yield 31% over 2 steps) as a white solid. ¹H NMR (300 MHZ, CD₃OD) δ: 4.9-4.7 (brs, OH, overlap with water), 4.29-4.24 (m, 1H), 3.92-3.78 (m, 2H), 3.72-3.63 (m, 1H), 3.61-3.45 (m, 3H), 0.83-0.70 (m, 1H), 0.62-0.49 (m, 2H), 0.49-0.38 (m, 1H). ¹³C NMR (101 MHZ, CD3OD) δ: 105.1, 74.3, 72.2, 69.7, 67.2, 53.0, 6.4, 5.4.

### Example a108. Synthesis of (R)-2-cyclopropyloxy-2-(2-hydroxyethoxy)ethan-1-ol (95)

Compound 94 (520 mg, 2.73 mmol, 1.0 equiv.) was dissolved in water (6 mL) and cooled down by a salt ice-bath (−5° C.). In the dark, NaOAc (290 mg, 3.5 mmol, 1.3 equiv.) dissolved in water (2 mL) was added followed by NalO₄ (1.46 g, 6.8 mmol, 2.5 equiv.) in portions (15 min). Additional water (3 mL) was added till a final concentration of 0.25 M was reached. The reaction mixture was stirred on ice for 15 minutes after which it was removed. TLC analysis (1:9 v/v MeOH-EtOAc) after 2 h showed full conversion of the starting compound 94 and the reaction mixture was cooled down by a salt ice-bath (−5° C.) and NaBH₄ (310 mg, 8.2 mmol, 3.0 equiv.) was added in portions (15 min, colour of the reaction mixture changed from dark red to colourless). TLC analysis (1:9 v/v MeOH-EtOAc) after an hour showed complete conversion of the intermediate aldehyde. EtOAc (30 mL) was added, and the organic layer was separated. The aqueous layer was extracted with EtOAc (30 mL) five times. The combined organic layers were dried over Na₂SO₄ and concentrated, to obtain product 95 (395 mg, 2.43 mmol, 89%) as a clear orange oil. ¹H NMR (300 MHz, CDCl₃) δ: 4.73 (dd, J=5.9, 3.8 Hz, 1H), 3.99-3.89 (m, 1H), 3.82 (t, J=4.3 Hz, 2H), 3.79-3.56 (m, 3H), 3.56-3.47 (m, 1H), 3.2-2.8 (brs, 2H), 0.72-0.48 (m, 4H). ¹³C NMR (101 MHZ, CDCl₃) δ: 103.3, 69.2, 62.7, 61.7, 51.4, 6.4, 5.7.

### Example a109. Synthesis of (R)-(2-iodo-1-(2-iodoethoxy)ethoxy)cyclopropane (7h)

Diol 95 (395 mg, 2.43 mmol, 1.0 equiv.) was dissolved in dry THF (13 mL, 0.19 M) and cooled down by a salt ice-bath (−5° C.). Imidazole (1.16 g, 17.04 mmol, 7.0 equiv.) was added at once followed by PPh3 (1.91 g, 7.28 mmol, 3.0 equiv.) in portions (10 min). Finally, 12 (1.85 g, 7.29 mmol, 3.0 equiv.) was added in portions (10 min). The mixture was stirred overnight in the dark (salt ice-bath was not removed and the reaction mixture was allowed to slowly warm up to room temperature). After dilution with EtOAc (25 mL), the organic layer was washed with Na₂S₂O₃ (10% aq., 20 mL). The organic layer was separated, and the aqueous layer was extracted with EtOAc (2×20 mL). The organic layers were combined, washed with brine (20 mL), and dried over Na₂SO₄. The mixture was concentrated, the crude product containing PPh₃/PPh₃O (3.7 g) were dissolved in DCM (4 mL), applied onto a silica, and purified using column chromatography (0→15% EtOAc in PE) to obtain pure compound 7h (540 mg, 1.41 mmol, 58%) as a light-yellow oil. ¹H NMR (300 MHz, CDCl₃) δ: 4.72 (t, J=5.4 Hz, 1H), 4.00-3.78 (m, 2H), 3.59-3.48 (m, 1H), 3.34-3.18 (m, 4H), 0.79-0.44 (m, 4H). 13C NMR (101 MHZ, CDCl₃) δ (ppm) 102.9, 67.9, 50.8, 6.3, 5.4, 5.2, 2.5.

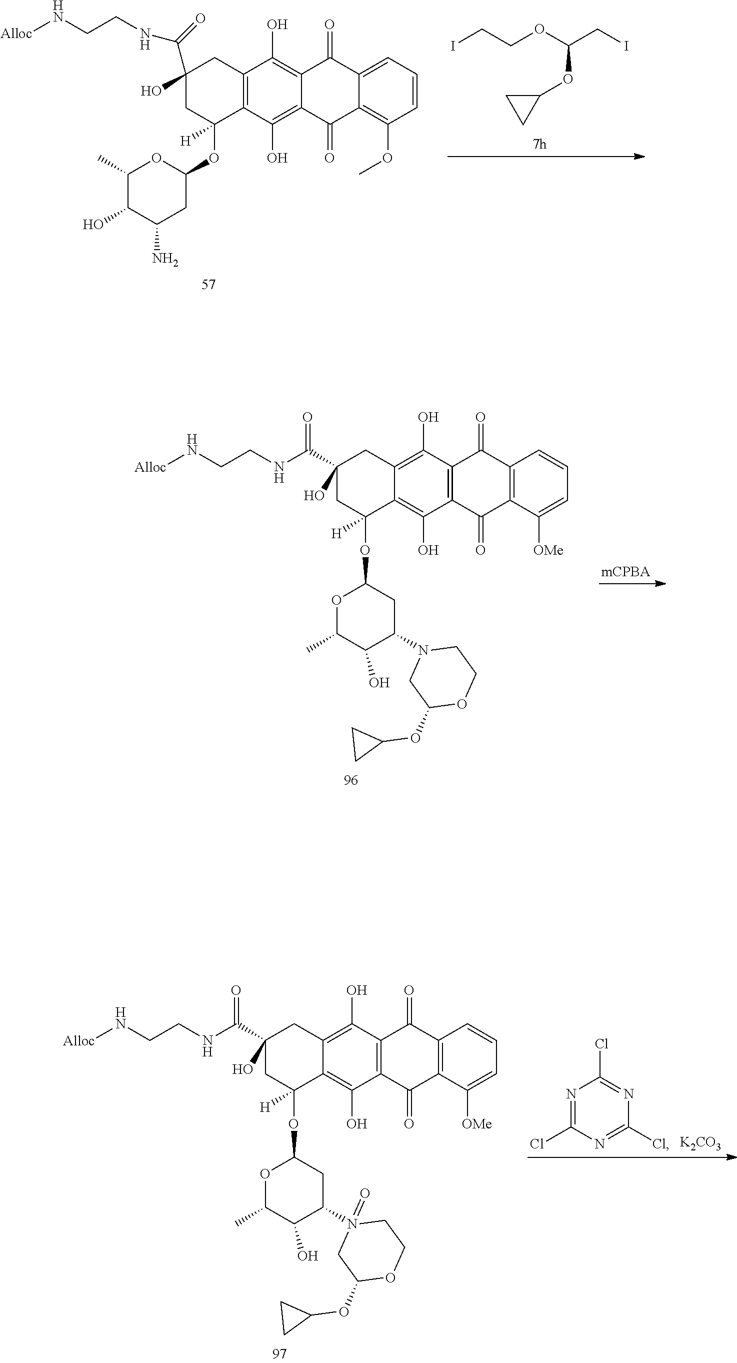

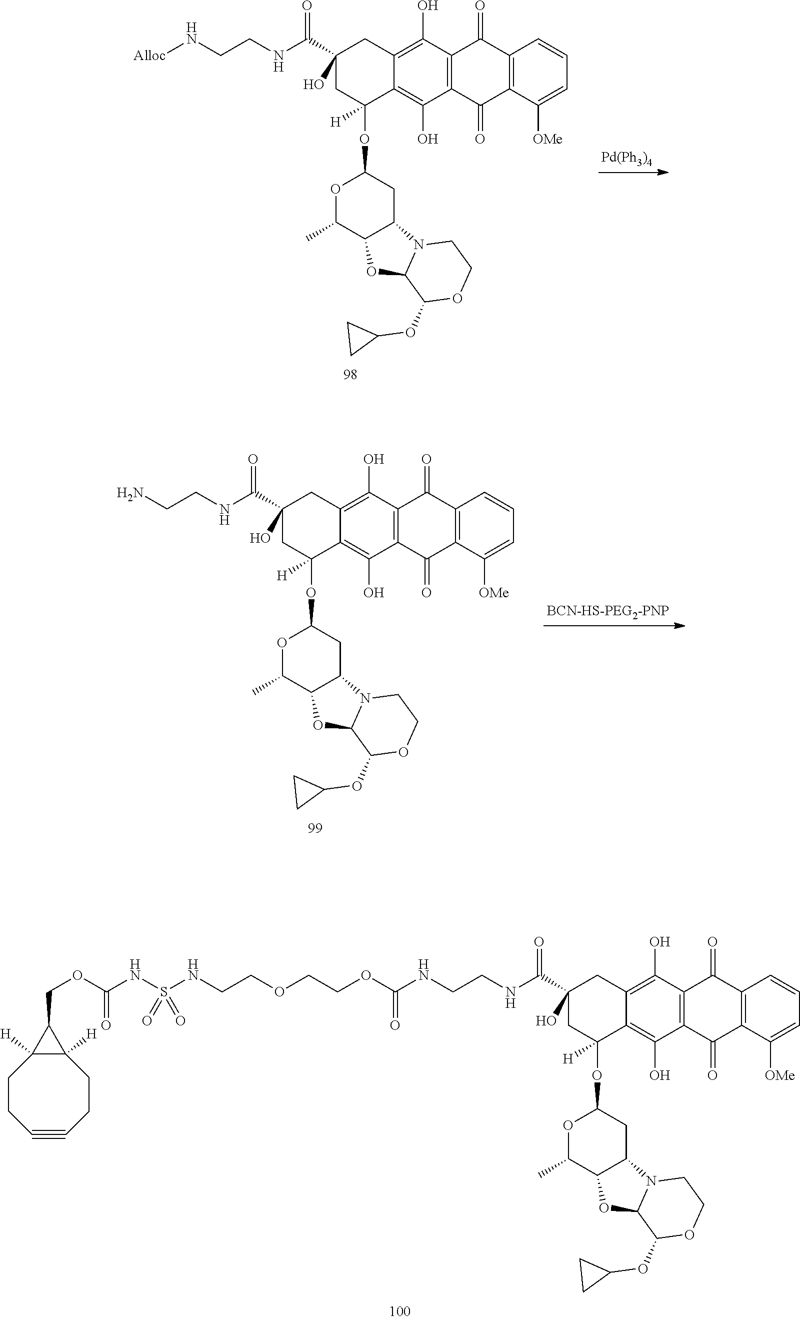

### Example a110. Synthesis of Compound 96

Intermediate 57 (389.7 mg, 81.9 wt %, 1 Eq, 486.8 μmol) was suspended in dry DMF (600 μL) and then heated and to 43° C. for 10 minutes to obtain a nearly clear solution. Next, bis-iodo-sugar 7h (464.9 mg, 2.5 Eq, 1.217 mmol) in dry DMF (200 μL) and DIPEA (188.8 mg, 254 μL, 3 Eq, 1.460 mmol) were added. The resulting mixture was stirred in the dark at 38° C. for 2 days, followed by another day at rt. The RM was then diluted with DCM (12 mL) and the resulting red solution was purified by flash column chromatography over silicagel (0→10% MeOH in DCM) to give compound 96 as a red foam (344.5 mg, 89% purity, 0.39 mmol, 81%). LCMS (ESI+) calculated for C₃₉H₄₈N₃O₁₄⁺ (M+H⁺) 782.32 found 782.64.

### Example a111. Synthesis of Compound 97

A solution of compound 96 (344.5 mg, 89 wt %, 1 Eq, 392.2 μmol) in a mixture of DCM (10 mL) was cooled by a dry ice/acetone-bath to a temperature of −78° C. The mixture was vigorously stirred after which a freshly made stock solution of mCPBA in DCM (845.2 μL, 580 mmolar, 1.25 Eq, 490.2 μmol) was added dropwise via the side of the flask. After stirring for 14 minutes, a 2^{nd} batch of mCPBA in DCM (135.2 μL, 580 mmolar, 0.2 Eq, 78.43 μmol) was added dropwise via the side of the flask and the RM was stirred for another 17 minutes. The RM was then quenched with cold (−78° C.) acetone (reagent-grade, 8.0 mL) and the RM was stirred at −78° C. After 1 hour the cooling-bath was removed, and the RM was allowed to warm up to rt over 35 minutes. The RM was diluted with DCM (30 mL) and saturated aqueous NaHCO₃ solution (15 mL) and transferred to a separation funnel. The resulting biphasic system was separated. The organic layer was dried over Na₂SO₄, filtered, and then concentrated to give compound 97 (334.2 mg, 88% purity, 0.37 mmol, 94% yield), which was used as such in the next step. LCMS (ESI+) calculated for C₃₉H₄₈N₃O₁₅⁺ (M+H⁺) 798.31 found 798.70.

### Example a112. Synthesis of Compound 98

To a solution of 97 (334.2 mg, 88 wt %, 1 Eq, 368.6 μmol) in a mixture of dry DCM (3.0 mL) and anhydrous acetonitrile (4.0 mL) was added potassium carbonate (203.8 mg, 4 Eq, 1.475 mmol). The RM was then cooled to 0° C. with an ice-bath after which a solution of cyanuric chloride (4.2 mL, 200 mmolar, 2.3 Eq, 0.84 mmol) in anhydrous acetonitrile was added. After stirring for 70 minutes at 0° C. the RM was quenched with a solution of 3-aminopropane-1,2 diol in H₂O (2.267 mL, 2 molar, 12.3 Eq, 4.534 mmol). The ice bath was removed after 15 min and was then allowed to warm to rt. The resulting bi-phasic system was then separated. The resulting water layer was extracted with DCM (10 mL). The combined organic layers were concentrated in vacuo and the residue was purified by flash column chromatography over silicagel (0→20% MeOH in DCM) to give compound 98 (8.2 mg, 11 μmol, 2.9%) as a red solid. LCMS (ESI+) calculated for C₃₉H₄₆N₃O₁₄⁺ (M+H⁺) 780.30 found 780.64.

### Example a113. Synthesis of Compound 99

To a vial containing compound 98 (8.2 mg, 83.5 wt %, 1 Eq, 8.8 μmol) was added anhydrous DCM (300 μL) and to the resulting red solution was added pyrrolidine (1.9 mg, 2.2 μL, 3 Eq, 26 μmol), followed by the addition of Pd(PPh₃)₄ in dry DCM (1.50 mg, 64.8 μL, 20 mmolar, 0.15 Eq, 1.30 μmol). The dark red solution was mixed and left at rt for 16 minutes. Additional 20 mmolar Pd(PPh₃)₄ in dry DCM (1.5 mg, 66 μL, 20 mmolar, 0.15 Eq, 1.3 μmol). was added, followed after 45 and 20 minutes by a third (1.5 mg, 66 μL, 20 mmolar, 0.15 Eq, 1.3 μmol)) and fourth (1.5 mg, 66 UL, 20 mmolar, 0.15 Eq, 1.3 μmol)) batch of Pd(PPh₃)₄ in dry DCM respectively. Finally, following the fourth addition, the RM was stirred at rt for 40 minutes and was then transferred to a separation funnel together with sat. aq. NH₄Cl (1 mL). The mixture was shaken, and the resulting bi-phasic system was separated. The water-layer was extracted twice with DCM (2 mL, 2×). To the combined organic layers was added DMF (250 μL) and the resulting solution was partially concentrated in vacuo until all the DCM was removed. The resulting solution was purified by prep-HPLC (20%→50% acetonitrile in 10 mM NH₄HCO₃ in water, column Xbridge prep C₁₈, 5 μM OBD, 30×150 mm). The fractions containing the product were combined and concentrated to give compound 99 (0.6 mg) as a red solid. LCMS (ESI+) calculated for C₃₅H₄₂N₃O₁₂⁺ (M+H⁺) 696.28 found 696.56.

### Example a114. Synthesis of Compound 100

To compound 99 (0.6 mg) in DMF was added BCN-HS-PEG₂-OPNP (0.6 mg, 9 μL, 131 mmolar, 1 μmol) followed by triethylamine (0.3 mg, 0.4 μL, 3 μmol). The resulting RM was mixed and left at rt for 4.5 hours to give compound 100 as a red solution that was not isolated. LCMS (ESI+) calculated for C₅₁H₆₄N₅O₁₉S⁺ (M+H⁺) 1082.39 found 1082.84.

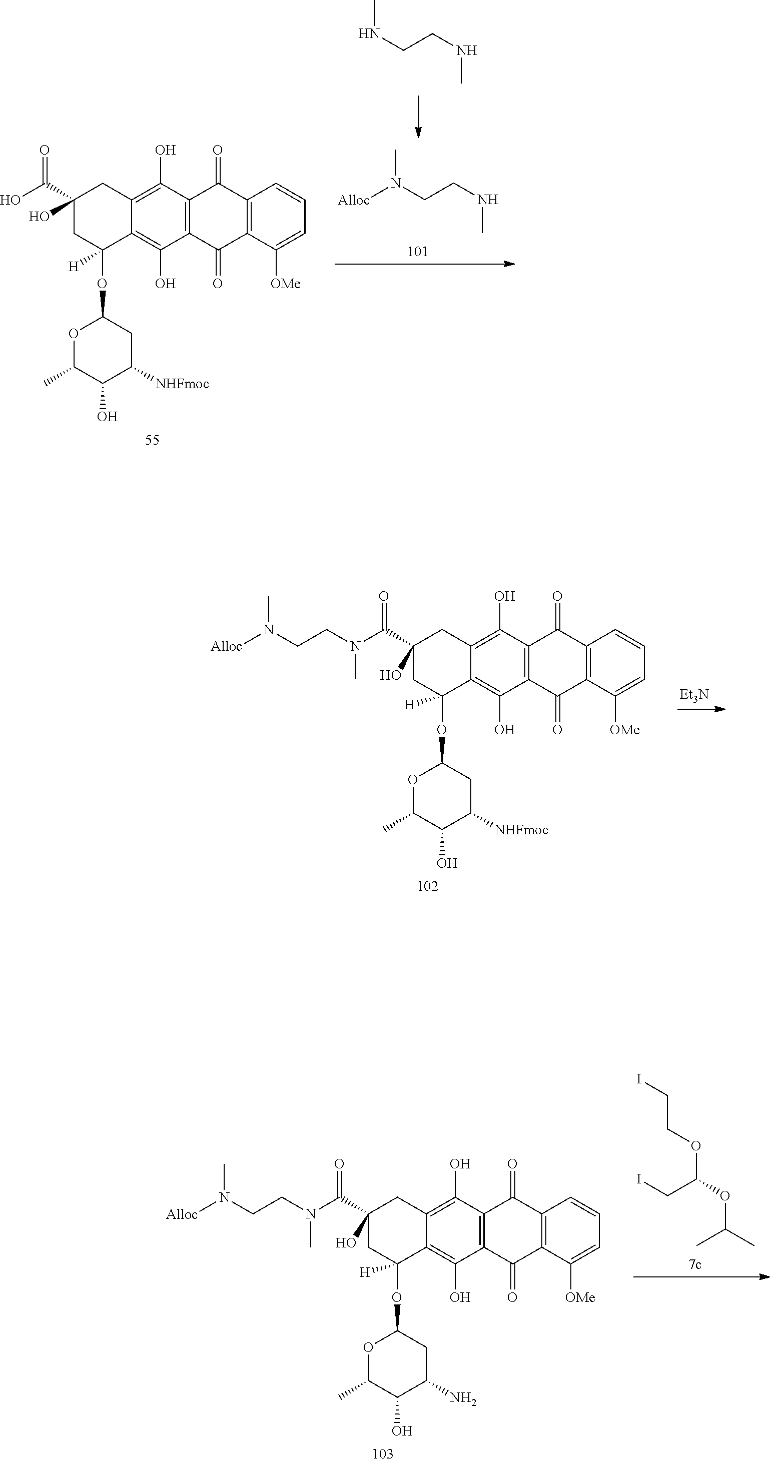

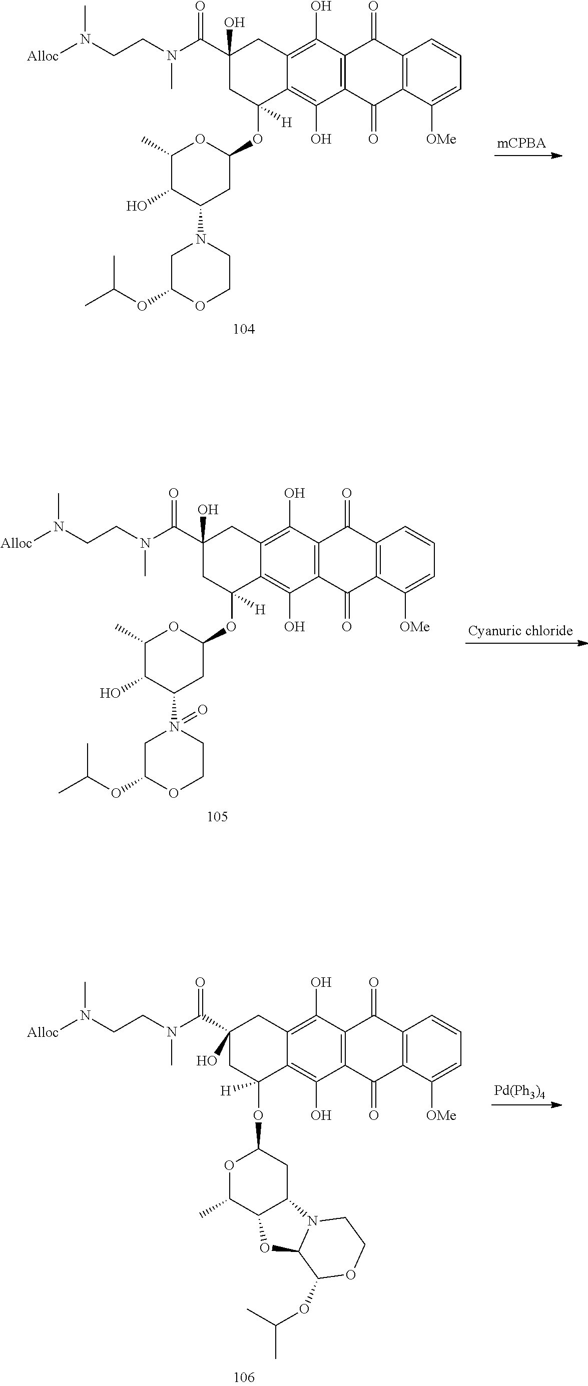

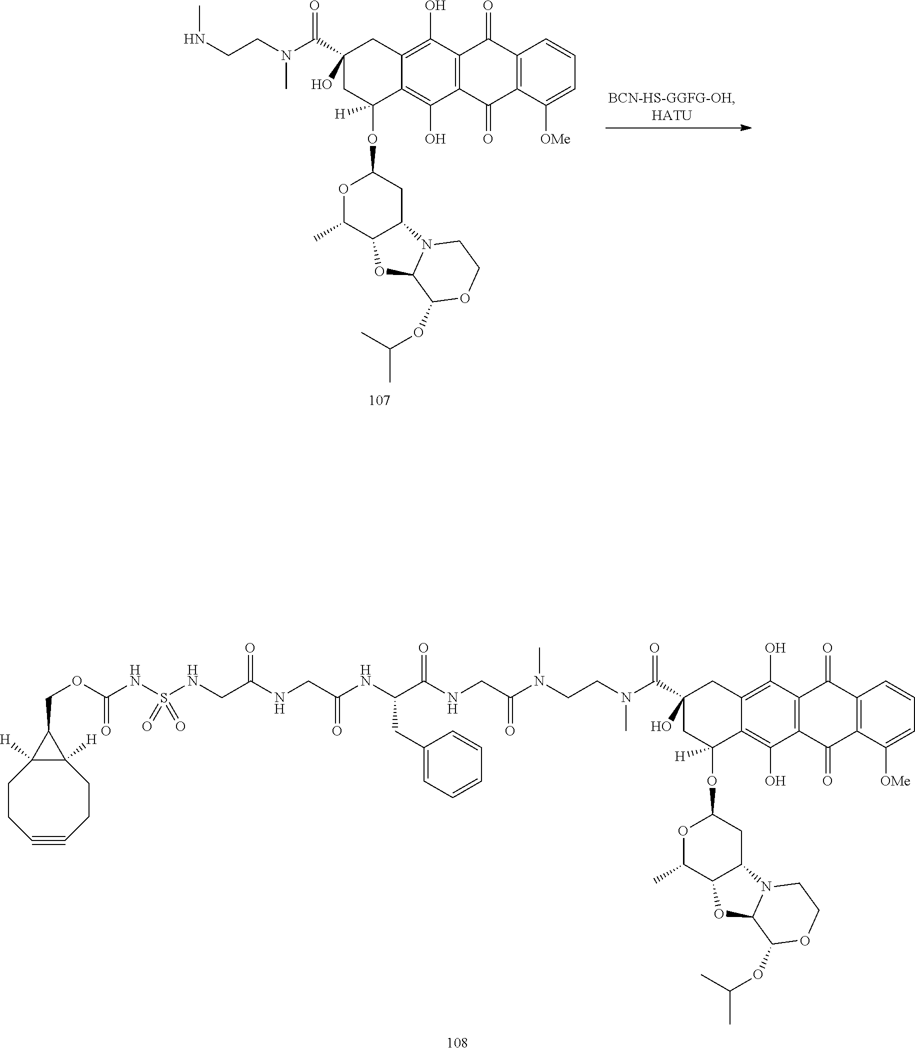 [SMILES: CNCCN(C)C(=O)[C@]1(O)Cc2c(O)c3c(c(O)c2[C@@H](O[C@H]2C[C@H]4[C@H](O[C@@H]5[C@@H](OC(C)C)OCCN54)[C@H](C)O2)C1)C(=O)c1c(OC)cccc1C3=O ; COc1cccc2c1C(=O)c1c(O)c3c(c(O)c1C2=O)C[C@@](O)(C(=O)N(C)CCN(C)C(=O)CNC(=O)[C@H](Cc1ccccc1)NC(=O)CNC(=O)CNS(=O)(=O)NC(=O)OC[C@@H]1[C@@H]2CCC#CCC[C@@H]21)C[C@@H]3O[C@H]1C[C@H]2[C@H](O[C@@H]3[C@@H](OC(C)C)OCCN32)[C@H](C)O1]

### Example a115. Synthesis of Compound 101

To a round-bottom flask of N,N′-Dimethylethane-1,2-diamine (3.02 g, 1.65 Eq, 34.2 mmol) in ethanol (148 mL) was added a solution of allyl (2,5-dioxopyrrolidin-1-yl) carbonate (4.13 g, 1 Eq, 20.7 mmol) in 30 mL ethanol dropwise over 15 min. The RM was stirred overnight at rt. The RM was then stored in the freezer for 3 days. Next, the RM was taken out of the freezer and allowed to warm to rt. And was then concentrated in vacuo and then re-dissolved in 200 ml water. The pH was adjusted to pH 3 by addition of aqueous HCl (1 Molar) and extracted with DCM (100 mL, 4×) to remove any bis-reacted amine. This organic layer was discarded. The aqueous layer was adjusted to a pH of 14 with the addition of aqueous NaOH (1 Molar) and extracted with DCM (100 mL, 4×). The combined organic layers were washed with aqueous NaOH (2 Molar, 200 mL, 2×) and dried over Na₂SO₄, filtrated and concentrated to give impure compound 101. The impure material was re-dissolved in 100 ml water, generating a milky white suspension with yellow solids. The aqueous layer was adjusted to a pH=3 with the addition of aqueous HCl (1 Molar). The solution became less milky, but still not clear. The aqueous layer was washed with MTBE (3×50 mL), followed by washing steps with Et₂O (50 mL, 3×). Finally, the aqueous layer was washed with DCM (50 mL, 3×). Next, the aqueous was adjusted to a pH 14 with the addition of aqueous NaOH (1 Molar) and was then extracted with DCM (50 mL, 4×). The combined organic layers were then washed with aqueous NaOH (2 Molar, 100 mL, 2×), dried over Na₂SO₄, filtrated, and concentrated to obtain a compound 101 (1.01 g, 5.87 mmol, 28.3%) as a clear oil. UPLC-MS (ESI+) calculated for C₈H₁₇N₂O₂⁺ [M+H⁺] 173.13 found 173.29.

### Example a116. Synthesis of Compound 102

To a round-bottom flask containing compound 55 in DMF (1.61 g, 36.9 mL, 58 mmolar, 1 equiv., 2.14 mmol) was added a solution of allyl methyl (2-(methylamino) ethyl) carbamate (101) (1.00 g, 5.82 mmol, 2.72 equiv.) in dry DMF (3.7 mL). The RM was then placed in a water-bath and HATU (854 mg, 1.05 equiv., 2.25 mmol) was added, followed within one minute by the addition of DIPEA (830 mg, 1.12 mL, 3 equiv., 6.42 mmol) and the resulting dark red solution was stirred at rt for 10 min. The RM was then partially concentrated in vacuo until a volume of 6 ml. The residue was then diluted with DCM (80 mL) and loaded onto a pre-wetted column. The residue was then purified using flash column chromatography over silicagel (0→20% MeOH in DCM). Fractions containing product were combined and concentrated in vacuo, affording compound 102 (1.44 g, 88.1% purity, 1.40 mmol, 65.6%) as a dark red thick oil. UPLC-MS (ESI+) calculated for C₄₉H₅₂N₃O₁₄⁺ [M+H⁺] 906.34 found 906.90.

### Example a117. Synthesis of Compound 103

To a dark red solution of compound 102 (1.44 g, 88.1 wt %, 1.40 mmol, 1 equiv.) in a total volume of DMF (5.85 mL) was added triethylamine (979 μL, 7.02 mmol, 5.0 equiv.). The RM turned very dark red (near black) and was left to stir at rt for circa 18 hours. The RM was treated with Et₂O (78 mL) slowly in one go while stirring rapidly. The ether-layer was decanted off and the residue was washed a few more times with Et₂O (3×30 ml). The residue was concentrated in vacuo to give compound 103 (619 mg, 60.5% purity, 547.0 μmol, 39.0%) as a dark red solid, which was used without further purification. UPLC-MS (ESI+) calculated for C₃₄H₄₂N₃O₁₂⁺ [M+H⁺] 684.28 found 684.65.

### Example a118. Synthesis of Compound 104

Compound 103 (598.6 mg, 60.5 wt %, 529.3 μmol, 1.00 equiv.) was dissolved in dry DMF (2.41 mL) after which (R)-2-(2-iodo-1-(2-iodoethoxy)ethoxy) propane (7c) (613.5 mg, 1.598 mmol, 3.02 Eq,) was added. The vial was covered in aluminium foil and the reaction was stirred in the dark for 6 days at 40° C. Next, the RM was diluted with DCM (17 mL) and the resulting red solution was purified by flash column chromatography over silicagel (0%→5% MeOH in DCM). The fractions containing pure product were combined and concentrated to give pure compound 104 (110.9 mg, 213.9 μmol, 40.4%) as a dark red residue. In addition, fractions containing impure product were combined, affording impure compound 104 (149.3 mg, 89.7% purity, 165 μmol, 31.2%). LCMS (ESI+) calculated for C₄₁H₅₃N₃O₁₄⁺ (M+H⁺) 812.36 found 812.83.

### Example a119. Synthesis of Compound 105

Compound 104 (149.3 mg, 89.7 wt %, 165.0 μL, 1.00 equiv.) was dissolved in dry DCM (3.0 mL) and dry MeOH (1.0 mL). The mixture was cooled down by a dry ice/acetone cooling-bath (−78° C.). Then mCPBA (31.31 mg, 312.8 μL, 580 mmolar, 1.1 Eq, 181.4 μmol) was dropwise added by using a microman pipette. The RM was stirred vigorously for 5 min. Meanwhile, reagent-grade acetone was cooled in the dry ice/acetone cooling-bath. The RM was then quenched with cold reagent-grade acetone (3.33 mL, 275 Eq, 45.4 mmol) and left stirring in the cold bath for 90 minutes. The dry ice bath was then removed, and the RM was allowed to warm up to room temperature. This was followed by the addition of 20 mL DCM. The RM was then transferred to a separation funnel and washed twice with 15 mL sat. NaHCO₃ aq. solution. The organic layer was dried over Na₂SO₄ and filtered through a membrane filter. The solution was concentrated under vacuo to afford compound 105 (136.3 mg, 82.8% purity, 136 μmol, 82.6%) as a red solid. LCMS (ESI+) calculated for C₄₁H₅₄N₃O₁₅⁺ (M+H⁺) 828.35 found 828.75.

### Example a120. Synthesis of Compound 106

To a solution of 105 (136.3 mg, 82.8 wt %, 1.0 Eq, 136.3 μmol) in dry MeCN (2.5 mL) and dry DCM (1.0 mL) was added potassium carbonate (75.36 mg, 4.00 Eq, 545.3 μmol) and the RM was cooled down with an ice bath (ice bath 0° C.). Then cyanuric chloride in dry MeCN (62.84 mg, 3.408 mL, 100 mmolar, 2.5 Eq, 340.8 μmol) was added at once and the mixture was stirred vigorously for 1 h. The RM was then quenched with 3-aminopropane-1,2 diol in H₂O (152.8 mg, 838.4 μL, 2 molar, 12.3 Eq, 1.677 mmol). A red-purple precipitate was formed on the sides of the flask. The RM was transferred to a separation funnel, 20 mL of DCM and 20 ml of water were added. Both layers were separated. The organic layer was dried over Na₂SO₄, filtered, and concentrated in vacuo to afford a dark red solid. The residue was redissolved in DCM and purified using flash column chromatography over silicagel (0→10% MeOH in DCM). Fractions containing the product were concentrated in vacuo to afford impure compound 106. The material was subjected to a 2^{nd} purification using flash column chromatography over silicagel (0→10% MeOH in DCM). Fractions containing the product were concentrated in vacuo to yield compound 106 (26.3 mg, 55% purity, 18 μmol, 13%) as a dark red solid. LCMS (ESI+) calculated for C₄₁H₅₂N₃O₁₄⁺ (M+H⁺) 810.34 found 810.69.

### Example a121. Synthesis of Compound 107

Compound 106 (26.3 mg, 55 wt %, 1.0 Eq, 17.9 μmol) was dissolved in degassed anhydrous DCM (700 μL, degassed for 10 min with N₂). To this solution was added pyrrolidine (3.81 mg, 4.40 μL, 3 Eq, 53.6 μmol), a dark red solution was then obtained, followed by Pd(PPh₃)₄ in degassed anhydrous DCM (3.10 mg, 134 μL, 20 mmolar, 0.15 Eq, 2.68 μmol). The RM was stirred for 25 min, followed by the addition of additional Pd(PPh₃)₄ in degassed anhydrous DCM (3.10 mg, 134 μL, 20 mmolar, 0.15 Eq, 2.68 μmol). The RM was stirred for another 1 h. Next, pyrrolidine (3.81 mg, 4.40 μL, 3 Eq, 53.6 μmol) was added, followed after 2 minutes by a 3^{rd} batch of Pd(PPh₃)₄ (3.10 mg, 0.15 Eq, 2.68 μmol) as a solid. The RM was stirred for 90 minutes. Finally, a fourth batch of Pd(PPh₃)₄ (3.10 mg, 0.15 Eq, 2.68 μmol) was added as a solid. The RM was stirred for another 3.5 h. The RM was then transferred to a separation funnel and 10 mL sat. aq. NH₄Cl were added. The resulting bi-phasic system was separated, and the water layer was extracted once with DCM (10 mL). The second organic layer was lighter red, but the water layer stayed dark red. No DP was observed in the water layer. The combined organic layers were dried over Na₂SO₄ and filtered. Next, 300 μL DMF was added, and the solution was partially concentrated until all the DCM was gone. This solution was diluted to 800 μL with additional DMF and purified by prep-HPLC (Column Xbridge prep C₁₈, 5 μm OBD, 30×100 mm, 20%→50% MeCN in 10 mM aq. NH₄HCO₃). Fractions containing the product were combined and concentrated in vacuo. After the fractions were combined and completely concentrated, the red solid was redissolved twice in MeCN and then concentrated and once in DCM and concentrated to yield compound 107 (3.9 mg, 5.4 μmol, 30%) as a dark red solid. LCMS (ESI+) calculated for C₃₇H₄₈N₃O₁₂⁺ (M+H⁺) 726.32 found 726.76.

### Example a122. Synthesis of Compound 108

To BCN-HS-GGFG-OH (62) (6.3 mg, 66 wt %, 1.3 Eq, 7.0 μmol) was added first DIPEA (2.1 mg, 2.8 μL, 3 Eq, 16 μmol) and then HATU (2.7 mg, 1.3 Eq, 7.0 μmol) in 100 μL of DMF. The RM was stirred for 5 minutes. Then, intermediate 107 in 400 μL of DMF (3.9 mg, 1.0 Eq, 5.4 μmol) was added. The RM was stirred for 1 h. Because of partial conversion, BCN-HS-GGFG-OH (62) (4.1 mg, 1.3 Eq, 7.0 μmol) in DMF (150 μL), DIPEA (6.1 mg, 3 Eq, 16 μmol) and HATU (2.7 mg, 1.3 Eq, 7.0 μmol) were pre-mixed for 5 min and added to the main RM, which was stirred for another 2 h. The RM was then diluted to 800 μL with additional DMF and purified by Prep-HPLC (Column Xbridge prep C₁₈, 5 μm OBD, 30×100 mm, 30%→95% MeCN in 10 mM aq. NH₄HCO₃). The fraction containing the product was concentrated. After the fraction was concentrated, the red solid was redissolved twice in MeCN and then concentrated and once in DCM and concentrated to yield compound 108 (1.2 mg, 0.92 μmol, 17%) as a red solid. LCMS (ESI+) calculated for C₆₃H₇₉N₈O₂₀S⁺ (M+H⁺) 1299.51 found 1300.15.

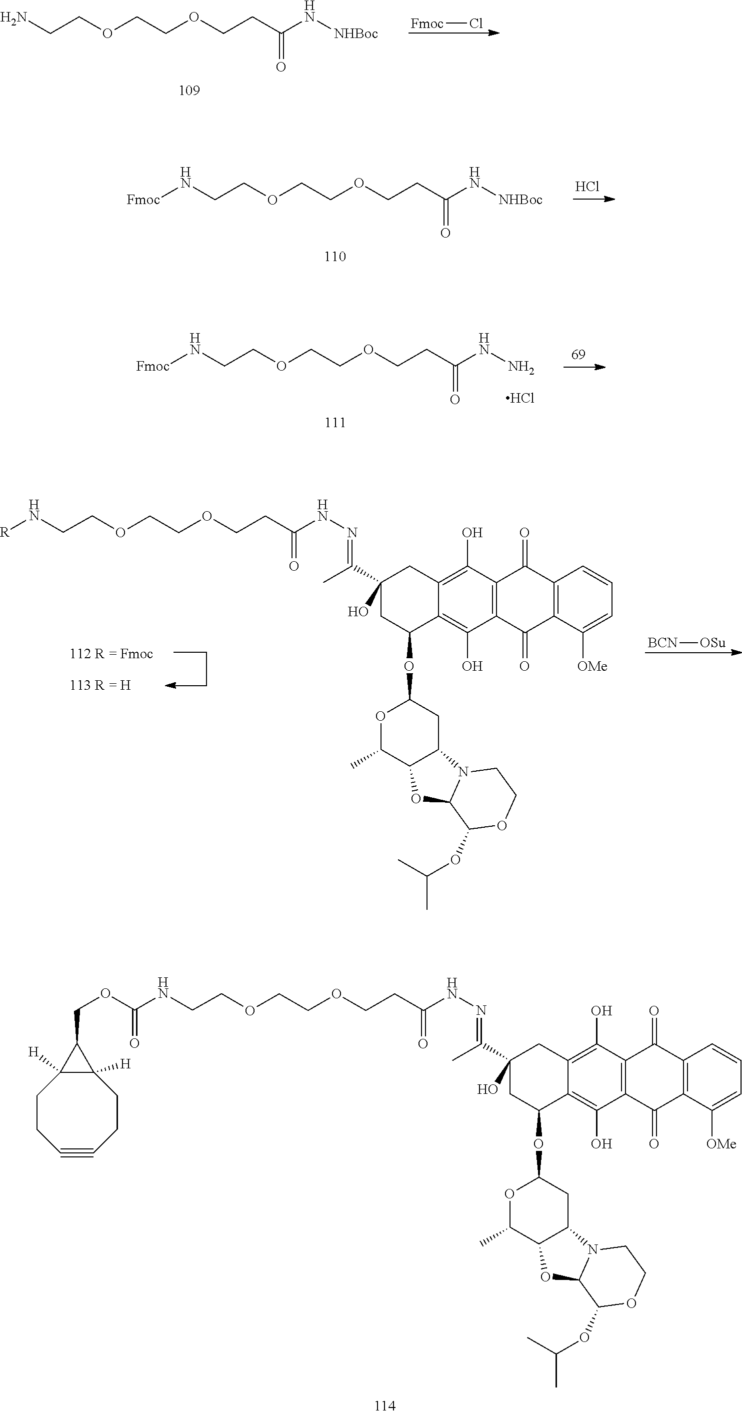

### Example a123. Synthesis of Compound 110

In a vial, amino-PEG2-t-Boc-hydrazide 109 (41.0 mg, 1 Eq, 141 μmol) and sodium carbonate (16.6 mg, 1.11 Eq, 157 μmol) were dissolved in 1,4-dioxane (1.00 mL) and water (1.00 mL). The RM was stirred at room temperature for 10 minutes, followed by the addition of Fmoc-chloride (37.8 mg, 1.04 Eq, 146 μmol). The RM was stirred at rt for 4.5 hours. Then additional sodium carbonate (11.6 mg, 0.778 Eq, 109 μmol) and Fmoc-chloride (7.1 mg, 0.20 Eq, 27 μmol) were added and left stirring for 19 hours. The pH was adjusted to pH=3 with 4 M aq. HCl and the aqueous phase was extracted thrice with ethyl acetate (3×5 mL). The organic phases were combined and washed twice with saturated brine (2×20 mL), dried over anhydrous magnesium sulfate, and concentrated in vacuo. The residue was then dissolved in DCM (1.0 mL) and then purified using flash column chromatography over silicagel (0→40% aceton in DCM). Fractions containing product were combined and concentrated in vacuo, affording compound 110 (43.4 mg, 84.5 μmol, 60.0%) as a clear oil. LCMS (ESI+) calculated for C₂₇H₃₆N₃O₇⁺ (M+H⁺) 514.25 found 514.65.

### Example a124. Synthesis of Compound 111

To a solution of compound 110 (43.4 mg, 1 Eq, 84.5 μmol) in DCM (1.50 mL) was added 4 M HCl in 1,4-dioxane (46.2 mg, 317 μL, 15 Eq, 1.27 mmol) and the RM was stirred at rt for 3 hours. Then, additional 4 M HCl in 1,4-dioxane (61.6 mg, 423 μL, 20 Eq, 1.69 mmol) was added and left stirring for 2 hours. The RM was concentrated in vacuo and thrice co-evaporated with toluene, affording compound 111 (35.7 mg, 79.3 μmol, 93.9%) as a clear oil. Compound 111 was then dissolved in anhydrous MeOH (1 mL) and used without further purification in the next step. LCMS (ESI+) calculated for C₂₂H₂₈N₃O₅⁺ (M+H⁺) 414.20 found 414.53.

### Example a125. Synthesis of Compound 112

250 μL of compound 111 in MeOH solution (˜8.9 mg, 19.8 μmol) was loaded onto a cartridge (PL-HCO3 MP SPE, 500 mg/6 ml tube) and flushed thrice with anhydrous MeOH. The filtrate was concentrated in vacuo, affording an acid-free version of compound 111 (6.9 mg, 2.3 Eq, 15 μmol). This was then dissolved in anhydrous MeOH (0.250 mL) and added to a vial containing a mixture of compound 69 (5.1 mg, 85% Wt, 1.0 Eq, 6.6 μmol) in anhydrous MeOH (0.250 mL). The resulting suspension was swirled in a 40° C. water-bath for a few minutes until everything dissolved. The RM was stirred for 90 minutes, followed by the addition of acetic acid (0.40 mg, 0.38 μL, 1 Eq, 6.6 μmol) and the RM was left stirring for 42 hours. In parallel, another batch of compound 111 in MeOH solution (250 μL, ˜8.9 mg, 19.8 μmol) was subjected to the procedure described above, but with a reaction time of 18 hours, following the addition of compound 69. Both reaction-mixtures were combined and then diluted with DMF (500 μL). The resulting mixture was partially concentrated in vacuo to evaporate the majority of MeOH to give a solution of crude 112 in mainly DMF that was used in the next step without further purification. LCMS (ESI+) calculated for C₅₆H₆₅N₄O₁₆⁺ (M+H⁺) 1049.44 found 1049.90.

### Example a126. Synthesis of Compound 113

To a vial containing a dark red solution of compound 112, (14 mg, 1 Eq. 13 μmol) in DMF (500 μL) was added triethylamine (14 mg, 19 μL, 10 Eq, 0.13 mmol) and the RM was stirred for 3 hours at rt. Then, additional triethylamine (14 mg, 19 μL, 10 Eq, 0.13 mmol) was added and the RM was heated in a 40° C. water-bath for 2.5 hours. This solution was diluted to 800 μL with additional DMF and purified by prep-HPLC (Column Xbridge prep C₁₈, 5 μm OBD, 30×150 mm, 30%→95% MeCN in 10 mM aq. NH₄HCO₃). Fractions containing the product were combined and concentrated in vacuo. After the fractions were combined and completely concentrated, the red solid was redissolved twice in MeCN and then concentrated and once in DCM and concentrated to yield compound 113 (3.6 mg, 4.4 μmol, 33%) LCMS (ESI+) calculated for C₄₁H₅₅N₄O₁₄⁺ (M+H⁺) 827.37 found 827.86.

### Example a127. Synthesis of Compound 114

To a vial containing a dark red solution of compound 113 (3.6 mg, 1 Eq, 4.4 μmol) in DMF (0.300 mL), was added a solution of BCN-OSu (1.5 mg, 347 mmolar, 1.2 Eq, 5.2 μmol) in DMF (15 μL), followed by the addition of triethylamine (1.3 mg, 1.8 μL, 3 Eq, 13 μmol). The RM stirred for 75 minutes at rt. This solution was diluted to 800 μL with additional DMF and purified by prep-HPLC (Column Xbridge prep C₁₈, 5 μm OBD, 30×150 mm, 30%→95% MeCN in 10 mM aq. NH₄HCO₃). Fractions containing the product were combined and concentrated in vacuo. After the fractions were combined and completely concentrated, the red solid was redissolved twice in MeCN and then concentrated and once in DCM and concentrated to yield compound 114 (0.9 mg, 0.9 μmol, 20%) LCMS (ESI+) calculated for C₅₂H₆₇N₄O₁₆⁺ (M+H⁺) 1003.45 found 1003.94.

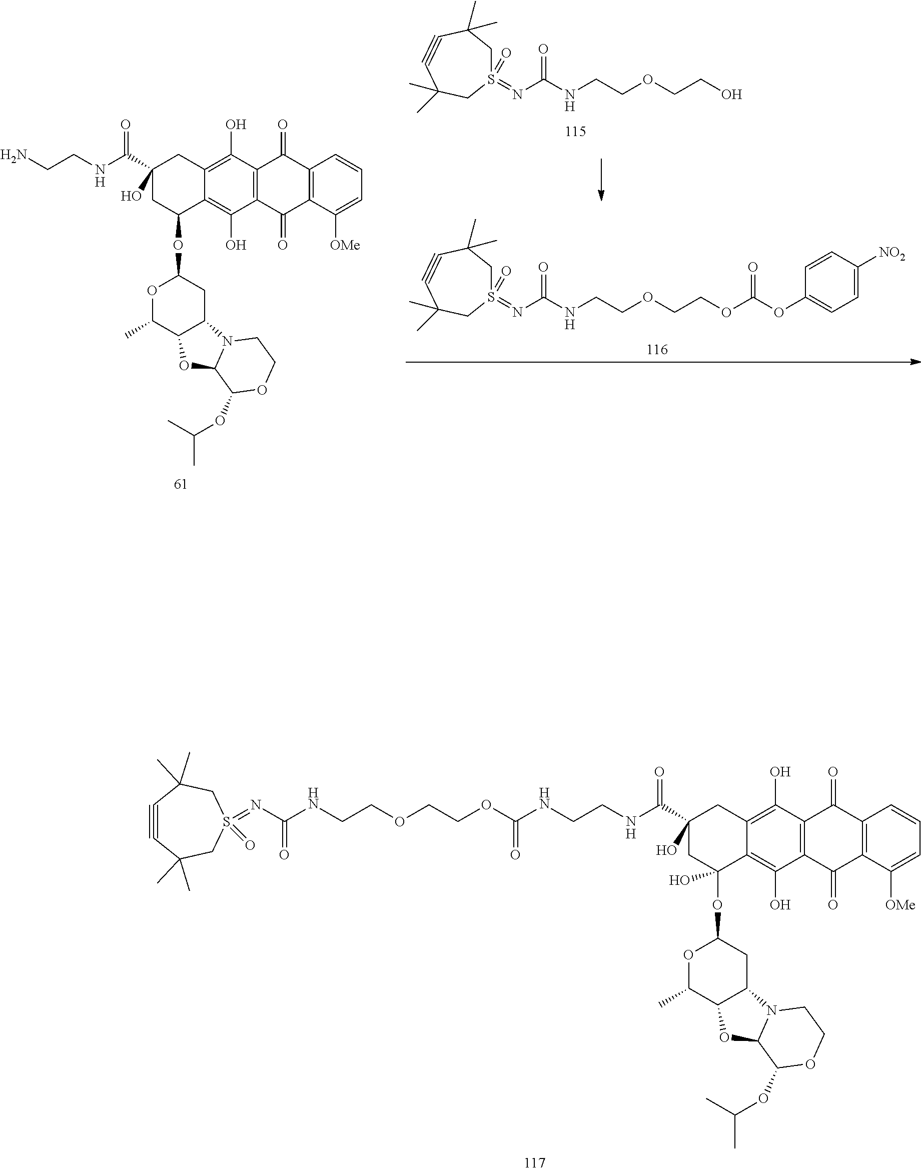 [SMILES: CC1(C)C#CC(C)(C)CS(=O)(=NC(=O)NCCOCCO)C1 ; COc1cccc2c1C(=O)c1c(O)c3c(c(O)c1C2=O)C[C@@](O)(C(=O)NCCN)C[C@@H]3O[C@H]1C[C@H]2[C@H](O[C@@H]3[C@@H](OC(C)C)OCCN32)[C@H](C)O1 ; CC1(C)C#CC(C)(C)CS(=O)(=NC(=O)NCCOCCOC(=O)Oc2ccc([N+](=O)[O-])cc2)C1 ; COc1cccc2c1C(=O)c1c(O)c3c(c(O)c1C2=O)C[C@@](O)(C(=O)NCCNC(=O)OCCOCCNC(=O)N=S1(=O)CC(C)(C)C#CC(C)(C)C1)C[C@]3(O)O[C@H]1C[C@H]2[C@H](O[C@@H]3[C@@H](OC(C)C)OCCN32)[C@H](C)O1]

### Example a128. Synthesis of Compound 115

This compound was synthesized according to a literature procedure described by Weterings et al. in Chemical Science, Volume 11, Issues 33, Pages 9011-9016.

### Example a129. Synthesis of Compound 116

To a vial containing compound 115 (168.2 mg, 1.0 Eq, 509.0 μmol) in DCM (3.00 mL) was added bis(4-nitrophenyl) carbonate (309.7 mg, 2 Eq, 1.018 mmol) and triethylamine (154.5 mg, 213 μL, 3.0 Eq, 1.527 mmol). The solution was stirred for 2 h at rt. The RM was then purified using flash column chromatography over silicagel (0→30% aceton in DCM). Fractions containing product were combined and concentrated in vacuo, affording compound 116 (262.5 mg, 77% purity 0.41 mmol, 80%) that was used without further purification in the next step. ¹H NMR (400 MHZ, DMSO-d6) δ 8.11-8.01 (m, 2H), 6.78 (m, 2H), 4.04 (t, J=4.8 Hz, 2H), 3.88 (d, J=13.9 Hz, 2H), 3.62 (d, J=13.9 Hz, 2H), 3.54 (t, J=4.8 Hz, 2H), 3.47 (m, 1H), 3.38 (m, 2H), 3.34-3.25 (m, 2H), 3.15-3.02 (m, 2H), 2.86-2.95 (m, 1H), 1.34 (s, 5H), 1.25-1.14 (m, 6H). LCMS (ESI+) calculated for C₂₂H₃₀N₃O₈S⁺ (M+H⁺) 496.17 found 496.16.

### Example a130. Synthesis of Compound 117

To vial containing compound 61 (4.5 mg, 1 Eq, 6.4 μmol) was added a solution of intermediate 116 (6.4 mg, 2 Eq, 13 μmol) in DMF (100 μL), followed by the addition of triethylamine (2.0 mg, 2.7 μL, 3 Eq, 19 μmol) and additional DMF (50 μL). The resulting solution was mixed and left at rt for circa 1 hour and was then purified by prep-HPLC (30%→95% acetonitrile in 10 mM NH₄HCO₃ in water, column Xbridge prep C₁₈, 5 μM OBD, 30×150 mm). The fractions containing product were combined and concentrated to give compound 117 (1.6 mg) as a red solid. LCMS (ESI+) calculated for C₅₁H₆₈N₅O₁₇S⁺ (M+H⁺) 1054.43 found 1054.93.

### B. Conjugation of Linker-Payloads to Antibodies

### Example b1. Conjugation of Azide-Modified Trastuzumab with Compound 9g to Obtain Conjugate Trast-9g

A bioconjugate according to the invention was prepared by conjugation of compound 9g as linker-conjugate to trastuzumab-(6-N₃-GalNAc)₂ as biomolecule. To a solution of trastuzumab-(6-N₃-GalNAc)₂ (145.6 μL, 3.0 mg, 20.6 mg/ml in TBS pH 7.4), prepared according to WO2016170186, was added TBS pH 7.4 (34.4 μL), DMF (8.8 μL) compound 9g (11.3 μL, 14.22 mM solution in DMF). The reaction was incubated at rt overnight followed by purification on a Superdex200 Increase 10/300 GL (GE Healthcare) on an AKTA Purifier-10 (GE Healthcare). Mass spectral analysis of the fabricator-digested sample showed one major product (observed mass 25742 Da, approximately 80% of total Fc/2 fragment, calculated mass 25740 Da), corresponding to the conjugated Fc/2 fragment, and one minor product (observed mass 25514 Da, approximately 20% of total Fc/2 fragment, calculated mass 25511 Da), corresponding to the conjugated Fc/2 fragment containing a fragmentation product of PNU.

### Example b2. Conjugation of Azide-Modified Trastuzumab with Compound 9d to Obtain Conjugate Trast-9d

A bioconjugate according to the invention was prepared by conjugation of compound 9d as linker-conjugate to trastuzumab-(6-N₃-GalNAc)₂ as biomolecule. To a solution of trastuzumab-(6-N₃-GalNAc)₂ (73.1 μL, 3.0 mg, 41.1 mg/ml in TBS pH 7.4), prepared according to WO2016170186, was added TBS pH 7.4 (46.9 μL), sodium deoxycholate (20 μL 110 mM), PG (40.3 μL) and compound 9d (19.7 μL, 10.14 mM solution in DMF). The reaction was incubated at rt overnight followed by purification on a Superdex200 Increase 10/300 GL (GE Healthcare) on an AKTA Purifier-10 (GE Healthcare). Mass spectral analysis of the fabricator-digested sample showed one major product (observed mass 25817 Da, approximately 80% of total Fc/2 fragment, calculated mass 25815 Da), corresponding to the conjugated Fc/2 fragment, and one minor product (observed mass 25514 Da, approximately 20% of total Fc/2 fragment, calculated mass 25511 Da), corresponding to the conjugated Fc/2 fragment containing a fragmentation product of PNU.

### Example b3. Conjugation of Azide-Modified Trastuzumab with Compound 9c to Obtain Conjugate Trast-9c

A bioconjugate according to the invention was prepared by conjugation of compound 9c as linker-conjugate to trastuzumab-(6-N₃-GalNAc)₂ as biomolecule. To a solution of trastuzumab-(6-N₃-GalNAc)₂ (145.6 μL, 3.0 mg, 20.6 mg/ml in TBS pH 7.4), prepared according to WO2016170186, was added TBS pH 7.4 (14.4 μL), DMF (8.7 μL) and compound 9c (31.3 μL, 3.83 mM solution in DMF). The reaction was incubated at rt overnight followed by purification on a Superdex200 Increase 10/300 GL (GE Healthcare) on an AKTA Purifier-10 (GE Healthcare). Mass spectral analysis of the fabricator-digested sample showed one major product (observed mass 25770 Da, approximately 80% of total Fc/2 fragment, calculated mass 25768 Da), corresponding to the conjugated Fc/2 fragment, and one minor product (observed mass 25517 Da, approximately 20% of total Fc/2 fragment, calculated mass 25511 Da), corresponding to the conjugated Fc/2 fragment containing a fragmentation product of PNU.

### Example b4. Conjugation of Azide-Modified Trastuzumab with Compound 9f to Obtain Conjugate Trast-9f

A bioconjugate according to the invention was prepared by conjugation of compound 9f as linker-conjugate to trastuzumab-(6-N₃-GalNAc)₂ as biomolecule. To a solution of trastuzumab-(6-N₃-GalNAc)₂ (728.2 μL, 15 mg, 20.6 mg/ml in TBS pH 7.4), prepared according to WO2016170186, was added TBS pH 7.4 (71.8 μL), sodium deoxycholate (71.8 μL 110 mM), DMF (51.2 μL) and compound 9f (48.8 μL, 8.2 mM solution in DMF). The reaction was incubated at rt overnight followed by purification on a Superdex200 Increase 10/300 GL (GE Healthcare) on an AKTA Purifier-10 (GE Healthcare). Mass spectral analysis of the fabricator-digested sample showed one major product (observed mass 25755 Da, approximately 80% of total Fc/2 fragment, calculated mass 25752 Da), corresponding to the conjugated Fc/2 fragment, and one minor product (observed mass 25514 Da, approximately 20% of total Fc/2 fragment, calculated mass 25509 Da), corresponding to the conjugated Fc/2 fragment containing a fragmentation product of PNU.

### Example b5. Conjugation of Azide-Modified Trastuzumab with Compound 36 to Obtain Conjugate Trast-36

A bioconjugate according to the invention was prepared by conjugation of compound 36 as linker-conjugate to trastuzumab-(6-N₃-GalNAc)₂ as biomolecule. To a solution of trastuzumab-(6-N₃-GalNAc)₂ (145.6 μL, 3 mg, 20.6 mg/ml in TBS pH 7.4), prepared according to WO2016170186, was added TBS pH 7.4 (454.4 μL), sodium deoxycholate (80 μL 110 mM) and compound 36 (120 μL, 0.64 mM solution in DMF). The reaction was incubated at rt overnight followed by purification on a Superdex200 Increase 10/300 GL (GE Healthcare) on an AKTA Purifier-10 (GE Healthcare). Mass spectral analysis of the fabricator-digested sample showed one major product (observed mass 25744 Da, approximately 60% of total Fc/2 fragment, calculated mass 25741 Da), corresponding to the conjugated Fc/2 fragment, and one minor product (observed mass 25329 Da, approximately 40% of total Fc/2 fragment, calculated mass 25327 Da), corresponding to the conjugated Fc/2 fragment containing a fragmentation product of PNU.

### Example b6. Conjugation of Azide-Modified Trastuzumab with Compound 47 to Obtain Conjugate Trast-47

A bioconjugate according to the invention was prepared by conjugation of compound 47 as linker-conjugate to trastuzumab-(6-N₃-GalNAc)₂ as biomolecule. To a solution of trastuzumab-(6-N₃-GalNAc)₂ (485.4 μL, 10.0 mg, 20.6 mg/ml in TBS pH 7.4), prepared according to WO2016170186, was added TBS pH 7.4 (124.1 μL), sodium deoxycholate (76.2 μL 110 mM) and compound 47 (76.2 μL, 3.5 mM solution in DMF). The reaction was incubated at rt overnight followed by purification on a Superdex200 Increase 10/300 GL (GE Healthcare) on an AKTA Purifier-10 (GE Healthcare). Mass spectral analysis of the fabricator-digested sample showed one major product (observed mass 25622 Da, approximately 80% of total Fc/2 fragment, calculated mass 25620 Da), corresponding to the conjugated Fc/2 fragment, and one minor product (observed mass 25382 Da, approximately 20% of total Fc/2 fragment, calculated mass 25378 Da), corresponding to the conjugated Fc/2 fragment containing a fragmentation product of PNU.

### Example b7. Conjugation of Azide-Modified Trastuzumab with Compound 53 to Obtain Conjugate Trast-53

A bioconjugate according to the invention was prepared by conjugation of compound 53 as linker-conjugate to trastuzumab-(6-N₃-GalNAc)₂ as biomolecule. To a solution of trastuzumab-(6-N₃-GalNAc)₂ (447.82 μL, 9.225 mg, 20.6 mg/ml in TBS pH 7.4), prepared according to WO2016170186, was added TBS pH 7.4 (352.2 μL), sodium deoxycholate (100 μL 110 mM) and compound 53 (100 μL, 2.46 mM solution in DMF). The reaction was incubated at rt overnight followed by purification on a Superdex200 Increase 10/300 GL (GE Healthcare) on an AKTA Purifier-10 (GE Healthcare). Mass spectral analysis of the fabricator-digested sample showed one major product (observed mass 25475 Da, approximately 90% of total Fc/2 fragment, calculated mass 25473 Da), corresponding to the conjugated Fc/2 fragment, and one minor product (observed mass 25234 Da, approximately 10% of total Fc/2 fragment, calculated mass 25230 Da), corresponding to the conjugated Fc/2 fragment containing a fragmentation product of PNU.

### Example b8. Conjugation of Azide-Modified Trastuzumab with Compound 39 to Obtain Conjugate Trast-39

A bioconjugate according to the invention was prepared by conjugation of compound 39 as linker-conjugate to trastuzumab-(6-N₃-GalNAc)₂ as biomolecule. To a solution of trastuzumab-(6-N₃-GalNAc)₂ (109.2 μL, 2.25 mg, 20.6 mg/ml in TBS pH 7.4), prepared according to WO2016170186, was added TBS pH 7.4 (370.8 μL), sodium deoxycholate (60 μL 110 mM) and compound 39 (60 μL, 1.5 mM solution in DMF). The reaction was incubated at rt overnight followed by purification on a Superdex200 Increase 10/300 GL (GE Healthcare) on an AKTA Purifier-10 (GE Healthcare). Mass spectral analysis of the fabricator-digested sample showed one major product (observed mass 25653 Da, approximately 70% of total Fc/2 fragment, calculated mass 25652 Da), corresponding to the conjugated Fc/2 fragment, and one minor product (observed mass 25240 Da, approximately 30% of total Fc/2 fragment, calculated mass 25238 Da), corresponding to the conjugated Fc/2 fragment containing a fragmentation product of PNU.

### Example b9. Transient Expression and Purification of Trast (HC-E152C,S375C)

A trastuzumab variant with heavy chain comprising E152C and S375C mutations, (identified by SEQ ID NO: 2), and non-modified light chain (identified by SEQ ID NO: 3) was transiently expressed in CHO K1 cells by Evitria (Zurich, Switzerland) at 500 mL scale. The supernatant was purified using a XK 16/20 column packed with 25 mL protein A sepharose. In a single run 500 mL supernatant was loaded onto the column followed by washing with at least 10 column volumes of 20 mM Tris pH 7.5, 150 mM NaCl. Retained protein was eluted with 0.1 M sodium acetate pH 2.7. The eluted trast (HC-E152C,S375C) was immediately neutralized with 2.5 M Tris-HCl pH 7.2 and dialyzed against PBS pH 7.4. Next the IgG was concentrated to 20.76 mg/ml using a Vivaspin Turbo 15 ultrafiltration unit (Sartorius) and stored at −80° C. prior to further use.

### Example b10. Reduction and Re-Oxidation of Trast (HC-E152C,S375C)

Trast (HC-E152C,S375C) (20 mg, 20.76 mg/mL in PBS pH 7.4) was incubated with EDTA pH 8.0 (54 μL, 200 mM in MQ) and DTT (54 μL, 200 mM in MQ) for 2 hours at 37° C. The reduced antibody was buffer exchanged to PBS pH 7.4 using a HiTrap desalting column (Cytiva, 2×5 mL columns connected in series) on an 100F NGC system (Bio-Rad). Next, the reduced antibody (17.8 mg, 6.13 mg/mL in PBS pH 7.4) was incubated with EDTA pH 8.0 (145 μL, 200 mM in MQ) and dehydroascorbic acid (119 μL, 10 mM in MQ: DMSO (9:1)) for 3 hours at room temperature. The re-oxidized antibody was buffer exchanged to PBS pH 7.4 using a HiTrap desalting column (Cytiva, 2×5 mL columns connected in series) on an 100F NGC system (Bio-Rad). LC-MS analysis of the fabricator-digested sample showed one major product for the Fc/2-fragment (observed mass 25249 Da, approximately 60% of total Fc/2 fragment), corresponding to the Fc/2 fragment with G0F-glycoform and free Cys₃₇₅, and one minor product (observed mass 25411 Da, approximately 40% of total Fc/2 fragment), corresponding to the Fc/2 fragment with G0F-glycoform and free Cys₃₇₅. LC-MS analysis of the fabricator-digested sample showed one major product for the Fab-fragment (observed mass 97639 Da, approximately 70% of total Fab-fragment), corresponding to the Fab-fragment with both Cys₁₅₂-residues in the free reduced form. Re-oxidized trast (HC-E152C,S375C) was stored at −80° C. prior to further use.

### Example b11. Conjugation of Re-Oxidized Trast (HC-E152C,S375C) with Compound 70 to Obtain Conjugate Trast (HC-E152C,S375C)-70

A bioconjugate according to the invention was prepared by conjugation of compound 70 as linker-conjugate to reduced and re-oxidized trast (HC-E152C,S375C) as biomolecule. To a solution of reduced and re-oxidized trast (HC-E152C,S375C) (60 μL, 232 μg, 3.87 mg/ml in PBS pH 7.4), prepared as described above, was added compound 70 (9.4 μL, 1 mM solution in DMF). The reaction was incubated at rt for 1 hour followed by buffer exchange using spinfiltration (Amicon Ultra-0.5, Ultracel-10 Membrane, Millipore), 3× with 400 μL PBS pH 7.4. Conjugation to Cys₃₇₅ was evaluated using a fabricator-digested sample via analysis of the Fc/2-fragment. LC-MS analysis showed two products corresponding to the conjugated Fc-2 fragment with G0F and G1F glycan (observed masses 26067 and 26231 Da, approximately 15% and 10% of total Fc/2-fragment, respectively), and two products corresponding to the conjugated Fc-2 fragment with G0F and G1F glycan and the conjugated Fc-2 fragment with subsequent hydrolysis or fragmentation of the hydrazone (observed masses 25433 and 25595 Da, approximately 30% and 20% of total Fc/2-fragment, respectively). Conjugation to Cys₁₅₂ was evaluated using a fabricator-digested sample followed by reduction via analysis of the Fd-fragment. LC-MS analysis showed one major product corresponding to the conjugated Fd-fragment (observed mass 26201 Da, approximately 60% of total Fd-fragment), and one minor product corresponding to the conjugated Fd fragment with hydrolysis or fragmentation of the hydrazone (observed mass 25565 Da, approximately 40% of total Fd-fragment).

### Example b12. Conjugation of Azide-Modified Trastuzumab with Compound 75 to Obtain Conjugate Trast-75

A bioconjugate according to the invention was prepared by conjugation of compound 75 as linker-conjugate to trastuzumab-(6-N₃-GalNAc)₂ as biomolecule. To a solution of trastuzumab-(6-N₃-GalNAc)₂ (652 μL, 15.0 mg, 23.0 mg/ml in TBS pH 7.5), prepared according to WO2016170186, was added TBS pH 7.5 (148 L), sodium deoxycholate (100 μL 110 mM) and compound 75 (100 μL, 6 mM solution in DMF). The reaction was incubated at rt overnight followed by purification on a Superdex200 Increase 10/300 GL (GE Healthcare) on an AKTA Purifier-10 (GE Healthcare). LC-MS analysis of the fabricator-digested sample showed one major product (observed mass 25726 Da, approximately 60% of total Fc/2 fragment, calculated mass 25725 Da), corresponding to the conjugated Fc/2 fragment, and one minor product (observed mass 25327 Da, approximately 20% of total Fc/2 fragment, calculated mass 25328 Da), corresponding to the conjugated Fc/2 fragment containing a fragmentation product of PNU.

### Example b13. Conjugation of Azide-Modified Trastuzumab with Compound 63a to Obtain Conjugate Trast-63a

A bioconjugate according to the invention was prepared by conjugation of compound 63a as linker-conjugate to trastuzumab-(6-N₃-GalNAc)₂ as biomolecule. To a solution of trastuzumab-(6-N₃-GalNAc)₂ (87 μL, 2.0 mg, 23.0 mg/ml in TBS pH 7.5), prepared according to WO2016170186, was added TBS pH 7.5 (73 μL), sodium deoxycholate (20 μL 110 mM) and compound 63a (20 μL, 4 mM solution in DMF). The reaction was incubated at rt overnight followed by purification on a Superdex200 Increase 10/300 GL (GE Healthcare) on an AKTA Purifier-10 (GE Healthcare). LC-MS analysis of the fabricator-digested sample showed one major product (observed mass 25636 Da, approximately 80% of total Fc/2 fragment, calculated mass 25636 Da), corresponding to the conjugated Fc/2 fragment.

### Example b14. Conjugation of Azide-Modified Palavizumab with Compound 63a to Obtain Conjugate Palav-63a

A bioconjugate according to the invention was prepared by conjugation of compound 63a as linker-conjugate to palavizumab-(6-N₃-GalNAc)₂ as biomolecule. To a solution of palavizumab-(6-N₃-GalNAc)₂ (19.6 μL, 0.5 mg, 25.6 mg/ml in TBS pH 7.5), prepared according to WO2016170186, was added TBS pH 7.5 (20.4 μL), sodium deoxycholate (5 μL 110 mM) and compound 63a (5 μL, 4 mM solution in DMF). The reaction was incubated at rt overnight. LC-MS analysis of the fabricator-digested sample showed one major product (observed mass 25636 Da, approximately 80% of total Fc/2 fragment, calculated mass 25636 Da), corresponding to the

### Example b15. Conjugation of Azide-Modified Rituximab with Compound 63a to Obtain Conjugate Rit-63a

A bioconjugate according to the invention was prepared by conjugation of compound 63a as linker-conjugate to rituximab-(6-N₃-GalNAc)₂ as biomolecule. To a solution of rituximab-(6-N₃-GalNAc)₂ (20.4 μL, 0.5 mg, 24.5 mg/ml in TBS pH 7.5), prepared according to WO2016170186, was added TBS pH 7.5 (19.6 L), sodium deoxycholate (5 μL 110 mM) and compound 63a (5 μL, 4 mM solution in DMF). The reaction was incubated at rt overnight. LC-MS analysis of the fabricator-digested sample showed one major product (observed mass 25604 Da, approximately 80% of total Fc/2 fragment, calculated mass 25604 Da), corresponding to the conjugated Fc/2 fragment.

### Example b16. Conjugation of Azide-Modified Trastuzumab with Compound 63b to Obtain Conjugate Trast-63b

A bioconjugate according to the invention was prepared by conjugation of compound 63b as linker-conjugate to trastuzumab-(6-N₃-GalNAc)₂ as biomolecule. To a solution of trastuzumab-(6-N₃-GalNAc)₂ (87 μL, 2.0 mg, 23.0 mg/ml in TBS pH 7.5), prepared according to WO2016170186, was added TBS pH 7.5 (73 μL), sodium deoxycholate (20 μL 110 mM) and compound 63b (20 μL, 4 mM solution in DMF). The reaction was incubated at rt overnight followed by purification on a Superdex200 Increase 10/300 GL (GE Healthcare) on an AKTA Purifier-10 (GE Healthcare). LC-MS analysis of the fabricator-digested sample showed one major product (observed mass 25449 Da, approximately 70% of total Fc/2 fragment, calculated mass 25449 Da), corresponding to the conjugated Fc/2 fragment.

### Example b17. Conjugation of Azide-Modified Trastuzumab with Compound 63c to Obtain Conjugate Trast-63c

A bioconjugate according to the invention was prepared by conjugation of compound 63c as linker-conjugate to trastuzumab-(6-N₃-GalNAc)₂ as biomolecule. To a solution of trastuzumab-(6-N₃-GalNAc)₂ (21.7 μL, 0.5 mg, 23.0 mg/ml in TBS pH 7.5), prepared according to WO2016170186, was added TBS pH 7.5 (5.0 μL), sodium deoxycholate (3.3 μL 110 mM) and compound 63c (3.3 μL, 4 mM solution in DMF). The reaction was incubated at rt overnight followed by purification on a Superdex200 Increase 10/300 GL (GE Healthcare) on an AKTA Purifier-10 (GE Healthcare). LC-MS analysis of the fabricator-digested sample showed one major product (observed mass 25446 Da, approximately 80% of total Fc/2 fragment, calculated mass 25447 Da), corresponding to the conjugated Fc/2 fragment.

### Example b18. Conjugation of Azide-Modified Trastuzumab with Compound 80 to Obtain Conjugate Trast-80

A bioconjugate according to the invention was prepared by conjugation of compound 80 as linker-conjugate to trastuzumab-(6-N₃-GalNAc)₂ as biomolecule. To a solution of trastuzumab-(6-N₃-GalNAc)₂ (21.7 μL, 0.5 mg, 23.0 mg/ml in TBS pH 7.5), prepared according to WO2016170186, was added TBS pH 7.5 (5.0 μL), sodium deoxycholate (3.3 μL 110 mM) and compound 80 (3.3 μL, 8 mM solution in DMF). The reaction was incubated at rt overnight. LC-MS analysis of the fabricator-digested sample showed one major product (observed mass 25656 Da, approximately 60% of total Fc/2 fragment, calculated mass 25655 Da), corresponding to the conjugated Fc/2 fragment, and one minor product (observed mass 24366 Da, approximately 25% of total Fc/2 fragment, calculated mass 24365 Da), corresponding to the remaining azido-modified Fc/2 fragment.

### Example b19. Conjugation of Azide-Modified Trastuzumab with Compound 81 to Obtain Conjugate Trast-81

A bioconjugate according to the invention was prepared by conjugation of compound 81 as linker-conjugate to trastuzumab-(6-N₃-GalNAc)₂ as biomolecule. To a solution of trastuzumab-(6-N₃-GalNAc)₂ (21.7 μL, 0.5 mg, 23.0 mg/ml in TBS pH 7.5), prepared according to WO2016170186, was added TBS pH 7.5 (5.0 μL), sodium deoxycholate (3.3 μL 110 mM) and compound 81 (3.3 μL, 8 mM solution in DMF). The reaction was incubated at rt overnight. LC-MS analysis of the fabricator-digested sample showed one major product (observed mass 25671 Da, approximately 50% of total Fc/2 fragment, calculated mass 25671 Da), corresponding to the conjugated Fc/2 fragment, and various minor products (observed masses 25256 Da, 25530 Da and 26246 Da, approximately 40% of total Fc/2 fragment).

### Example b20. Conjugation of Azide-Modified Trastuzumab with Compound 108 to Obtain Conjugate Trast-108

A bioconjugate according to the invention was prepared by conjugation of compound 108 as linker-conjugate to trastuzumab-(6-N₃-GalNAc)₂ as biomolecule. To a solution of trastuzumab-(6-N₃-GalNAc)₂ (21.7 μL, 0.5 mg, 23.0 mg/ml in TBS pH 7.5), prepared according to WO2016170186, was added TBS pH 7.5 (5.0 μL), sodium deoxycholate (3.3 μL 110 mM) and compound 108 (3.3 μL, 6 mM solution in DMF). The reaction was incubated at rt for 4 hours. LC-MS analysis of the fabricator-digested sample showed one major product (observed mass 25664 Da, approximately 80% of total Fc/2 fragment, calculated mass 25664 Da), corresponding to the conjugated Fc/2 fragment.

### Example b21. Conjugation of Azide-Modified Trastuzumab with Compound 117 to Obtain Conjugate Trast-117

A bioconjugate according to the invention was prepared by conjugation of compound 117 as linker-conjugate to trastuzumab-(6-N₃-GalNAc)₂ as biomolecule. To a solution of trastuzumab-(6-N₃-GalNAc)₂ (21.7 μL, 0.5 mg, 23.0 mg/ml in TBS pH 7.5), prepared according to WO2016170186, was added TBS pH 7.5 (5.0 μL), sodium deoxycholate (3.3 μL 110 mM) and compound 117 (3.3 μL, 3 mM solution in DMF). The reaction was incubated at rt for 5 minutes. LC-MS analysis of the fabricator-digested sample showed one major product (observed mass 25491 Da, approximately 80% of total Fc/2 fragment, calculated mass 25419 Da), corresponding to the

### Example b22. Conjugation of Azide-Modified Trastuzumab with Compound 86 to Obtain Conjugate Trast-86

A bioconjugate according to the invention was prepared by conjugation of compound 86 as linker-conjugate to trastuzumab-(6-N₃-GalNAc)₂ as biomolecule. To a solution of trastuzumab-(6-N₃-GalNAc)₂ (21.7 μL, 0.5 mg, 23.0 mg/ml in TBS pH 7.5), prepared according to WO2016170186, was added TBS pH 7.5 (8.3 μL), sodium deoxycholate (5 μL 110 mM) and compound 86 (15 μL, 2.7 mM solution in PG). The reaction was incubated at rt for 4 hours. LC-MS analysis of the fabricator-digested sample showed one major product (observed mass 25698 Da, approximately 50% of total Fc/2 fragment, calculated mass 25699 Da), corresponding to the conjugated Fc/2 fragment, and one major product (observed mass 25255 Da, approximately 50% of total Fc/2 fragment), corresponding to the conjugated Fc/2 fragment with fragmentation of PNU.

### Example b23. Conjugation of Azide-Modified Trastuzumab with Compound 63d to Obtain Conjugate Trast-63d

A bioconjugate according to the invention was prepared by conjugation of compound 63d as linker-conjugate to trastuzumab-(6-N₃-GalNAc)₂ as biomolecule. To a solution of trastuzumab-(6-N₃-GalNAc)₂ (21.7 μL, 0.5 mg, 23.0 mg/ml in TBS pH 7.5), prepared according to WO2016170186, was added TBS pH 7.5 (18.3 L), sodium deoxycholate (5.0 μL 110 mM) and compound 63d (5 μL, 8 mM solution in DMF). The reaction was incubated at rt for 4 hours. Mass spectral analysis of the fabricator-digested sample showed one major product (observed mass 25577 Da, approximately 70% of total Fc/2 fragment, calculated mass 25578 Da), corresponding to the conjugated Fc/2 fragment.

### Example b24. Conjugation of Azide-Modified Trastuzumab with Compound 114 to Obtain Conjugate Trast-114

A bioconjugate according to the invention was prepared by conjugation of compound 114 as linker-conjugate to trastuzumab-(6-N₃-GalNAc)₂ as biomolecule. To a solution of trastuzumab-(6-N₃-GalNAc)₂ (21.7 μL, 0.5 mg, 23.0 mg/ml in TBS pH 7.5), prepared according to WO2016170186, was added TBS pH 7.5 (8.3 μL), sodium deoxycholate (5.0 μL 110 mM) and compound 114 (15.0 μL, 2.7 mM solution in PG). The reaction was incubated at rt for 4 hours. Mass spectral analysis of the fabricator-digested sample showed one major product (observed mass 25367 Da, approximately 40% of total Fc/2 fragment, calculated mass 25368 Da), corresponding to the conjugated Fc/2 fragment, and one major products (observed mass 25112 Da, approximately 40% of total Fc/2 fragment), corresponding to the conjugated Fc/2 fragment with fragmentation of PNU.

### Example b25. Conjugation of Azide-Modified Trastuzumab with Compound 92 to Obtain Conjugate Trast-92

A bioconjugate according to the invention was prepared by conjugation of compound 92 as linker-conjugate to trastuzumab-(6-N₃-GalNAc)₂ as biomolecule. To a solution of trastuzumab-(6-N₃-GalNAc)₂ (21.7 μL, 0.5 mg, 23.0 mg/ml in TBS pH 7.5), prepared according to WO2016170186, was added TBS pH 7.5 (8.3 μL), sodium deoxycholate (5.0 μL 110 mM) and compound 92 (15 μL, 2.7 mM solution in PG). The reaction was incubated at rt for 4 hours. Mass spectral analysis of the fabricator-digested sample showed one major products (observed mass 25529 Da, approximately 50% of total Fc/2 fragment, calculated mass 25530 Da), corresponding to the conjugated Fc/2 fragment, and one major products (observed mass 25114 Da, approximately 50% of total Fc/2 fragment, calculated mass 25118 Da), corresponding to the conjugated Fc/2 fragment with fragmentation of PNU.

### Example b26 MV0323. Conjugation of Azide-Modified Trastuzumab with Compound 92 to Obtain Conjugate Trast-92

A bioconjugate according to the invention was prepared by conjugation of compound 92 as linker-conjugate to trastuzumab-(6-N₃-GalNAc)₂ as biomolecule. To a solution of trastuzumab-(6-N₃-GalNAc)₂ (21.7 μL, 0.5 mg, 23.0 mg/ml in TBS pH 7.5), prepared according to WO2016170186, was added TBS pH 7.5 (8.3 μL), sodium deoxycholate (5.0 μL 110 mM) and compound 92 (15 UL, 0.9 mM solution in PG). The reaction was incubated at rt overnight. Mass spectral analysis of the fabricator-digested sample showed one minor product (observed mass 25482 Da, approximately 25% of total Fc/2 fragment, calculated mass 25484 Da), corresponding to the conjugated Fc/2 fragment, and two other products (observed masses 24723 Da and 25067 Da, approximately 50% and 25% of total Fc/2 fragment, respectively), corresponding to fragmentation products of the conjugated Fc/2 fragment.

C. In vitro evaluation

### Example c1. In Vitro Studies Compounds 6a, 6b, 6c, 6d and 6e

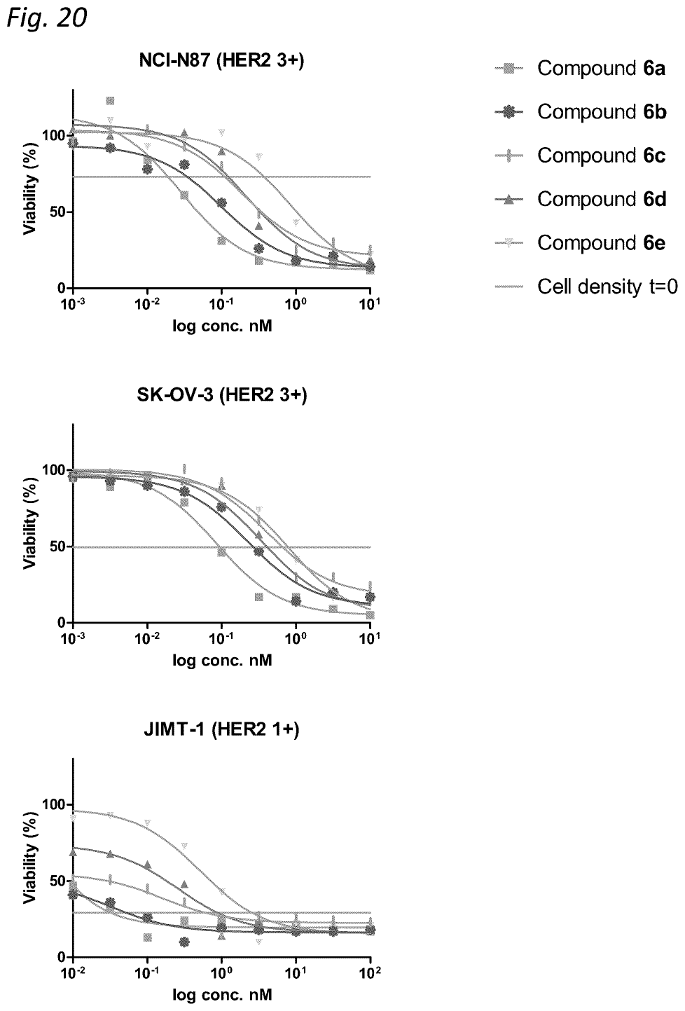

SK-OV-3, NCI-N87, JIMT-1 and MDA-MB-231 cells were plated in 96-well plates (5000 cells/well) in RPMI-1640 GlutaMAX (Invitrogen) supplemented with 10% fetal bovine serum (FBS) (Invitrogen, 150 μL/well) and incubated overnight in a humidified atmosphere at 37° C. and 5% CO2. Compounds 6a, 6b, 6c, 6d and 6e were added in triplo in a square root of 10-fold dilution series to obtain a final concentration ranging from 1 pM to 10 nM (for SK-OV-3 and NCI-N87) or 10 pM to 100 nM (for JIMT-1 and MDA-MB-231) and a final volume of 200 μL/well. The cells were incubated for 3 days in a humidified atmosphere at 37° C. and 5% CO2. The culture medium was replaced by 0.01 mg/mL resazurin (Sigma Aldrich) in RPMI-1640 GlutaMAX supplemented with 10% FBS (200 μL/well). After 4 hours in a humidified atmosphere at 37° C. and 5% CO₂ the fluorescence was detected with a fluorescence plate reader (Infinite® M1000 Tecan) at 560 nm excitation and 590 nm emission. The relative fluorescent units (RFU) were normalized to cell viability percentage by setting wells without cells at 0% viability and wells with untreated cells at 100% viability. Viability was plotted using Graphpad prism software (see FIG. 20). IC₅₀ values calculated by non-linear regression using Graphpad prism software and are shown in the table below. For compound 6a and 6b no accurate IC50 value could be calculated in JIMT-1 and MDA-MB-231 cells since no plateau was reached at the lowest tested concentration.

TABLE 1
IC₅₀ values of free payloads in various HER2
positive and HER2 negative cell lines.

| | | | | | MDA-MB- |
|---|---|---|---|---|---|
| | SK-OV-3 | | NCI-N87 | JIMT-1 | 231 |
| | (HER2 3+) | | (HER2 3+) | (HER2 1+) | (HER2−) |
| Compound 6a | 28 | pM | 85 pM | No IC₅₀ | No IC₅₀ |
| Compound 6b | 99 | pM | 224 pM | No IC₅₀ | No IC₅₀ |
| Compound 6c | 187 | pM | 416 pM | 159 pM | 174 pM |
| Compound 6d | 217 | pM | 339 pM | 259 pM | 169 pM |
| Compound 6e | 845 | pM | 795 pM | 520 pM | 705 pM |

### Example c2. In Vitro Studies with Trast-9g, Trast-9d, Trast-9c and Trast-36

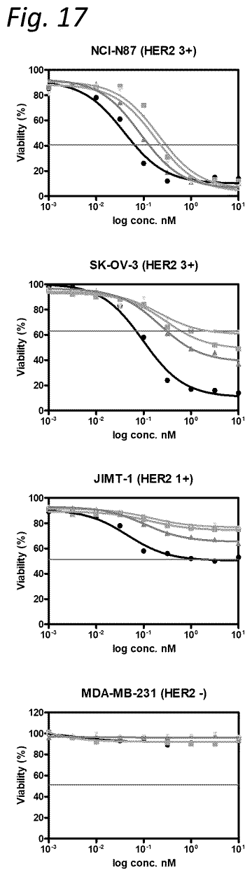

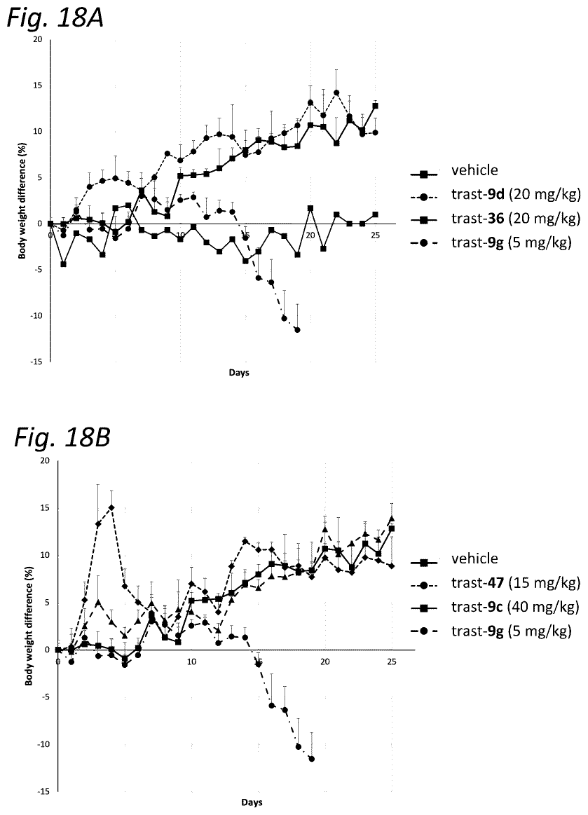

SK-OV-3 (Her2 3+), NCI-N87 (Her2 3+), JIMT-1 (Her2 1+) and MDA-MB-231 (Her2−) cells were plated in 96-well plates (5000 cells/well) in RPMI-1640 GlutaMAX (Invitrogen) supplemented with 10% fetal bovine serum (FBS) (Invitrogen, 150 μL/well) and incubated overnight in a humidified atmosphere at 37° C. and 5% CO2. ADCs were added in triplo in a square root of 10-fold dilution series to obtain a final concentration ranging from 1 pM to 10 nM and a final volume of 200 L/well. The cells were incubated for 5 days in a humidified atmosphere at 37° C. and 5% CO2. The culture medium was replaced by 0.01 mg/mL resazurin (Sigma Aldrich) in RPMI-1640 GlutaMAX supplemented with 10% FBS (200 μL/well). After 4 hours in a humidified atmosphere at 37° C. and 5% CO₂ the fluorescence was detected with a fluorescence plate reader (Infinite® M1000 Tecan) at 560 nm excitation and 590 nm emission. The relative fluorescent units (RFU) were normalized to cell viability percentage by setting wells without cells at 0% viability and wells with untreated cells at 100% viability. Viability was plotted using Graphpad prism software (see FIG. 17). IC50 values for ADCs on SKOV-3, N87 and JIMT-1 were calculated by non-linear regression using Graphpad prism software and are shown in the table below.

TABLE 2
IC₅₀ values of ADCs in various HER2 positive
and HER2 negative cell lines.

| | SK-OV-3 | NCI-N87 | JIMT-1 | MDA-MB-231 |
|---|---|---|---|---|
| | (HER2 3+) | (HER2 3+) | (HER2 1+) | (HER2−) |
| trast-9g | 0.097 nM | 0.039 nM | 0.046 nM | No IC₅₀ |
| trast-9d | 0.287 nM | 0.207 nM | 0.133 nM | |
| trast-9c | 0.211 nM | 0.179 nM | 0.145 nM | |
| trast-36 | 0.222 nM | 0.086 nM | 0.015 nM | |

### Example c3. In Vitro Studies with Trast-9g, Trast-63a, Trast-63b and Trast-75

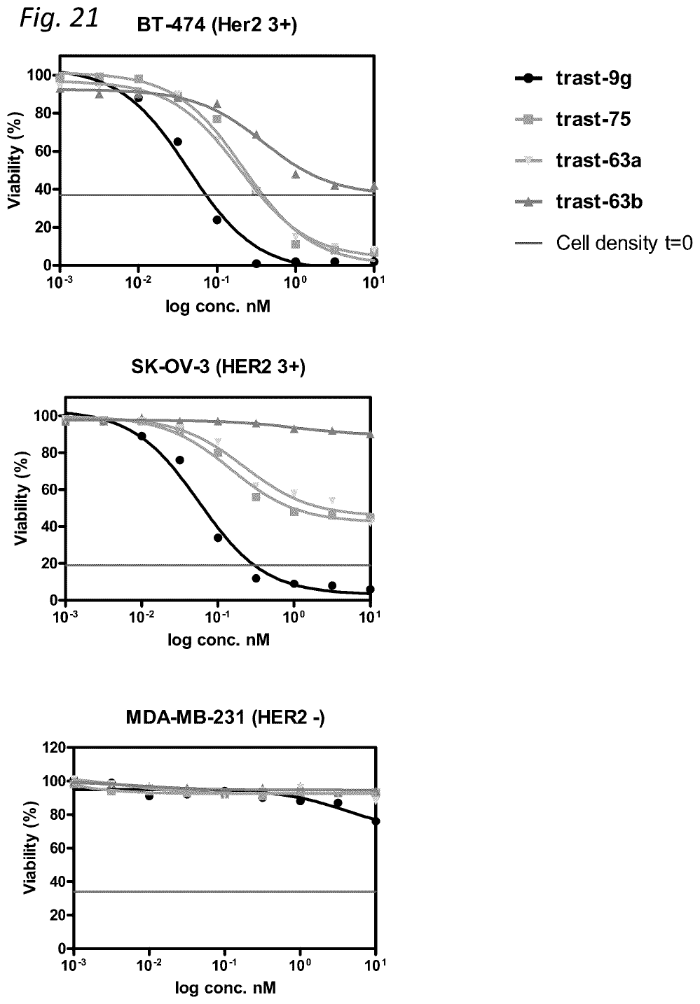

BT-474 (Her2 3+), SK-OV-3 (Her2 3+) and MDA-MB-231 (Her2-) cells were plated in 96-well plates (5000 cells/well) in RPMI-1640 GlutaMAX (Invitrogen) supplemented with 10% fetal bovine serum (FBS) (Invitrogen, 150 μL/well) and incubated overnight in a humidified atmosphere at 37° C. and 5% CO2. ADCs were added in triplo in a square root of 10-fold dilution series to obtain a final concentration ranging from 1 pM to 10 nM and a final volume of 200 L/well. The cells were incubated for 5 days in a humidified atmosphere at 37° C. and 5% CO2. The culture medium was replaced by 0.01 mg/mL resazurin (Sigma Aldrich) in RPMI-1640 GlutaMAX supplemented with 10% FBS (200 μL/well). After 4 hours in a humidified atmosphere at 37° C. and 5% CO2 the fluorescence was detected with a fluorescence plate reader (Infinite® M1000 Tecan) at 560 nm excitation and 590 nm emission. The relative fluorescent units (RFU) were normalized to cell viability percentage by setting wells without cells at 0% viability and wells with untreated cells at 100% viability. Viability was plotted using Graphpad prism software (see FIG. 21). IC50 values for ADCs on SKOV-3, N87 and JIMT-1 were calculated by non-linear regression using Graphpad prism software and are shown in the table below. For trast-6b no accurate IC50 value could be calculated in SK-OV3 cells since not more than 50% of the cells died at the highest tested concentration.

TABLE 3
IC₅₀ values of ADCs in various HER2 positive
and HER2 negative cell lines.

| | BT-474 | SK-OV-3 | MDA-MB-231 |
|---|---|---|---|
| | (HER2 3+) | (HER2 3+) | (HER2−) |
| trast-9g | 43 pM | 57 pM | No IC₅₀ |
| trast-75 | 219 pM | 149 pM | |
| trast-63a | 196 pM | 224 pM | |
| trast-63b | 390 pM | No IC₅₀ | |

### D. In Vitro Evaluation

### Example d1. In Vivo Tolerability in CD-1 Mice

Naïve female CD-1 mice (n=3), obtained from Beijing Vital River Laboratory Animal Technology Co, were treated with vehicle or a single dose of antibody-drug conjugate trast-9g (5 mg/kg), trast-9d (at 20 mg/kg), trast-36 (20 mg/kg), trast-47 (20 mg/kg) or trast-9c (40 mg/kg). After dosing, animals were checked daily for morbidity and mortality for 25 days. During routine monitoring, the animals were checked for any effects of treatments on behavior such as mobility, food and water consumption, body weight gain/loss, eye/hair matting and any other abnormalities. Mortality and observed clinical signs were recorded for individual animals in detail (depicted in (FIGS. 18A and 18B).

### Example d2. In Vivo Efficacy in JIMT-1 CDX Model

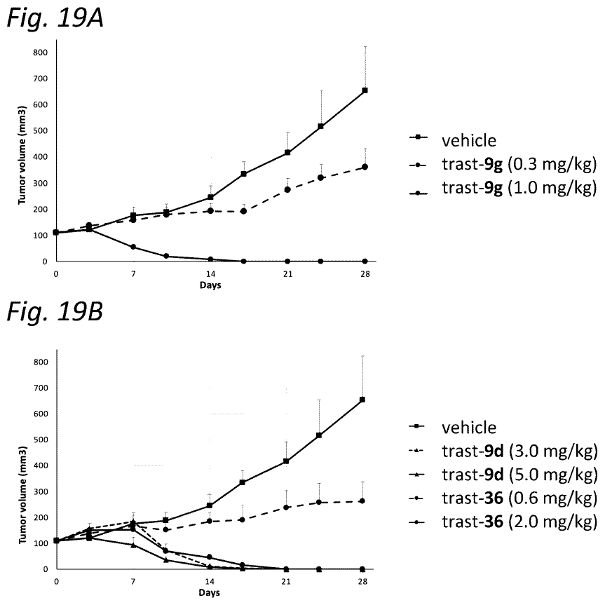

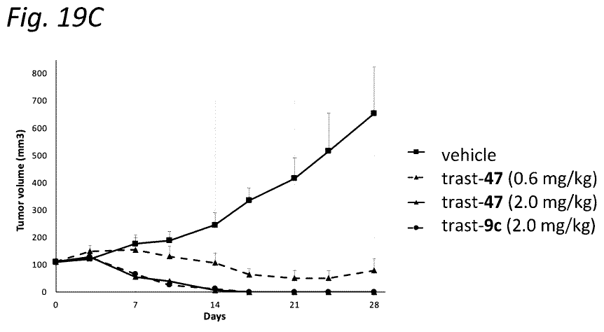

Female NOD/SCID mice (5- to 8-week-old at study initiation, obtained from GemPharmatech Co. Ltd., China) were inoculated subcutaneously it the right front flank region with 5×10⁶ JIMT-1 human breast cancer cells in 0.1 ml of PBS for tumor development. When the tumor volume was in the range of 100 to 150 mm³, groups of five mice were injected i.v. with either vehicle, trast-9g (at 0.3 or 1 mg/kg), trast-9d (at 3.0 or 5.0 mg/kg), trast-36 (at 0.6 or 2.0 mg/kg), trast-47 (at 0.6 or 2.0 mg/kg) and trast-9c (at 2.0 mg/kg). In all cases a single dose was administered on day 0. Tumor volume and body weight was measured twice per week after randomization (FIGS. 19A, 19B and 19C).

### Sequence List

Sequence identification of GGFG cleavable linker
(SEQ. ID NO: 1):
GGFG
Sequence identification of trastuzumab(HC-E152C,
S375C) heavy chain (SEQ. ID NO: 2):
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPCPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPCDIAVEWESNGQPENNYKTTPPV
LDSDGSFFLYSKLTVDKSRWQQGNVFSCSVMHEALHNHYTQKSLSLSPGK
Sequence identification of trastuzumab(HC-E152C,
S375C) light chain (SEQ. ID NO: 3):
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

What is claimed is:

1. A conjugate, comprising a compound according to structure (1) conjugated to a cell-binding agent via a linker, wherein structure (1) is as follows: 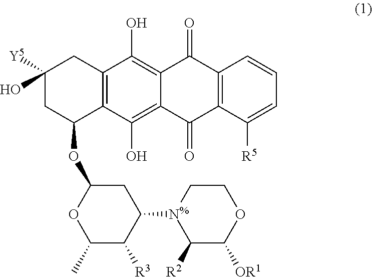
wherein:
R¹ is optionally substituted Et, i-Pr, n-Pr, t-Bu, i-Bu, n-Bu, n-pentyl, cyclopropyl, cyclobutyl, cyclopentyl, C₆₋₁₅ alkyl, C₂₋₁₅ alkenyl, C₂₋₁₅ alkynyl, heterocyclyl, (hetero)aryl, Sp-(hetero)aryl, Sp-heterocyclyl, Sp—X²R⁴, Sp—N₃, Sp—X²—Sp—R¹², or Sp—N(R⁴)₂, wherein the optional substituent is selected from a halogen, C₁₋₁₂ (hetero)alkyl, (hetero)aryl, C₂₋₁₅ alkenyl, C₂₋₁₅ alkynyl, X²R⁴, N(R⁴)₂, or NO₂, and wherein substituents C₁₋₁₂ (hetero)alkyl and (hetero)aryl may optionally be further substituted with C₁₋₆ (hetero)alkyl, X²R⁴ and N(R⁴)₂; wherein each Sp is individually C₁₋₁₂ (hetero)alkylene, (hetero)arylene, C₁₋₁₂ (hetero)alkylene-(hetero)arylene, or (hetero)arylene-C₁₋₁₂ (hetero)alkylene, wherein the (hetero)alkylene or the (hetero)arylene is optionally substituted with one or more substituents selected from a halogen, X²R⁴, N(R⁴)₂, C₁₋₄ alkyl, and NO₂, wherein each R⁴ is individually H, C₁₋₄ alkyl, or adamantly, and X² is C(O), C(O)O, C(O)NH, O, S, S(O), S(O)₂, S(O)NH, or S(O)₂NH, and wherein R¹² is β-glucuronide acid, PO₃⁽²⁻⁾, OPO₃⁽²⁻⁾, CO₂⁽⁻⁾, SO₃⁽⁻⁾, or N(C₁₋₄ alkyl)₃⁽⁺⁾;
R² is H, S(O)₂OH, or P(O)₂OH, and R³ is OH, or R² and R³ are fused together via an ether moiety to form an oxazolidine ring;
R⁵ is H or OCH₃;
N % is Nor N→O;
Y⁵ is CH₂—Y, C(O)—Y, C(═N(R²⁰))—Y, or C(R⁹)═N—Y, wherein R⁹ is selected from C₁₋₄ alkyl optionally substituted with an OH or O(CO)C₁₋₆ alkyl group, and R²⁰ is NR⁴—C(O)—N(R⁴)₂, NR⁴—C(O)—Sp—N(R⁴)₂, NR⁴—C(O)—R¹², or NR⁴—C(O)—Sp—R¹², wherein Sp, R⁴, and R¹² are as defined above;
the compound is connected to the linker via Y,
or a salt thereof, wherein each ion if present is balanced with one or more pharmaceutically acceptable counter-ions.

2. The conjugate according to claim 1, having the structure (2) 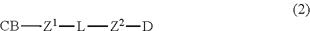
wherein:
CB is the cell-binding agent;
D is the compound according to structure (1);
L is a linker;
Z¹ is a connecting group that connects the cell-binding agent CB to the linker; and
Z² is a connecting group that connects the compound D to the linker.

3. The conjugate according to claim 2, wherein the connecting group Z¹ is formed by a conjugation reaction selected from amide bond formation, carbamate bond formation, thiol alkylation, thiol arylation, and cycloaddition reaction.

4. The conjugate according to claim 2, wherein the connecting group Z¹ is connected to the cell-binding agent CB via a lysine residue of CB, a glutamine residue of CB, a threonine residue of CB, a cysteine residue of CB, a tyrosine residue of CB, or a glycan of CB.

5. The conjugate according to claim 2, wherein the connecting group Z² is an amide moiety, an ester moiety, a thioether moiety, an ether moiety, a carbamate moiety, a [2.2.2]bicyclic structure, a [2.2.1]bicyclic structure, a disulfide, a carbonate moiety, or a (hetero)aryl moiety.

6. The conjugate according to claim 2, wherein L-Z² has the structure: 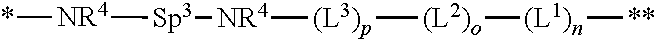
wherein:
the bond labelled with * is connected to the C(O) moiety adjacent to Y of the compound according to structure (1);
the bond labelled with ** is connected to connecting group Z¹;
—Sp³ is a is C₁₋₁₂ (hetero)alkylene, (hetero)arylene, C₁₋₁₂ alkylene-(hetero)arylene, or (hetero)arylene-C₁₋₁₂ alkylene, wherein the alkylene or the (hetero)arylene may be optionally substituted with one or more substituents selected from a halogen, X²R⁴, N(R⁴)₂, C₁₋₄ alkyl, or NO₂, wherein the C₁₋₄ alkyl substituent may optionally form a cyclic structure by being joined with an NR⁴ moiety, and the alkylene may optionally be interrupted with one or more heteroatoms selected from X² and NR⁴;
R⁴ and X² are as defined in claim 1;
L¹, L², and L³ are each individually linkers that together link Z¹ to D;
n, o, and p are each individually 0 or 1, provided that n+o+p=1, 2, or 3.

7. The conjugate according to claim 1, wherein the cell-binding agent is an antibody, a peptide, a small molecule, or an aptamer.

8. The conjugate according to claim 1, wherein R¹ is selected from Et, i-Pr, t-Bu, Bz, Bn, Sp—N₃, or Sp—NH₂, wherein Sp is C₁₋₄ alkylene or C₁₋₄ alkylene-arylene.

9. The conjugate according to claim 1, wherein R² and R³ are joined together via an ether moiety to form an oxazolidine ring.

10. The conjugate according to claim 1, wherein R¹ is not CH₂CH₂SH, unsubstituted ethyl, or benzyl.

11. A compound according to structure (1): 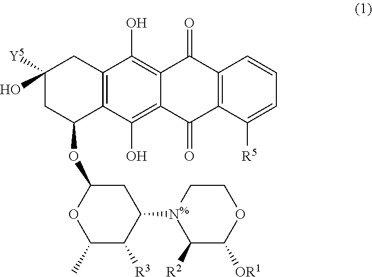
wherein:
R¹ is optionally substituted Et, i-Pr, n-Pr, t-Bu, i-Bu, n-Bu, n-pentyl, cyclopropyl, cyclobutyl, cyclopentyl, C₆₋₁₅ alkyl, C₂₋₁₅ alkenyl, C₂₋₁₅ alkynyl, heterocyclyl, (hetero)aryl, Sp-(hetero)aryl, Sp-heterocyclyl, Sp—X²R⁴, Sp—N₃, Sp—X²—Sp—R¹², or Sp—N(R⁴)₂, wherein the optional substituent is selected from a halogen, C₁₋₁₂ (hetero)alkyl, (hetero)aryl, C₂₋₁₅ alkenyl, C₂₋₁₅ alkynyl, X²R⁴, N(R⁴)₂, or NO₂, and wherein substituents C₁₋₁₂ (hetero)alkyl and (hetero)aryl may optionally be further substituted with C₁₋₆ (hetero)alkyl, X²R⁴, and N(R⁴)₂; wherein each Sp is individually C₁₋₁₂ (hetero)alkylene, (hetero)arylene, C₁₋₁₂ (hetero)alkylene-(hetero)arylene, or (hetero)arylene-C₁₋₁₂ (hetero)alkylene, wherein the (hetero)alkylene or the (hetero)arylene is optionally substituted with one or more substituents selected from halogen, X²R⁴, N(R⁴)₂, C₁₋₄ alkyl, and NO₂, wherein each R⁴ is individually H, C₁₋₄ alkyl, or adamantly, and X² is C(O), C(O)O, C(O)NH, O, S, S(O), S(O)₂, S(O)NH, or S(O)₂NH, and wherein R¹² is β-glucuronide acid, PO₃⁽²⁻⁾, OPO₃⁽²⁻⁾, CO₂⁽⁻⁾, SO₃⁽⁻⁾ or N(C₁₋₄ alkyl)₃⁽⁺⁾;
R² is H, S(O)₂OH, or P(O)₂OH, and R³ is OH, or R² and R³ are fused together via an ether moiety to form an oxazolidine ring;
R⁵ is H or OCH₃;
N % is N or N→O;
Y⁵ is CH₂—Y, C(O)—Y, C(═N(R²⁰))—Y, C(R⁹)═N—Y, or C(R⁹)═N(R²⁰), wherein R⁹ is selected from C₁₋₄ alkyl optionally substituted with an OH or O(CO)C₁₋₆ alkyl group, and R²⁰ is NR⁴—C(O)—N(R⁴)₂, NR⁴—C(O)—Sp—N(R⁴)₂, NR⁴—C(O)—R¹², or NR⁴—C(O)—Sp—R¹², wherein Sp, R⁴, and R¹² are as defined above;
Y is NR⁴—Sp³—N(R⁴)₂, NR⁴—Sp³—X²(R⁴), N(R⁴)₂, CH₃, R¹², Sp³R¹², NR⁴—Sp³—X²—Sp³—R¹², OH, or CH₂OH, wherein each Sp³ is a spacer; and
R¹ is not unsubstituted ethyl, CH₂CH₂SH, or benzyl when Y⁵ is C(O)—CH₂OH;
or a salt thereof, wherein each ion if present is balanced with one or more pharmaceutically acceptable counter-ions.

12. The compound according to claim 11, wherein:
R¹ is selected from i-Pr, t-Bu, Bn, Sp—N₃, or Sp—NH₂; and
Sp is C₁₋₄ alkylene or C₁₋₄ alkylene-arylene.

13. The compound according to claim 11, wherein R² and R³ are joined together via an ether moiety to form an oxazolidine ring.

14. The compound according to claim 11, wherein Y is CH₂OH.

15. The compound according to claim 11, wherein N^{%} is N.

16. A conjugate, wherein a compound according to claim 15 is conjugated to a cell-binding agent via a linker.

17. The conjugate according to claim 16, wherein the compound according to structure (1) is conjugated to the cell-binding agent through:
(i) R¹; or
(ii) Y.

18. The conjugate according to claim 16, having structure (2) as defined in claim 2, wherein L-Z² has a structure selected from (L1)-(L4): 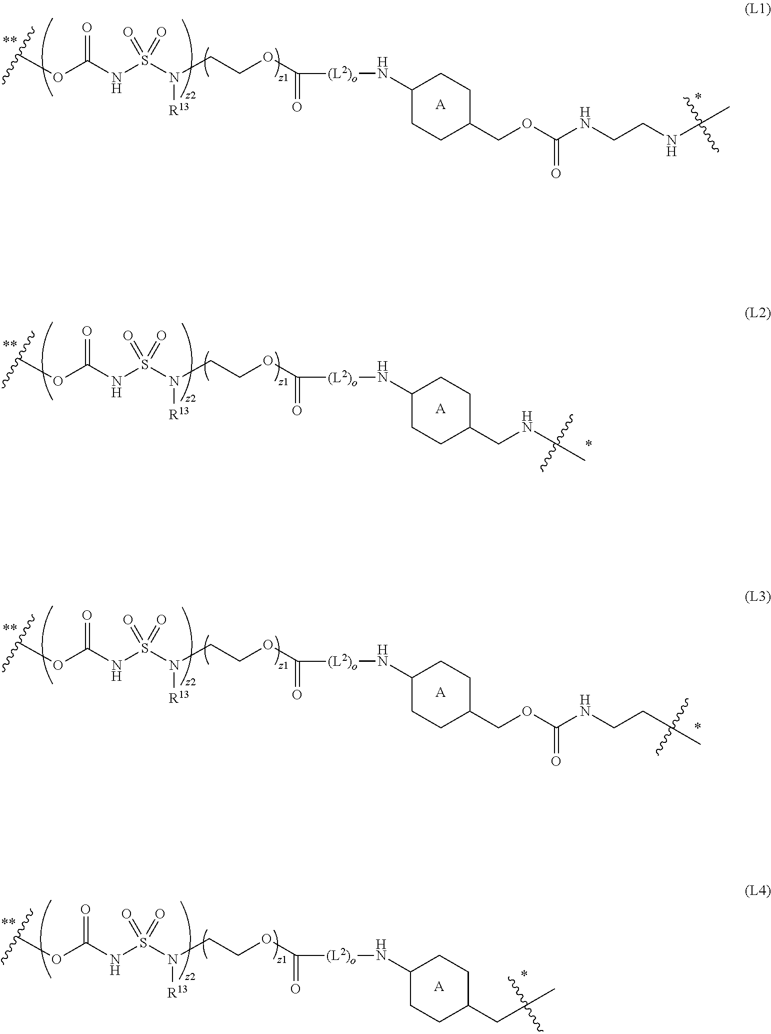
wherein:
the bond labelled with * is connected:
(a) for (L1) and (L2) to the C(O) moiety adjacent to Y of the compound according to structure (1), and
(b) for (L3) and (L4) to the O atom of the OR¹ moiety of the compound according to structure (1);
the bond labelled with ** is connected to the cell-binding agent;
R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups, and C₃-C₂₄ (hetero)arylalkyl groups, wherein the C₁-C₂₄ alkyl groups, C₃-C₂₄ cycloalkyl groups, C₂-C₂₄ (hetero)aryl groups, C₃-C₂₄ alkyl(hetero)aryl groups, and C₃-C₂₄ (hetero)arylalkyl groups are optionally substituted and optionally interrupted by one or more heteroatoms selected from O, S, and NR¹⁴, wherein R¹⁴ is independently selected from the group consisting of hydrogen and C₁-C₄ alkyl groups, or R¹³ is D connected to N optionally via a spacer moiety, or R¹³ is connected to elsewhere in the linker, optionally via a spacer moiety, to form a cyclic structure;
L² is a dipeptide, a tripeptide, or a tetrapeptide;
is 0 or 1;
ring A is an optionally substituted 5- or 6-membered aromatic or heteroaromatic ring;
z1 is an integer in the range of 1-4; and
z2 is 0 or 1.

19. A pharmaceutical composition comprising:
the conjugate according to claim 1; and
a pharmaceutically acceptable carrier.

20. A method for targeting a tumour cell expressing a specific extracellular receptor, the method comprising:
contacting the conjugate according to claim 1 with cells that may possibly express the extracellular receptor, wherein the antibody specifically targets the extracellular receptor.

21. A method for treating cancer, the method comprising:
administering to a subject in need thereof the conjugate according to claim 1, wherein the cancer cells specifically express an extracellular receptor.

22. The method according to claim 20, wherein the extracellular receptor is selected from the group consisting of 5T4, ADAM-9, AMHRII, ASCT2, ASLG659, +ASPHD1, av-integrin, Ax1, B7-H3, B7-H4, BAFF-R, BCMA, BMPR1B, Brevican, c-KIT, c-Met, C4.4a, CA-IX, cadherin-6, CanAg, CD123, CD13, CD133, CD138/syndecan-1, CD166, CD19, CD20, CD203c, CD205, CD21, CD22, CD228, CD25, CD30, CD324, CD33, CD37, CD38, CD45, CD46, CD48a, CD56, CD70, CD71, CD72, CD74, CD79a, CD79b, CEACAM5, claudin-18.2, claudin-6, CLEC12A, CLL-1, Cripto, CRIPTO, CS1, CXCR5, DLK-1, DLL3, DPEP3, E16, EGFR, ENPP3, EpCAM, EphA2, EphB2R, ETBR, FAP, FcRH1, FcRH2, FcRH5, FGFR2, fibronectin, FLT3, folate receptor alpha, Gal-3BP, GD3, GDNF-Ra1, GEDA, GFRA1, Globo H, gpNMB, GPR172A, GPR19, GPR54, guanyl cyclase C, HER2, HER3, HLA-DOB, IGF-1R, IL13R, IL20Ra, Lewis Y, LGR5, LIV-1, LRRC15, LY64, Ly6E, Ly6G6D, LY6K, MDP, MFI2, MICA/B, MOSPD2, MPF, MSG783, MUC1, MUC16, NaPi2b, NCA, nectin-4, Notch3, P-cadherin, P2X5, PD-L1, PMEL17, PRLR, PSCA, PSCA hlg, PSMA, PTK7, RET, RNF43, RON, ROR1, ROR2, Sema 5b, SLITRK6, SSTR2, STEAP1, STEAP2, TAG72, TENB2, TF, TIM-1, TM4SF, TMEFF, TMEM118, TMEM46, transferrin, TROP-2, TrpM4, TWEAKR, receptor tyrosine kinases (RTK), and tenascin.


SEQUENCE LISTING (10 sequences)

SEQ ID NO: 1 - protein, 4 aa, synthetic construct
GGFG

SEQ ID NO: 2 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 4 aa, synthetic construct
GGGG

SEQ ID NO: 5 - protein, 4 aa, synthetic construct
GFGG

SEQ ID NO: 6 - protein, 4 aa, Synthetic construct
FGGG

SEQ ID NO: 7 - protein, 4 aa, Synthetic construct
GGGF

SEQ ID NO: 8 - protein, 4 aa, Synthetic construct
FFGG

SEQ ID NO: 9 - protein, 4 aa, Synthetic construct
GGFF

SEQ ID NO: 10 - protein, 5 aa, Synthetic construct
GGGFG